SEQUENCE LISTING (91 sequences)

SEQ ID NO: 1 - DNA, 3182 bp, Hepatitis B virus
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 2 - DNA, 13 bp, Artificial Sequence
  contiguous nucleotide sequence
gcgtaaagagagg

SEQ ID NO: 3 - DNA, 14 bp, Artificial Sequence
  contiguous nucleotide sequence
gcgtaaagagaggt

SEQ ID NO: 4 - DNA, 15 bp, Artificial Sequence
  contiguous nucleotide sequence
cgcgtaaagagaggt

SEQ ID NO: 5 - DNA, 15 bp, Artificial Sequence
  contiguous nucleotide sequence
agaaggcacagacgg

SEQ ID NO: 6 - DNA, 16 bp, Artificial Sequence
  contiguous nucleotide sequence
gagaaggcacagacgg

SEQ ID NO: 7 - DNA, 16 bp, Artificial Sequence
  contiguous nucleotide sequence
agcgaagtgcacacgg

SEQ ID NO: 8 - DNA, 13 bp, Artificial Sequence
  contiguous nucleotide sequence
gaagtgcacacgg

SEQ ID NO: 9 - DNA, 15 bp, Artificial Sequence
  contiguous nucleotide sequence
gcgaagtgcacacgg

SEQ ID NO: 10 - DNA, 15 bp, Artificial Sequence
  contiguous nucleotide sequence
agcgaagtgcacacg

SEQ ID NO: 11 - DNA, 13 bp, Artificial Sequence
  contiguous nucleotide sequence
cgaagtgcacacg

SEQ ID NO: 12 - DNA, 16 bp, Artificial Sequence
  contiguous nucleotide sequence
aggtgaagcgaagtgc

SEQ ID NO: 13 - DNA, 15 bp, Artificial Sequence
  contiguous nucleotide sequence
aggtgaagcgaagtg

SEQ ID NO: 14 - DNA, 14 bp, Artificial Sequence
  contiguous nucleotide sequence
aggtgaagcgaagt

SEQ ID NO: 15 - DNA, 15 bp, Artificial Sequence
  oligonucleotide sequence
cagcgtaaagagagg

SEQ ID NO: 16 - DNA, 16 bp, Artificial Sequence
  oligonucleotide sequence
cagcgtaaagagaggt

SEQ ID NO: 17 - DNA, 17 bp, Artificial Sequence
  oligonucleotide sequence
cacgcgtaaagagaggt

SEQ ID NO: 18 - DNA, 17 bp, Artificial Sequence
  oligonucleotide sequence
caagaaggcacagacgg

SEQ ID NO: 19 - DNA, 18 bp, Artificial Sequence
  oligonucleotide sequence
cagagaaggcacagacgg

SEQ ID NO: 20 - DNA, 18 bp, Artificial Sequence
  oligonucleotide sequence
caagcgaagtgcacacgg

SEQ ID NO: 21 - DNA, 15 bp, Artificial Sequence
  oligonucleotide sequence
cagaagtgcacacgg

SEQ ID NO: 22 - DNA, 17 bp, Artificial Sequence
  oligonucleotide sequence
cagcgaagtgcacacgg

SEQ ID NO: 23 - DNA, 17 bp, Artificial Sequence
  oligonucleotide sequence
caagcgaagtgcacacg

SEQ ID NO: 24 - DNA, 15 bp, Artificial Sequence
  oligonucleotide sequence
cacgaagtgcacacg

SEQ ID NO: 25 - DNA, 18 bp, Artificial Sequence
  oligonucleotide sequence
caaggtgaagcgaagtgc

SEQ ID NO: 26 - DNA, 17 bp, Artificial Sequence
  oligonucleotide sequence
caaggtgaagcgaagtg

SEQ ID NO: 27 - DNA, 16 bp, Artificial Sequence
  oligonucleotide sequence
caaggtgaagcgaagt

SEQ ID NO: 28 - DNA, 69 bp, Hepatitis B virus
cctctctttacgcggactccccgtctgtgccttctcatctgccggaccgtgtgcacttcgcttcacctc

SEQ ID NO: 29 - DNA, 20 bp, Artificial Sequence
  Oligonucleotide motif
gcagaggtgaagcgaagtgc

SEQ ID NO: 30 - DNA, 19 bp, Artificial Sequence
  primer
ctgtgccttgggtggcttt

SEQ ID NO: 31 - DNA, 24 bp, Artificial Sequence
  primer
aaggaaagaagtcagaaggcaaaa

SEQ ID NO: 32 - DNA, 34 bp, Artificial Sequence
  probe
agctccaaattctttataagggtcgatgtccatg

SEQ ID NO: 33 - RNA, 18 nt, Artificial Sequence
  Synthetic Polynucleotide
  misc_feature (5)..(5): n is a, c, g, or u
acaanaauccucacaaua

SEQ ID NO: 34 - RNA, 15 nt, Artificial Sequence
  synthetic polynucleotide
  misc_feature (3)..(3): n is a, c, g, or u
  misc_feature (15)..(15): n is a, c, g, or u
uunuugugaggauun

SEQ ID NO: 35 - RNA, 20 nt, Artificial Sequence
  synthetic polynucleotide
  misc_feature (15)..(15): n is a, c, g, or u
uuauugugaggauunuuguc

SEQ ID NO: 36 - RNA, 22 nt, Artificial Sequence
  synthetic polynucleotide
  misc_feature (15)..(15): n is a, c, g, or u
uuauugugaggauunuugucgg

SEQ ID NO: 37 - RNA, 22 nt, Artificial Sequence
  synthetic polynucleotide
uuauugugaggauucuugucgg

SEQ ID NO: 38 - RNA, 22 nt, Artificial Sequence
  synthetic polynucleotide
uuauugugaggauuuuugucgg

SEQ ID NO: 39 - RNA, 19 nt, Artificial Sequence
  synthetic polynucleotide
  misc_feature (5)..(5): n is a, c, g, or u
acaanaauccucacaauaa

SEQ ID NO: 40 - RNA, 36 nt, Artificial Sequence
  synthetic polynucleotide
  misc_feature (6)..(6): n is a, c, g, or u
gacaanaauccucacaauaagcagccgaaaggcugc

SEQ ID NO: 41 - RNA, 36 nt, Artificial Sequence
  synthetic polynucleotide
gacaaaaauccucacaauaagcagccgaaaggcugc

SEQ ID NO: 42 - RNA, 36 nt, Artificial Sequence
  synthetic polynucleotide
gacaagaauccucacaauaagcagccgaaaggcugc

SEQ ID NO: 43 - DNA, 10 bp, Artificial Sequence
  synthetic polynucleotide
aatcctcaca

SEQ ID NO: 44 - RNA, 10 nt, Artificial Sequence
  synthetic polynucleotide
ugugaggauu

SEQ ID NO: 45 - DNA, 10 bp, Artificial Sequence
  synthetic polynucleotide
tgtgaggatt

SEQ ID NO: 46 - DNA, 20 bp, Artificial Sequence
  synthetic polynucleotide
tattgtgaggattcttgtca

SEQ ID NO: 47 - DNA, 20 bp, Artificial Sequence
  synthetic polynucleotide
cggtattgtgaggattcttg

SEQ ID NO: 48 - DNA, 20 bp, Artificial Sequence
  synthetic polynucleotide
tgtgaggattcttgtcaaca

SEQ ID NO: 49 - RNA, 21 nt, Artificial Sequence
  synthetic polynucleotide
uauugugaggauuuuugucaa

SEQ ID NO: 50 - DNA, 21 bp, Artificial Sequence
  synthetic polynucleotide
ugcgguauugugaggauuctt

SEQ ID NO: 51 - DNA, 23 bp, Artificial Sequence
  synthetic polynucleotide
acagcattgtgaggattcttgtc

SEQ ID NO: 52 - RNA, 23 nt, Artificial Sequence
  synthetic polynucleotide
uauugugaggauuuuugucaaca

SEQ ID NO: 53 - RNA, 23 nt, Artificial Sequence
  synthetic polynucleotide
auugugaggauuuuugucaacaa

SEQ ID NO: 54 - RNA, 23 nt, Artificial Sequence
  synthetic polynucleotide
uugugaggauuuuugucaacaag

SEQ ID NO: 55 - RNA, 36 nt, Artificial Sequence
  synthetic polynucleotide
gugguggacuucucucaauagcagccgaaaggcugc

SEQ ID NO: 56 - RNA, 22 nt, Artificial Sequence
  synthetic polynucleotide
uauugagagaaguccaccacgg

SEQ ID NO: 57 - RNA, 22 nt, Artificial Sequence
  synthetic polynucleotide
uuugugaggauuuuugucaagg

SEQ ID NO: 58 - RNA, 22 nt, Artificial Sequence
  synthetic polynucleotide
ucugagagaaguccaccacggg

SEQ ID NO: 59 - RNA, 22 nt, Artificial Sequence
  synthetic polynucleotide
uacugagagaaguccaccacgg

SEQ ID NO: 60 - RNA, 22 nt, Artificial Sequence
  synthetic polynucleotide
uaaaacugagagaaguccacgg

SEQ ID NO: 61 - DNA, 19 bp, Artificial Sequence
  synthetic polynucleotide
gacaagaatcctcacaata

SEQ ID NO: 62 - DNA, 19 bp, Artificial Sequence
  synthetic polynucleotide
cgtggtggacttctctcaa

SEQ ID NO: 63 - DNA, 19 bp, Artificial Sequence
  synthetic polynucleotide
gtggacttctctcaatttt

SEQ ID NO: 64 - DNA, 19 bp, Artificial Sequence
  synthetic polynucleotide
  misc_feature (6)..(6): n is a, c, g, or t
gacaanaatcctcacaata

SEQ ID NO: 65 - DNA, 19 bp, Artificial Sequence
  synthetic polynucleotide
  misc_feature (19)..(19): n is a, c, g, or t
cgtggtggacttctctcan

SEQ ID NO: 66 - DNA, 19 bp, Artificial Sequence
  synthetic polynucleotide
  misc_feature (15)..(15): n is a, c, g, or t
gtggacttctctcantttt

SEQ ID NO: 67 - DNA, 22 bp, Artificial Sequence
  synthetic polynucleotide
tgttgacaagaatcctcacaat

SEQ ID NO: 68 - DNA, 21 bp, Artificial Sequence
  synthetic polynucleotide
tcgtggtggacttctctcaat

SEQ ID NO: 69 - DNA, 22 bp, Artificial Sequence
  synthetic polynucleotide
  misc_feature (10)..(10): n is a, c, g, or t
tgttgacaanaatcctcacaat

SEQ ID NO: 70 - DNA, 21 bp, Artificial Sequence
  synthetic polynucleotide
  misc_feature (20)..(20): n is a, c, g, or t
tcgtggtggacttctctcant

SEQ ID NO: 71 - RNA, 36 nt, Artificial Sequence
  synthetic polynucleotide
ugcacuucgcgucaccucuagcagccgaaaggcugc

SEQ ID NO: 72 - RNA, 22 nt, Artificial Sequence
  synthetic polynucleotide
uagaggugacgcgaagugcagg

SEQ ID NO: 73 - RNA, 36 nt, Artificial Sequence
  synthetic polynucleotide
gcaccucucuuuacgcggaagcagccgaaaggcugc

SEQ ID NO: 74 - RNA, 22 nt, Artificial Sequence
  synthetic polynucleotide
uuccgcguaaagagaggugcgg

SEQ ID NO: 75 - RNA, 36 nt, Artificial Sequence; identical to SEQ ID NO: 41
  synthetic polynucleotide
  misc_feature (1)..(1): may be modified by 2′-O-methyl ribonucleotide
  misc_feature (1)..(2): may be modified by phosphorothioate
  misc_feature (2)..(2): may be modified by 2′-O-methyl ribonucleotide
  misc_feature (3)..(3): may be modified by 2′-fluoro- deoxyribonucleotide
  misc_feature (4)..(7): may be modified by 2′-O-methyl ribonucleotide
  misc_feature (8)..(10): may be modified by 2′-fluoro- deoxyribonucleotide
  misc_feature (11)..(11): may be modified by 2′-O-methyl ribonucleotide
  misc_feature (12)..(13): may be modified by 2′-fluoro- deoxyribonucleotide
  misc_feature (14)..(16): may be modified by 2′-O-methyl ribonucleotide
  misc_feature (17)..(17): may be modified by 2′-fluoro- deoxyribonucleotide
  misc_feature (18)..(26): may be modified by 2′-O-methyl ribonucleotide
  misc_feature (27)..(27): may be modified by 2′-modified-GalNAc guanosine
  misc_feature (28)..(30): may be modified by 2′-modified-GalNAc adenosine
  misc_feature (31)..(36): may be modified by 2′-O-methyl ribonucleotide

SEQ ID NO: 76 - RNA, 23 nt, Artificial Sequence
  synthetic polynucleotide
  misc_feature (1)..(1): may be modified by 4′-O-monomethylphosphonate- 2′-O-methyl uridine
  misc_feature (1)..(4): may be modified by phosphorothioate
  misc_feature (2)..(3): may be modified by 2′-fluoro- deoxyribonucleotide
  misc_feature (4)..(4): may be modified by 2′-O-methyl ribonucleotide
  misc_feature (5)..(5): may be modified by 2′-fluoro- deoxyribonucleotide
  misc_feature (6)..(6): may be modified by 2′-O-methyl ribonucleotide
  misc_feature (7)..(8): may be modified by 2′-fluoro- deoxyribonucleotide
  misc_feature (9)..(9): may be modified by 2′-O-methyl ribonucleotide
  misc_feature (10)..(10): may be modified by 2′-fluoro- deoxyribonucleotide
  misc_feature (11)..(11): may be modified by 2′-O-methyl ribonucleotide
  misc_feature (12)..(12): may be modified by 2′-fluoro- deoxyribonucleotide
  misc_feature (13)..(13): may be modified by 2′-O-methyl ribonucleotide
  misc_feature (14)..(14): may be modified by 2′-fluoro- deoxyribonucleotide
  misc_feature (15)..(15): may be modified by 2′-O-methyl ribonucleotide
  misc_feature (16)..(16): may be modified by 2′-fluoro- deoxyribonucleotide
  misc_feature (17)..(18): may be modified by 2′-O-methyl ribonucleotide
  misc_feature (19)..(19): may be modified by 2′-fluoro- deoxyribonucleotide
  misc_feature (20)..(22): may be modified by 2′-O-methyl ribonucleotide
  misc_feature (20)..(22): may be modified by phosphorothioate
uuauugugaggauuuuuugucgg

SEQ ID NO: 77 - DNA, 50 bp, Artificial Sequence
  synthetic polynucleotide
tgttgacaagaatcctcacaatacctcgtggtggacttctctcaattttc

SEQ ID NO: 78 - RNA, 22 nt, Artificial Sequence
  synthetic polynucleotide
ggaacuguuuuuaggaguguuu

SEQ ID NO: 79 - RNA, 22 nt, Artificial Sequence
  synthetic polynucleotide
ggaacuguucuuaggaguguuu

SEQ ID NO: 80 - RNA, 22 nt, Artificial Sequence
  synthetic polynucleotide
ggcuguuuuuaggaguguuauu

SEQ ID NO: 81 - RNA, 22 nt, Artificial Sequence
  synthetic polynucleotide
ggcuguucuuaggaguguuauu

SEQ ID NO: 82 - DNA, 22 bp, Artificial Sequence
  synthetic polynucleotide
ttgacaagaatcctcacaatac

SEQ ID NO: 83 - RNA, 22 nt, Artificial Sequence
  synthetic polynucleotide
gggcaccaccugaagagagucu

SEQ ID NO: 84 - RNA, 22 nt, Artificial Sequence
  synthetic polynucleotide
gggcaccaccugaagagaguuu

SEQ ID NO: 85 - DNA, 22 bp, Artificial Sequence
  synthetic polynucleotide
ctcgtggtggacttctctcaat

SEQ ID NO: 86 - RNA, 22 nt, Artificial Sequence
  synthetic polynucleotide
ggcaccaccugaagagagucau

SEQ ID NO: 87 - RNA, 22 nt, Artificial Sequence
  synthetic polynucleotide
ggcaccaccugaagagaguuau

SEQ ID NO: 88 - DNA, 22 bp, Artificial Sequence
  synthetic polynucleotide
tcgtggtggacttctctcaatt

SEQ ID NO: 89 - RNA, 22 nt, Artificial Sequence
  synthetic polynucleotide
ggcaccugaagagagucaaaau

SEQ ID NO: 90 - RNA, 22 nt, Artificial Sequence
  synthetic polynucleotide
ggcaccugaagagaguuaaaau

SEQ ID NO: 91 - DNA, 22 bp, Artificial Sequence
  synthetic polynucleotide
tggtggacttctctcaattttc

(19) United States
(12) Patent Application Publication
(10) Pub. No.: US 2023/0119360 A1
(43) Pub. Date: Apr. 20, 2023

(54) PHARMACEUTICAL COMBINATION OF A THERAPEUTIC OLIGONUCLEOTIDE TARGETING HBV AND A TLR7 AGONIST FOR TREATMENT OF HBV

(71) Applicant: Hoffmann-La Roche Inc., Little Falls, NJ (US)

(72) Inventors: Lu GAO, Shanghai (CN); Yonghong ZHU, Shanghai (CN); Søren OTTOSEN, Hørsholm (DK); Henrik MUELLER, Basel (CH); Xue ZHOU, Shanghai (CN); Julie Elisabeth Françoise BLAISING, Basel (CH); Yuyan JIN, Shanghai (CN); Qingyan BO, Shanghai (CN); Gaurav TYAGI, New York, NY (US)

(21) Appl. No.: 17/843,740

(22) Filed: Jun. 17, 2022

Related U.S. Application Data
(63) Continuation of application No. PCT/EP2020/087697, filed on Dec. 22, 2020.

(30) Foreign Application Priority Data
Dec. 24, 2019 (CN) PCT/CN2019/127972
Aug. 5, 2020 (CN) PCT/CN2020/107002

(51) Int. Cl. A61K 31/519; A61K 48/00; A61P 31/20

(52) U.S. Cl. CPC A61K 31/519; A61K 48/005; A61P 31/20

(57) ABSTRACT

The present invention is directed to compositions and methods for treating hepatitis B virus infection. In particular, the present invention is directed to a combination therapy comprising administration of a therapeutic oligonucleotide targeting HBV and a TLR7 agonist for use in the treatment of a chronic hepatitis B patient.

### SEQUENCE LISTING

The instant application contains a Sequence Listing which has been submitted electronically in ASCII format and is hereby incorporated by reference in its entirety. Said ASCII copy, created on Jun. 16, 2022, is named 51551-015001_Sequence_Listing_6_16_22_ST25 and is 26,809 bytes in size.

### FIELD OF INVENTION

The present invention is directed to compositions and methods for treating hepatitis B virus infection. In particular, the present invention is directed to a combination therapy comprising administration of a therapeutic oligonucleotide targeting HBV and a TLR7 agonist for use in the treatment of a chronic hepatitis B patient.

### BACKGROUND

HBV infection remains a major health problem worldwide which concerns an estimated 350 million chronic carriers. Approximately 25% of carriers can be predicted to die from chronic hepatitis, cirrhosis, or liver cancer. Hepatitis B virus is the second most significant carcinogen behind tobacco, causing from 60% to 80% of all primary liver cancer.

The outer envelope proteins of HBV are collectively known as hepatitis B surface antigen (HBsAg). HBsAg consists of three related polypeptides called S, M, and L encoded by overlapping open reading frames (ORF). The smallest envelope protein is S with 226 amino acids, called the S—ORF. M and L are produced from upstream translation initiation sites and add 55 and 108 amino acids, respectively, to S. HBV S, M, and L glycoproteins are found in the viral envelope of intact, infectious HBV virions, named Dane particles, and all three are produced and secreted in a vast excess that forms non-infectious subviral spherical and filamentous particles (both referred to as decoy particles) found in the blood of chronic HBV patients. The abundance of HBsAg on the surface of decoy particles is believed to inhibit humoral immunity and spontaneous clearance in patients with chronic HBV infection (CHB).

The current standard of care for chronic HBV infection is treatment with oral nucleos(t)ide analogues such as entecavir or tenofovir which provide suppression of HBV replication by inhibiting HBV DNA synthesis but do not act directly on viral antigens, such as HBsAg. Nucleos(t)ide analogs, even with prolonged therapy, only show low levels of HBsAg clearance. In this respect, patients with chronic hepatitis B exhibit very weak HBV T-cell responses and lack anti-HBs antibodies, which is believed to be one of the reasons that these patients are not able to clear the virus.

A clinically important goal is to achieve a functional cure of chronic HBV infection, defined as HBsAg seroconversion and serum HBV-DNA elimination. This is expected to result in a durable response thereby preventing development of cirrhosis and liver cancer, and prolonging survival. Currently, chronic HBV infection cannot be eradicated completely due to the long term or permanent persistence of the viral genome as a covalently closed circular DNA (cccDNA) in the nuclei of infected hepatocytes. A complete cure from chronic HBV infection would require the elimination of this cccDNA from aa infected hepatocytes.

The review article Soriano et al 2017 Expert Opinion on Investigational Drugs Vol. 26, pp 843 describes the current status in drug development aiming to achieve either a functional cure of HBV or a complete cure. This article highlights some of the more than 30 drugs that are currently being tested in HBV therapy, also mentioning that any effective treatment leading to a cure will probably require a combination of a virus targeting therapy and an immunotherapy.

The toll-like receptor TLR7 is a component of the innate immune response to viral infection and is predominately expressed on plasmacytoid cells and on B-cells. Altered responsiveness of such immune cells might contribute to the reduced innate immune responses during chronic viral infections. Agonist-induced activation of TLR7 therefore represents a possible approach for the treatment of chronic viral infections using immunotherapy. Several TLR agonists are being tested in clinical trials, including GS-9620. Alternative TLR7 agonists are described in WO 2006/066080, WO 2016/055553 and WO 2016/91698.

Antisense oligonucleotides are essentially single stranded oligonucleotides capable of modulating expression of a target gene by hybridizing to a target nucleic acid. Target modulation can be down-regulation via RNase H mediated degradation or by blockage of the transcription. Antisense oligonucleotides can also up-regulate a target e.g. via splice switching or micro RNA repression. For targets in the liver GalNAc conjugation has proven very effective for delivering antisense oligonucleotides. WO 2014/179627 and WO2015/173208 describe HBV treatment through degradation of HBV mRNA in hepatocytes using single stranded antisense oligonucleotides in combination with GalNAc conjugation. Various combination therapies, including TLR7 agonist GS-9620, are briefly mentioned in WO2015/173208.

WO2016/077321 describes HBV treatment through degradation of HBV mRNA in hepatocytes using double stranded siRNA in combination with GalNAc conjugation on the sense strand. Various combination therapies including TLR7 agonists are briefly mentioned.

To our knowledge no specific combinations of therapeutic oligonucleotides and TLR7 agonists have been tested in vitro or in vivo.

### OBJECTIVE OF THE INVENTION

The present invention identifies novel combinations of therapeutic oligonucleotides targeting HBV and TLR7 agonists, which provide an advantage over the mono-compound treatments in terms of prolonged serum HBV-DNA reduction and delayed rebound in HBsAg. Furthermore, an increase in the therapeutic window can be achieved with the combination treatment, since a significantly improved effect can be achieved with 3-5 times lower dose when using the combination treatment compared to drug concentrations used in mono-treatment, and essentially the same effect can be achieved with the 3-5 times lower dose combination treatment when compared to the same combination at the high dose.

### SUMMARY OF INVENTION

An aspect of the present invention is a pharmaceutical combination which comprises or consists of a first medical compound which is a therapeutic oligonucleotide, and a second medical compound which is a TLR7 agonist of formula (I) or (II) as defined below. A preferred embodiment of the present invention is a pharmaceutical combination which comprises or consists of a first medical compound which is an RNAi oligonucleotide, preferably an oligonucleotide for reducing expression of HBsAg mRNA, the oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33), and a second medical compound which is a TLR7 agonist of formula (I) or (II) as defined below. Another embodiment of the present invention is a pharmaceutical combination which comprises or consists of a first medical compound which is an antisense oligonucleotide, preferably a GalNAc conjugated antisense oligonucleotide of 13 to 22 nucleotides in length with a contiguous nucleotide sequence of at least 12 nucleotides which is 100% complementary to a contiguous sequence from position 1530 to 1602 of SEQ ID NO: 1, and a second medical compound which is a TLR7 agonist of formula (I) or (II) as defined below.

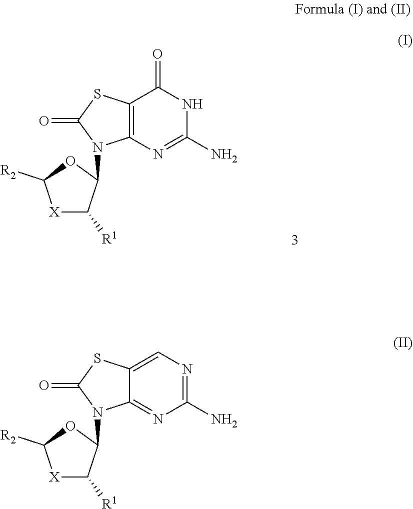

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or
1-hydroxymethyl or acetoxy(cyclopropyl)methyl or acetoxy(propyn-1-yl)methyl,
   or a pharmaceutically acceptable salt, enantiomer or diastereomer thereof.

A further aspect of the invention relates to the pharmaceutical combination for use in the treatment of a HBV infected individual, in particular an individual with chronic HBV.

A further aspect of the invention is use of a therapeutic oligonucleotide in the manufacture of a first medicament for treating a hepatitis B virus infection, wherein the first medicament is a therapeutic oligonucleotide as described in the application and wherein the first medicament is to be administered in combination with a second medicament, wherein the second medicament is a TLR7 agonist as described in the application.

In one embodiment the therapeutic oligonucleotide compound (first medicament or first medical compound) is formulated for subcutaneous injection and the TLR7 agonist compound (second medicament or second medical compound) is formulated for oral administration. Since the medical compounds will be administered through two different routes of administration they can follow different administration regiments. For optimal combination effects the first and the second medical compound are administered less than a month apart, such as less than a week apart, such as two day apart, such as on the same day.

A further aspect of the invention is a kit of parts including the first medical compound (first medicament) and a package insert with instruction for administration of the second medical compound (second medicament) in the treatment of HBV. In one embodiment the kits of part comprise both the first and the second medical compound.

A further aspect of the invention is a method for treating a hepatitis B virus infection comprising administering a therapeutically effective amount of a therapeutic oligonucleotide (first medicament) as described in the application in combination with a therapeutically effective amount of a TLR7 agonist (second medicament) as described in the application to a subject infected with a hepatitis B virus, such as a chronically infected individual.

In a highly preferred embodiment, the therapeutic oligonucleotide mentioned in the application is an RNAi oligonucleotide, preferably small interfering RNA (siRNA), preferably an RNAi oligonucleotide or siRNA for reducing expression of HBsAg mRNA. In a different embodiment, the therapeutic oligonucleotide is an antisense oligonucleotide, preferably a GalNAc conjugated antisense oligonucleotide, preferably an antisense oligonucleotide or GalNAc conjugated antisense oligonucleotide targeting HBV.

### BRIEF DESCRIPTION OF FIGURES

FIG. 1: Illustrates exemplary antisense oligonucleotide conjugates, showing various stereoisomers, where the oligonucleotide either is represented as a wavy line (A-D) or as “oligonucleotide” (E-H and K) or as T₂ (I-J) and the asialoglycoprotein receptor targeting conjugate moieties are trivalent N-acetylgalactosamine moieties. Compounds A to D comprise a di-lysine brancher molecule, a PEG3 spacer and three terminal GalNAc carbohydrate moieties. In compound A and B the oligonucleotide is attached directly to the asialoglycoprotein receptor targeting conjugate moiety without a linker. In compound C and D the oligonucleotide is attached to the asialoglycoprotein receptor targeting conjugate moiety via a C6 linker. Compounds E-J comprise a commercially available trebler brancher molecule and spacers of varying length and structure and three terminal GalNAc carbohydrate moieties. Compound K is composed of monomeric GalNAc phosphoramidites added to the oligonucleotide while still on the solid support as part of the synthesis, X═S or O and n=1−3 (see WO 2017/178656). FIGS. 1B and 1D are also termed GalNAc2 or GN2 herein, without and with C6 linker respectively.

FIG. 2: Structural formula of CMP ID NO: 29_1. Pharmaceutical salts thereof include monovalent or divalent cations, such as Na+, K+, and Ca²⁺ or a mixture of these being associated with the compound.

FIG. 3: Structural formula of CMP ID NO: 23_1. Pharmaceutical salts thereof include monovalent or divalent cations, such as Na+, K+, and Ca²⁺ or a mixture of these being associated with the compound.

FIG. 4: Structural formula of CMP ID NO: 16_1. Pharmaceutical salts thereof include monovalent or divalent cations, such as Na+, K+, and Ca²⁺ or a mixture of these being associated with the compound.

FIG. 5: Structural formula of CMP ID NO: 15_1. Pharmaceutical salts thereof include monovalent or divalent cations, such as Na+, K⁺, and Ca²⁺ or a mixture of these being associated with the compound.

FIG. 6: Structural formula of CMP ID NO: 15_2. Pharmaceutical salts thereof include monovalent or divalent cations, such as Na+, K⁺, and Ca²⁺ or a mixture of these being associated with the compound.

FIG. 7: Structural formula of CMP ID NO: 26_1. Pharmaceutical salts thereof include monovalent or divalent cations, such as Na+, K⁺, and Ca²⁺ or a mixture of these being associated with the compound.

FIG. 8: Structural formula of CMP ID NO: 20_1. Pharmaceutical salts thereof include monovalent or divalent cations, such as Na⁺, K⁺, and Ca²⁺ or a mixture of these being associated with the compound.

FIG. 9: Shows the effect of various mono- and combination treatments on HBV-DNA in serum from AAV/HBV mice. Panel A following treatment with either Saline (Vehicle, dash line and circles); CMP ID NO: VI (TLR7 agonist) administered at 100 mg/kg every other day (QOD) (dashed line; rectangle); CMP ID NO: 15_1 (anti-HBV ASO) dosed at 1.5 mg/kg (dashed line; triangle); or the combination of both (solid line and squares). Panel B following treatment with either Saline (Vehicle, dash line and circles); CMP ID NO: VI (TLR7 agonist) administered at 100 mg/kg weekly (QW) (dashed line, rectangle); CMP ID NO: 15_1 (anti-HBV ASO) dosed at 1.5 mg/kg (dashed line; triangle); or the combination of both (solid line and squares). Panel C following treatment with either Saline (Vehicle, dash line and circles); CMP ID NO: VI (TLR7 agonist) administered at 100 mg/kg every other day (QOD) (dashed line; rectangle); CMP ID NO: 15_1 (anti-HBV ASO) dosed at 7.5 mg/kg (dashed line; triangle); or the combination of both (solid line and squares). Panel D following treatment with either Saline (Vehicle, dash line and circles); CMP ID NO: VI (TLR7 agonist) administered at 100 mg/kg weekly (QW) (dashed line, rectangle); CMP ID NO: 15_1 (anti-HBV ASO) dosed at 7.5 mg/kg (dashed line; triangle); or the combination of both (solid line and squares).

FIG. 10: Shows the effect of various mono- and combination treatments on HBsAg in serum from AAV/HBV mice. Panel A following treatment with either Saline (Vehicle, dash line and circles); CMP ID NO: VI (TLR7 agonist) administered at 100 mg/kg every other day (QOD) (dashed line; rectangle); CMP ID NO: 15_1 (anti-HBV ASO) dosed at 1.5 mg/kg (dashed line; triangle); or the combination of both (solid line and squares). Panel B mice following treatment with either Saline (Vehicle, dash line and circles); CMP ID NO: VI (TLR7 agonist) administered at 100 mg/kg weekly (QW) (dashed line, rectangle); CMP ID NO: 15_1 (anti-HBV ASO) dosed at 1.5 mg/kg (dashed line; triangle); or the combination of both (solid line and squares). Panel C following treatment with either Saline (Vehicle, dash line and circles); CMP ID NO: VI (TLR7 agonist) administered at 100 mg/kg every other day (QOD) (dashed line; rectangle); CMP ID NO: 15_1 (anti-HBV ASO) dosed at 7.5 mg/kg (dashed line; triangle); or the combination of both (solid line and squares). Panel D following treatment with either Saline (Vehicle, dash line and circles); CMP ID NO: VI (TLR7 agonist) administered at 100 mg/kg weekly (QW) (dashed line, rectangle); CMP ID NO: 15_1 (anti-HBV ASO) dosed at 7.5 mg/kg (dashed line; triangle); or the combination of both (solid line and squares).

FIG. 11: Shows an example of an RNAi target site on a schematic representation of the organization of the HBV genome.

FIG. 12: Shows a single dose evaluation of an oligonucleotide for reducing HBsAg expression in HDI-mice.

FIG. 13: Shows a graphical representation of plasma HBsAg levels over time during a specified dosing regimen with an HBsAg-targeting oligonucleotide. As shown in this example, the oligonucleotide demonstrated preclinical potency and maintained decreased levels well beyond the dosing period.

FIG. 14: Shows graphs depicting the results of HBsAg mapping in HeLa cells using a reporter assay. An unmodified siRNA targeting position 254 of the HBV genome was used as a positive control at the specified concentrations. A commercially available Silencer siRNA from Thermo Fisher served as the negative control for these experiments. Error bars represent the SEM.

FIG. 15: Shows a genotype conservation comparison showing that the designed mismatch in the HBsAg-targeting oligonucleotide, HBV-219, increases coverage across HBV genotypes.

FIG. 16: Illustrates a vector designed for psiCHECK2 reporter assays using HBV Genotype A as a prototype sequence.

FIG. 17: Shows several examples of oligonucleotides designed to evaluate the effects of introducing mismatches. Oligonucleotide sequences for parent and mismatch strands are shown aligned and with mismatch positions in boxes. The corresponding reporter sequences used in psiCHECK2 reporter assays are further depicted.

FIG. 18: Shows a single-dose titration plot for an oligonucleotide evaluated in mismatch studies, which demonstrates that a mismatch in the guide strand is tolerated in vivo.

FIG. 19: Shows an in vivo dose titration plot demonstrating that incorporation of a mismatch into an HBsAg-targeting oligonucleotide does not adversely affect in vivo potency.

FIG. 20: Shows an example of an HBsAg-targeting oligonucleotide (HBV(s)-219) with chemical modifications and in duplex form. Darker shade indicates 2′-O-methyl ribonucleotide. Lighter shade indicates 2′-fluoro-deoxyribonucleotide.

FIG. 21A: Depicts immunohistochemical staining results detecting the subcellular distribution of HBV core antigen (HBcAg) in hepatocytes.

FIG. 21B: Depicts RNA sequencing results mapping detected RNA transcript sequences against the HBV pgRNA.

FIG. 22A: Depicts a time course of HBsAg mRNA expression following treatment with the HBV(s)-219 oligonucleotide precursor HBV(s)-219P2 targeting HBsAg mRNA compared with vehicle control and an RNAi oligonucleotide targeting HBV X antigen (HBxAg) mRNA in a hydrodynamic injection (HDI) model of HBV.

FIG. 22B: Depicts a time course of HBsAg mRNA expression following treatment with the HBV(s)-219 oligonucleotide precursor HBV(s)-219P2 targeting HBsAg mRNA compared with vehicle control and an RNAi oligonucleotide targeting HBxAg mRNA in an AAV-HBV model.

FIG. 23: Shows immunohistochemical staining results showing the subcellular distribution of HBcAg in hepatocytes obtained from AAV-HBV model and HDI model of HBV following treatment with the HBV(s)-219 oligonucleotide targeting HBsAg mRNA compared with vehicle control and an RNAi oligonucleotide targeting HBxAg mRNA (GalXC—HBVX).

FIGS. 24A-24D: Show antiviral activity of HBV(s)-219 precursor 1 (HBV(s)-219 P1) in a PXB—HBV model. Cohorts of 9 mice were given 3 weekly doses of either 0 or 3 mg/kg of HBV(s)-219P1 in PBS, administered subcutaneously. Six mice from each cohort were analyzed by non-terminal mandibular cheek bleeds at each of the time points indicated (FIGS. 24A and 24B) for serum HBsAg and serum HBV DNA. At Day 28 (starting from the first dose of HBV(s)-219P1), all remaining mice were euthanized and liver biopsies were collected for hepatic HBV DNA (FIG. 24C) and hepatic cccDNA (FIG. 24D) by RT-qPCR.

FIGS. 25A-25C: Show that HBV(s)-219 precursor 2 (HBV(s)-219P2) potentiates the antiviral activity of entecavir. In a HBV mouse hydrodynamic injection (HDI) model, a single dose of HBV(s)-219P2 was administered to mice subcutaneously on Day 1 followed by daily oral dosing of 500 ng/kg Entecavir (ETV) for 14 days. Circulating viral load (HBV DNA) was measured by qPCR (FIG. 25A). Plasma HBsAg level was measured by ELISA (FIG. 25B). Liver HBV mRNA and pgRNA levels were measured by qPCR (FIG. 25C). The results show clear additive effects with combination therapy. ETV therapy alone shows no efficacy against circulating HBsAg or liver viral RNAs. The antiviral activity of HBV(s)-219P2 as measured by HBsAg or HBV RNA is not impacted by co-dosing of ETV. “BLOD” means “below limit of detection.”

FIGS. 26A-26B: Show a comparison of HBsAg suppression activity of GaINac conjugated oligonucleotide targeting the S antigen (HBV(s)-219P2) or the X antigen (designated GalXC—HBVX). The result shows that HBVS-219P2 suppresses HBsAg for a longer duration than GalXC—HBVX or an equimolar combination of both RNAi Agents. FIG. 26A shows the location of RNAi target site in HBV genome affects HBsAg recovery kinetics in HBV-expressing mice.

FIG. 26B shows plasma HBsAg level 2 weeks post-dose (left panel) and 9 weeks pose-dose (right panel), indicating that targeting the HBVX coding region, either alone or in combination with HBV(s)-219P2, results in shorter duration of activity. Individual animal data was shown. Several data points (lightest grey circles) were below limit of detection.

FIGS. 27A-27C: Show the subcellular location of HBV core antigen (HBcAg) in HBV-expressing mice treated with HBV(s)-219P2, GalXC—HBVX or a 1:1 combination. FIG. 27A shows representative hepatocytes in liver sections obtained at weeks 1, 2, 6, 9, and 13 post administration and stained for HBcAg. FIG. 27B shows the percentage of HBcAg-positive-cells with nuclear staining in each animal (n=3/group, 50 cells counted per animal, 2 weeks after dosing). Alternative sequences were designed and tested targeting within the X and S open reading frames. FIG. 27C shows subcellular distribution of HBcAg in hepatocytes obtained at weeks 2, 3, and 9 post administration of an alternative RNAi oligo targeting either the S antigen or the X antigen.

FIG. 28: Shows the dose by cohort information for a study designed to evaluate the safety and tolerability of HBV(s)-219 in healthy patients and the therapeutic efficacy of HBV(s)-219 in HBV patients.

FIGS. 29A-29B: Show the chemical structure of HBV(s)-219 and HBV(s)-219P2. (FIG. 29A) Chemical structure for HBV(s)-219. (FIG. 29B) Chemical structure for HBV(s)-219P2.

FIG. 30: Shows the effects of HBV-LNA (CMP ID NO: 15_1, an antisense oligonucleotide according to the present invention) and DCR-5219 (an RNAi oligonucleotide, specifically a siRNA, according to the present invention) on reducing HBsAg titre over time. “DCR-AUD1” (a control siRNA targeting a sequence other than HBV) and “Vehicle” (sterile water) are negative controls. The dose of HBV-LNA in FIG. 30 is 6.6 mg/kg, whereas the dose of DCR-5219 is 9 mg/kg, but the molar dose of HBV-LNA is around three times higher than that of DCR-5219.

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

Further, an oligonucleotide is a short nucleic acid, e.g., of less than 100 nucleotides in length. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Synthetic

As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

Double-Stranded Oligonucleotide

As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Strand

As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Duplex

As used herein, the term “duplex,” in reference to nucleic acids (e.g., oligonucleotides), refers to a structure formed through complementary base-pairing of two antiparallel sequences of nucleotides.

Overhang

As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Loop

As used herein, the term “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

RNAi Oligonucleotide

As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

RNAi Agent

The terms ‘iRNA,” “RNAi agent,” ‘iRNA agent,” and “RNA interference agent” as used interchangeably herein, refer to an agent, e.g. an RNAi oligonucleotide, that contains RNA nucleosides herein and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g. inhibits, the expression of the target nucleic acid in a cell, e.g. a cell within a subject, such as a mammalian subject. RNAi agents include single stranded RNAi agents and double stranded siRNAs, as well as short hairpin RNAs (shRNAs). The oligonucleotide of the invention or contiguous nucleotide sequence thereof may be in the form of an RNAi agent, or form part of an RNAi agent, such as an siRNA or shRNA. In some embodiments of the invention, the oligonucleotide of the invention or contiguous nucleotide sequence thereof is an RNAi agent, such as a siRNA.

siRNAs

The term siRNA refers to small interfering ribonucleic acid RNAi agents and is a class of double-stranded RNA molecules, also known in the art as short interfering RNA or silencing RNA. siRNAs typically comprise a sense strand (also referred to as a passenger strand) and an antisense strand (also referred to as the guide strand), wherein each strand are of 17-30 nucleotides in length, typically 19-25 nucleosides in length, wherein the antisense strand is complementary, such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and antisense strand 3′ ends may form a 3′ overhang of e.g. 1, 2 or 3 nucleosides. In some embodiments, both the sense strand and antisense strand have a 2 nt 3′ overhang. The duplex region may therefore be, for example 17-25 nucleotides in length, such as 21-23 nucleotide in length.

Once inside a cell the antisense strand is incorporated into the RISC complex which mediates target degradation or target inhibition of the target nucleic acid. siRNAs typically comprise modified nucleosides in addition to RNA nucleosides, or in some embodiments all of the nucleotides of an siRNA strand may be modified (the sense 2′ sugar modified nucleosides such as LNA (see WO2004083430, WO2007085485 for example), 2′-fluoro, 2′-O-methyl or 2′-O-methoxyethyl may be incorporated into siRNAs). In some embodiments the passenger stand of the siRNA may be discontinuous (see WO2007107162 for example). The incorporation of thermally destabilizing nucleotides occurring at a seed region of the antisense strand of siRNAs have been reported as useful in reducing off-target activity of siRNAs (see WO18098328 for example).

In some embodiments, the dsRNA agent, such as the siRNA of the invention, comprises at least one modified nucleotide. In some embodiments, substantially all of the nucleotides of the sense strand comprise a modification; substantially all of the nucleotides of the antisense strand comprise a modification; or substantially all of the nucleotides of the sense strand and substantially all of the nucleotides of the antisense strand comprise a modification. In yet other embodiments, all of the nucleotides of the sense strand comprise a modification; all of the nucleotides of the antisense strand comprise a modification; or all of the nucleotides of the sense strand and all of the nucleotides of the antisense strand comprise a modification.

In some embodiments, the modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, an unlinked nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide, and combinations thereof. Suitably the siRNA comprises a 5′ phosphate group or a 5′-phosphate mimic at the 5′ end of the antisense strand. In some embodiments the 5′ end of the antisense strand is a RNA nucleoside.

In one embodiment, the dsRNA agent further comprises at least one phosphorothioate or methylphosphonate internucleotide linkage. The phosphorothioate or methylphosphonate internucleotide linkage may be at the 3′-terminus one or both strand (e.g., the antisense strand; or the sense strand); or the phosphorothioate or methylphosphonate internucleoside linkage may be at the 5′-terminus of one or both strands (e.g., the antisense strand; or the sense strand); or the phosphorothioate or methylphosphonate internucleoside linkage may be at the both the 5′- and 3′-terminus of one or both strands (e.g., the antisense strand; or the sense strand). In some embodiments the remaining internucleoside linkages are phosphodiester linkages.

The dsRNA agent may further comprise a ligand. In some embodiments, the ligand is conjugated to the 3′ end of the sense strand. For biological distribution, siRNAs may be conjugated to a targeting ligand, and/or be formulated into lipid nanoparticles, for example. Other aspects of the invention relate to pharmaceutical compositions comprising these dsRNA, such as siRNA molecules suitable for therapeutic use, and methods of inhibiting the expression of the target gene by administering the dsRNA molecules such as siRNAs of the invention, e.g., for the treatment of various disease conditions as disclosed herein.

Tetraloop

As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., Nature 1990 Aug. 16; 346(6285):680-2; Heus and Pardi, Science 1991 Jul. 12; 253(5016):191-4). In some embodiments, a tetraloop comprises 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) Nucl. Acids Res. 13: 3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., Proc Natl Acad Sci USA. 1990 November; 87(21):8467-71; Antao et al., Nucleic Acids Res. 1991 Nov. 11; 19(21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al. Biochemistry, 41 (48), 14281-14292, 2002. SHINJI et al. Nippon Kagakkai Koen Yokoshu VOL. 78th; NO. 2; PAGE. 731 (2000), which are incorporated by reference herein for their relevant disclosures. In some embodiments, the tetraloop is contained within a nicked tetraloop structure.

Nicked Tetraloop Structure

A “nicked tetraloop structure” is a structure of an RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide of the invention does not contain RNA nucleosides, since this will decrease nuclease resistance.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as an F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Deoxyribonucleotide

As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Ribonucleotide

As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Nucleotide

As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region G of a gapmer oligonucleotide, as well as in regions of modified nucleosides, such as region F and F′.

In an embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

With the oligonucleotides of the invention it is advantageous to use phosphorothioate internucleoside linkages.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

In some embodiments, the oligonucleotide of the invention comprises both phosphorothioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages of the contiguous nucleotide sequence of the oligonucleotide are phosphorothioate, or all the internucleoside linkages of the oligonucleotide are phosphorothioate linkages. In particular, all the internucleoside linkages of the contiguous nucleotide sequence of the antisense oligonucleotide are phosphorothioate, or all the internucleoside linkages of the antisense oligonucleotide are phosphorothioate linkages.

It is recognized that, as disclosed in EP 2 742 135, therapeutic oligonucleotides may comprise other internucleoside linkages (other than phosphodiester and phosphorothioate), for example alkyl phosphonate/methyl phosphonate internucleoside, which according to EP 2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term “chimeric” oligonucleotide is a term that has been used in the literature to describe oligonucleotides with modified nucleosides.

Complementarity

As used herein, “complementary” refers to a structural relationship between two nucleotides (e.g., on two opposing nucleic acids or on opposing regions of a single nucleic acid strand), or between two sequences of nucleotides, that permits the two nucleotides, or two sequences of nucleotides, to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, is complementary to a reference sequence (e.g. a target sequence or sequence motif). The percentage of complementarity is thus calculated by counting the number of aligned nucleobases that are complementary (from e.g. Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (e.g. form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form e.g. Watson Crick base pairing is retained (e.g. 5′-methyl cytosine is considered identical to a cytosine for the purpose of calculating % identity).

The term “fully complementary”, refers to 100% complementarity.

The following is an example of a contiguous nucleotide sequence that is fully complementary to a region of the HBV transcript.

The following is an example of a contiguous nucleotide sequence (SEQ ID NO: 6) that is fully complementary to a region of the HBV target (SEQ ID NO: 28).

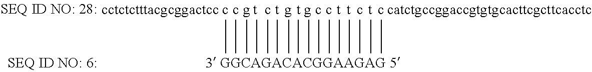

In some embodiments, two nucleic acids may have regions of multiple nucleotides that are complementary with each other so as to form regions of complementarity, as described herein.

Region of Complementarity

As used herein, the term “region of complementarity” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions, e.g., in a phosphate buffer, in a cell, etc.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, is identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation of the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes Hepatitis B virus and may for example be a gene, a RNA, a mRNA, viral mRNA or a cDNA sequence. The target nucleic acid is represented by SEQ ID NO: 1 and naturally occurring variants thereof.

For in vivo or in vitro application, the oligonucleotide of the invention is typically capable of inhibiting the expression of the HBV target nucleic acid in a cell which is expressing the HBV target nucleic acid. The contiguous sequence of nucleobases of the oligonucleotide of the invention is typically complementary to the HBV target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″).

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides of the invention.

Described herein is an HBV mRNA target region for a therapeutic oligonucleotide represented by the sequence from position 1530 to 1602 of SEQ ID NO: 1 or SEQ ID NO: 28. This target region can be split into smaller target sequences and selected from the group consisting of position 1530 to 1602; 1530 to 1598; 1530-1543; 1530-1544; 1531-1543; 1551-1565; 1551-1566; 1577-1589; 1577-1591; 1577-1592; 1578-1590; 1578-1592; 1583-1598; 1584-1598; 1585-1598 or 1583-1602 of SEQ ID NO: 1.

In an embodiment, the therapeutic oligonucleotide of the invention comprises a contiguous nucleotide sequence which is complementary to or hybridizes to the target sequence from position 1530 to 1602 of SEQ ID NO: 1 or SEQ ID NO: 28. In particular to a target sequence selected from the group consisting of 1530-1544; 1531-1543; 1585-1598 and 1583-1602.

The target sequence to which the antisense oligonucleotide is complementary or hybridizes to generally comprises a contiguous nucleobase sequence of at least 10 nucleotides. The contiguous nucleotide sequence of the target region is between 10 to 50 nucleotides, such as 12 to 30, such as 14 to 20, such as 15 to 18 contiguous nucleotides.

Target Cell

The term a “target cell” as used herein refers to a cell which is expressing the target nucleic acid. In some embodiments the target cell may be in vivo or in vitro. In some embodiments the target cell is a HBV infected mammalian cell such as a rodent cell, such as a mouse cell or a human cell, in particular a HBV infected hepatocyte.

In preferred embodiments the target cell expresses HBV mRNA and secretes HBsAg and HBeAg.

Hepatocyte

As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include, but are not limited to: transthyretin (Ttr), glutamine synthetase (GluI), hepatocyte nuclear factor 1a (Hnf1a), and hepatocyte nuclear factor 4a (Hnf4a). Markers for mature hepatocytes may include, but are not limited to: cytochrome P450 (Cyp3a11), fumarylacetoacetate hydrolase (Fah), glucose 6-phosphate (G6p), albumin (Alb), and 002-2F8. See, e.g., Huch et al., (2013), Nature, 494(7436): 247-250, the contents of which relating to hepatocyte markers is incorporated herein by reference.

Reduced Expression

As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a pharmaceutical combination or a double-stranded oligonucleotide (e.g., one having an antisense strand that is complementary to an HBsAg mRNA sequence) may result in a decrease in the amount of RNA transcript, protein and/or enzymatic activity (e.g., encoded by the S gene of an HBV genome) compared to a cell that is not treated with the pharmaceutical combination or double-stranded oligonucleotide respectively. Similarly, “reducing expression” as used herein refers to an act that results in reduced expression of a gene (e.g., the S gene of an HBV genome).

Naturally Occurring Variant

The term “naturally occurring variant thereof” refers to variants of the target nucleic acid which exist naturally within the defined taxonomic group, such as HBV genotypes A-H. Typically, when referring to “naturally occurring variants” of a polynucleotide the term may also encompass any allelic variant of the target sequence encoding genomic DNA which are found by chromosomal translocation or duplication, and the RNA, such as mRNA derived therefrom. “Naturally occurring variants” may also include variants derived from alternative splicing of the target sequence mRNA. When referenced, e.g. to a specific polypeptide sequence, the term also includes naturally occurring forms of the protein which may therefore be processed, e.g. by co- or post-translational modifications, such as signal peptide cleavage, proteolytic cleavage, glycosylation, etc.

High Affinity Modified Nucleosides

A high affinity modified nucleoside is a modified nucleotide which, when incorporated into the oligonucleotide, enhances the affinity of the oligonucleotide for its complementary target, for example as measured by the melting temperature (Tₘ). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

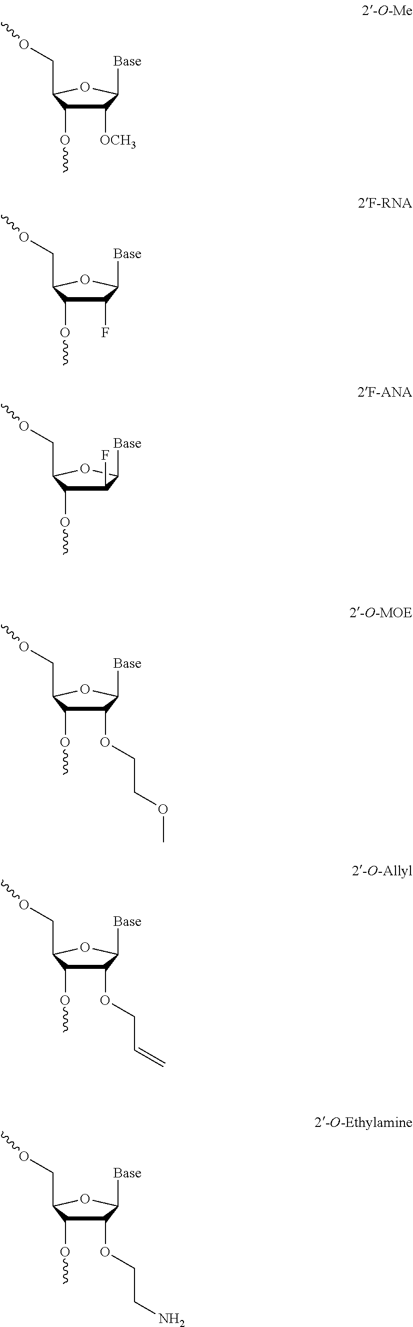

In relation to the present invention 2′ substituted sugar modified nucleosides does not include 2′ bridged nucleosides like LNA.

Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

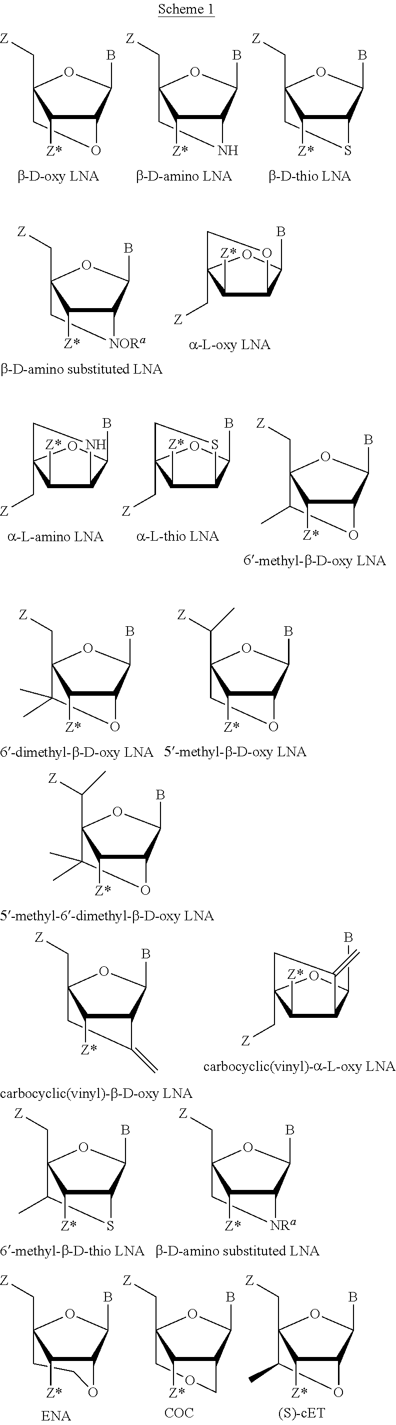

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

Phosphate Analog

As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, U.S. Provisional Application numbers 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), Nucleic Acids Res., 43(6):2993-3011, the contents of each of which relating to phosphate analogs are incorporated herein by reference).

Nuclease Mediated Degradation

Nuclease mediated degradation refers to an oligonucleotide capable of mediating degradation of a complementary nucleotide sequence when forming a duplex with such a sequence.

In some embodiments, the antisense oligonucleotide may function via nuclease mediated degradation of the target nucleic acid, where the oligonucleotides of the invention are capable of recruiting a nuclease, particularly an endonuclease, preferably an endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

RNase H Activity and Recruitment

In one embodiment, the therapeutic oligonucleotide is an antisense oligonucleotide capable of recruiting RNase H. The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RNase H activity, recombinant human RNase H1 is available from Lubio Science GmbH, Lucerne, Switzerland.

Gapmer

In some embodiments where the therapeutic oligonucleotide of the present invention is an antisense oligonucleotide, the nucleic acid molecule of the invention, or contiguous nucleotide sequence thereof are gapmer antisense oligonucleotides. The antisense gapmers are commonly used to inhibit a target nucleic acid via RNase H mediated degradation. In an embodiment of the invention the antisense oligonucleotide of the invention is capable of recruiting RNase H.

A gapmer antisense oligonucleotide comprises at least three distinct structural regions: a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 30 nucleosides, such as 13 to 24, such as 14 to 22 nucleosides, Such as from 13 to 17, such as 14 to 16 nucleosides. By way of example, the gapmer oligonucleotide of the present invention can be represented by the following formulae:

F₁₋₆−G₆₋₁₆−F′₁₋₆,such as

F₁₋₄−G₇₋₁₀−F′₂₋₄

with the proviso that the overall length of the gapmer regions F-G-F′ is at least 12, such as at least 13 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region of between 6 and 16 nucleosides which are capable of recruiting RNase H.

In one embodiment of the invention the contiguous nucleotide sequence is a gapmer of formula 5′-F-G-F′-3′, where region F and F′ independently consist of 2-4 2′ sugar modified nucleotides and defines the 5′ and 3′ end of the F and F′ region, and G is a region between 6 and 10 DNA nucleosides which are capable of recruiting RNase H.

In some embodiments the gap region G may consist of 6, 7, 8, 9, 10, 11, 12, 13, 14, 15 or 16 contiguous phosphorothioate linked DNA nucleosides. In some embodiments the gap region G consist of 7 to 10 DNA nucleosides. In some embodiments, all internucleoside linkages in the gap are phosphorothioate linkages.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

Further gapmer designs are disclosed in WO2004/046160, WO2007/146511 and WO2008/113832, hereby incorporated by reference.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅−[region G]₆₋₁₀−[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]₅₋₁₆-[MOE]₁₋₈, such as [MOE]₂₋₇−[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety it can serve as a biocleavable linker. Alternatively, it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide. The transition between region D′ and F region and between region F′ and D″ region is characterized by a nucleoside with a phosphodiester linkage towards the D′ or D″ region and a phosphorothioate linkage towards the F or F′ region, and the nucleoside is considered to be a part of the gapmer (contiguous nucleotide sequence which is complementary to the target nucleic acid).

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D″ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. In some embodiments region D′ or D″ is not complementary to or comprises at least 50% mismatches to the target nucleic acid.

In some embodiments region D′ or D″ comprises or consists of a dinucleotide of sequence AA, AT, AC, AG, TA, TT, TC, TG, CA, CT, CC, CG, GA, GT, GC, or GG, wherein C may be 5-methylcytosine, and/or T may be replaced with U. The internucleoside linkage in the dinucleotide is a phosphodiester linkage. In some embodiments region D′ or D″ comprises or consists of a trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, and GGG wherein C may be 5-methylcytosine and/or T may be replaced with U. The internucleoside linkages are phosphodiester linkages. It will be recognized that when referring to (naturally occurring) nucleobases A (adenine, T (thymine), U (uracil), G (guanine), C (cytosine), these may be substituted with nucleobase analogues which function as the equivalent natural nucleobase (e.g. base pair with the complementary nucleoside).

In one embodiment the antisense oligonucleotide of the invention comprises a region D′ and/or D″ in addition to the contiguous nucleotide sequence which constitutes the gapmer.

In some embodiments, the antisense oligonucleotide of the present invention can be represented by the following formulae:

D′-F-G-F′, in particular D′₁₋₃-F₁₋₄-G₆₋₁₀-F′₂₋₄

F-G-F′-D″, in particular F₁₋₄-G₆₋₁₀-F′₂₋₄-D″₁₋₃

D′-F-G-F′-D″, in particular D′₁₋₃-F₁₋₄-G₆₋₁₀-F′₂₋₄-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to a non-nucleotide moiety (conjugate), such as a GalNAc cluster, which can be covalently linked to a therapeutic oligonucleotide. The term conjugate and cluster or conjugate moiety may be used interchangeably. In some instances the conjugated therapeutic oligonucleotide may also be termed an oligonucleotide conjugate. In an embodiment, the conjugate is a targeting ligand.

Targeting Ligand

As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Oligonucleotide Linkers

A linkage or linker is a connection between two atoms that links one chemical group or segment of interest to another chemical group or segment of interest via one or more covalent bonds. Conjugate groups can be attached to the oligonucleotide directly or through a linking moiety (e.g. linker or tether). Linkers serve to covalently connect a conjugate group, to an oligonucleotide or contiguous nucleotide sequence complementary to the target nucleic acid.

In some embodiments of the invention the therapeutic oligonucleotide may optionally comprise a linker region which is positioned between the oligonucleotide or contiguous nucleotide sequence complementary to the target nucleic acid and the conjugate.

Such linkers can be biocleavable linkers comprising or consisting of a physiologically labile bond that is cleavable under conditions normally encountered or analogous to those encountered within a mammalian body. In one embodiment the biocleavable linker is susceptible to 51 nuclease cleavage.

For biocleavable linkers placed between the conjugate and the therapeutic oligonucleotide, it is preferred that the cleavage rate seen in the target tissue (for example muscle, liver, kidney or a tumor) is greater than that found in blood serum. Suitable methods for determining the level (%) of cleavage in target tissue versus serum or cleavage by 51 nuclease are described in the “Materials and methods” section. In some embodiments, the biocleavable linker is at least about 20% cleaved, such as at least about 30% cleaved, such as at least about 40% cleaved, such as at least about 50% cleaved, such as at least about 60% cleaved, such as at least about 70% cleaved, such as at least about 75% cleaved when compared against a standard.

In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers (PO linkers) are described in more detail in WO 2014/076195 (hereby incorporated by reference).

Additional or alternative linkers that are not necessarily biocleavable but primarily serve to covalently connect a conjugate to the oligonucleotide may also be used either alone or in combination with PO linkers. The non-cleavable linkers may comprise a chain structure or an oligomer of repeating units such as ethylene glycol, amino acid units or amino alkyl groups. In some embodiments the non-cleavable linker is an amino alkyl, such as a C2-C36 amino alkyl group, including, for example C6 to C12 amino alkyl groups. In a preferred embodiment the linker is a C6 amino alkyl group.

Hepatitis B Virus

As used herein, “hepatitis B virus” or “HBV” refers to a member of the Hepadnaviridae family having a small double-stranded DNA genome of approximately 3,200 base pairs and a tropism for liver cells. “HBV” includes hepatitis B virus that infects any of a variety of mammalian (e.g., human, non-human primate, etc.) and avian (duck, etc.) hosts. “HBV” includes any known HBV genotype, e.g., serotype A, B, C, D, E, F, and G; any HBV serotype or HBV subtype; any HBV isolate; HBV variants, e.g., HBeAg-negative variants, drug-resistant HBV variants (e.g., lamivudine-resistant variants; adefovir-resistant mutants; tenofovir-resistant mutants; entecavir-resistant mutants; etc.); and the like.

“HBV” is a small DNA virus belonging to the Hepadnaviridae family and classified as the type species of the genus Orthohepadnavirus. HBV virus particles (virions) comprise an outer lipid envelope and an icosahedral nucleocapsid core composed of protein. The nucleocapsid generally encloses viral DNA and a DNA polymerase that has reverse transcriptase activity similar to retroviruses. The HBV outer envelope contains embedded proteins which are involved in viral binding of, and entry into, susceptible cells. HBV, which attacks the liver, has been classified according to at least ten genotypes (A-J) based on sequence. In general, there are four genes encoded by the genome, which genes are referred to as C, P, S, and X. The core protein is encoded by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S encodes surface antigen (HBsAg). The HBsAg gene is one long open reading frame but contains three in frame “start” (ATG) codons that divide the gene into three sections, pre-S1, pre-S₂, and S. Because of the multiple start codons, polypeptides of three different sizes called large, middle, and small (pre-S1+pre-S₂+S, pre-S₂+S, or S) are produced. These may have a ratio of 1:1:4 (Heermann et al, 1984).

Hepatitis B Virus (HBV) proteins can be organized into several categories and functions. Polymerases function as a reverse transcriptase (RT) to make viral DNA from pregenomic RNA (pgRNA), and also as a DNA-dependent polymerase to make covalently closed circular DNA (cccDNA) from viral DNA. They are covalently attached to the 5′ end of the minus strand. Core proteins make the viral capsid and the secreted E antigen. Surface antigens are the hepatocyte internalization ligands, and also the primary component of aviral spherical and filamentous particles. Aviral particles are produced >1000-fold over Dane particles (infectious virions) and may act as immune decoys.

Hepatitis B Virus Surface Antigen

As used herein, the term “hepatitis B virus surface antigen” or “HBsAg” refers to an S-domain protein encoded by gene S (e.g., ORF S) of an HBV genome. Hepatitis B virus particles carry viral nucleic acid in core particles enveloped by three proteins encoded by gene S, which are the large surface, middle surface, and major surface proteins. Among these proteins, the major surface protein is generally about 226 amino acids and contains just the S-domain.

Infection

As used herein, the term “infection” refers to the pathogenic invasion and/or expansion of microorganisms, such as viruses, in a subject. An infection may be lysogenic, e.g., in which viral DNA lies dormant within a cell. Alternatively, an infection may be lytic, e.g., in which the virus actively proliferates and causes destruction of infected cells. An infection may or may not cause clinically apparent symptoms. An infection may remain localized, or it may spread, e.g., through a subject's blood or lymphatic system. An individual having, for example, an HBV infection can be identified by detecting one or more of viral load, surface antigen (HBsAg), e-antigen (HBeAg), and various other assays for detecting HBV infection known in the art. Assays for detection of HBV infection can involve testing serum or blood samples for the presence of HBsAg and/or HBeAg, and optionally further screening for the presence of one or more viral antibodies (e.g., IgM and/or IgG) to compensate for any periods in which an HBV antigen may be at an undetectable level.

HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Some infected persons have no symptoms during the initial infection and some develop a rapid onset of sickness with vomiting, yellowish skin, tiredness, dark urine and abdominal pain (“Hepatitis B Fact sheet N°204”. who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public-Transmission”, U.S. Centers for Disease Control and Prevention (CDC), retrieved 2011 Nov., 29). Most of those with chronic disease have no symptoms; however, cirrhosis and liver cancer may eventually develop (Chang, 2007, Semin Fetal Neonatal Med, 12: 160-167). These complications result in the death of 15 to 25% of those with chronic disease (“Hepatitis B Fact sheet N°204”. who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

Liver Inflammation

As used herein, the term “liver inflammation” or “hepatitis” refers to a physical condition in which the liver becomes swollen, dysfunctional, and/or painful, especially as a result of injury or infection, as may be caused by exposure to a hepatotoxic agent. Symptoms may include jaundice (yellowing of the skin or eyes), fatigue, weakness, nausea, vomiting, appetite reduction, and weight loss. Liver inflammation, if left untreated, may progress to fibrosis, cirrhosis, liver failure, or liver cancer.

Liver Fibrosis

As used herein, the term “liver fibrosis” or “fibrosis of the liver” refers to an excessive accumulation in the liver of extracellular matrix proteins, which could include collagens (I, Ill, and IV), fibronectin, undulin, elastin, laminin, hyaluronan, and proteoglycans resulting from inflammation and liver cell death. Liver fibrosis, if left untreated, may progress to cirrhosis, liver failure, or liver cancer.

TLR7

As used herein, “TLR7” refers to the Toll-like receptor 7 of any species of origin (e.g., human, murine, woodchuck etc.).

TLR7 Agonist

As used herein, “TLR7 agonist” refers to a compound that acts as an agonist of TLR7. Unless otherwise indicated, a TLR7 agonist can include the compound in any pharmaceutically acceptable form, including any isomer (e.g., diastereomer or enantiomer), salt, solvate, polymorph, and the like. The TLR agonism for a particular compound may be determined in any suitable manner. For example, assays for detecting TLR agonism of test compounds are described, for example, in U.S. Provisional Patent Application Ser. No. 60/432,650, filed Dec. 11, 2002, and recombinant cell lines suitable for use in such assays are described, for example, in U.S. Provisional Patent Application Ser. No. 60/432,651, filed Dec. 11, 2002. A further assay for evaluating TLR7 agonists is the HEK293-Blue-hTLR-7 cell assay described in Example 43 of WO2016/091698 (the assay is hereby incorporated by reference).

Diastereomer

As used herein, the term “diastereomer” refers to a stereoisomer with two or more centers of chirality and whose molecules are not mirror images of one another. Diastereomers have different physical properties, e.g. melting points, boiling points, spectral properties, activities and reactivities.

Compounds of the general formulas (I)-(V) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

Pharmaceutically Acceptable Salts

The compounds according to the present invention may exist in the form of their pharmaceutically acceptable salts.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein.

Alternatively, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

Pharmaceutical Combination

As used herein a pharmaceutical combination is understood as the combination at least two different active compounds or prodrugs (medical compounds or medicaments) for treatment of a disease. A pharmaceutical combination can involve compounds that are physically, chemically, or otherwise combined (e.g., in the same vial); compounds that are packaged together (e.g., as two separate objects in the same package (kit of parts) either for simultaneous administration or separate administration); or compounds that are provided separately but intended to be used together (e.g. the combination is expressly stated on the compound label or package insert). In one embodiment the pharmaceutical combination consists of a medical compound formulated for oral administration and a medical compound formulated for subcutaneous injection.

Approximately

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering

As used herein, the terms “administering” or “administration” means to provide a substance (e.g., a pharmaceutical combination or an oligonucleotide) to a subject in a manner that is pharmacologically useful (e.g., to treat a condition in the subject).

Asialoglycoprotein Receptor (ASGPR)

As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

Prodrug

As used herein, the term “prodrug” refers to a form or derivative of a compound which is metabolized in vivo, e.g., by biological fluids or enzymes by a subject after administration, into a pharmacologically active form of the compound in order to produce the desired pharmacological effect. Prodrugs are described e.g. in the Organic Chemistry of Drug Design and Drug Action by Richard B. Silverman, Academic Press, San Diego, 2004, Chapter 8 Prodrugs and Drug Delivery Systems, pp. 497-558.

Subject

As used herein, the term “subject” means any mammal, including mice, rabbits, and humans. In one embodiment, the subject is a human or non-human primate. The terms “individual” or “patient” may be used interchangeably with “subject.”

Treatment

The terms “treatment”, “treating”, “treats” or the like are used herein generally mean obtaining a desired pharmacological and/or physiological effect. This effect is therapeutic in terms of partially or completely curing a disease and/or adverse effect attributed to the disease. The effect is provided through the administration a therapeutic agent (e.g., a pharmaceutical combination or an oligonucleotide) to the subject, for purposes of improving the health and/or well-being of the subject with respect to an existing condition (e.g., an existing HBV infection) or to prevent or decrease the likelihood of the occurrence of a condition (e.g., preventing liver fibrosis, hepatitis, liver cancer or other condition associated with an HBV infection). The term “treatment” as used herein covers any treatment of HBV infection in a subject and includes: (a) inhibiting the disease, i.e. arresting its development like the inhibiting of increase of HBsAg and/or HBeAg; or (b) ameliorating (i.e. relieving) the disease, i.e. causing regression of the disease, like the repression of HBsAg and/or HBeAg production. Thus, a compound or compound combination that ameliorates and/or inhibits a HBV infection is a compound or compound combination that treats a HBV invention. Preferably, the term “treatment” as used herein relates to medical intervention of an already manifested disorder, like the treatment of an already defined and manifested HBV infection, in particular a chronic HBV infection.

In some embodiments, treatment involves reducing the frequency or severity of at least one sign, symptom or contributing factor of a condition (e.g., HBV infection or related condition) experienced by a subject. During an HBV infection, a subject may exhibit symptoms such as yellowing of the skin and eyes (jaundice), dark urine, extreme fatigue, nausea, vomiting and abdominal pain. Accordingly, in some embodiments, a treatment, e.g. a pharmaceutical combination, provided herein may result in a reduction in the frequency or severity of one or more of such symptoms. However, HBV infection can develop into one or more liver conditions, such as cirrhosis, liver fibrosis, liver inflammation or liver cancer. Accordingly, in some embodiments, a treatment, e.g. pharmaceutical combination, provided herein may result in a reduction in the frequency or severity of, or prevent or attenuate, one or more of such conditions.

Therapeutic Effective Amount

The term “therapeutically effective amount” denotes an amount of a compound the pharmaceutical combination of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

Excipient

As used herein, the term “excipient” refers to a non-therapeutic agent that may be included in one or more of the compositions comprising a medicament which is part of a pharmaceutical combination, for example, to provide or contribute to a desired consistency or stabilizing effect.

### DETAILED DESCRIPTION OF THE INVENTION

The present invention relates to a pharmaceutical combination comprising two categories of compounds i) a therapeutic oligonucleotide and ii) a TLR7 agonist each in a pharmaceutically acceptable carrier. The pharmaceutical combination is for use in treatment of Hepatitis B virus infections, in particular treatment of patients with chronic HBV.

Below each category of compounds in the combination will be described separately, it is however to be understood that a least one compound from each category are present in the pharmaceutical combination. The compound can either be administered simultaneously or separately. The compounds in the category of therapeutic oligonucleotides targeting HBV may be administered parenterally (such as intravenous, subcutaneous, or intra-muscular). The TLR7 agonists may be administered enterally (such as orally or through the gastrointestinal tract).

In a first embodiment the therapeutic oligonucleotide targeting HBV is an RNAi oligonucleotide, preferably an RNAi oligonucleotide for reducing the expression of HBsAg mRNA. In a second embodiment the therapeutic oligonucleotide targeting HBV is an antisense oligonucleotide, preferably a GalNAc conjugated antisense oligonucleotide targeting HBV.

1. RNAi Oligonucleotide of the Invention

In some embodiments, the first medicament in the pharmaceutical combination of the invention is an oligonucleotide-based inhibitor of HBV surface antigen expression that can be used to achieve a therapeutic benefit. Through examination of HBV surface antigen mRNA and testing of different oligonucleotides, potent oligonucleotides have been developed for reducing expression of HBV surface antigen (HBsAg) to treat HBV infection. Oligonucleotides provided herein, in some embodiments, are designed to target HBsAg mRNA sequences covering >95% of known HBV genomes across all known genotypes. In some embodiments, such oligonucleotides result in more than 90% reduction of HBV pre-genomic RNA (pgRNA) and HBsAg mRNAs in liver. In some embodiments, the reduction in HBsAg expression persists for an extended period of time following a single dose or treatment regimen.

Accordingly, in some embodiments, oligonucleotides provided herein are designed so as to have regions of complementarity to HBsAg mRNA for purposes of targeting the transcripts in cells and inhibiting their expression. The region of complementarity is generally of a suitable length and base content to enable annealing of the oligonucleotide (or a strand thereof) to HBsAg mRNA for purposes of inhibiting its expression. In some embodiments, the region of complementarity is at least 12, at least 13, at least 14, at least 15, at least 16, at least 17, at least 18, at least 19 or at least 20 nucleotides in length. In some embodiments, an oligonucleotide provided herein has a region of complementarity to HBsAg mRNA that is in the range of 12 to 30 (e.g., 12 to 30, 12 to 22, 15 to 25, 17 to 21, 18 to 27, 19 to 27, or 15 to 30) nucleotides in length. In some embodiments, an oligonucleotide provided herein has a region of complementarity to HBsAg mRNA that is 12, 13, 14, 15, 16, 17, 18, 19, 20, 21, 22, 23, 24, 25, 26, 27, 28, 29 or 30 nucleotides in length.

In some embodiments, oligonucleotides provided herein are designed to target mRNA sequences encoding HBsAg. For example, in some embodiments, an oligonucleotide is provided that has an antisense strand having a region of complementarity to a sequence set forth as: ACAANAAUCCUCACAAUA (SEQ ID NO: 33), which N refers to any nucleotide (A, G, T, or C). In some embodiments, the oligonucleotide further comprises a sense strand that forms a duplex region with the antisense strand. In some embodiments, the sense strand has a region of complementarity to a sequence set forth as: UUNUUGUGAGGAUUN (SEQ ID NO: 34). In some embodiments, the sense strand comprises a region of complementarity to a sequence as set forth in (shown 5′ to 3′): UUAUUGUGAGGAUUNUUGUC (SEQ ID NO: 35).

In some embodiments, the antisense strand comprises, or consists of, a sequence set forth as: UUAUUGUGAGGAUUNUUGUCGG (SEQ ID NO: 36). In some embodiments, the antisense strand comprises, or consists of, a sequence set forth as: UUAUUGUGAGGAUUCUUGUCGG (SEQ ID NO: 37). In some embodiments, the antisense strand comprises, or consists of, a sequence set forth as: UUAUUGUGAGGAUUUUUGUCGG (SEQ ID NO: 38). In some embodiments, the sense strand comprises, or consists of, a sequence set forth as: ACAANAAUCCUCACAAUAA (SEQ ID NO: 39). In some embodiments, the sense strand comprises, or consists of, a sequence set forth as: GACAANAAUCCUCACAAUAAGCAGCCGAAAGGCUGC (SEQ ID NO: 40). In some embodiments, the sense strand comprises, or consists of, a sequence set forth as: GACAAAAAUCCUCACAAUAAGCAGCCGAAAGGCUGC (SEQ ID NO: 41). In some embodiments, the sense strand comprises, or consists of, a sequence set forth as: GACAAGAAUCCUCACAAUAAGCAGCCGAAAGGCUGC (SEQ ID NO: 42).

In some embodiments, an oligonucleotide for reducing expression of HBsAg mRNA comprises a sense strand forming a duplex region with an antisense strand, where the sense strand comprises a sequence as set forth in any one of SEQ ID NOs: 39-42, and the antisense strand comprises a sequence as set forth in any one of SEQ ID NOs: 36-38. In some embodiments, the sense strand comprises 2′-fluoro and 2′-O-methyl modified nucleotides and at least one phosphorothioate internucleotide linkage. In some embodiments, the sense strand is conjugated to an N-acetylgalactosamine (GalNAc) moiety. In some embodiments, the antisense strand comprises 2′-fluoro and 2′-O-methyl modified nucleotides and at least one phosphorothioate internucleotide linkage. In some embodiments, the 4′-carbon of the sugar of the 5′-nucleotide of the antisense strand comprises a phosphate analog. In some embodiments, each of the antisense strand and the sense strand comprises 2′-fluoro and 2′-O-methyl modified nucleotides and at least one phosphorothioate internucleotide linkage, where the 4′-carbon of the sugar of the 5′-nucleotide of the antisense strand comprises a phosphate analog, and the sense strand is conjugated to an N-acetylgalactosamine (GalNAc) moiety.

In some embodiments, a sense strand comprising a sequence as set forth in any one of SEQ ID NOs: 40-42 comprises 2′-fluoro modified nucleotides at positions 3, 8-10, 12, 13, and 17. In some embodiments, the sense strand comprises 2′-O-methyl modified nucleotides at positions 1, 2, 4-7, 11, 14-16, 18-26, and 31-36. In some embodiments, the sense strand comprises one phosphorothioate internucleotide linkage. In some embodiments, the sense strand comprises a phosphorothioate internucleotide linkage between nucleotides at positions 1 and 2. In some embodiments, the sense strand is conjugated to an N-acetylgalactosamine (GalNAc) moiety.

In some embodiments, an antisense strand comprising a sequence as set forth in any one of SEQ ID NOs: 36-38 comprises 2′-fluoro modified nucleotides at positions 2, 3, 5, 7, 8, 10, 12, 14, 16, and 19. In some embodiments, the antisense strand comprises 2′-O-methyl modified nucleotides at positions 1, 4, 6, 9, 11, 13, 15, 17, 18, and 20-22. In some embodiments, the antisense strand comprises three phosphorothioate internucleotide linkages. In some embodiments, the antisense strand comprises phosphorothioate internucleotide linkages between nucleotides at positions 1 and 2, between nucleotides at positions 2 and 3, between nucleotides at positions 3 and 4, between nucleotides at positions 20 and 21, and between nucleotides at positions 21 and 22. In some embodiments, the 4′-carbon of the sugar of the 5′-nucleotide of the antisense strand comprises a phosphate analog.

I. Double-Stranded Oligonucleotides for Targeting HBsAg mRNA

There are a variety of structures of oligonucleotides that are useful for targeting HBsAg mRNA expression in the pharmaceutical combinations of the present disclosure, including RNAi, antisense, miRNA, etc. Any of the structures described herein or elsewhere may be used as a framework to incorporate or target a sequence described herein. Double-stranded oligonucleotides for targeting HBV antigen expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked.

In some embodiments of the present invention, double-stranded oligonucleotides for reducing the expression of HBsAg mRNA expression engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions (on one or both sides of the molecule) as well as double-stranded extensions.

In some embodiments, oligonucleotides provided herein are cleavable by Dicer enzymes. Such oligonucleotides may have an overhang (e.g., of 1, 2, or 3 nucleotides in length) in the 3′ end of the sense strand. Such oligonucleotides (e.g., siRNAs) may comprise a 21 nucleotide guide strand that is antisense to a target RNA and a complementary passenger strand, in which both strands anneal to form a 19-bp duplex and 2 nucleotide overhangs at either or both 3′ ends. Longer oligonucleotide designs are also available including oligonucleotides having a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. See, for example, U.S. Pat. Nos. 9,012,138, 9,012,621, and 9,193,753, each of which are incorporated herein for their relevant disclosures.

In some embodiments, oligonucleotides as disclosed herein may comprise sense and antisense strands that are both in the range of 17 to 26 (e.g., 17 to 26, 20 to 25, 19 to 21 or 21-23) nucleotides in length. In some embodiments, the sense and antisense strands are of equal length. In some embodiments, for oligonucleotides that have sense and antisense strands that are both in the range of 21-23 nucleotides in length, a 3′ overhang on the sense, antisense, or both sense and antisense strands is 1 or 2 nucleotides in length. In some embodiments, the oligonucleotide has a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. In some embodiments, an oligonucleotide comprises a 25 nucleotide sense strand and a 27 nucleotide antisense strand that when acted upon by a dicer enzyme results in an antisense strand that is incorporated into the mature RISC.

Other oligonucleotide designs for use with the compositions and methods disclosed herein include: 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al. Methods Mol. Biol. 2010; 629:141-158), blunt siRNAs (e.g., of 19 bps in length; see: e.g., Kraynack and Baker, RNA Vol. 12, p 163-176 (2006)), asymmetrical siRNAs (aiRNA; see, e.g., Sun et al., Nat. Biotechnol. 26, 1379-1382 (2008)), asymmetric shorter-duplex siRNA (see, e.g., Chang et al., Mol Ther. 2009 April; 17(4): 725-32), fork siRNAs (see, e.g., Hohjoh, FEBS Letters, Vol 557, issues 1-3; January 2004, p 193-198), single-stranded siRNAs (Elsner; Nature Biotechnology 30, 1063 (2012)), dumbbell-shaped circular siRNAs (see, e.g., Abe et al. J Am Chem Soc 129: 15108-15109 (2007)), and small internally segmented interfering RNA (sisiRNA; see, e.g., Bramsen et al., Nucleic Acids Res. 2007 September; 35(17): 5886-5897). Each of the foregoing references is incorporated by reference in its entirety for the related disclosures therein. Further non-limiting examples of oligonucleotide structures that may be used in some embodiments in a pharmaceutical combination to reduce or inhibit the expression of HBsAg are microRNA (miRNA), short hairpin RNA (shRNA), and short siRNA (see, e.g., Hamilton et al., Embo J., 2002, 21(17): 4671-4679; see also U.S. Application No. 20090099115).
a. Antisense Strands

In some embodiments, an antisense strand of an oligonucleotide may be referred to as a “guide strand”. For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments, a sense strand complementary with a guide strand may be referred to as a “passenger strand”.

In some embodiments, an oligonucleotide provided herein comprises an antisense strand that is up to 50 nucleotides in length (e.g., up to 30, up to 27, up to 25, up to 21, or up to 19 nucleotides in length). In some embodiments, an oligonucleotide provided herein comprises an antisense strand that is at least 12 nucleotides in length (e.g., at least 12, at least 15, at least 19, at least 21, at least 25, or at least 27 nucleotides in length). In some embodiments, an antisense strand of an oligonucleotide disclosed herein is in the range of 12 to 50 or 12 to 30 (e.g., 12 to 30, 11 to 27, 11 to 25, 15 to 21, 15 to 27, 17 to 21, 17 to 25, 19 to 27, or 19 to 30) nucleotides in length. In some embodiments, an antisense strand of any one of the oligonucleotides disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the antisense strand comprises a region of complementarity to a sequence as set forth in (shown 5′ to 3′): AATCCTCACA (SEQ ID NO: 43). In some embodiments, the antisense strand comprises a sequence as set forth in (shown 5′ to 3′): UGUGAGGAUU (SEQ ID NO: 44). In some embodiments, the antisense strand comprises a sequence as set forth in (shown 5′ to 3′): TGTGAGGATT (SEQ ID NO: 45).

In some embodiments, an oligonucleotide for reducing expression of HBsAg mRNA can comprise an antisense strand having a region of complementarity to a sequence as set forth in SEQ ID NO: 43, and one or two non-complementary nucleotides at its 3′ terminus. In some embodiments, the antisense strand comprises the nucleotide sequence set forth in any one of SEQ ID NOs: 36-38.

In some embodiments, an oligonucleotide for reducing expression of HBsAg mRNA can comprise an antisense strand that has a region of complementarity to a sequence as set forth in SEQ ID NO: 43, where the antisense strand does not have a sequence as set forth in any one of the following (shown 5′ to 3′): TATTGTGAGGATTCTTGTCA (SEQ ID NO: 46); CGGTATTGTGAGGATTCTTG (SEQ ID NO: 47); TGTGAGGATTCTTGTCAACA (SEQ ID NO: 48); UAUUGUGAGGAUUUUUGUCAA (SEQ ID NO: 49); UGCGGUAUUGUGAGGAUUCTT (SEQ ID NO: 50); ACAGCATTGTGAGGATTCTTGTC (SEQ ID NO: 51); UAUUGUGAGGAUUUUUGUCAACA (SEQ ID NO: 52); AUUGUGAGGAUUUUUGUCAACAA (SEQ ID NO: 53); and UUGUGAGGAUUUUUGUCAACAAG (SEQ ID NO: 54). In some embodiments, the antisense strand differs from the nucleotide sequence set forth in SEQ ID NOs: 36, 37, or 38 by no more than three nucleotides.
b. Sense Strands

In some embodiments, a double-stranded oligonucleotide may have a sense strand of up to 40 nucleotides in length (e.g., up to 40, up to 35, up to 30, up to 27, up to 25, up to 21, up to 19, up to 17, or up to 12 nucleotides in length). In some embodiments, an oligonucleotide may have a sense strand of at least 12 nucleotides in length (e.g., at least 12, at least 15, at least 19, at least 21, at least 25, at least 27, at least 30, at least 35, or at least 38 nucleotides in length). In some embodiments, an oligonucleotide may have a sense strand in a range of 12 to 50 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a sense strand of an oligonucleotide is longer than 27 nucleotides (e.g., 28, 29, 30, 31, 32, 33, 34, 35, 36, 37, 38, 39 or 40 nucleotides). In some embodiments, a sense strand of an oligonucleotide is longer than 25 nucleotides (e.g., 26, 27, 28, 29 or 30 nucleotides).

In some embodiments, a sense strand comprises a stem-loop at its 3′-end. In some embodiments, a sense strand comprises a stem-loop at its 5′-end. In some embodiments, a strand comprising a stem loop is in the range of 2 to 66 nucleotides long (e.g., 2 to 66, 10 to 52, 14 to 40, 2 to 30, 4 to 26, 8 to 22, 12 to 18, 10 to 22, 14 to 26, or 14 to 30 nucleotides long). In some embodiments, a strand comprising a stem loop is 8, 9, 10, 11, 12, 13, 14, 15, 16, 17, 18, 19, 20, 21, 22, 23, 24, 25, 26, 27, 28, 29, or 30 nucleotides in length. In some embodiments, a stem comprises a duplex of 1, 2, 3, 4, 5, 6, 7, 8, 9, 10, 11, 12, 13, or 14 nucleotides in length. In some embodiments, a stem-loop provides the molecule better protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S₁ and S₂ of up to 10 nucleotides in length (e.g., 1, 2, 3, 4, 5, 6, 7, 8, 9, or 10 nucleotides in length).

In some embodiments, a loop (L) of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides.
c. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 20, 21, 22, 23, 24, 25, 26, 27, 28, 29, or 30 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.
d. Oligonucleotide Ends

In some embodiments, an oligonucleotide comprises sense and antisense strands, such that there is a 3′-overhang on either the sense strand or the antisense strand, or both the sense and antisense strand. In some embodiments, oligonucleotides provided herein have one 5′ end that is thermodynamically less stable compared to the other 5′ end. In some embodiments, an asymmetry oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of an antisense strand are modified. In some embodiments, the last nucleotide at the 3′ end of an antisense strand is modified, e.g., comprises 2′-modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, a double stranded oligonucleotide is provided that has a nicked tetraloop structure at the 3′ end sense strand, and two terminal overhang nucleotides at the 3′ end of its antisense strand. In some embodiments, the two terminal overhang nucleotides are GG. Typically, one or both of the two terminal GG nucleotides of the antisense strand is or are not complementary with the target.

In some embodiments, the 5′ end and/or the 3′ end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.
e. Mismatches

In some embodiments, an oligonucleotide may have one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′ terminus of the sense strand. In some embodiments, base mismatches or destabilization of segments at the 3′-end of the sense strand of the oligonucleotide improved the potency of synthetic duplexes in RNAi, possibly through facilitating processing by Dicer.

In some embodiments, an antisense strand may have a region of complementarity to an HBsAg transcript that contains one or more mismatches compared with a corresponding transcript sequence. A region of complementarity on an oligonucleotide may have up to 1, up to 2, up to 3, up to 4, up to 5, etc. mismatches provided that it maintains the ability to form complementary base pairs with the transcript under appropriate hybridization conditions. Alternatively, a region of complementarity of an oligonucleotide may have no more than 1, no more than 2, no more than 3, no more than 4, or no more than 5 mismatches provided that it maintains the ability to form complementary base pairs with HBsAg mRNA under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with HBsAg mRNA under appropriate hybridization conditions.

II. Single-Stranded Oligonucleotides

In some embodiments, an RNAi oligonucleotide for reducing HBsAg expression as described herein is a single-stranded oligonucleotide having complementarity with HBsAg mRNA. Such structures may include, but are not limited to single-stranded RNAi oligonucleotides. Recent efforts have demonstrated the activity of single-stranded RNAi oligonucleotides (see, e.g., Matsui et al. (May 2016), Molecular Therapy, Vol. 24(5), 946-955).

While such a single-stranded RNAi oligonucleotide may technically be considered an antisense oligonucleotide, it can still function through the mechanism of RNA interference and will have the characteristics as described herein for an RNAi oligonucleotide.

2. Specific RNAi Oligonucleotides of the Invention

For ease of reference, and to avoid unnecessary repetition, the definitions of some of the RNAi oligonucleotides of the present invention set forth herein are also referred to by the following “RNAi ID NOs”.

In one embodiment, the RNAi oligonucleotide in the pharmaceutical combination of the present invention is an oligonucleotide targeting HBV. This RNAi oligonucleotide is also referred to herein as RNAi ID NO: 1.

In one embodiment, the RNAi oligonucleotide in the pharmaceutical combination of the present invention is an oligonucleotide targeting HBsAg mRNA. This RNAi oligonucleotide is also referred to herein as RNAi ID NO: 2.

In one embodiment, the RNAi oligonucleotide in the pharmaceutical combination of the present invention is an oligonucleotide which reduces expression of HBsAg mRNA. This RNAi oligonucleotide is also referred to herein as RNAi ID NO: 3.

In one embodiment the RNAi oligonucleotide in the pharmaceutical combination of the present invention is an oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33). This RNAi oligonucleotide is also referred to herein as RNAi ID NO: 4.

In one embodiment the RNAi oligonucleotide in the pharmaceutical combination of the present invention is an oligonucleotide for reducing expression of HBsAg mRNA, the oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33). This RNAi oligonucleotide is also referred to herein as RNAi ID NO: 5.

In one embodiment the RNAi oligonucleotide in the pharmaceutical combination of the present invention is an oligonucleotide for reducing expression of hepatitis B virus surface antigen (HBsAg) mRNA, the oligonucleotide comprising a sense strand forming a duplex region with an antisense strand, wherein:

the sense strand consists of a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein each of the nucleotides of the -GAAA-sequence on the sense strand is conjugated to a monovalent GaINac moiety; and

the antisense strand consists of a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,

wherein the 4′-carbon of the sugar of the 5′-nucleotide of the antisense strand comprises a methoxy phosphonate (MOP). This RNAi oligonucleotide is also referred to herein as RNAi ID NO: 6.

In one embodiment the RNAi oligonucleotide in the pharmaceutical combination of the present invention is an oligonucleotide comprising a sense strand forming a duplex region with an antisense strand, wherein:

the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GaINac moiety, wherein the -GAAA- sequence comprises the structure:

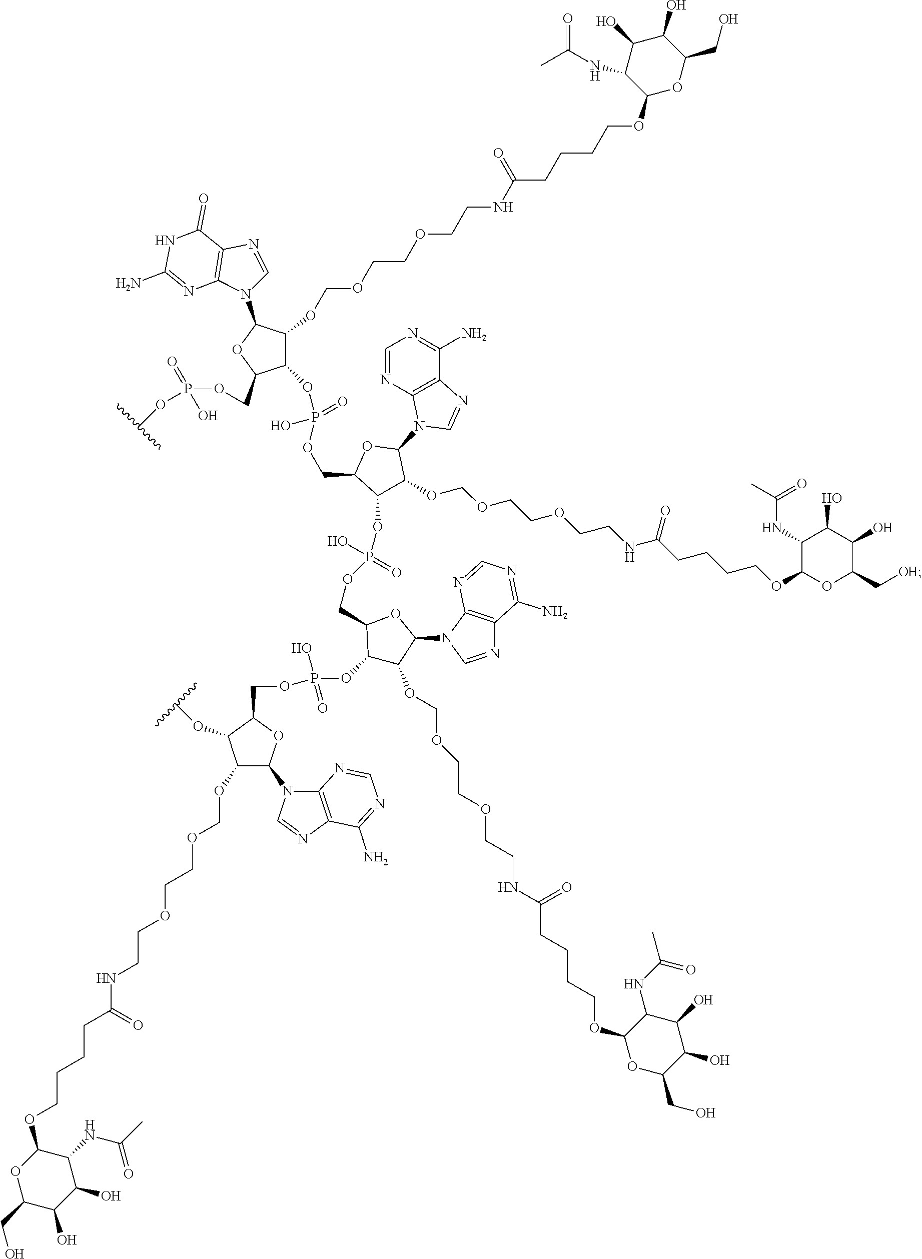 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)C2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and

the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

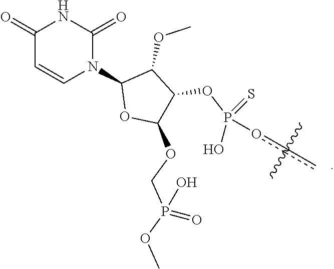

This RNAi oligonucleotide is also referred to herein as RNAi ID NO: 7. In one embodiment RNAi ID NO: 7 is an oligonucleotide for reducing expression of HBsAg mRNA. In one embodiment the sense strand or the antisense strand or both the antisense and sense strands of RNAi ID NO: 7 consist of the respective sequences described above for these strands in RNAi ID NO: 7. In one embodiment in RNAi ID NO: 7, SEQ ID NO: 41 is 5′-GACAAAAAUCCUCACAAUAAGCAGCCGAAAGGCUGC-3′ and/or SEQ ID NO: 38 is 5′-UUAUUGUGAGGAUUUUUGUCGG-3′.

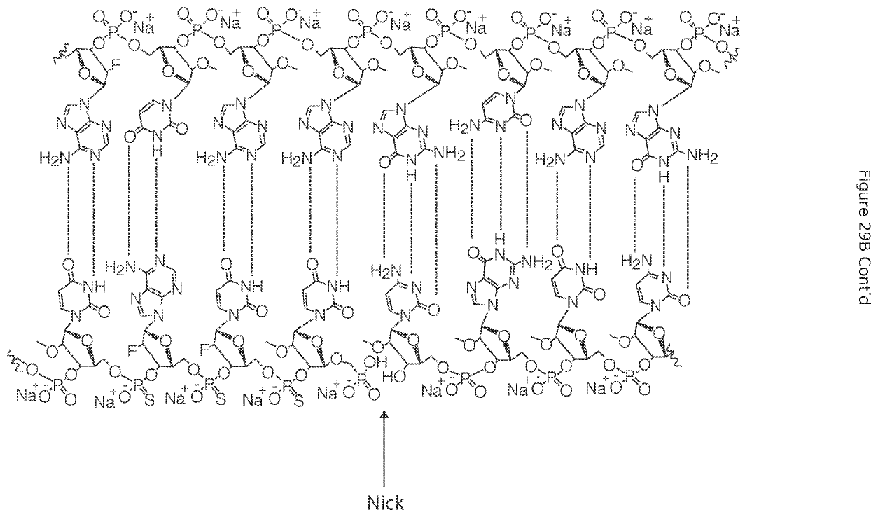

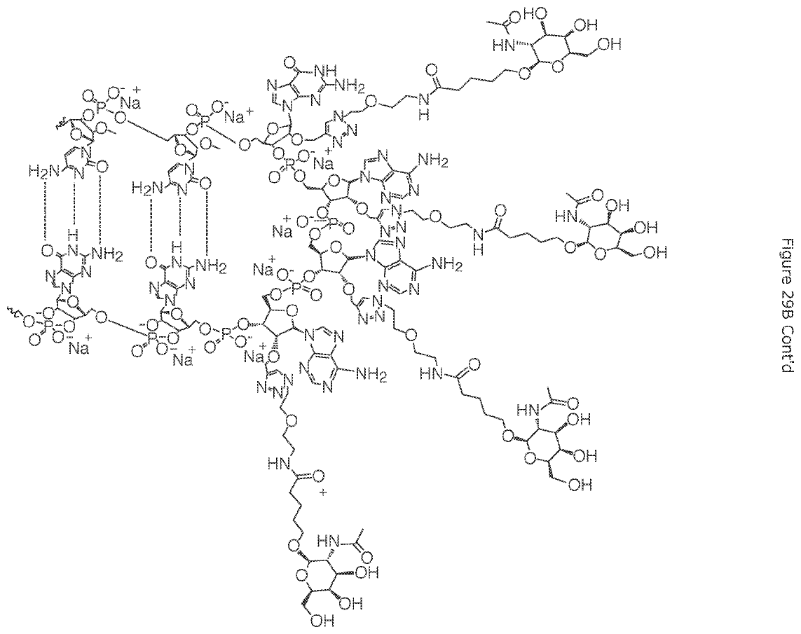

In one embodiment the RNAi oligonucleotide in the pharmaceutical combination of the present invention has the structure depicted in FIG. 29A. This RNAi oligonucleotide is also referred to herein as RNAi ID NO: 8.

In one embodiment the RNAi oligonucleotide in the pharmaceutical combination of the present invention is the oligonucleotide HBV(s)-219. This RNAi oligonucleotide is also referred to herein as RNAi ID NO: 9.

3. Oligonucleotide Modifications of the RNAi Agent of the Invention The modifications discussed in this section are especially preferable for implementation in the RNAi oligonucleotide of the present invention.

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., Nucleic Acids Res., 2009, 37, 2867-2881; Bramsen and Kjems (Frontiers in Genetics, 3 (2012): 1-22). Accordingly, in some embodiments, therapeutic oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of its nucleotides to be modified. Accordingly, in certain embodiments of any of the therapeutic oligonucleotides provided herein, all or substantially all of the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

I. Sugar Modifications

In some embodiments, a modified sugar (also referred to herein as a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al. (1998), Tetrahedron 54, 3607-3630), unlocked nucleic acids (“UNA”) (see, e.g., Snead et al. (2013), Molecular Therapy—Nucleic Acids, 2, e103), and bridged nucleic acids (“BNA”) (see, e.g., Imanishi and Obika (2002), The Royal Society of Chemistry, Chem. Commun., 1653-1659). Koshkin et al., Snead et al., and Imanishi and Obika are incorporated by reference herein for their disclosures relating to sugar modifications.

In some embodiments, a nucleotide modification in a sugar comprises a 2′-modification. A 2′-modification may be 2′-aminoethyl, 2′-fluoro, 2′-O-methyl, 2′-O-methoxyethyl, and 2′-deoxy-2′-fluoro-8-d-arabinonucleic acid. Typically, the modification is 2′-fluoro, 2′-O-methyl, or 2′-O-methoxyethyl. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the terminal 3′-end group (e.g., a 3′-hydroxyl) is a phosphate group or other group, which can be used, for example, to attach linkers, adapters or labels or for the direct ligation of an oligonucleotide to another nucleic acid.

II. 5′ Terminal Phosphates

In some embodiments, 5′-terminal phosphate groups of oligonucleotides enhance the interaction with Argonaut 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to a chemical moiety that mimics the electrostatic and steric properties of a natural 5′-phosphate group (“phosphate mimic”) (see, e.g., Prakash et al. (2015), Nucleic Acids Res., Nucleic Acids Res. 2015 Mar. 31; 43(6): 2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”). See, for example, U.S. Provisional Application numbers 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or—O—CH₂—PO(OR)₂, in which R is independently selected from H, CHs, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CHs, or CH₂CH₃.

In certain embodiments, a phosphate analog attached to the oligonucleotide is a methoxy phosphonate (MOP). In certain embodiments, a phosphate analog attached to the oligonucleotide is a 5′ mono-methyl protected MOP. In some embodiments, the following uridine nucleotide comprising a phosphate analog may be used, e.g., at the first position of a guide (antisense) strand:

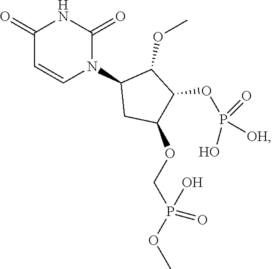 [SMILES: CO[C@@H]1[C@H](OP(=O)(O)O)[C@@H](OCP(=O)(O)OC)C[C@H]1n1ccc(=O)[nH]c1=O]

which modified nucleotide is referred to as [MePhosphonate-40-mU] or 5′-Methoxy, Phosphonate-4′oxy- 2′-O-methyluridine.

III. Modified Internucleoside Linkages

In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorothioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

IV. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain a nitrogen atom. See e.g., U.S. Published Patent Application No. 20080274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution that, when present in a duplex, can be positioned opposite more than one type of base without substantially altering the structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside, Nucleic Acids Res. 1995 Nov. 11; 23(21):4363-70; Loakes et al., 3-Nitropyrrole and 5-nitroindole as universal bases in primers for DNA sequencing and PCR, Nucleic Acids Res. 1995 Jul. 11; 23(13):2361-6; Loakes and Brown, 5-Nitroindole as an universal base analogue, Nucleic Acids Res. 1994 Oct. 11; 22(20):4039-43. Each of the foregoing is incorporated by reference herein for their disclosures relating to base modifications).

V. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”), PCT Publication No. WO 2015/188197 to Solstice Biologics, Ltd. (“Solstice”), Meade et al., Nature Biotechnology, 2014, 32:1256-1263 (“Meade”), PCT Publication No. WO 2014/088920 to Merck Sharp & Dohme Corp, each of which are incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. Am. Chem. Soc. 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of a sugar, particularly when the modified nucleotide is the 3′-terminal nucleotide of the oligonucleotide. In some embodiments, the glutathione-sensitive moiety comprises a sulfonyl group. See, e.g., U.S. Prov. Appl. No. 62/378,635, entitled Compositions Comprising Reversibly Modified Oligonucleotides and Uses Thereof, which was filed on Aug. 23, 2016, the contents of which are incorporated by reference herein for its relevant disclosures.

IV. Targeting Ligands

In some embodiments, it may be desirable to target the oligonucleotides of the disclosure to one or more cells or one or more organs. Such a strategy may help to avoid undesirable effects in other organs, or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand.

In some embodiments, it is desirable to target an oligonucleotide that reduces the expression of HBV antigen to the hepatocytes of the liver of a subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand, where each GalNAc moiety is conjugated to one nucleotide.

In some embodiments, an oligonucleotide herein comprises a monovalent GalNAc attached to a Guanidine nucleotide, referred to as [ademG-GalNAc] or 2′-aminodiethoxymethanol-Guanidine-GalNAc, as depicted below:

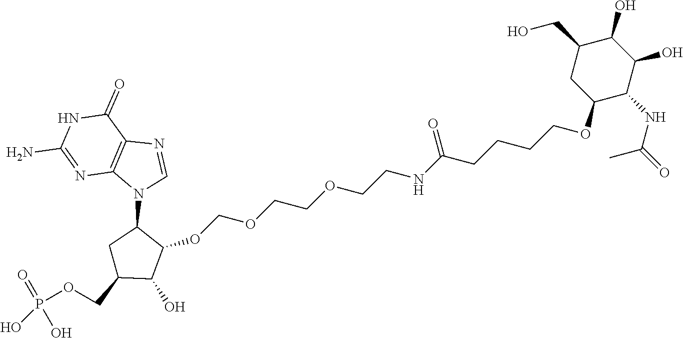 [SMILES: CC(=O)N[C@H]1[C@H](O)[C@H](O)[C@H](CO)C[C@@H]1OCCCCC(=O)NCCOCCOCO[C@@H]1[C@H](O)[C@@H](COP(=O)(O)O)C[C@H]1n1cnc2c(=O)[nH]c(N)nc21]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

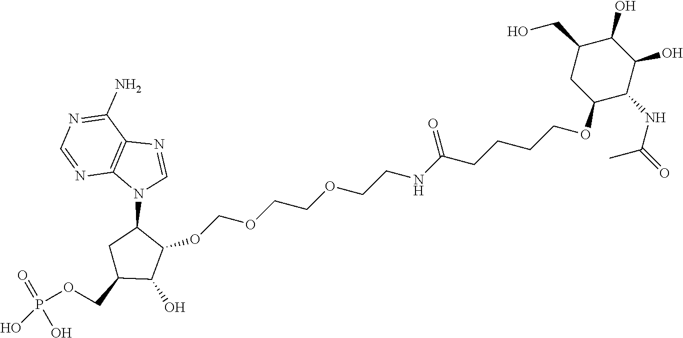

An example of such conjugation is shown below for a loop comprising from 5′ to 3′ the nucleotide sequence GAAA (L=linker, X=heteroatom) stem attachment points are shown. Such a loop may be present, for example, at positions 27-30 of the molecule shown in FIG. 20. In the chemical formula,

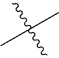

is an attachment point to the oligonucleotide strand.

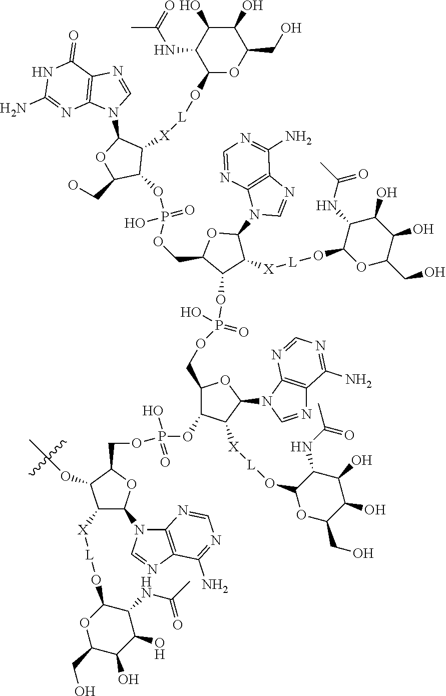

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which relating to such linkers are incorporated herein by reference. In some embodiments, the linker is a labile linker. However, in other embodiments, the linker is fairly stable.

An example is shown below for a loop comprising from 5′ to 3′ the nucleotides GAAA, in which GalNAc moieties are attached to nucleotides of the loop using an acetal linker. Such a loop may be present, for example, at positions 27-30 of the molecule shown in FIG. 20. In the chemical formula,

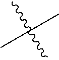

is an attachment point to the oligonucleotide strand.

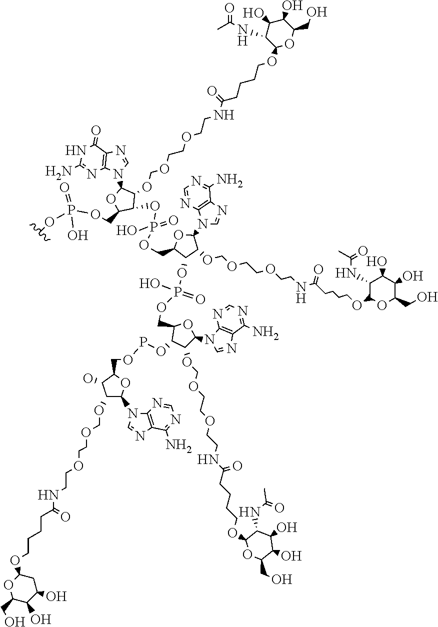

4. Further GalNAc Conjugated Therapeutic Oligonucleotides Targeting HBV

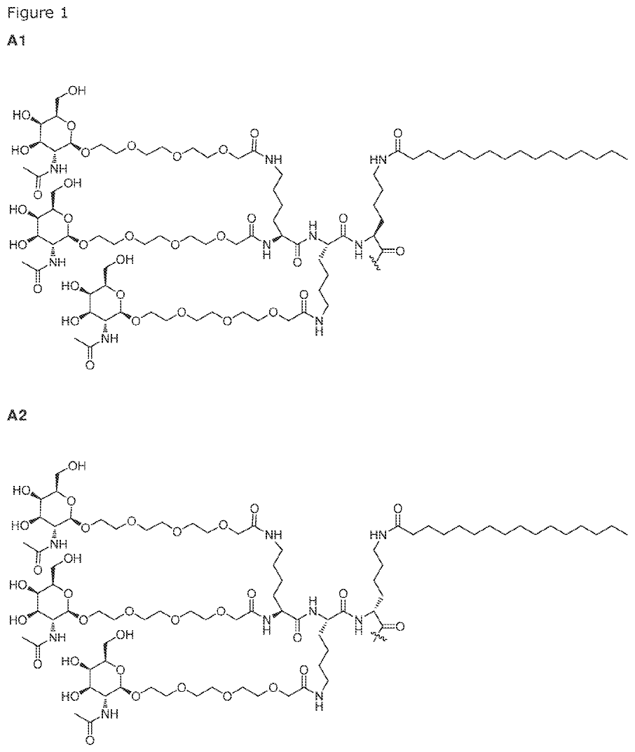

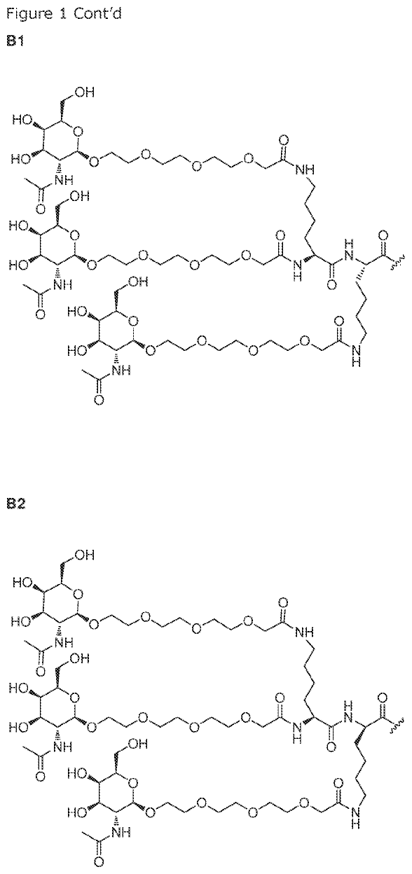

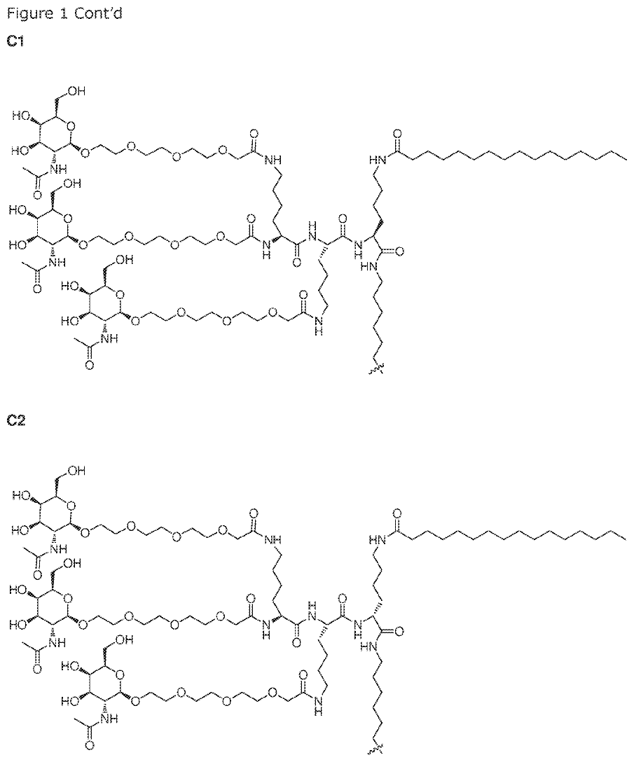

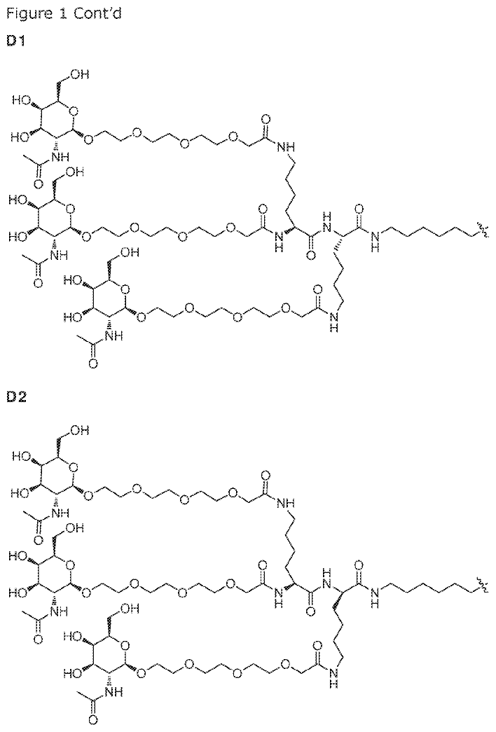

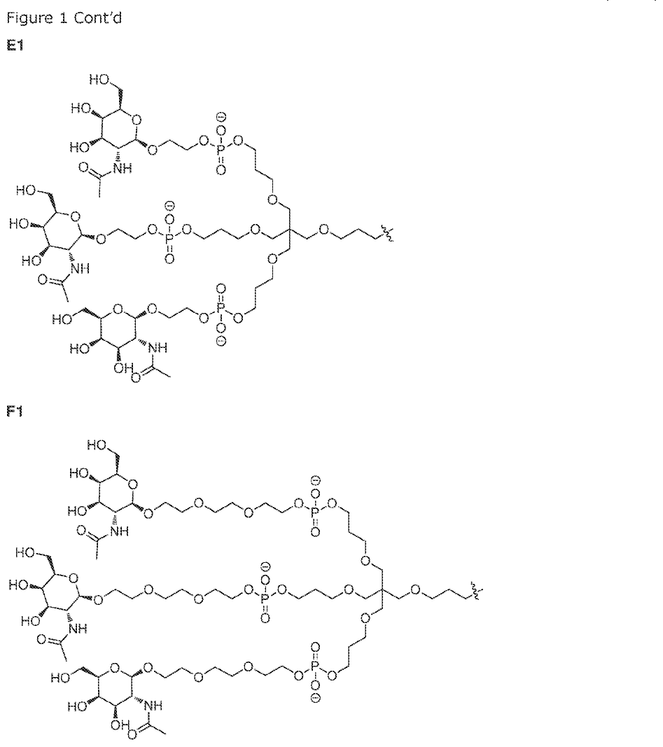

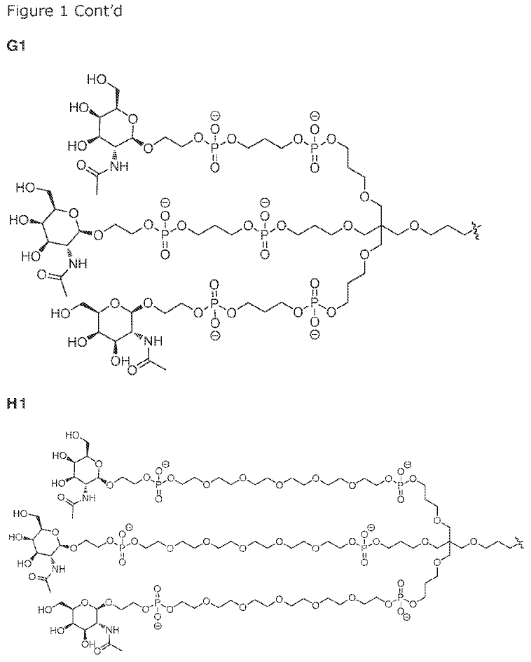

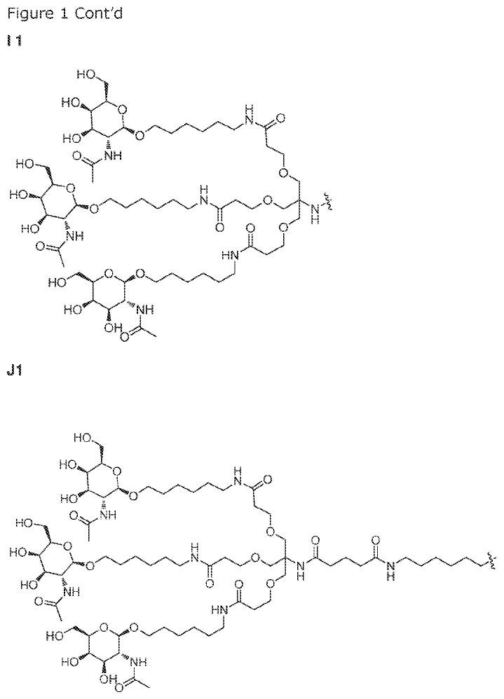

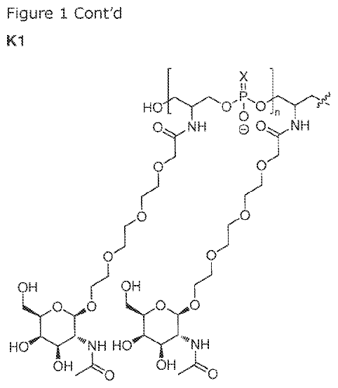

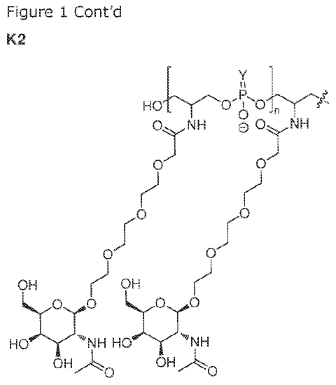

In an embodiment, the oligonucleotide of the invention is a therapeutic oligonucleotide which targets HBV mRNA, and which has improved delivery to the liver, in particular to hepatocytes through conjugation to an asialoglycoprotein receptor (ASGPR) targeting conjugate such as a di-valent, tri-valent or tetra-valent GalNAc cluster (illustrative examples in FIG. 1). WO2015/173208 describes such GalNAc conjugated antisense oligonucleotides targeting HBV mRNA (SEQ ID NO: 1) and their production.

The GalNAc conjugated therapeutic oligonucleotide in the pharmaceutical combination of the invention is capable of reducing the expression from HBV mRNA (the target nucleic acid), in particular the expression of HBsAg and HBx of Hepatitis B virus, both encoded from SEQ ID NO: 1. Furthermore, the GalNAc conjugated therapeutic oligonucleotide of the invention is preferably capable of reducing HBsAg expression from chromosomally integrated HBV fragments.

In some embodiments, the GalNAc conjugated therapeutic oligonucleotide of the invention binds to the target nucleic acid and reduces expression by at least 10% or 20% compared to the normal expression level, more preferably at least 30%, 40%, 50%, 60%, 70%, 80%, 90% or 95% compared to the normal expression level (such as the expression level in the absence of the GalNAc conjugated therapeutic oligonucleotide).

In one embodiment, the GalNAc conjugated therapeutic oligonucleotide of the invention is capable of down-regulating (e.g. inhibiting, reducing or removing) expression of the HBx or HBsAg gene. Such down-regulation may typically occur in a target cell, such as a mammalian cell such as a human cell, such as a liver cell, such as a hepatocyte, in particular in an HBV infected hepatocyte. In some embodiments, the GalNAc conjugated therapeutic oligonucleotides of the invention bind to the target nucleic acid and affect inhibition of expression of at least 50% compared to the normal expression level, more preferably at least 60%, 70%, 80%, 90% or 95% inhibition compared to the normal expression level (such as the expression level in the absence of the GalNAc conjugated therapeutic oligonucleotide). Modulation of expression levels of HBV mRNA and HBsAg and HBV DNA may be determined using the methods described in the Materials and Methods section.

An aspect of the present invention relates to a therapeutic oligonucleotide which comprises a contiguous nucleotide sequence of 12 to 30 nucleotides in length with at least 90% complementarity to position 1530 to 1602 of SEQ ID NO: 1.

In one embodiment of the present invention the therapeutic oligonucleotide is complementary to a sequence selected from position 1530 to 1602; 1530 to 1598; 1530-1543; 1530-1544; 1531-1543; 1551-1565; 1551-1566; 1577-1589; 1577-1591; 1577-1592; 1578-1590; 1578-1592; 1583-1598; 1584-1598; 1585-1598 and 1583-1602 of SEQ ID NO: 1. In particular therapeutic oligonucleotides with 100% complementarity to the target sequences from position 1530-1544, 1531-1543, 1583-1602 and 1583-1598 are advantageous.

In some embodiments, the therapeutic oligonucleotide comprises a contiguous sequence of 12 to 30 nucleotides in length, which is at least 91% complementary, such as at least 92%, such as at least 93%, such as at least 94%, such as at least 95%, such as at least 96%, such as at least 97%, such as at least 98%, 99% or 100% complementary with a region of the target nucleic acid or a target sequence.

It is advantageous if the contiguous nucleotide sequence is fully complementary (100% complementary) to a contiguous sequence in a target sequence selected from the group consisting of position 1530 to 1602; 1530 to 1598; 1530-1543; 1530-1544; 1531-1543; 1551-1565; 1551-1566; 1577-1589; 1577-1591; 1577-1592; 1578-1590; 1578-1592; 1583-1598; 1584-1598; 1585-1598 or 1583-1602 of SEQ ID NO: 1, or in some embodiments may comprise one or two mismatches between the therapeutic oligonucleotide and the target sequence.

In an embodiment of the present invention the GalNAc conjugated antisense oligonucleotide is of 13 to 20 nucleotides in length with a contiguous nucleotide sequence of at least 12 nucleotides which is 100% complementary to a contiguous sequence from position 1530 to 1602 of SEQ ID NO: 1 or SEQ ID NO: 28. It is understood that this compound is combined with a TLR7 agonist as described in the section relating to TLR7 agonists

In some embodiments, the antisense oligonucleotide of the invention comprises or consists of 13 to 24 nucleotides in length, such as from 13 to 22, such as 14 to 20 contiguous nucleotides in length. In a preferred embodiment, the antisense oligonucleotide comprises or consists of 13 to 18, such as from 15 to 18 nucleotides in length.

In some embodiments, the contiguous nucleotide sequence thereof comprises or consists of 12-20 nucleotides, such as 12 to 18, such as 13 to 17, such as 13 to 15 nucleotides in length, such as 13, 14, 15, 16 or 17 nucleotides in length. It is to be understood that the contiguous nucleotide sequence is always equal to or shorter than the total length of the antisense oligonucleotide since the antisense oligonucleotide may comprise additional nucleosides serving as for example biocleavable linker between the contiguous nucleotide sequence and the conjugate. It is also understood that any range given herein includes the range endpoints. Accordingly, if an antisense oligonucleotide is said to include from 12 to 30 nucleotides, both 12 and 30 nucleotides are included.

In some embodiments, the contiguous nucleotide sequence comprises or consists of a sequence selected from the group consisting of

(SEQ ID NO: 2)
gcgtaaagagagg;
(SEQ ID NO: 3)
gcgtaaagagaggt;
(SEQ ID NO 4)
cgcgtaaagagaggt;
(SEQ ID NO 5)
agaaggcacagacgg;
(SEQ ID NO 6)
gagaaggcacagacgg;
(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc;
(SEQ ID NO: 13)
aggtgaagcgaagtg;
(SEQ ID NO 14)
aggtgaagcgaagt;
and
(SEQ ID NO: 29)
gcagaggtgaagcgaagtgc.

In some embodiments, the antisense oligonucleotide comprises or consists of 12 to 22 nucleotides in length with a contiguous nucleotide sequence of at least 12 nucleotides with at least 90% identity, preferably 100% identity, to a sequence selected from the group consisting of

(SEQ ID NO: 2)
gcgtaaagagagg;
(SEQ ID NO: 3)
gcgtaaagagaggt;
and
(SEQ ID NO 4)
cgcgtaaagagaggt.

In some embodiments, the antisense oligonucleotide comprises or consists of 12 to 22 nucleotides in length with a contiguous nucleotide sequence of at least 12 nucleotides with at least 90% identity, preferably 100% identity, to a sequence selected from

(SEQ ID NO 5)
agaaggcacagacgg;
or
(SEQ ID NO 6)
gagaaggcacagacgg.

In some embodiments, the antisense oligonucleotide comprises or consists of 12 to 22 nucleotides in length with a contiguous nucleotide sequence of at least 12 nucleotides with at least 90% identity, preferably 100% identity, to a sequence selected from the group consisting of

(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc.

In some embodiments, the antisense oligonucleotide comprises or consists of 12 to 22 nucleotides in length with a contiguous nucleotide sequence of at least 12 nucleotides with at least 90% identity, preferably 100% identity, to a sequence selected from the group consisting of

(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc.

5. Oligonucleotide Modifications for the Antisense Oligonucleotide of the Invention

The modifications discussed in this section are especially preferable for implementation in the antisense oligonucleotide of the present invention.

It is understood that the contiguous nucleobase sequences (motif sequence) can be modified to for example increase nuclease resistance and/or binding affinity to the target nucleic acid.

In one embodiment the contiguous nucleobase sequence of the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are internucleoside linkages. In some embodiments all the internucleotide linkages in the contiguous sequence of the oligonucleotide are phosphorothioate linkages.

The oligonucleotides of the invention are designed with modified nucleosides and DNA nucleosides. Advantageously, high affinity modified nucleosides are used.

In an embodiment, the oligonucleotide comprises at least 3 modified nucleosides, such as at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 3 to 8 modified nucleosides, such as from 4 to 6 modified nucleosides, such as 4, 5 or 6 nucleosides, such as from 5 or 6 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more or all of the modified nucleoside(s) is a locked nucleic acid (LNA).

In some embodiments, the oligonucleotide of the invention, such as the contiguous nucleotide sequence, comprises at least one LNA nucleoside, such as 1, 2, 3, 4, 5, 6, 7, or 8 LNA nucleosides, such as from 2 to 6 LNA nucleosides, such as from 3 to 6 LNA nucleosides, 4 to 6 LNA nucleosides or 4, 5 or 6 LNA nucleosides.

In some embodiments, at least 75% of the modified nucleosides in the oligonucleotide are LNA nucleosides, such as at least 80%, such as at least 85%, such as at least 90% of the modified nucleosides are LNA nucleosides. In a still further embodiment all the modified nucleosides in the oligonucleotide are LNA nucleosides. In a further embodiment, the LNA nucleosides are selected from beta-D-oxy-LNA, thio-LNA, amino-LNA, oxy-LNA, ScET and/or ENA in either the beta-D or alpha-L configurations or combinations thereof. In a further embodiment, all LNA nucleosides are beta-D-oxy-LNA. In a further embodiment cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

6. Antisense Oligonucleotide Design for RNase H Recruitment

In an embodiment of the invention wherein the therapeutic oligonucleotide is an antisense oligonucleotide the oligonucleotide of the invention is capable of recruiting RNase H when hybridized to a target nucleic acid.

The pattern in which the modified nucleosides (such as high affinity modified nucleosides) are incorporated into the oligonucleotide sequence is generally termed oligonucleotide design.

In an embodiment of the current invention wherein the therapeutic oligonucleotide is an antisense oligonucleotide an advantageous structural design is a gapmer design as described in the “Definitions” section under for example “Gapmer”, “LNA Gapmer”, “MOE gapmer” and “Mixed Wing Gapmer”. The gapmer design includes gapmers with uniform flanks and mixed wing flanks. In the present invention it is advantageous if the contiguous nucleotide sequence of the invention is a gapmer with an F-G-F′ design. In some embodiments the gapmer is an LNA or MOE gapmer with the following uniform flank designs 3-7-3, 3-8-2, 3-8-3, 2-9-4, 3-9-3, 3-10-3 or 5-10-5.

In some embodiments, the antisense oligonucleotide comprises or consists of 12 to 22 nucleotides in length with a contiguous nucleotide sequence selected from the group consisting of:

(SEQ ID NO: 2)
GCGtaaagagaGG;
(SEQ ID NO: 2)
GCGtaaagagAGG;
(SEQ ID NO: 3)
GCGtaaagagaGGT;
(SEQ ID NO: 4)
CGCgtaaagagaGGT;
(SEQ ID NO: 5)
AGAaggcacagaCGG;
(SEQ ID NO: 6)
GAGaaggcacagaCGG;
(SEQ ID NO: 7)
AGCgaagtgcacaCGG;
(SEQ ID NO: 8)
GAAgtgcacacGG;
(SEQ ID NO: 8)
GAAgtgcacaCGG;
(SEQ ID NO: 9)
GCGaagtgcacaCGG;
(SEQ ID NO: 10)
AGCgaagtgcacACG;
(SEQ ID NO: 11)
CGAagtgcacaCG;
(SEQ ID NO: 12)
AGGtgaagcgaagTGC;
(SEQ ID NO: 13)
AGGtgaagcgaaGTG
(SEQ ID NO: 13)
AGgtgaagcgaAGTG;
and
(SEQ ID NO: 14)
AGGtgaagcgaAGT;

wherein uppercase letters denote LNA nucleosides, such as beta-D-oxy-LNA, and lower case letters denote DNA nucleosides.

In some embodiments, the antisense oligonucleotide comprises or consists of 12 to 22 nucleotides in length with a contiguous nucleotide sequence selected from the group consisting of:

(SEQ ID NO: 2)
GCGtaaagagaGG;
(SEQ ID NO: 2)
GCGtaaagagAGG;
(SEQ ID NO: 3)
GCGtaaagagaGGT;
and
(SEQ ID NO: 4)
CGCgtaaagagaGGT;

wherein uppercase letters denote LNA nucleosides, such as beta-D-oxy-LNA, and lower case letters denote DNA nucleosides.

In some embodiments, the antisense oligonucleotide comprises or consists of 12 to 22 nucleotides in length with a contiguous nucleotide sequence consists of:

(SEQ ID NO 5)
agaaggcacagacgg;
or
(SEQ ID NO 6)
gagaaggcacagacgg.

wherein uppercase letters denote LNA nucleosides, such as beta-D-oxy-LNA, and lower case letters denote DNA nucleosides.

In some embodiments, the antisense oligonucleotide comprises or consists of 12 to 22 nucleotides in length with a contiguous nucleotide sequence selected from the group consisting of:

(SEQ ID NO: 7)
AGCgaagtgcacaCGG;
(SEQ ID NO: 8)
GAAgtgcacacGG;
(SEQ ID NO: 8)
GAAgtgcacaCGG;
(SEQ ID NO: 9)
GCGaagtgcacaCGG;
(SEQ ID NO: 10)
AGCgaagtgcacACG;
(SEQ ID NO: 11)
CGAagtgcacaCG;
(SEQ ID NO: 12)
AGGtgaagcgaagTGC;
(SEQ ID NO: 13)
AGGtgaagcgaaGTG
(SEQ ID NO: 13)
AGgtgaagcgaAGTG;
and
(SEQ ID NO: 14)
AGGtgaagcgaAGT;

wherein uppercase letters denote LNA nucleosides, such as beta-D-oxy-LNA, and lower case letters denote DNA nucleosides.

In some embodiments, the antisense oligonucleotide comprises or consists of 12 to 22 nucleotides in length with a contiguous nucleotide sequence selected from the group consisting of:

(SEQ ID NO: 12)
AGGtgaagcgaagTGC;
(SEQ ID NO: 13)
AGGtgaagcgaaGTG
(SEQ ID NO: 13)
AGgtgaagcgaAGTG;
and
(SEQ ID NO: 14)
AGGtgaagcgaAGT;

wherein uppercase letters denote LNA nucleosides, such as beta-D-oxy-LNA, and lower case letters denote DNA nucleosides.

In some embodiments, the antisense oligonucleotide comprises or consists of 20 to 24 nucleotides in length with a contiguous nucleotide sequence of

(SEQ ID NO: 29)
GCAGAggtgaagcgaAGTGC

wherein uppercase underlined letters denote MOE nucleosides, and lower case letters denote DNA nucleosides.

Below table 1 is a summary of the motif sequences of the contiguous nucleotide sequences of the antisense oligonucleotides targeting position 1530 to 1602 of SEQ ID NO: 1 as well as gapmer designs of these intended for the use of the present invention.

TABLE 1

| SEQID | Position on | | |
|---|---|---|---|
| NO | SEQ ID NO: 1 | Motif sequence | Designs |
| 2 | 1531-1543 | gcgtaaagagagg | GCGtaaagagaGG |
| | | | GCGtaaagagAGG |
| 3 | 1530-1543 | gcgtaaagagaggt | GCGtaaagagaGGT |
| 4 | 1530-1544 | cgcgtaaagagaggt | CGCgtaaagagaGGT |
| 5 | 1551-1565 | agaaggcacagacgg | AGAaggcacagaCGG |
| 6 | 1551-1566 | gagaaggcacagacgg | GAGaaggcacagaCGG |
| 7 | 1577-1592 | agcgaagtgcacacgg | AGCgaagtgcacaCGG |
| 8 | 1577-1589 | gaagtgcacacgg | GAAgtgcacacGG |
| | | | GAAgtgcacaCGG |
| 9 | 1577-1591 | gcgaagtgcacacgg | GCGaagtgcacaCGG |
| 10 | 1578-1592 | agcgaagtgcacacg | AGCgaagtgcacACG |
| 11 | 1578-1590 | cgaagtgcacacg | CGAagtgcacaCG |
| 12 | 1583-1598 | aggtgaagcgaagtgc | AGGtgaagcgaagTGC |
| 13 | 1584-1598 | aggtgaagcgaagtg | AGGtgaagcgaaGTG |
| | | | AGgtgaagcgaAGTG |
| 14 | 1585-1598 | aggtgaagcgaagt | AGGtgaagcgaAGT |
| 29 | 1583-1602 | gcagaggtgaagcgaagtgc | GCAGAggtgaagcgaAGTGC |

In the Designs column of table 1 upper case letters denote 2′-sugar modified nucleosides, in particular LNA nucleosides, such as beta-D-oxy-LNA, or MOE nucleosides and lowercase letters denote DNA nucleosides. Internucleoside linkages can be phosphodiester or phosphorothioate. In some embodiments all the internucleoside linkages are phosphorothioate.

In all instances the antisense oligonucleotide may further include region D′ and/or D″ at the 5′ or 3′ end of the F-G-F′ design, as described in the “Definitions” section under “Region D′ or D” in an oligonucleotide”. In some embodiments the antisense oligonucleotide of the invention has 1 to 5 such as 1, 2 or 3 phosphodiester linked nucleoside units, such as DNA units, at the 5′ or 3′ end of the gapmer region. The DNA nucleosides generally have nucleobases as defined in the nucleobase definition, such as naturally occurring DNA nucleosides with a nucleobase selected from purine (e.g. adenine and guanine) and pyrimidine (e.g. uracil, thymine and cytosine). In some embodiments the antisense oligonucleotide of the invention consists of two 5′ phosphodiester linked DNA nucleosides followed by a F-G-F′ gapmer region as defined in the “Definitions” section. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties.

In some embodiments, the antisense oligonucleotide comprises or consists of a sequence selected from the group consisting of:

(SEQ ID NO: 15)
cagcgtaaagagagg
(SEQ ID NO: 16)
cagcgtaaagagaggt
(SEQ ID NO: 17)
cacgcgtaaagagaggt
(SEQ ID NO: 18)
caagaaggcacagacgg
(SEQ ID NO: 19)
cagagaaggcacagacgg
(SEQ ID NO: 20)
caagcgaagtgcacacgg
(SEQ ID NO: 21)
cagaagtgcacacgg
(SEQ ID NO: 22)
cagcgaagtgcacacgg
(SEQ ID NO: 23)
caagcgaagtgcacacg
(SEQ ID NO: 24)
cacgaagtgcacacg
(SEQ ID NO: 25)
caaggtgaagcgaagtgc
(SEQ ID NO: 26)
caaggtgaagcgaagtg
(SEQ ID NO: 27)
caaggtgaagcgaagt

wherein the internucleoside linkage between the nucleosides from position 1 to 3 (staring from the 5′ end) are phosphodiester linkages and the internucleoside linkage between the nucleoside in position 3 and 4 is a phosphorothioate linkage (where the nucleoside at position 3 is the 5′ end of the contiguous nucleotide sequence). It is advantages if all the internucleoside linkages after position 4 to the 3′ end of the oligonucleotide are phosphorothioate linkages. In one embodiment the contiguous nucleotide sequence has the design of the corresponding sequence in table 1.

7. Conjugates Binding to Asialoglycoprotein

Conjugates capable of binding to the asialoglycoprotein receptor (ASGPR) are particular useful for targeting hepatocytes in liver. Conjugates comprising at least two carbohydrate moieties selected from group consisting of galactose, galactosamine, N-formyl-galactosamine, N-acetylgalactosamine, N-propionyl-galactosamine, N-n-butanoyl-galactosamine and N-isobutanoylgalactosamine are generally capable of binding the ASGPR. The N-acetylgalactosamine (GalNAc) moiety has shown to be advantageous in targeting the ASGPR, but alternatives from the list above can also be used, e.g. galactose. In one embodiment the conjugate consists of two to four terminal GalNAc moieties linked to a spacer which links each GalNAc moiety to a brancher molecule thereby forming a cluster that can be conjugated to the therapeutic oligonucleotide.

The GalNAc cluster can for example be generated by linking the GalNAc moiety to the spacer through its C—I carbon. A preferred spacer is a flexible hydrophilic spacer (U.S. Pat. No. 5,885,968; Biessen et al. J. Med. Chem. 1995 Vol. 39 p. 1538-1546). A preferred flexible hydrophilic spacer is a PEG spacer. A preferred PEG spacer is a PEG3 spacer. The branch point can be any small molecule which permits attachment of two to three GalNAc moieties (or other asialoglycoprotein receptor targeting moieties) and further permits attachment of the branch point to the oligonucleotide, such constructs are termed GalNAc clusters or GalNAc conjugates. An exemplary branch point group is a di-lysine. A di-lysine molecule contains three amine groups through which three GalNAc moieties or other asialoglycoprotein receptor targeting moieties may be attached and a carboxyl reactive group through which the di-lysine may be attached to the oligomer. Khorev, et al 2008 Bioorg. Med. Chem. Vol 16, pp. 5216 also describes the synthesis of a suitable trivalent brancher. Other commercially available branchers are 1,3-bis-[5-(4,4′-dimethoxytrityloxy)pentylamido]propyl-2-[(2-cyanoethyl)-(N,N-diisopropyl)] phosphoramidite (Glen Research Catalogue Number: 10-1920-xx); tris-2,2,2-[3-(4,4′-dimethoxytrityloxy)propyloxymethyl]ethyl-[(2-cyanoethyl)-(N,N-diisopropyl)]-phosphoramidite (Glen Research Catalogue Number: 10-1922-xx); and tris-2,2,2-[3-(4,4′-dimethoxytrityloxy)propyloxymethyl]methyleneoxypropyl-[(2-cyanoethyl)-(N,N-diisopropyl)]-phosphoramidite; and 1-[5-(4,4′-dimethoxy-trityloxy)pentylamido]-3-[5-fluorenomethoxy-carbonyl-oxy-pentylamido]-propyl-2-[(2-cyanoethyl)-(N,N-diisopropyl)]-phosphoramidite (Glen Research Catalogue Number: 10-1925-xx). Other GalNAc clusters may be small peptides with GalNAc moieties attached such as Tyr-Glu-Glu-(aminohexyl GalNAc)3 (YEE(ahGalNAc)3; a glycotripeptide that binds to asialoglycoprotein receptor on hepatocytes, see, e.g., Duff, et al., Methods Enzymol, 2000, 313, 297; lysine-based galactose clusters (e.g., L3G4; Biessen, et al., Cardovasc. Med., 1999, 214); and cholane-based galactose clusters (e.g., carbohydrate recognition motif for asialoglycoprotein receptor).

In an embodiment of the present invention the therapeutic oligonucleotide of the invention is conjugated to a GalNAc cluster to improve the pharmacology of the oligonucleotide, e.g. by affecting, cellular distribution, in particular the cellular uptake in hepatocytes of the oligonucleotide.

Suitable GalNAc conjugates are those capable of binding to the asialoglycoprotein receptor (ASGPR), such as di-valent, tri-valent or tetra-valent GalNAc clusters. In particular, tri-valent N-acetylgalactosamine conjugates are suitable for binding to the ASGPR, see for example WO 2014/076196, WO 2014/207232, WO 2014/179620, WO 2016/055601 and WO 2017/178656 (hereby incorporated by reference). FIG. 1 is a representation of suitable GalNAc conjugates, which have been subject to at least in vitro testing. Alternative GalNAc conjugates may however also be suitable if they are capable of binding the asialoglycoprotein receptor. Such conjugates serve to enhance uptake of the oligonucleotide to the liver while reducing its presence in the kidney, thereby increasing the liver/kidney ratio of the GalNAc conjugated oligonucleotide compared to the unconjugated version of the same oligonucleotide.

The GalNAc cluster may be attached to the 3′- or 5′-end of the oligonucleotide using methods known in the art. In one embodiment the GalNAc cluster is linked to the 5′-end of the oligonucleotide.

One or more linkers may be inserted between the conjugate (such as at the brancher part of the conjugate moiety) and the oligonucleotide. It is advantageous to have a biocleavable linker between the conjugate moiety and the therapeutic oligonucleotide, optionally in combination with a non-cleavable linker such as a C6 linker. The linker(s) may be selected from the linkers described in the “Definitions” section under “Linkers” in particular biocleavable region D′ or D″ linkers are advantageous. A GalNAc conjugated oligonucleotide with a biocleavable linker between the conjugate and the gapmer or contiguous nucleotide sequence is effectively a prodrug, since the GalNAc cluster and the biocleavable PO linker is removed from the gapmer or contiguous nucleotide sequence upon entry into the cell.

In one embodiment the conjugate moiety is a tri-valent N-acetylgalactosamine (GalNAc), such as those shown in FIG. 1.

In one embodiment wherein the GalNAc conjugated antisense oligonucleotide is selected from the group consisting of:

SEQ ID NO: 15
5′-GN2-C6ₒcₒaₒGₛ^{m}CₛGₛtₛaₛaₛaₛgₛaₛgₛaₛGₛG-3′
SEQ ID NO: 15
5′-GN2-C6ₒcₒaₒGₛ^{m}CₛGₛtₛaₛaₛaₛgₛaₛgₛAₛGₛG-3′
SEQ ID NO: 16
5-GN2-C6ₒcₒaₒGₛ^{m}CₛGₛtₛaₛaₛaₛgₛaₛgₛaₛGₛGₛT-3′
SEQ ID NO: 17
5′-GN2-C6ₒcₒaₒ^{m}CₛGₛ^{m}CₛgₛtₛaₛaₛaₛgₛaₛgₛaₛGₛGₛT-3′
SEQ ID NO: 18
5′-GN2-C6ₒcₒaₒGₛAₛGₛaₛaₛgₛgₛcₛaₛcₛaₛgₛaₛ^{m}CₛGₛG-3′
SEQ ID NO: 19
5′-GN2-C6ₒcₒaₒGₛAₛGₛaₛaₛgₛgₛcₛaₛcₛaₛgₛaₛ^{m}CₛGₛG-3′
SEQ ID NO: 20
5′-GN2-C6₀c₀a₀AₛGₛ^{m}Cₛgₛaₛaₛgₛtₛgₛcₛaₛcₛaₛ^{m}CₛGₛG-3
SEQ ID NO: 21
5′-GN2-C6₀c₀a₀GₛAₛAₛgₛtₛgₛcₛaₛcₛaₛ^{m}cₛGₛG-3′
SEQ ID NO: 21
5′-GN2-C6₀c₀a₀GsAₛAₛgₛtₛgₛcₛaₛcₛaₛ^{m}CₛGₛG-3′
SEQ ID NO: 22
5′-GN2-C6₀c₀a₀Gₛ^{m}CₛGₛaₛaₛgₛtₛgₛcₛaₛcₛaₛ^{m}CₛGₛG-3′
SEQ ID NO: 23
5′-GN2-C6ₒcₒaₒAₛGₛ^{m}CₛgₛaₛaₛgₛtₛgₛcₛaₛcₛAₛ^{m}CₛG-3′;
SEQ ID NO: 24
5′-GN2-C6₀c₀a₀^{m}CₛGₛAₛaₛgₛtₛgₛcₛaₛcₛaₛ^{m}CₛG-3′
SEQ ID NO: 25
5′-GN2-C6ₒcₒaₒAₛGₛGₛtₛgₛaₛaₛgₛ^{m}cₛgₛaₛaₛgₛTₛGₛ^{m}c-3′
SEQ ID NO: 26
5′-GN2-C6ₒcₒaₒAₛGₛgₛtₛgₛaₛaₛgₛ^{m}cₛgₛaₛAₛGₛTₛG-3′
SEQ ID NO: 26
5′-GN2-C6₀c₀a₀AₛGₛGₛtₛgₛaₛaₛgₛ^{m}cₛgₛaₛaₛGₛTₛG-3′;
and
SEQ ID NO: 27
5′-GN2-C6ₒcₒaₒAₛGₛGₛtₛgₛaₛaₛgₛ^{m}cₛgₛaₛAₛGₛT-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In one embodiment wherein the GalNAc conjugated antisense oligonucleotide is selected from the group consisting of:

SEQ ID NO: 15
5′-GN2-C6ₒcₒaₒGₛ^{m}CₛGₛtₛaₛaₛaₛgₛaₛgₛaₛGₛG-3′
SEQ ID NO: 15
5′-GN2-C6ₒcₒaₒGₛ^{m}CₛGₛtₛaₛaₛaₛgₛaₛgₛAₛGₛG-3′
SEQ ID NO: 16
5-GN2-C6ₒcₒaₒGₛ^{m}CₛGₛtₛaₛaₛaₛgₛaₛgₛaₛGₛGₛT-3′;
and
SEQ ID NO: 17
5′-GN2-C6ₒcₒaₒ^{m}CₛGₛ^{m}CₛgₛtₛaₛaₛaₛgₛaₛgₛaₛGₛGₛT-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In one embodiment wherein the GalNAc conjugated antisense oligonucleotide is

SEQ ID NO: 18
5′-GN2-C6ₒcₒaₒGₛAₛGₛaₛaₛgₛgₛcₛaₛcₛaₛgₛaₛ^{m}CₛGₛG-3′
or
SEQ ID NO: 19
5′-GN2-C6ₒcₒaₒGₛAₛGₛaₛaₛgₛgₛcₛaₛcₛaₛgₛaₛ^{m}CₛGₛG-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In one embodiment wherein the GalNAc conjugated antisense oligonucleotide is selected from the group consisting of:

SEQ ID NO: 20
5′-GN2-C6₀c₀a₀AₛGₛ^{m}Cₛgₛaₛaₛgₛtₛgₛcₛaₛcₛaₛ^{m}CₛGₛG-3
SEQ ID NO: 21
5′-GN2-C6₀c₀a₀GₛAₛAₛgₛtₛgₛcₛaₛcₛaₛ^{m}cₛGₛG-3′
SEQ ID NO: 21
5′-GN2-C6₀c₀a₀GsAₛAₛgₛtₛgₛcₛaₛcₛaₛ^{m}CₛGₛG-3′
SEQ ID NO: 22
5′-GN2-C6₀c₀a₀Gₛ^{m}CₛGₛaₛaₛgₛtₛgₛcₛaₛcₛaₛ^{m}CₛGₛG-3′
SEQ ID NO: 23
5′-GN2-C6ₒcₒaₒAₛGₛ^{m}CₛgₛaₛaₛgₛtₛgₛcₛaₛcₛAₛ^{m}CₛG-3′;
SEQ ID NO: 24
5′-GN2-C6₀c₀a₀^{m}CₛGₛAₛaₛgₛtₛgₛcₛaₛcₛaₛ^{m}CₛG-3′
SEQ ID NO: 25
5′-GN2-C6ₒcₒaₒAₛGₛGₛtₛgₛaₛaₛgₛ^{m}cₛgₛaₛaₛgₛTₛGₛ^{m}c-3′
SEQ ID NO: 26
5′-GN2-C6ₒcₒaₒAₛGₛgₛtₛgₛaₛaₛgₛ^{m}cₛgₛaₛAₛGₛTₛG-3′
SEQ ID NO: 26
5′-GN2-C6₀c₀a₀AₛGₛGₛtₛgₛaₛaₛgₛ^{m}cₛgₛaₛaₛGₛTₛG-3′;
and
SEQ ID NO: 27
5′-GN2-C6ₒcₒaₒAₛGₛGₛtₛgₛaₛaₛgₛ^{m}cₛgₛaₛAₛGₛT-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In one embodiment wherein the GalNAc conjugated antisense oligonucleotide is selected from the group consisting of:

SEQ ID NO: 25
5′-GN2-C6ₒcₒaₒAₛGₛGₛtₛgₛaₛaₛgₛ^{m}cₛgₛaₛaₛgₛTₛGₛ^{m}c-3′
SEQ ID NO: 26
5′-GN2-C6ₒcₒaₒAₛGₛgₛtₛgₛaₛaₛgₛ^{m}cₛgₛaₛAₛGₛTₛG-3′
SEQ ID NO: 26
5′-GN2-C6ₒcₒaₒAₛGₛGₛtₛgₛaₛaₛgₛ^{m}cₛgₛaₛaₛGₛTₛG-3′;
and
SEQ ID NO: 27
5′-GN2-C6ₒcₒaₒAₛGₛGₛtₛgₛaₛaₛgₛ^{m}cₛgₛaₛAₛGₛT-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In one embodiment wherein the GalNAc conjugated antisense oligonucleotide is selected from the group consisting of:

SEQ ID NO: 15
5′-GN2-C6ₒcₒaₒGₛ^{m}CₛGₛtₛaₛaₛaₛgₛaₛgₛaₛGₛG-3′
SEQ ID NO: 15
5′-GN2-C6ₒcₒaₒGₛ^{m}CₛGₛtₛaₛaₛaₛgₛaₛgₛAₛGₛG-3′
SEQ ID NO: 16
5-GN2-C6ₒcₒaₒGₛ^{m}CₛGₛtₛaₛaₛaₛgₛaₛgₛaₛGₛGₛT-3′
SEQ ID NO: 20
5′-GN2-C6ₒcₒaₒAₛGₛ^{m}Cₛgₛaₛaₛgₛtₛgₛcₛaₛcₛaₛ^{m}CₛGₛG-3′
SEQ ID NO: 23
5′-GN2-C6ₒcₒaₒAₛGₛ^{m}CₛgₛaₛaₛgₛtₛgₛcₛaₛcₛAₛ^{m}CₛG-3′;
and
SEQ ID NO: 26
5′-GN2-C6ₒcₒaₒAₛGₛgₛtₛgₛaₛaₛgₛ^{m}cₛgₛaₛAₛGₛTₛG-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker.

In table 2 below the antisense oligonucleotide sequence with the biocleavable CA linker (if present) in the 5 end are shown as well as the GalNAc conjugated antisense oligonucleotides targeting position 1530 to 1602 of SEQ ID NO: 1 are shown.

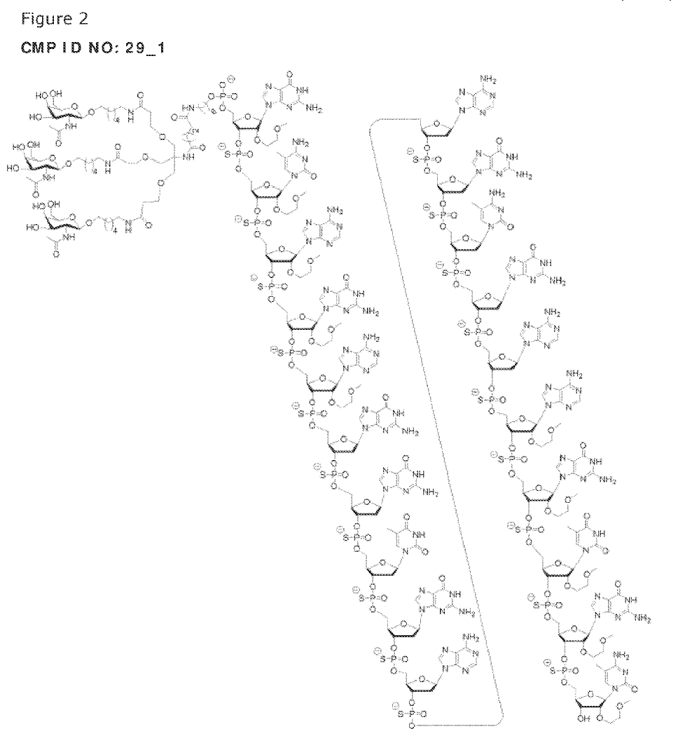

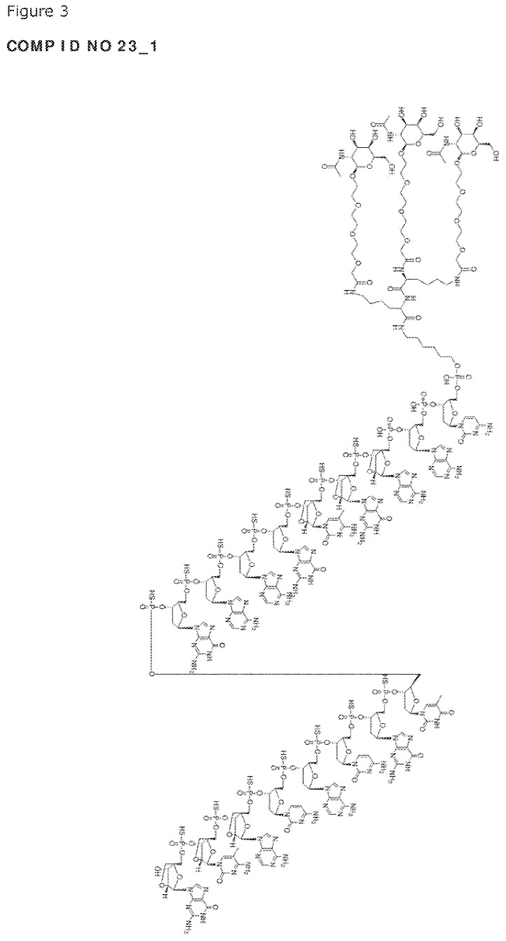

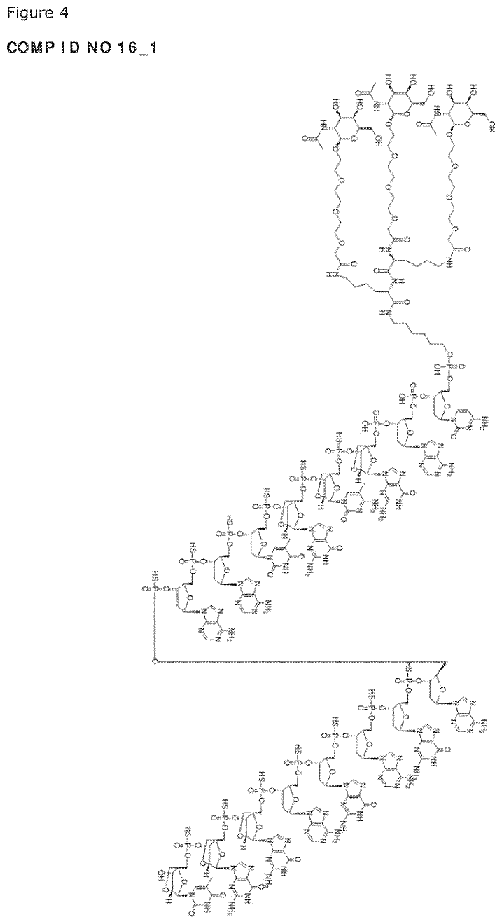

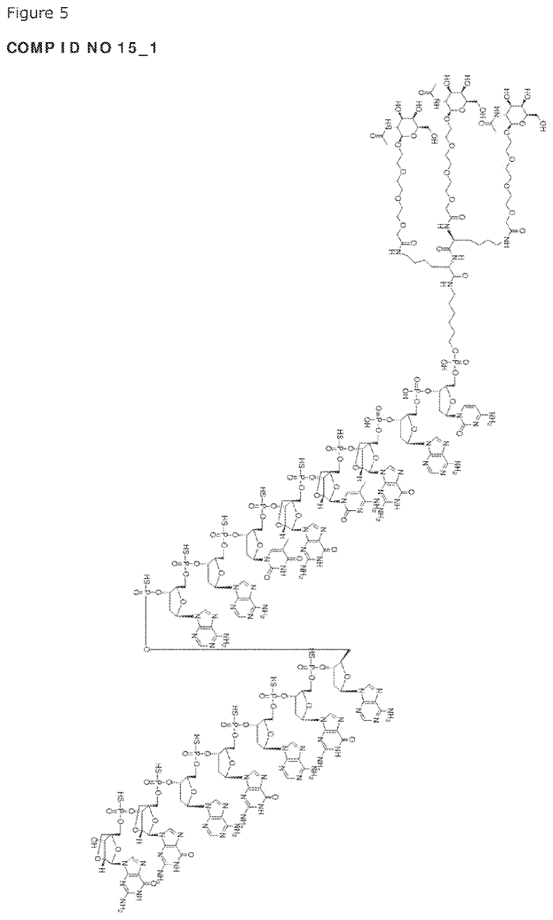

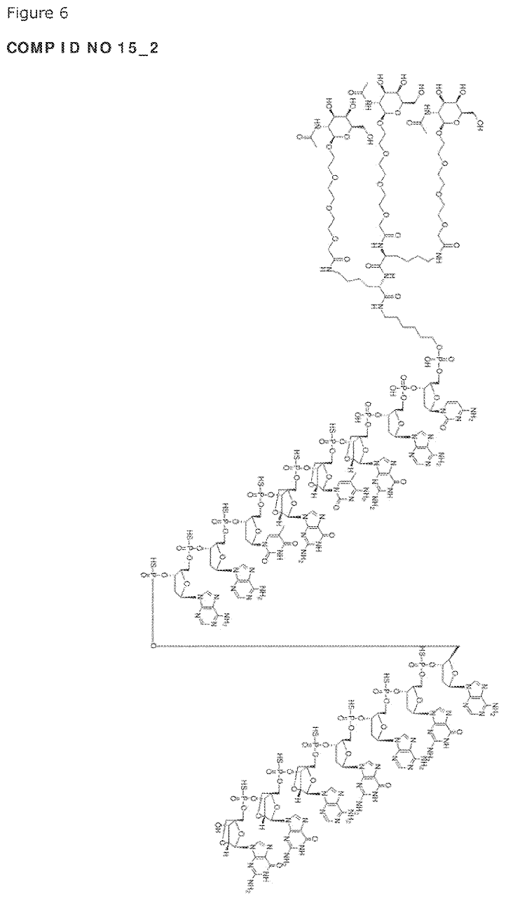

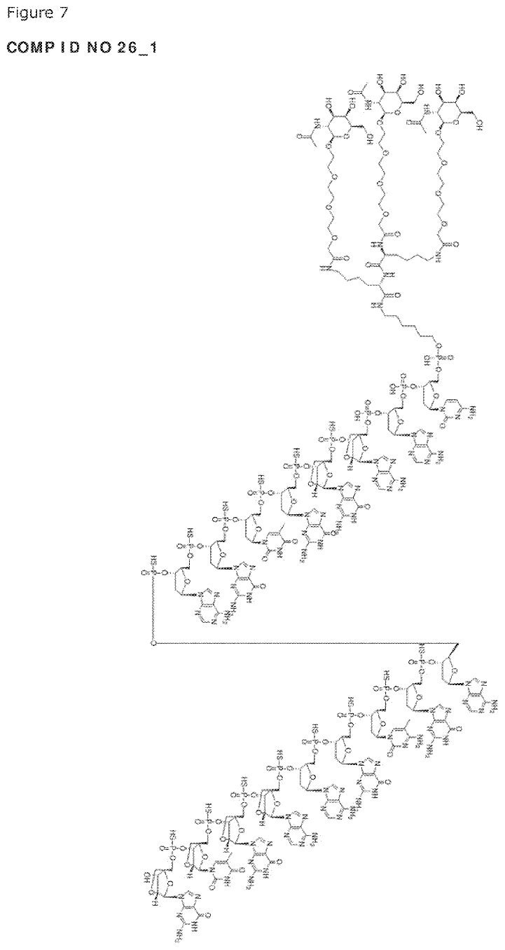

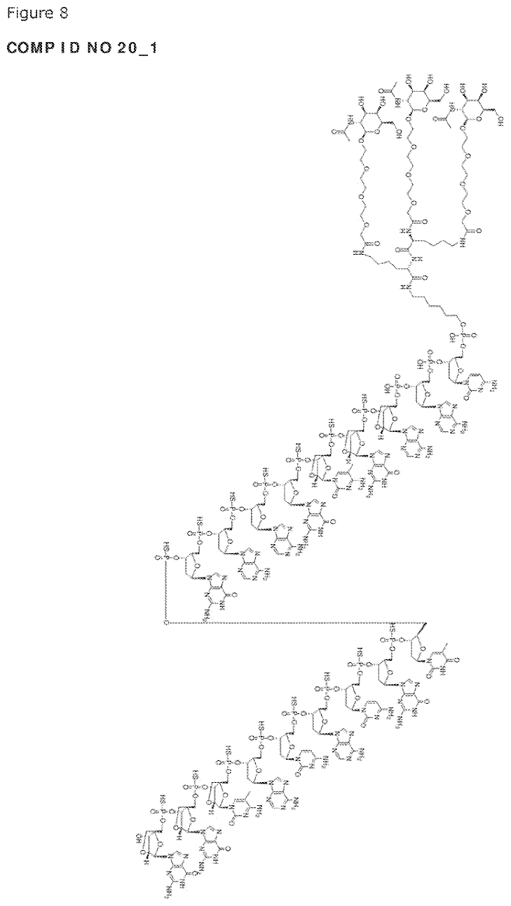

TABLE 2
GalNAc conjugated antisense oligonucleotides of the invention identified with
individual compound identification numbers (CMP ID NO).

| SEQ | Antisense | | CMP |
|---|---|---|---|
| ID | oligonucleotide | | ID |
| NO | sequence | Compound | NO |
| 15 | cagcgtaaagagagg | 5′-GN2-C6ₒcₒaₒGₛ^{m}CₛGₛtₛaₛaₛaₛgₛaₛgₛaₛGₛG-3′ | 15_1 |
| | | | FIG. 5 |
| | | 5′-GN2-C6ₒcₒaₒGₛ^{m}CₛGₛtₛaₛaₛaₛgₛaₛgₛGₛG-3′ | 15_2 |
| | | | FIG. 6 |
| 16 | cagcgtaaagagaggt | 5′-GN2-C6ₒcₒaₒGₛ^{m}CₛGₛtₛaₛaₛaₛgₛaₛgₛaₛGₛGₛT-3′ | 16_1 |
| | | | FIG. 4 |
| 17 | cacgcgtaaagagaggt | 5′-GN2-C6ₒcₒaₒ^{m}CₛGₛ^{m}CₛgₛtₛaₛaₛaₛgₛaₛgₛaₛGₛGₛT-3′ | 17_1 |
| 18 | caagaaggcacagacgg | 5′-GN2-C6ₒcₒaₒAₛGₛAₛaₛgₛgₛcₛaₛcₛaₛgₛaₛ^{m}CₛGₛG-3′ | 18_1 |
| 19 | cagagaaggcacagacgg | 5′-GN2-C6ₒcₒaₒGₛAₛGₛaₛaₛgₛgₛcₛaₛcₛaₛgₛaₛ^{m}CₛGₛG-3′ | 19_1 |
| 20 | caagcgaagtgcacacgg | 5′-GN2- | 20_1 |
| | | C6₀c₀a₀AₛGₛ^{m}Cₛgₛaₛaₛgₛtₛgₛcₛaₛcₛaₛ^{m}CₛGₛG-3 | FIG. 8 |
| 21 | cagaagtgcacacgg | 5′-GN2-C6₀c₀a₀GₛAₛAₛgₛtₛgₛcₛaₛcₛaₛ^{m}cₛGₛG-3′ | 21_1 |
| | | 5′-GN2-C6₀c₀a₀GsAₛAₛgₛtₛgₛcₛaₛcₛaₛ^{m}CₛGₛG-3′ | 21_2 |
| 22 | cagcgaagtgcacacgg | 5′-GN2-C6₀c₀a₀Gₛ^{m}CₛGₛaₛaₛgₛtₛgₛcₛaₛcₛaₛ^{m}CₛGₛG-3′ | 22_1 |
| 23 | caagcgaagtgcacacg | 5′-GN2-C6₀c₀a₀AₛGₛ^{m}CₛgₛaₛaₛgₛtₛgₛcₛaₛcₛAₛ^{m}CₛG-3′ | 23_1 |
| | | | FIG. 3 |
| 24 | cacgaagtgcacacg | 5′-GN2-C6₀c₀a₀^{m}CₛGₛAₛaₛgₛtₛgₛcₛaₛcₛaₛ^{m}CₛG-3′ | 24_1 |
| 25 | caaggtgaagcgaagtgc | 5′-GN2-C6₀c₀a₀AₛGₛGₛtₛgₛaₛaₛgₛ^{m}cₛgₛaₛaₛgₛTₛGₛ^{m}C- | 25_1 |
| | | 3′ | |
| 26 | caaggtgaagcgaagtg | 5′-GN2-C6₀c₀a₀AₛGₛgₛtₛgₛaₛaₛgₛ^{m}cₛgₛaₛAₛGₛTₛG-3′ | 26_1 |
| | | | FIG. 7 |
| | | 5′-GN2-C6₀c₀a₀AₛGₛGₛtₛgₛaₛaₛgₛ^{m}cₛgₛaₛaₛGₛTₛG-3′ | 26_2 |
| 27 | caaggtgaagcgaagt | 5′-GN2-C6₀c₀a₀AₛGₛGₛtₛgₛaₛaₛgₛ^{m}cₛgₛaₛAₛGₛT-3′ | 27_1 |
| 29 | gcagaggtgaagcgaagtg | 5′-Fig1J- | 29_1 |
| | c | ₒGₛCₛAₛGₛAₛgₛgₛtₛgₛaₛaₛgₛcₛgₛaₛAₛGₛTₛGₛC-3′ | FIG. 2 |

wherein UPPERCASE bold letters denote beta-D-oxy-LNA units; UPPERCASE underlined letters denote MOE, lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate (FIG. 1D) with a C6 linker. Compounds 15_ to 27_1 are all described in WO2015/173208 and Compound 29_1 is described in WO2014/179627, some of the compounds are also presented in the figures as indicated in table 2.

8. TLR7 Agonists The TLR7 agonist as of the invention are 3-substituted 5-amino-6H-thiazolo[4,5-d]pipyrimidine-2, 7-dione compounds, that have Toll-like receptor agonism activity as well as prodrugs thereof. WO 2006/066080, WO 2016/055553 and WO 2016/091698 describe such TLR7 agonists and their prodrug and their manufacture (hereby incorporated by reference).

In an aspect of the invention the TLR7 agonist in the pharmaceutical combination of the invention is represented by formula (I):

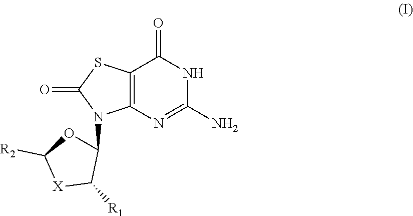

wherein X is CH₂ or S;

R₁ is —OH or —H and

R₂ is 1-hydroxypropyl or hydroxymethyl;

or formula (II):

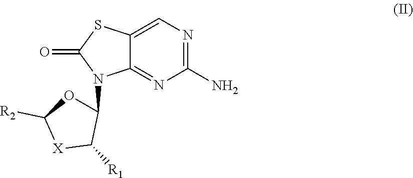

wherein X is CH₂ or S;
R₁ is —OH or —H or acetoxy and
R₂ is 1-acetoxypropyl or 1-hydroxypropyl or 1-hydroxymethyl or acetoxy(cyclopropyl)methyl
or acetoxy(propyn-1-yl)methyl,
   or a pharmaceutically acceptable salt, enantiomer or diastereomer thereof.

Compounds of formula (I) are active TLR7 agonists.

In one embodiment of the invention a subset of the active TLR7 agonist of formula (I) are represented by formula (V):

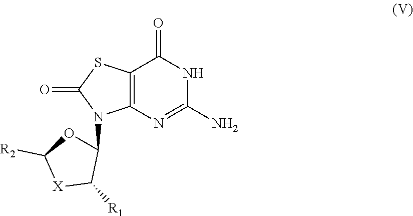

wherein R₁ is —OH and R₂ is 1-hydroxypropyl or hydroxymethyl, or a pharmaceutically acceptable salt, enantiomer or diastereomer thereof.

In one embodiment of the invention the substituent at R₂ in formula (I) or (V) is selected from:

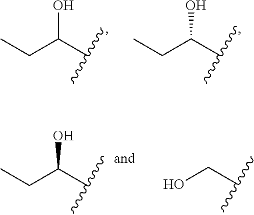

Compounds of formula (II) are TLR7 agonist prodrugs. In one embodiment the prodrug is a single prodrug with substituent at R₂ selected from:

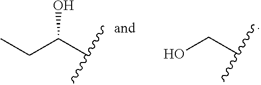

In another embodiment the prodrug is a double prodrug with substituent at R₂ selected from:

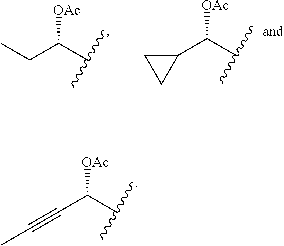

A subset of the TLR7 agonist prodrugs of formula (II) is represented by formula (III):

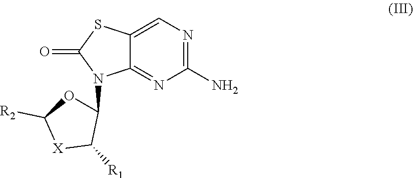

wherein R₁ is —OH or acetoxy and R₂ is 1-acetoxypropyl or 1-hydroxypropyl or 1-hydroxymethyl or

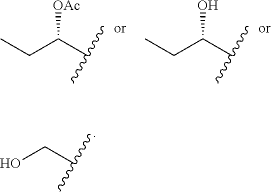

or a pharmaceutically acceptable salt, enantiomer or diastereomer thereof;

or formula (IV):

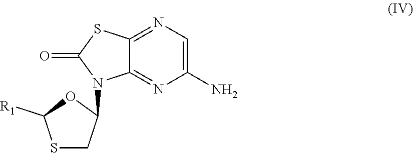

wherein R₁ is acetoxy(cyclopropyl)methyl or acetoxy(propyn-1-yl)methyl or

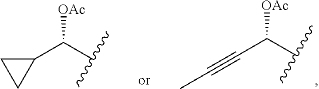

or a pharmaceutically acceptable salt, enantiomer or diastereomer thereof.

The compounds of formula (IV) are double prodrugs as is the compound of formula (III) where R₁ is OH and R₂ is 1-acetoxypropyl. The compound of formula (III) where R₁ is acetoxy and R₂ is a triple prodrug.

After administration, compounds of formula (II), (Ill) or formula (IV) are metabolized into their active forms which are useful TLR7 agonists.

In one embodiment the TLR7 agonists to be used in the pharmaceutical combination of the invention are selected from the group consisting of:
[(1S)-1-[(2S,4R,5R)-5-(5-amino-2-oxo-thiazolo[4,5-d]pyrimidin-3-yl)-4-hydroxy-tetrahydrofuran-2-yl]propyl] acetate (CMP ID NO: VI);
5-amino-3-[(2R,3R,5S)-3-hydroxy-5-[(1S)-1-hydroxypropyl]tetrahydrofuran-2-yl]-6H-thiazolo[4,5-d]pyrimidine-2,7-dione (CMP ID NO: VII);
5-amino-3-[(2R,3R,5S)-3-hydroxy-5-[(1S)-1-hydroxypropyl]tetrahydrofuran-2-yl]thiazolo[4,5-d]pyrimidin-2-one (CMP ID NO: VIII);
5-amino-3-(3′-deoxy-8-D-ribofuranosyl)-3H-thiazolo[4,5-d]pyrimidin-2-one (CMP ID NO: IX);
5-amino-3-(2′-O-acetyl-3′-deoxy-β-D-ribofuranosyl)-3H-thiazolo[4,5-d]pyrimidin-2-one (CMP ID NO: X);
5-amino-3-(3′-deoxy-β-D-ribofuranosyl)-3H,6H-thiazolo[4,5-d]pyrimidin-2,7-dione (CMP ID NO: XI);
[(S)-[(2S,5R)-5-(5-amino-2-oxo-thiazolo[4,5-d]pyrimidin-3-yl)-1,3-oxathiolan-2-yl]-cyclopropyl-methyl] acetate (CMP ID NO: XII); and
(1S)-1-[(2S,5R)-5-(5-amino-2-oxo-thiazolo[4,5-d]pyrimidin-3-yl)-1,3-oxathiolan-2-yl]but-2-ynyl] acetate (CMP ID NO: XIII)
and their pharmaceutically acceptable salt, enantiomer or diastereomer.

Table 3 lists the TLR7 agonists in the present invention, including reference to documents that describe their manufacture.

TABLE 3
TLR7 agonist compounds identified with individual compound identification numbers
(CMP ID NO)

| CMP | | | |
|---|---|---|---|
| ID | | | |
| NO | Compound Name | Structure | reference |
| VI | [(1S)-1-[(2S,4R,5R)-5-(5- amino-2-oxo-thiazolo[4,5- d]pyrimidin-3-yl)-4-hydroxy- tetrahydrofuran-2-yl]propyl] acetate | 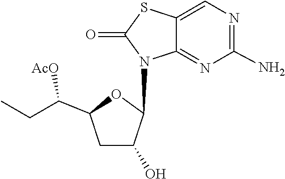 [SMILES: CC[C@H](OC(C)=O)[C@@H]1C[C@@H](O)[C@H](n2c(=O)sc3cnc(N)nc32)O1] | WO2016091698 |
| VII | 5-amino-3-[(2R,3R,5S)-3- hydroxy-5-[(1S)-1- hydroxypropyl]tetrahydrofuran- 2-yl]-6H-thiazolo [4,5-d]pyrimidine-2,7-dione | 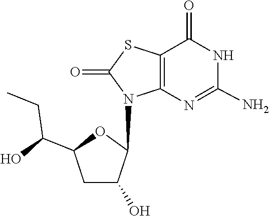 [SMILES: CC[C@H](O)[C@@H]1C[C@@H](O)[C@H](n2c(=O)sc3c(=O)[nH]c(N)nc32)O1] | WO2016091698 |
| VIII | 5-amino-3-[(2R,3R,5S)-3- hydroxy-5-[(1S)-1- hydroxypropyl]tetrahydrofuran- 2-yl]thiazolo[4,5-d]pyrimidin- 2-one | 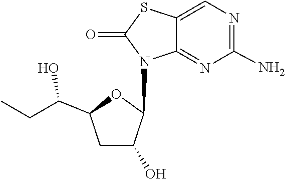 [SMILES: CC[C@H](O)[C@@H]1C[C@@H](O)[C@H](n2c(=O)sc3cnc(N)nc32)O1] | WO2016091698 |
| IX | 5-amino-3-(3′-deoxy-β-D- ribofuranosyl)-3H- thiazolo[4,5-d]pyrimidin-2-one | 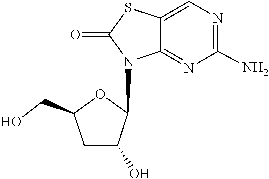 [SMILES: Nc1ncc2sc(=O)n([C@@H]3O[C@H](CO)C[C@H]3O)c2n1] | WO2006066080 |
| X | 5-amino-3-(2′-O-acetyl-3′- deoxy-β-D-ribofuranosyl)-3H- thiazolo[4,5-d]pyrimidin-2-one | 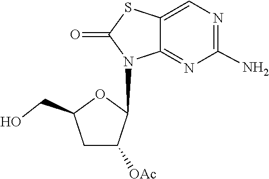 [SMILES: CC(=O)O[C@@H]1C[C@@H](CO)O[C@H]1n1c(=O)sc2cnc(N)nc21] | WO2006066080 |
| XI | 5-amino-3-(3′-deoxy-β-D- ribofuranosyl)-3H,6H- thiazolo[4,5-d]pyrimidin-2,7- dione | 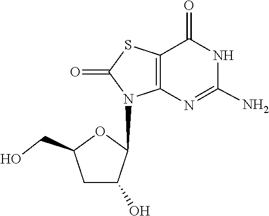 [SMILES: Nc1nc2c(sc(=O)n2[C@@H]2O[C@H](CO)C[C@H]2O)c(=O)[nH]1] | WO2006066080 |
| XII | [(S)-[(2S,5R)-5-(5-amino-2- oxo-thiazolo[4,5-d]pyrimidin- 3-yl)-1,3-oxathiolan-2-yl]- cyclopropyl-methyl] acetate | 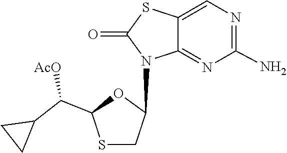 [SMILES: CC(=O)O[C@@H](C1CC1)[C@H]1O[C@@H](n2c(=O)sc3cnc(N)nc32)CS1] | WO2016055553 |
| XIII | (1S)-1-[(2S,5R)-5-(5-amino-2- oxo-thiazolo[4,5-d]pyrimidin- 3-yl)-1,3-oxathiolan-2-yl]but- 2-ynyl] acetate | 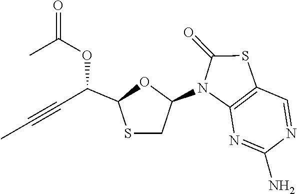 [SMILES: CC#C[C@H](OC(C)=O)[C@H]1O[C@@H](n2c(=O)sc3cnc(N)nc32)CS1] | WO2016055553 |

In a particularly preferred embodiment, the TLR7 agonist is CMP ID NO: VI.

9. Pharmaceutical Compositions

In a further aspect, the invention provides pharmaceutical compositions comprising any of the aforementioned therapeutic oligonucleotides or TLR7 agonists or salts thereof and a pharmaceutically acceptable diluent, carrier, salt and/or adjuvant. In an embodiment, the therapeutic oligonucleotide and TLR7 agonist in the pharmaceutical combination of the present invention are administered in separate compositions. In an embodiment, the therapeutic oligonucleotide is formulated in phosphate buffered saline for subcutaneous administration and the TLR7 agonist is formulated as a tablet for oral administration.

A therapeutic oligonucleotide in the pharmaceutical combination of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. A pharmaceutically acceptable diluent of therapeutic oligonucleotides includes phosphate-buffered saline (PBS) and pharmaceutically acceptable salts include, but are not limited to, sodium and potassium salts. In some embodiments the pharmaceutically acceptable diluent of the therapeutic oligonucleotide is sterile phosphate buffered saline. In some embodiments the oligonucleotide is used in the pharmaceutically acceptable diluent at a concentration of 50-150 mg/ml solution.

The therapeutic oligonucleotide or pharmaceutical composition comprising the therapeutic oligonucleotide is administered by a parenteral route including intravenous, intraarterial, subcutaneous or intramuscular injection or infusion. In one embodiment the oligonucleotide conjugate is administered intravenously. For therapeutic oligonucleotides it is advantageous if they are administered subcutaneously. In some embodiments, the oligonucleotide conjugate or pharmaceutical composition of the invention is administered at a dose of 0.5-6.0 mg/kg, such as from 0.75-5.0 mg/kg, such as from 1.0-4 mg/kg. The administration can be once a week, every 2^{nd} week (biweekly), every third week, once a month or at a longer interval.

For TLR7 agonists in the pharmaceutical combination of the invention, the pharmaceutically effective amount of the compound of the invention is administered enterally (such as orally or through the gastrointestinal tract). The TLR7 agonist compounds in the present invention may be administered in unit doses of any convenient administrative form, e.g., tablets, powders, capsules, solutions, dispersions, suspensions, syrups, sprays, suppositories, gels, emulsions. In particular oral unit dosage forms, such as tablets and capsules, can be used. In one example, the pharmaceutically effective amount of the TLR7 agonist compound of the invention will be in the range of about 75-250 mg, such as 100 to 200 mg such as 150 to 170 mg pr. dose. The administration can be daily, every other day (QOD) or weekly (QW).

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules.

10. Formulations of Therapeutic Oligonucleotides

Various formulations have been developed to facilitate therapeutic oligonucleotide use, which may be applicable to therapeutic oligonucleotides used in the pharmaceutical combinations of the present invention. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are pharmaceutical combinations comprising a first medicament which is a composition comprising an oligonucleotide (e.g., a single-stranded or double-stranded oligonucleotide) to reduce the expression of HBV antigen (e.g., HBsAg). Such compositions can be suitably formulated such that when administered to a subject, either into the immediate environment of a target cell or systemically, a sufficient portion of the oligonucleotides enter the cell to reduce HBV antigen expression. Any of a variety of suitable oligonucleotide formulations can be used to deliver oligonucleotides for the reduction of HBV antigen as disclosed herein. In some embodiments, an oligonucleotide of the pharmaceutical combination of the present invention is formulated in buffer solutions such as phosphate-buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, an oligonucleotide formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: The Science and Practice of Pharmacy, 22nd edition, Pharmaceutical Press, 2013).

In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin). In some embodiments of the pharmaceutical combination of the present invention, the composition comprising an oligonucleotide is formulated to be compatible with its intended route of administration. Examples of routes of administration include parenteral, e.g., intravenous, intradermal, subcutaneous, oral (e.g., inhalation), transdermal (topical), transmucosal, and rectal administration. Formulation for subcutaneous is particularly advantageous where the oligonucleotide in the pharmaceutical combination of the present invention is an RNAi oligonucleotide.

Pharmaceutical compositions suitable for injectable use include sterile aqueous solutions (where water soluble) or dispersions and sterile powders for the extemporaneous preparation of sterile injectable solutions or dispersions. Suitable carriers include physiological saline, bacteriostatic water, Cremophor EL. TM. (BASF, Parsippany, N.J.) or phosphate buffered saline (PBS). The carrier may be water or a solvent or dispersion medium. The solvent or dispersion medium may contain, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

In some embodiments of the pharmaceutical combination of the present invention, a composition in the combination may contain at least about 0.1% of the therapeutic agent (e.g., an oligonucleotide for reducing HBV antigen expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

11. Pharmaceutical Combinations and Kits of Parts

One aspect of present invention relates to a pharmaceutical combination a therapeutic oligonucleotide targeting HBV and a TLR7 agonist as described herein each formulated in a pharmaceutically acceptable carrier.

The pharmaceutical combination of the present invention can be used to treat an HBV infection more effectively than the comprised therapeutic oligonucleotide or TLR7 agonist alone. In an embodiment, the pharmaceutical combination of the present invention can be used to inhibit HBV more rapidly, to inhibit HBV with an increased duration and/or to inhibit HBV with greater effect than the comprised therapeutic oligonucleotide or TLR7 agonist alone. These effects may be measured by a reduction in HBsAg titre. In an embodiment, the pharmaceutical combination of the present invention causes a more rapid reduction in HBsAg titre than the comprised therapeutic oligonucleotide or TLR7 agonist alone. In an embodiment, the pharmaceutical combination of the present invention causes a more prolonged reduction in HBsAg titre than the comprised therapeutic oligonucleotide or TLR7 agonist alone. In an embodiment, the pharmaceutical combination of the present invention causes a greater decrease in HBsAg titre than the comprised therapeutic oligonucleotide or TLR7 agonist alone.

In a preferred embodiment of the present invention, the pharmaceutical combination comprises or consists of an RNAi oligonucleotide and a TLR7 agonist as described herein.

In an embodiment of the present invention, the pharmaceutical combination comprises or consists of an RNAi oligonucleotide and a TLR7 agonist of formula (I) or (II):

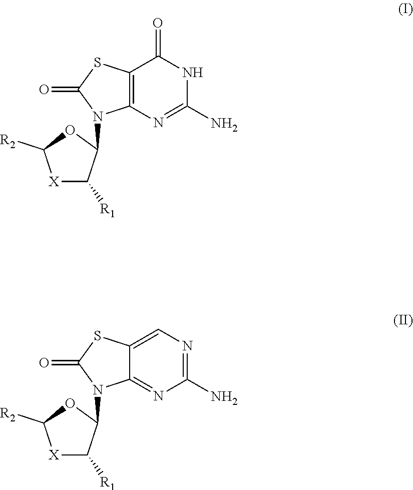

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or
1-hydroxymethyl or acetoxy(cyclopropyl)methyl or acetoxy(propyn-1-yl)methyl,
   or a pharmaceutically acceptable salt, enantiomer or diastereomer thereof.

The RNAi oligonucleotides and TLR7 agonists of the invention have been described individually above, e.g. in sections 1-3 and 8 above.

In one embodiment of invention the pharmaceutical combination can be selected from a compound in the vertical column and a compound in the horizontal column in Table 4. Each possible combination is indicated by an “x”.

TABLE 4
Possible RNAi oligonucleotide, TLR7 agonist combinations

| | | TLR7 Agonist CMP ID NO | | | | | | | |
|---|---|---|---|---|---|---|---|---|---|
| | | VI | VII | VIII | IX | X | XI | XII | XIII |
| RNAi | 1 | x | x | x | x | x | x | x | x |
| ID NO | 2 | x | x | x | x | x | x | x | x |
| | 3 | x | x | x | x | x | x | x | x |
| | 4 | x | x | x | x | x | x | x | x |
| | 5 | x | x | x | x | x | x | x | x |
| | 6 | x | x | x | x | x | x | x | x |
| | 7 | x | x | x | x | x | x | x | x |
| | 8 | x | x | x | x | x | x | x | x |
| | 9 | x | x | x | x | x | x | x | x |

Table 5 and 6 below show selected combinations of RNAi oligonucleotides (vertical) and TLR7 agonists (horizontal).

TABLE 5

| | TLR7 agonist CMP ID NO | |
|---|---|---|
| | VI | VII |
| RNAi ID NO | 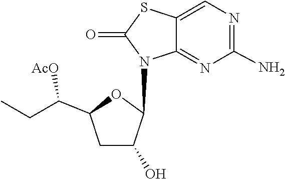 | 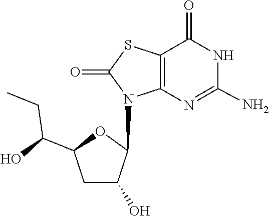 |
| 1 | X | X |
| 2 | X | X |
| 3 | X | X |
| 4 | X | X |
| 5 | X | X |
| 6 | X | X |
| 7 | X | X |
| 8 | X | X |
| 9 | X | X |

TABLE 6

| | TLR7 agonist CMP ID NO | |
|---|---|---|
| | VIII | XIII |
| RNAi ID NO | 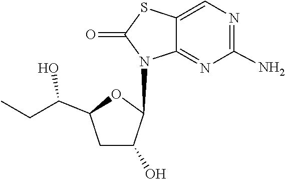 | 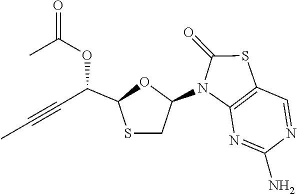 |
| 1 | X | X |
| 2 | X | X |
| 3 | X | X |
| 4 | X | X |
| 5 | X | X |
| 6 | X | X |
| 7 | X | X |
| 8 | X | X |
| 9 | X | X |

In one embodiment of the invention the pharmaceutical combination is selected from the group consisting of:
RNAi ID NO: 1 and CMP ID NO: VI; RNAi ID NO: 2 and CMP ID NO: VI; RNAi ID NO: 3 and CMP ID NO: VI; RNAi ID NO: 4 and CMP ID NO: VI; RNAi ID NO: 5 and CMP ID NO: VI; RNAi
ID NO: 6 and CMP ID NO: VI; RNAi ID NO: 7 and CMP ID NO: VI; RNAi ID NO: 8 and CMP ID NO: VI; RNAi ID NO: 9 and CMP ID NO: VI;
RNAi ID NO: 1 and CMP ID NO: VII, RNAi ID NO: 2 and CMP ID NO: VII; RNAi ID NO: 3 and CMP ID NO: VII; RNAi ID NO: 4 and CMP ID NO: VII; RNAi ID NO: 5 and CMP ID NO: VII; RNAi ID NO: 6 and CMP ID NO: VII; RNAi ID NO: 7 and CMP ID NO: VII; RNAi ID NO: 8 and CMP ID NO: VII; RNAi ID NO: 9 and CMP ID NO: VII;
RNAi ID NO: 1 and CMP ID NO: VIII, RNAi ID NO: 2 and CMP ID NO: VIII; RNAi ID NO: 3 and CMP ID NO: VIII; RNAi ID NO: 4 and CMP ID NO: VIII; RNAi ID NO: 5 and CMP ID NO: VIII; RNAi ID NO: 6 and CMP ID NO: VIII; RNAi ID NO: 7 and CMP ID NO: VIII; RNAi ID NO: 8 and CMP ID NO: VIII; RNAi ID NO: 9 and CMP ID NO: VIII;
RNAi ID NO: 1 and CMP ID NO: XIII, RNAi ID NO: 2 and CMP ID NO: XIII; RNAi ID NO: 3 and CMP ID NO: XIII; RNAi ID NO: 4 and CMP ID NO: XIII; RNAi ID NO: 5 and CMP ID NO: XIII; RNAi ID NO: 6 and CMP ID NO: XIII; RNAi ID NO: 7 and CMP ID NO: XIII; RNAi ID NO: 8 and CMP ID NO: XIII; RNAi ID NO: 9 and CMP ID NO: XIII;

or a pharmaceutically acceptable salt, enantiomer or diastereomer thereof.

In one embodiment, the therapeutic oligonucleotide of the pharmaceutical combination of the invention consists of the RNAi oligonucleotide which is RNAi ID NO: 7:

An oligonucleotide for reducing expression of hepatitis B virus surface antigen (HBsAg) mRNA, the oligonucleotide comprising a sense strand forming a duplex region with an antisense strand, wherein:

the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GaINac moiety, wherein the -GAAA- sequence comprises the structure:

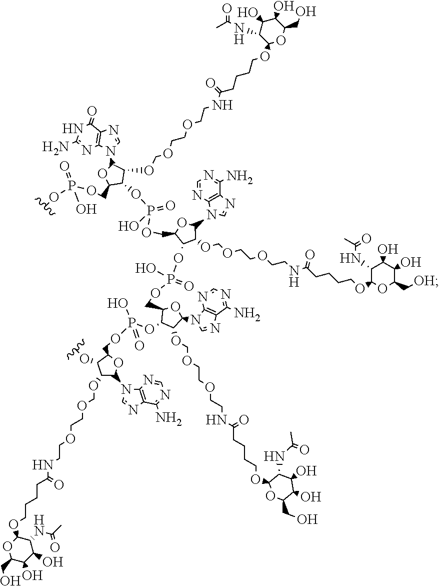

and

the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

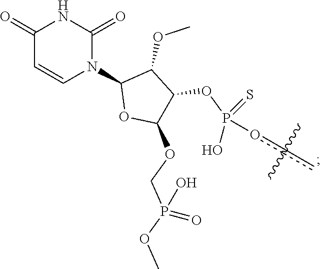

and the TLR7 agonist is CMP ID NO: VI:

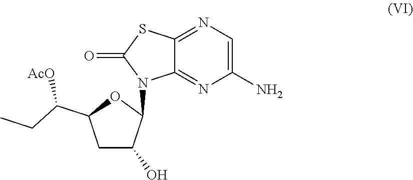 [SMILES: CC[C@H](OC(C)=O)[C@@H]1C[C@@H](O)[C@H](n2c(=O)sc3ncc(N)nc32)O1]

or a pharmaceutically acceptable salt, enantiomer or diastereomer thereof.

In particularly preferred embodiments of the pharmaceutical combinations comprising an RNAi oligonucleotide and a TLR7 agonist, the TLR7 agonist is CMP ID NO: VI.

In an embodiment, the pharmaceutical combinations comprising an RNAi oligonucleotide and a TLR7 agonist of the present invention further comprise a CpAM (core protein allosteric modulator).

In a preferred embodiment, the CpAM is according to compound (CpAM1). Compound (CpAM1) is a CpAM for treatment and/or prophylaxis of HBV in a human by targeting the HBV capsid, which is disclosed in WO2015132276. The structure of Compound (CpAM1) is shown below:

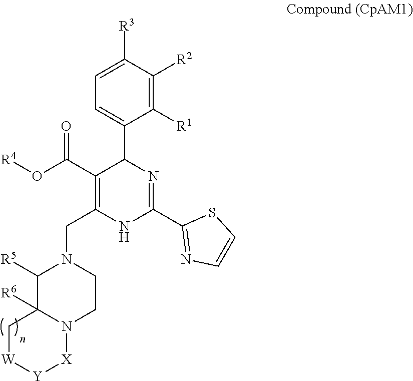

wherein

R¹ is hydrogen, halogen or C₁₋₆alkyl;

R² is hydrogen or halogen;

R³ is hydrogen or halogen;

R⁴ is C₁₋₆alkyl;

R⁵ is hydrogen, hydroxyC₁₋₆alkyl, aminocarbonyl, C₁₋₆alkoxycarbonyl or carboxy;

R⁶ is hydrogen, C₁₋₆alkoxycarbonyl or carboxy-CₘH₂ₘ—,

X is carbonyl or sulfonyl;

Y is —CH₂—, —O— or —N(R⁷)—,

wherein R⁷ is hydrogen, C₁₋₆alkyl, haloC₁₋₆alkyl, C₃₋₇cycloalkyl-CₘH₂ₘ—, C₁₋₆alkoxycarbonyl-CₘH₂ₘ—, —CₜH₂ₜ—COOH, -haloC₁₋₆ alkyl-COOH, —(C₁₋₆alkoxy)C₁₋₆alkyl-COOH, —C₃₋₇ cycloalkyl-CₘH₂ₘ—COOH, —CₘH₂ₘ—C₃₋₇ cycloalkyl-COOH, hydroxy-CₜH₂ₜ—, carboxyspiro[3.3]heptyl or carboxyphenyl-CₘH₂ₘ—, carboxypyridinyl-CₘH₂ₘ—;

W is —CH₂—, —C(C₁₋₆ alkyl)₂—, —O— or carbonyl;

n is 0 or 1;

m is 0-7;

t is 1-7;

or pharmaceutically acceptable salts, or enantiomers or diastereomers thereof.

In a further preferred embodiment, the CpAM is according to compound (CpAM2) or a pharmaceutically acceptable salt, enantiomer or diastereomer thereof. Compound (CpAM2) is a CpAM for treatment and/or prophylaxis of HBV in a human by targeting the HBV capsid, which is disclosed in Example 76 of WO2015132276 and can be prepared accordingly. The structure of Compound (CpAM2) is shown below:

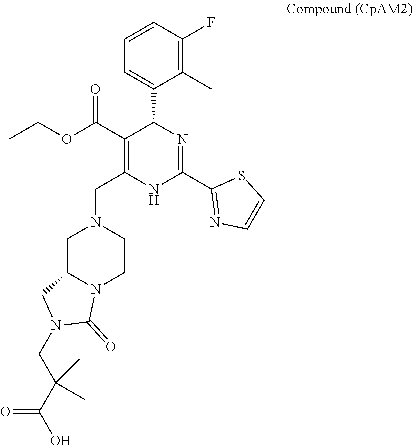 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

In a further preferred embodiment, the CpAM is 3-[(8a5)-7-[[(45)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid, which is disclosed in Example 76 of WO2015132276 and can be prepared accordingly.

In another embodiment of present invention, the pharmaceutical combination comprises or consists of a GalNAc conjugated antisense oligonucleotide of 13 to 22 nucleotides in length with a contiguous nucleotide sequence of at least 12 nucleotides which is 100% complementary to a contiguous sequence from position 1530 to 1602 of SEQ ID NO: 1, and a TLR7 agonist of formula (I) or (II):

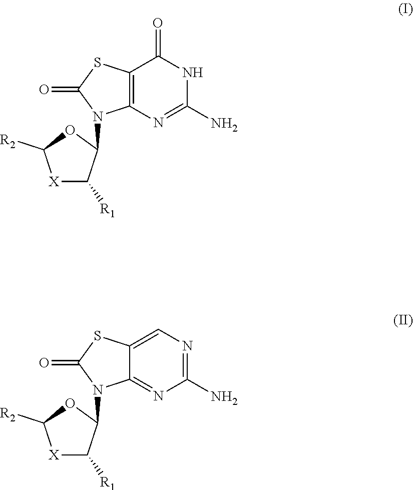

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or
1-hydroxymethyl or acetoxy(cyclopropyl)methyl or acetoxy(propyn-1-yl)methyl,

or a pharmaceutically acceptable salt, enantiomer or diastereomer thereof.

The GalNAc conjugated antisense oligonucleotides targeting HBV and the TLR7 agonists of the invention have been described individually above, e.g. in sections 4-6 and 8 above.

In one embodiment of the invention the pharmaceutical combination can be selected from a compound in the vertical column and a compound in the horizontal column in Table 7. Each possible combination is indicated by an “x”.

TABLE 7
Possible GalNAc conjugated antisense oligonucleotide,
TLR7 agonist combinations

| | | TLR7 Agonist | | | | | | | |
|---|---|---|---|---|---|---|---|---|---|
| CMP ID NO | | VI | VII | VIII | IX | X | XI | XII | XIII |
| GalNAc | 15_1 | x | x | x | x | x | x | x | x |
| conjugated | 15_2 | x | x | x | x | x | x | x | x |
| oligonucleotide | 16_1 | x | x | x | x | x | x | x | x |
| | 17_1 | x | x | x | x | x | x | x | x |
| | 18_1 | x | x | x | x | x | x | x | x |
| | 19_1 | x | x | x | x | x | x | x | x |
| | 20_1 | x | x | x | x | x | x | x | x |
| | 21_1 | x | x | x | x | x | x | x | x |
| | 21_2 | x | x | x | x | x | x | x | x |
| | 22_1 | x | x | x | x | x | x | x | x |
| | 23_1 | x | x | x | x | x | x | x | x |
| | 24_1 | x | x | x | x | x | x | x | x |
| | 25_1 | x | x | x | x | x | x | x | x |
| | 26_1 | x | x | x | x | x | x | x | x |
| | 26_2 | x | x | x | x | x | x | x | x |
| | 27_1 | x | x | x | x | x | x | x | x |

Table 8 and 9 below show selected combinations of GalNAc conjugated antisense oligonucleotides (vertical) and TLR7 agonists (horizontal).

TABLE 8

| | | VI | VII |
|---|---|---|---|
| CMP ID NO | Compound | 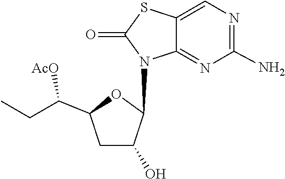 | 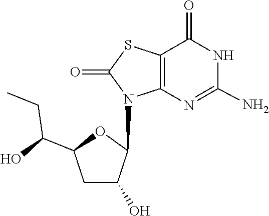 |
| 15_1 | GN2-C6ocoaoGsmCsGstₛaₛaₛaₛgₛaₛgₛaₛGsG′ | X | X |
| 15_2 | GN2-C6ocoaoGₛmCₛGₛtₛaₛaₛaₛgₛaₛgₛAₛGₛG | X | X |
| 16_1 | GN2-C6ocoaoGsmCsGstₛaₛaₛaₛgₛaₛgₛaₛGsGsT | X | X |
| 20_1 | GN2-C6ocoaoAsGsmCsgₛaₛaₛgₛtₛgₛcₛaₛcₛaₛmCsGsG | X | X |
| 23_1 | GN2-C6ocoaoAsGsmCsgₛaₛaₛgₛtₛgₛcₛaₛcₛAsmCsG | X | X |
| 26_1 | GN2-C6ocoaoAsGsGstₛgₛaₛaₛgₛ^{m}aₛGsTsG | X | X |
| 29_1 | 5′-Fig1J- | X | X |
| | oGₛCₛAₛGₛAₛgₛgₛtₛgₛaₛaₛgₛcₛgₛaₛAₛGₛTₛGₛC-3′ | | |

TABLE 9

| | | VIII | XIII |
|---|---|---|---|
| CMP ID NO | Compound | 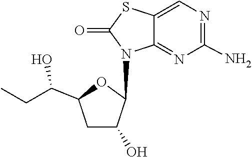 | 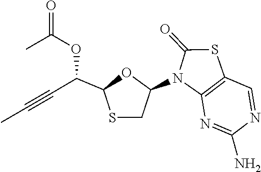 |
| 15_1 | GN2-C6ocoaoGsmCsGstₛaₛaₛaₛgₛaₛgₛaₛGsG′ | X | X |
| 15_2 | GN2-C6ocoaoGₛmCₛGₛtₛaₛaₛaₛgₛaₛgₛAₛGₛG | X | X |
| 16_1 | GN2- | X | X |
| | C6ocoaoGsmCsGstₛaₛaₛaₛgₛaₛgₛaₛGsGsT | | |
| 20_1 | GN2- | X | X |
| | C6ocoaoAsGsmCsgₛaₛaₛgₛtₛgₛcₛaₛcₛaₛmCsGsG | | |
| 23_1 | GN2- | X | X |
| | C6ocoaoAsGsmCsgₛaₛaₛgₛtₛgₛcₛaₛcₛAsmCsG | | |
| 26_1 | GN2-C6ocoaoAsGsGstₛgₛaₛaₛgₛ^{m}aₛGsTsG | X | X |
| 29_1 | 5′-Fig1J- | X | X |
| | oGₛCₛAₛGₛAₛgₛgₛtₛgₛaₛaₛgₛcₛgₛaₛAₛGₛTₛGₛC-3′ | | |

In one embodiment of the invention the pharmaceutical combination is selected from the group consisting of:
CMP ID NO: 15_ 1 and VI, CMP ID NO: 15_ 2 and VI; CMP ID NO: 16_ 1 and VI; CMP ID NO: 20_ 1 and VI; CMP ID NO: 23_ 1 and VI; CMP ID NO: 26_ 1 and VI; CMP ID NO: 29_1 and VI;
CMP CD NO: 15_ 1 and VII, CMP ID NO: 15_ 2 and VII; CMP ID NO: 16_ 1 and VII; CMP ID NO: 20_ 1 and VII; CMP ID NO: 23_ 1, VII; CMP ID NO: 26_ 1 and VII; CMP ID NO: 29_1 and VII;
CMP ID NO: 15_ 1 and VIII, CMP ID NO: 15_ 2 and VIII; CMP ID NO: 16_ 1 and VIII; CMP ID NO: 20_ 1 and VIII; CMP ID NO: 23_ 1 and VII; CMP ID NO: 26_ 1 and VIII; CMP ID NO: 29_1 and VIII; and
CMP ID NO: 15_ 1 and XIII, CMP ID NO: 15_ 2 and XIII; CMP ID NO: 16_ 1 and XIII; CMP ID NO: 20_ 1 and XIII; CMP ID NO: 23_ 1 and XIII; CMP ID NO: 26_ 1 and XIII and CMP ID NO: 29_1 and XIII;
or a pharmaceutically acceptable salt, enantiomer or diastereomer thereof.
In one embodiment the pharmaceutical combination consists of the GalNAc conjugated antisense oligonucleotide of CMP ID NO: 15_1 as shown in FIG. 5 and the TLR7 agonist is CMP ID NO: VI:

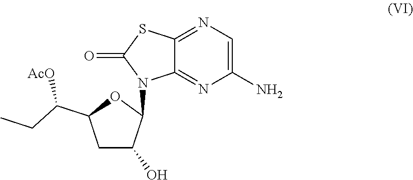 [SMILES: CC[C@H](OC(C)=O)[C@@H]1C[C@@H](O)[C@H](n2c(=O)sc3ncc(N)nc32)O1]

or a pharmaceutically acceptable salt, enantiomer or diastereomer thereof.

In particularly preferred embodiments of the pharmaceutical combinations comprising an antisense oligonucleotide, the TLR7 agonist is CMP ID NO: VI.

The term “kit” or “kit of parts” refers to an assembly of materials that are used in performing the treatment of an HBV infected individual, including a description of how to conduct the treatment.

An aspect of the invention is a kit of parts containing one, two or a plurality of therapeutically effective components (such as medical components or medicaments), where two of them are selected from the therapeutic oligonucleotide as described herein and the TLR7 agonist as described herein.

One embodiment of the invention is a kit of parts comprising a therapeutic oligonucleotide as described herein and a TLR7 agonist as described herein as medical components.

In one embodiment the kit of the invention contains a first medicament which is a therapeutic oligonucleotide as described herein formulated for subcutaneous injection and a second medicament which is a TLR7 agonist as described herein formulated for oral administration. The therapeutic oligonucleotide can be formulated as a liquid in a vial with one or multiple doses or in a prefilled syringe with one pharmaceutically effective dose. Alternatively, the therapeutic oligonucleotide can be in the form of lyophilized powder and the kit contains dissolvent for preparation of the therapeutic oligonucleotide for injection. It is understood that all medicaments for injection are sterile. The TLR7 agonist in the kit can be in tablet form (or alternative unit dose forms for oral administrations such as capsules and gels) with a single pharmaceutically effective dose pr. tablet, the kit can contain multiple tablets.

In a further embodiment the kit of parts of the present invention further comprises a package insert instructing administration of the therapeutic oligonucleotide in combination with the TLR7 agonist to treat a hepatitis B virus infection. In particular, the package insert describes the treatment of a chronic hepatitis B virus infection.

The kit may contain just one of the medical components and a package insert instructing its use in combination with the other medical component. In one embodiment the kit of parts of the invention comprises or contains a first medicament which is a therapeutic oligonucleotide as described herein and package insert instructing its use in combination with a TLR7 agonist as described herein as the second medicament, but which is purchased separately. In another embodiment the kit of parts of the invention comprises or contains a first medicament which is a TLR7 agonist as described herein and package insert instructing its use in combination with a therapeutic oligonucleotide as described herein as the second medicament, but which is purchased separately.

In some embodiments the pharmaceutical combination of the invention may be used in combination with a third or further therapeutic agent(s), which may be included in the kits of part or supplied separately. The further therapeutic agent can for example be the standard of care for the treatment of HBV infections, in particular chronic HBV infections.

12. Applications

The pharmaceutical combination of the present invention is for use in treatment of Hepatitis B virus infections, in particular treatment of patients with chronic HBV.

The pharmaceutical combination of the invention may be utilized as therapeutics and in prophylaxis.

The pharmaceutical combination of the invention can be used as a combined hepatitis B virus targeting therapy and an immunotherapy. In particular, the pharmaceutical combination of the invention is capable of affecting one or more of the following HBV infection parameters i) reducing cellular HBV mRNA, ii) reducing HBV DNA in serum and/or iii) reducing HBV viral antigens, such as HBsAg and HBeAg when used in the treatment of HBV in an infected cell. In an embodiment of the invention the effect on one or more of these parameters is improved compared to the effect achieved when performing the treatment with an individual medical component of the pharmaceutical combination.

The effect on a HBV infection may be measured in vitro using HBV infected primary human hepatocytes or HBV infected HepaRG cells or ASGPR-HepaRG cells (see for example PCT/EP2018/078136). The effect on a HBV infection may also be measured in vivo using AAV/HBV mouse model of mice infected with a recombinant adeno-associated virus (AAV) carrying the HBV genome (AAV/HBV) (Dan Yang, et al. 2014 Cellular & Molecular Immunology 11, 71-78) or HBV minicircle mouse (available at Covance Shanghai, see also Guo et al 2016 Sci Rep 6: 2552 and Yan et al 2017 J Hepatology 66(6):1149-1157) or humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

In one embodiment of the present invention the pharmaceutical combination of a therapeutic oligonucleotide targeting HBV mRNA as described herein and a TLR7 agonist as described herein provides an advantage over the mono-compound treatments (therapeutic oligonucleotide alone or TLR7 agonist alone). The advantage can for example be i) prolonged serum HBV-DNA reduction compared to mono-therapy; ii) delayed rebound in HBsAg compared to mono-therapy and/or iii) increased therapeutic window. The term “therapeutic window” or “pharmaceutical window” in relation to a drug is the range of drug dosages which can treat disease effectively without having toxic effects. In one embodiment of the invention, an increase in the therapeutic window can be achieved by the combination treatment as compared to mono-therapy.

In the study of the present application it has been observed that a significantly improved effect can be achieved with a 3-5 times lower dose when using the combination treatment compared to the dosages needed when using mono-therapy, and essentially the same effect can be achieved with a 3-5 times lower dose of the combination treatment when compared to the same combination at the higher dose. It has for example been shown that for mono-therapy the high dose (7.5 mg/kg anti-HBV antisense oligonucleotide or 100 mg every 2^{nd} day (QOD) TLR7 agonist) is needed to achieve efficient reduction of HBsAg, when using a combination at the lower dose (1.5 mg/kg and 100 mg weekly (QW)) HBsAg is reduced below the limit of detection and the time to rebound is prolonged significantly as compared to mono-therapy at the higher dose. Furthermore, the rebound of the viral parameter HBsAg can be delayed to the same extent when using a pharmaceutical combination of an anti-HBV therapeutic oligonucleotide in a 5 times lower dose (1.5 mg/kg vs 7.5 mg/kg) combined with a TLR7 agonist administered once weekly instead of every other day (corresponding to a 4 times reduction in dose). Similar results are observed for HBV-DNA reduction.

The invention provides methods for treating or preventing HBV infection, comprising administering a therapeutically or prophylactically effective amount of a pharmaceutical combination of the present invention to a subject suffering from or susceptible to HBV infection.

A further aspect of the invention relates to the use of the pharmaceutical combination of the present invention to inhibit development of or treat a chronic HBV infection.

One aspect of the present invention is a method of treating an individual infected with HBV, such as an individual with chronic HBV infection, comprising administering a pharmaceutically effective amount of a therapeutic oligonucleotide as defined herein, and a pharmaceutically effective amount of a TLR7 agonist of formula (I) or (II):

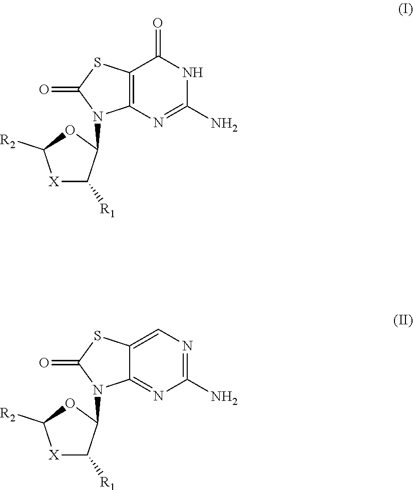

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or 1-hydroxymethyl or acetoxy(cyclopropyl)methyl or acetoxy(propyn-1-yl)methyl, to a HBV infected individual.

The invention also relates to a therapeutic oligonucleotide as described in the application for use as a medicament in a combination treatment. The invention also relates to a TLR7 agonist described in the application for use as a medicament in a combination treatment.

In particular, a therapeutic oligonucleotide as defined herein, and a TLR7 agonist of formula (I) or (II):

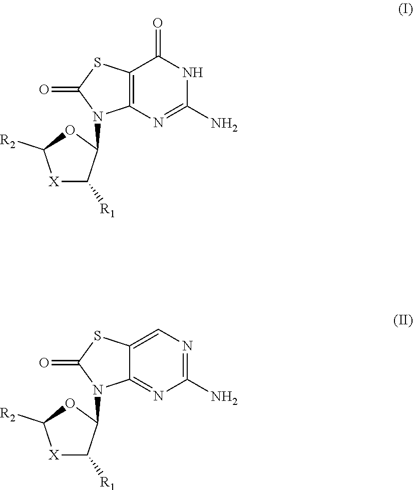

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or 1-hydroxymethyl or acetoxy(cyclopropyl)methyl or acetoxy(propyn-1-yl)methyl;

are for use in treatment of a hepatitis B virus infection.

One embodiment of the invention is the use of a therapeutic oligonucleotide in the manufacture of a first medicament for treating a hepatitis B virus infection, such as a chronic HBV virus infection, wherein the first medicament is a therapeutic oligonucleotide as described in the present application and wherein the first medicament is to be administered in combination with a second medicament, wherein the second medicament is a TLR7 agonist as described in the present application.

In one embodiment of the present invention the medical composition containing the therapeutic oligonucleotide is to be administered as a subcutaneous dose. In a further embodiment of the present invention the TLR7 agonist is to be administered as an oral dose. Since the medical composition will be administered through two different routes of administration they can follow different administration regiments.

The pharmaceutical combination according to the present invention is typically administered in an effective amount.

In one embodiment the therapeutic oligonucleotide as described in the present application is administered subcutaneously in a dose range of 1 mg/kg to 4 mg/kg with weekly or monthly dosing in between 24 and 72 weeks, such as between 36 and 60 weeks, such as 48 weeks and the TLR7 agonist as described in the present application is administered orally as a unit dose ranging between 150 and 170 mg every other day (QOD) for 8 to 26 weeks such as 10 to 24 weeks such as 12 or 13 weeks followed by a weekly administration (QW) for 24 to 48 weeks such as 30 to 40 weeks such as 35 weeks. In the period with administration every other day there may be a 10 to 14 week, such as a 12 week period off treatment. The number of doses administered of the TLR7 agonist is between 60 and 100 doses, such as between 75 and 90 doses, such as 81, 82, 83 or 84 doses throughout the treatment period. The number of doses administered of the therapeutic oligonucleotide is between 6 and 72, such as between 9 and 15, such as 12 or 48 doses.

For optimal combination effects the therapeutic oligonucleotide and the TLR7 agonist, are administered less than a month apart, such as less than a week apart, such as two day apart, such as on the same day.

13. Methods of Use

I. Reducing HBsAg Expression

In some embodiments, methods are provided for delivering to a cell an effective amount any one of the pharmaceutical combinations of the present invention, which comprise the oligonucleotides disclosed herein, particularly the RNAi oligonucleotides disclosed herein, for purposes of reducing expression of HBsAg. Methods provided herein are useful in any appropriate cell type. In some embodiments, a cell is any cell that expresses HBV antigen (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell a pharmaceutical combination comprising an effective amount any one of the oligonucleotides disclosed herein, particularly an RNAi oligonucleotide disclosed herein, for purposes of reducing expression of HBsAg solely in hepatocytes.

In some embodiments, oligonucleotides in the pharmaceutical combinations disclosed herein can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of HBV antigen expression (e.g., RNA, protein). In some embodiments, the extent to which an oligonucleotide of a pharmaceutical combination provided herein reduces levels of expression of HBV antigen is evaluated by comparing expression levels (e.g., mRNA or protein levels) of HBV antigen to an appropriate control (e.g., a level of HBV antigen expression in a cell or population of cells to which the pharmaceutical combination has not been delivered or to which a negative control has been delivered). In some embodiments, an appropriate control level of HBV antigen expression may be a predetermined level or value, such that a control level need not be measured every time. The predetermined level or value can take a variety of forms. In some embodiments, a predetermined level or value can be single cut-off value, such as a median or mean.

In some embodiments, administration of a pharmaceutical combination comprising an oligonucleotide as described herein, particularly an RNAi oligonucleotide described herein, results in a reduction in the level of HBV antigen (e.g., HBsAg) expression in a cell. In some embodiments, the reduction in levels of HBV antigen expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of HBV antigen. The appropriate control level may be a level of HBV antigen expression in a cell or population of cells that has not been contacted with a pharmaceutical combination comprising an oligonucleotide, particularly an RNAi oligonucleotide, as described herein. In some embodiments, the effect of delivery of an oligonucleotide of a pharmaceutical combination of the present invention to a cell according to a method disclosed herein is assessed after a finite period of time. For example, levels of HBV antigen may be analyzed in a cell at least 8 hours, 12 hours, 18 hours, 24 hours; or at least one, two, three, four, five, six, seven, fourteen, twenty-one, twenty-eight, thirty-five, forty-two, forty-nine, fifty-six, sixty-three, seventy, seventy-seven, eighty-four, ninety-one, ninety-eight, 105, 112, 119, 126, 133, 140, or 147 days after introduction of the oligonucleotide into the cell.

In some embodiments, the reduction in the level of HBV antigen (e.g., HBsAg) expression persists for an extended period of time following administration. In some embodiments, a detectable reduction in HBsAg expression persists within a period of 7 to 70 days following administration of an oligonucleotide of the pharmaceutical combination of the present invention, particularly where the oligonucleotide is an antisense oligonucleotide. For example, in some embodiments, the detectable reduction persists within a period of 10 to 70, 10 to 60, 10 to 50, 10 to 40, 10 to 30, or 10 to 20 days following administration of the oligonucleotide. In some embodiments, the detectable reduction persists within a period of 20 to 70, 20 to 60, 20 to 50, 20 to 40, or 20 to 30 days following administration of the oligonucleotide of the pharmaceutical combination of the present invention, particularly where the oligonucleotide is an antisense oligonucleotide. In some embodiments, the detectable reduction persists within a period of 30 to 70, 30 to 60, 30 to 50, or 30 to 40 days following administration of the oligonucleotide of the pharmaceutical combination of the present invention, particularly where the oligonucleotide is an antisense oligonucleotide. In some embodiments, the detectable reduction persists within a period of 40 to 70, 40 to 60, 40 to 50, 50 to 70, 50 to 60, or 60 to 70 days following administration of the oligonucleotide of the pharmaceutical combination of the present invention, particularly where the oligonucleotide is an antisense oligonucleotide.

In some embodiments, a detectable reduction in HBsAg expression persists within a period of 2 to 21 weeks following administration of an oligonucleotide of a pharmaceutical combination of the present invention, particularly where the oligonucleotide is an antisense oligonucleotide. For example, in some embodiments, the detectable reduction persists within a period of 2 to 20, 4 to 20, 6 to 20, 8 to 20, 10 to 20, 12 to 20, 14 to 20, 16 to 20, or 18 to 20 weeks following administration of the oligonucleotide of the pharmaceutical combination of the present invention, particularly where the oligonucleotide is an antisense oligonucleotide. In some embodiments, the detectable reduction persists within a period of 2 to 16, 4 to 16, 6 to 16, 8 to 16, 10 to 16, 12 to 16, or 14 to 16 weeks following administration of the oligonucleotide of the pharmaceutical combination of the present invention, particularly where the oligonucleotide is an antisense oligonucleotide. In some embodiments, the detectable reduction persists within a period of 2 to 12, 4 to 12, 6 to 12, 8 to 12, or 10 to 12 weeks following administration of the oligonucleotide of the pharmaceutical combination of the present invention, particularly where the oligonucleotide is an antisense oligonucleotide. In some embodiments, the detectable reduction persists within a period of 2 to 10, 4 to 10, 6 to 10, or 8 to 10 weeks following administration of the oligonucleotide of the pharmaceutical combination of the present invention, particularly where the oligonucleotide is an antisense oligonucleotide.

In some embodiments, an oligonucleotide of the pharmaceutical combination of the present invention, particularly where the oligonucleotide is an antisense oligonucleotide, is delivered in the form of a transgene that is engineered to express the oligonucleotide (e.g., its sense and antisense strands) in a cell. In some embodiments, an oligonucleotide of the pharmaceutical combination of the present invention, particularly where the oligonucleotide is an antisense oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes of the pharmaceutical combinations of the present invention can be injected directly to a subject.

II. Treatment Methods

Aspects of the disclosure relate to methods for reducing HBsAg expression (e.g., reducing HBsAg expression) for the treatment of HBV infection in a subject. In some embodiments, the methods may comprise administering to a subject in need thereof a pharmaceutical combination comprising an effective amount of any one of the oligonucleotides disclosed herein. The present disclosure provides for both prophylactic and therapeutic methods of treating a subject at risk of (or susceptible to) HBV infection and/or a disease or disorder associated with HBV infection.

In certain aspects, the disclosure provides a method for preventing in a subject, a disease or disorder as described herein by administering to the subject a therapeutic agent (e.g., a therapeutic combination, an oligonucleotide or vector or transgene encoding same). In some embodiments, particularly where the oligonucleotide of the therapeutic combination is an RNAi oligonucleotide, the subject to be treated is a subject who will benefit therapeutically from a reduction in the amount of HBsAg protein, e.g., in the liver. Subjects at risk for the disease or disorder can be identified by, for example, one or a combination of diagnostic or prognostic assays known in the art (e.g., identification of liver cirrhosis and/or liver inflammation). Administration of a prophylactic agent can occur prior to the detection of or the manifestation of symptoms characteristic of the disease or disorder, such that the disease or disorder is prevented or, alternatively, delayed in its progression.

Methods described herein typically involve administering to a subject an effective amount of an therapeutic combination, that is, an amount capable of producing a desirable therapeutic result.

A therapeutically acceptable amount may be an amount that is capable of treating a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently. For example, the dosage can be in the range of 0.1 mg/kg to 12 mg/kg. The dosage could also be in the range of 0.5 to 10 mg/kg. Alternatively, the dosage can be in the range of 1.0 to 6.0 mg/kg. The dosage could also be in the range of 3.0 to 5.0 mg/kg.

In some embodiments, a subject is administered any one of the compositions of the therapeutic combinations disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides of the therapeutic combinations disclosed herein are administered intravenously or subcutaneously. As a non-limiting set of examples, the oligonucleotides of the therapeutic combinations of the instant disclosure would typically be administered quarterly (once every three months), bi-monthly (once every two months), monthly, or weekly. For example, the oligonucleotides may be administered every one, two, or three weeks. The oligonucleotides may be administered daily.

In a preferred embodiment, the RNAi compound of the present invention is a siRNA targeting HBV, which is subcutaneously administered at a dose of between 0.1 mg/kg and 7 mg/kg, preferably between 0.5 mg/kg and 6.5 mg/kg, most preferably between 1 mg/kg and 6 mg/kg. In an embodiment, the dose is administered once every two weeks, once every four weeks or once every six weeks. In a preferred embodiment, the dose is administered once a month. In a particularly preferred embodiment, a dose of between 1 mg/kg and 6 mg/kg is administered once a month. Once a month is understood as meaning that consecutive doses are administered with an interval which is approximately the length of one calendar month.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EMBODIMENTS

The following embodiments of the present invention may be used in combination with any other embodiments described herein.

1. A pharmaceutical combination which comprises or consists of a therapeutic oligonucleotide, and a TLR7 agonist of formula (I) or (II):

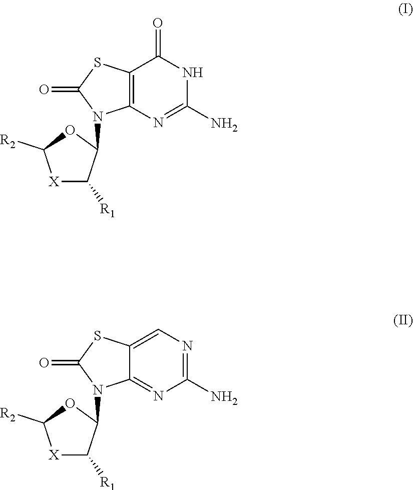

wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or
1-hydroxymethyl or acetoxy(cyclopropyl)methyl or acetoxy(propyn-1-yl)methyl, or a pharmaceutically acceptable salt, enantiomer or diastereomer thereof.

2. The pharmaceutical combination of embodiment 1, wherein the therapeutic oligonucleotide is an RNAi oligonucleotide.

3. The pharmaceutical combination of embodiment 2, wherein the RNAi oligonucleotide is an oligonucleotide targeting HBV (RNAi ID NO: 1).

4. The pharmaceutical combination of embodiment 2 or 3, wherein the RNAi oligonucleotide is an oligonucleotide targeting HBsAg mRNA (RNAi ID NO: 2).

5. The pharmaceutical combination of any one of embodiments 2-4, wherein the RNAi oligonucleotide is an oligonucleotide which reduces expression of HBsAg mRNA (RNAi ID NO: 3).

6. The pharmaceutical combination of any one of embodiments 2-5, wherein the RNAi oligonucleotide is an oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33) (RNAi ID NO: 4).

7. The pharmaceutical combination of any one of embodiments 2-5, wherein the RNAi oligonucleotide is an oligonucleotide for reducing expression of hepatitis B virus surface antigen (HBsAg) mRNA, the oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33) (RNAi ID NO: 5).

8. The pharmaceutical combination of embodiment 6 or 7, wherein the RNAi oligonucleotide further comprises a sense strand of 19 to 50 nucleotides in length, wherein the sense strand forms a duplex region with the antisense strand.

9. The pharmaceutical combination of embodiment 8, wherein the sense strand comprises a region of complementarity to a sequence as set forth in UUNUUGUGAGGAUUN (SEQ ID NO: 34).

10. The pharmaceutical combination of embodiment 8 or 9, wherein the sense strand comprises a region of complementarity to a sequence as set forth in 5′-UUAUUGUGAGGAUUNUUGUC (SEQ ID NO: 35)

11. The pharmaceutical combination of embodiment 9, wherein the antisense strand comprises a sequence as set forth in UUAUUGUGAGGAUUNUUGUCGG (SEQ ID NO: 36).

12. The pharmaceutical combination of embodiment 9, wherein the antisense strand consists of a sequence as set forth in UUAUUGUGAGGAUUCUUGUCGG (SEQ ID NO: 37).

13. The pharmaceutical combination of embodiment 9, wherein the antisense strand consists of a sequence as set forth in UUAUUGUGAGGAUUUUUGUCGG (SEQ ID NO: 38).

14. The pharmaceutical combination of any one of embodiments 8 to 12, wherein the sense strand comprises a sequence as set forth in ACAANAAUCCUCACAAUAA (SEQ ID NO: 39).

15. The pharmaceutical combination of any one of embodiments 8 to 14, wherein the sense strand comprises a sequence as set forth in GACAANAAUCCUCACAAUAAGCAGCCGAAAGGCUGC (SEQ ID NO: 40).

16. The pharmaceutical combination of any one of embodiments 8 to 14, wherein the sense strand consists of a sequence as set forth in GACAAAAAUCCUCACAAUAAGCAGCCGAAAGGCUGC (SEQ ID NO: 41).

17. The pharmaceutical combination of any one of embodiments 8 to 14, wherein the sense strand consists of a sequence as set forth in GACAAGAAUCCUCACAAUAAGCAGCCGAAAGGCUGC (SEQ ID NO: 42).

18. The pharmaceutical combination of any one of embodiments 2-5, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein the sense strand comprises a sequence as set forth in GACAAAAAUCCUCACAAUAAGCAGCCGAAAGGCUGC (SEQ ID NO: 41), wherein the antisense strand comprises a sequence as set forth in

(SEQ ID NO: 38)
UUAUUGUGAGGAUUUUUGUCGG,

wherein each of the antisense strand and the sense strand comprises one or more 2′-fluoro and 2′-O-methyl modified nucleotides and at least one phosphorothioate linkage, wherein the 4′-carbon of the sugar of the 5′-nucleotide of the antisense strand comprises a phosphate analog, and wherein the sense strand is conjugated to one or more N-acetylgalactosamine (GalNAc) moiety.

19. The pharmaceutical combination of any one of embodiments 2-5, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13 and 17; 2′-O-methyl modified nucleotides at positions 1, 2, 4-7, 11, 14-16, 18-26 and 31-36, and at least one phosphorothioate internucleotide linkage, wherein the sense strand is conjugated to one or more N-acetylgalactosamine (GalNAc) moiety; and
the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16 and 19; 2′-O-methyl modified nucleotides at positions 1, 4, 6, 9, 11, 13, 15, 17, 18 and 20-22, and at least three phosphorothioate internucleotide linkages, wherein the 4′-carbon of the sugar of the 5′-nucleotide of the antisense strand comprises a phosphate analog.

20. The pharmaceutical combination of embodiment 19, wherein the sense strand comprises a phosphorothioate linkage between the nucleotides at positions 1 and 2.

21. The pharmaceutical combination of embodiment 19 or 20, wherein the antisense strand comprises five phosphorothioate linkages between nucleotides 1 and 2, 2 and 3, 3 and 4, 20 and 21, and 21 and 22.

22. The pharmaceutical combination of any one of embodiments 19 to 21, wherein the 5′-nucleotide of the antisense strand has the following structure:

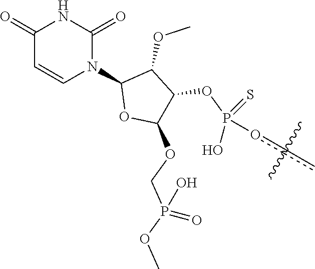

23. The pharmaceutical combination of any one of embodiments 19 to 22, wherein one or more of the nucleotides of the -GAAA- sequence on the sense strand is conjugated to a monovalent GalNAc moiety.

24. The pharmaceutical combination of embodiment 23, wherein each of the nucleotides of the -GAAA- sequence on the sense strand is conjugated to a monovalent GalNAc moiety.

25. The pharmaceutical combination of embodiment 24, wherein the -GAAA- motif comprises the structure:

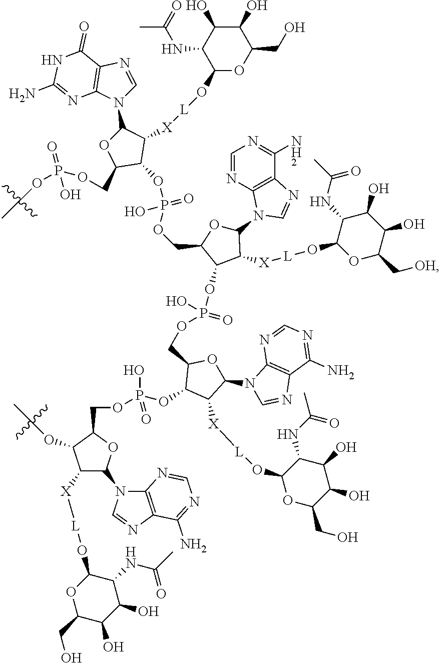

wherein:
L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and
X is a O, S, or N.

26. The pharmaceutical combination of embodiment 25, wherein L is an acetal linker.

27. The pharmaceutical combination of embodiment 25 or 26, wherein X is O.

28. The pharmaceutical combination of embodiment 20, wherein the -GAAA- sequence comprises the structure:

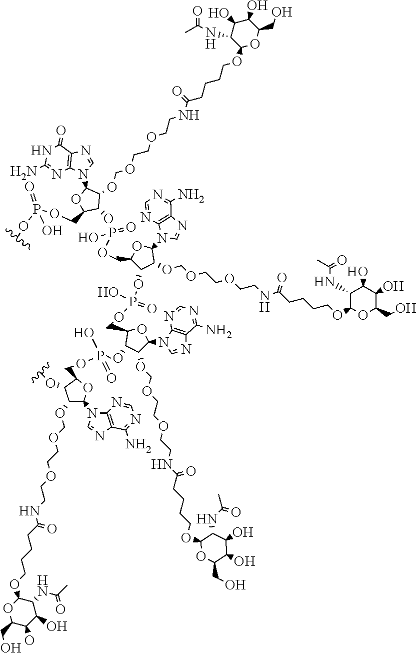 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

29. The pharmaceutical combination of embodiment 8, wherein the sense strand comprises at its 3′-end a stem-loop set forth as: S₁-L-S₂, wherein S₁ is complementary to S₂, and wherein L forms a loop between S₁ and S₂ of up to 6 nucleotides in length.

30. The pharmaceutical combination of embodiment 29, wherein L is a tetraloop.

31. The pharmaceutical combination of embodiment 29 or 30, wherein L forms a loop between S₁ and S₂ of 4 nucleotides in length.

32. The pharmaceutical combination of any one of embodiments 29 to 31, wherein L comprises a sequence set forth as GAAA.

33. The pharmaceutical combination of any one of embodiments 29 to 32, wherein up to 4 nucleotides of L of the stem-loop are each conjugated to a separate GalNAc.

34. The pharmaceutical combination of any one of embodiments 6 to 16, wherein the RNAi oligonucleotide comprises at least one modified nucleotide.

35. The pharmaceutical combination of embodiment 34, wherein the modified nucleotide comprises a 2′-modification.

36. The pharmaceutical combination of embodiment 35, wherein the 2′-modification is a modification selected from: 2′-aminoethyl, 2′-fluoro, 2′-O-methyl, 2′-O-methoxyethyl, and 2′-deoxy-2′-fluoro-β-d-arabinonucleic acid.

37. The pharmaceutical combination of any one of embodiments 6 to 16, wherein all of the nucleotides of the RNAi oligonucleotide are modified nucleotides.

38. The pharmaceutical combination of any one of embodiments 6 to 16, wherein the RNAi oligonucleotide comprises at least one modified internucleotide linkage.

39. The pharmaceutical combination of embodiment 38, wherein the at least one modified internucleotide linkage is a phosphorothioate linkage.

40. The pharmaceutical combination of any one of embodiments 6 to 16, wherein the 4′-carbon of the sugar of the 5′-nucleotide of the antisense strand comprises a phosphate analog.

41. The pharmaceutical combination of any one of embodiments 6 to 16, wherein at least one nucleotide of the oligonucleotide is conjugated to a targeting ligand.

42. The pharmaceutical combination of embodiment 41, wherein the targeting ligand is a N-acetylgalactosamine (GalNAc) moiety.

43. The pharmaceutical combination of any one of embodiments 2-5, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:

the sense strand consists of a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein each of the nucleotides of the -GAAA-sequence on the sense strand is conjugated to a monovalent GalNAc moiety; and

the antisense strand consists of a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22, wherein the 4′-carbon of the sugar of the 5′-nucleotide of the antisense strand comprises a methoxy phosphonate (MOP)(RNAi ID NO: 6).

44. The pharmaceutical combination of any one of embodiments 2-5, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13 and 17; 2′-O-methyl modified nucleotides at positions 1, 2, 4-7, 11, 14-16, 18-26 and 31-36, and one phosphorothioate internucleotide linkage between the nucleotides at positions 1 and 2, wherein each of the nucleotides of the -GAAA- sequence on the sense strand is conjugated to a monovalent GalNAc moiety, wherein the -GAAA- sequence comprises the structure:

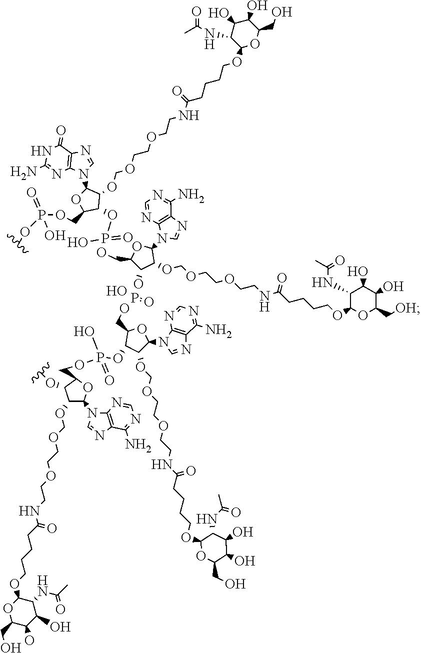 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and

the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16 and 19; 2′-O-methyl modified nucleotides at positions 1, 4, 6, 9, 11, 13, 15, 17, 18 and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

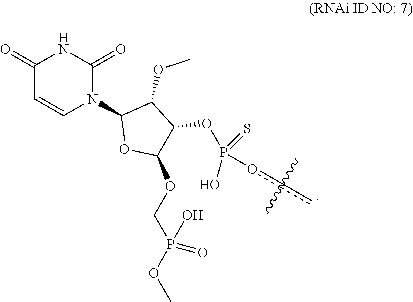

45. The pharmaceutical combination of any one of embodiments 2-5, wherein the RNAi oligonucleotide has the structure depicted in FIG. 29A (RNAi ID NO: 8).

46. The pharmaceutical combination of any one of embodiments 2-5, wherein the RNAi oligonucleotide is the oligonucleotide HBV(s)-219 (RNAi ID NO: 9).

47. The pharmaceutical combination of embodiment 1, wherein the therapeutic oligonucleotide is a GalNAc conjugated antisense oligonucleotide of 13 to 22 nucleotides in length with a contiguous nucleotide sequence of at least 12 nucleotides which is 100% complementary to a contiguous sequence from position 1530 to 1602 of SEQ ID NO: 1.

48. The pharmaceutical combination of embodiment 47, wherein the contiguous nucleotide sequence is 100% complementary to a target sequence selected from the group consisting of position 1530 to 1598; 1530-1543; 1530-1544; 1531-1543; 1551-1565; 1551-1566; 1577-1589; 1577-1591; 1577-1592; 1578-1590; 1578-1592; 1583-1598; 1584-1598; 1585-1598 and 1583-1602 of SEQ ID NO: 1.

49. The pharmaceutical combination of embodiment 47 or 48, wherein the contiguous nucleotide sequence is between 12 and 16 nucleotides in length.

50. The pharmaceutical combination of any one of embodiments 47 to 49, wherein the contiguous nucleotide sequence of the GalNAc conjugated antisense oligonucleotide is selected from the group consisting of

(SEQ ID NO: 2)
gcgtaaagagagg;
(SEQ ID NO: 3)
gcgtaaagagaggt;
(SEQ ID NO 4)
cgcgtaaagagaggt;
(SEQ ID NO 5)
agaaggcacagacgg;
(SEQ ID NO 6)
gagaaggcacagacgg;
(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc,

or a pharmaceutically acceptable salt thereof.

51. The pharmaceutical combination of any one of embodiments 47 to 50, wherein the contiguous nucleotide sequence of the GalNAc conjugated antisense oligonucleotide is a gapmer of formula 5′-F-G-F′-3′, where region F and F′ independently consists of 2-5 2′ sugar modified nucleotides and defines the 5′ and 3′ end of the F and F′ region, and G is a region between 6 and 10 DNA nucleosides which are capable of recruiting RNase H.

52. The pharmaceutical combination of embodiment 51, wherein the 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.

53. The pharmaceutical combination of embodiment 51 or 52, wherein the one or more 2′ sugar modified nucleoside is a MOE nucleoside.

54. The pharmaceutical combination of embodiment 51 or 52, wherein the one or more 2′ sugar modified nucleoside is a LNA nucleoside.

55. The pharmaceutical combination of embodiment 54, wherein the modified LNA nucleoside is selected from oxy-LNA, amino-LNA, thio-LNA, cET, and ENA.

56. The pharmaceutical combination of embodiment 54 or 55, wherein the modified LNA nucleoside is oxy-LNA with the following 2′-4′ bridge —O—CH₂—.

57. The pharmaceutical combination of embodiment 56, wherein the oxy-LNA is beta-D-oxy-LNA.

58. The pharmaceutical combination of embodiment 54 or 55, wherein the modified LNA nucleoside is cET with the following 2′-4′ bridge —O—CH(CH₃)—.

59. The pharmaceutical combination of embodiment 58, wherein the cET is (S)cET, i.e. 6′(S)methyl-beta-D-oxy-LNA.

60. The pharmaceutical combination of embodiment 54 or 55, wherein the LNA is ENA, with the following 2′-4′ bridge —O—CH₂—CH₂—.

61. The pharmaceutical combination of any one of embodiments 47 to 60, wherein the contiguous nucleotide sequence of the GalNAc conjugated antisense oligonucleotide is selected from the group consisting of:

(SEQ ID NO: 2)
gcgtaaagagagg;
(SEQ ID NO: 3)
gcgtaaagagaggt;
(SEQ ID NO 4)
cgcgtaaagagaggt;
(SEQ ID NO 5)
agaaggcacagacgg;
(SEQ ID NO 6)
gagaaggcacagacgg;
(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc,

wherein uppercase letters denote LNA or MOE nucleosides and lower case letters denote DNA nucleosides.

62. The pharmaceutical combination of any one of embodiments 47 to 61, wherein at least 50% of the internucleoside linkages within the contiguous nucleotide sequence are phosphorothioate internucleoside linkages.

63. The pharmaceutical combination of any one of embodiments 47 to 62, wherein all the internucleoside linkages within the contiguous nucleotide sequence of the GalNAc conjugated antisense oligonucleotide are phosphorothioate internucleoside linkages.

64. The pharmaceutical combination of any one of embodiments 47 to 63, wherein the GalNAc conjugate of the GalNAc conjugated antisense oligonucleotide is a di-valent, tri-valent or tetra-valent GalNAc cluster.

65. The pharmaceutical combination of embodiment 64, wherein the GalNAc conjugate is selected from FIG. 1B, 1D or 1J.

66. The pharmaceutical combination of any one of embodiments 47 to 65, wherein the GalNAc conjugate and the contiguous nucleotide sequence of the GalNAc conjugated antisense oligonucleotide is covalently linked by way of a PO linker comprising two, three, four or five phosphodiester linked DNA nucleosides.

67. The pharmaceutical combination of embodiment of embodiment 66, wherein the PO linker is part of the antisense oligonucleotide and consists of the dinucleotide sequence of cytosine and adenine (CA) with at least two phosphodiester linkages one between the C and A and one being to the GalNAc cluster.

68. The pharmaceutical combination of any one of embodiments 47 to 67, wherein the GalNAc conjugated antisense oligonucleotide is 12 to 18 nucleotides in length.

69. The pharmaceutical combination of any one of embodiments 47 to 68, wherein the GalNAc conjugated antisense oligonucleotide is selected from the group consisting of:

SEQ ID NO: 15
5′-GN2-C6ₒcₒaₒGₛ^{m}CₛGₛtₛaₛaₛaₛgₛaₛgₛaₛGₛG-3′
SEQ ID NO: 15
5′-GN2-C6ₒcₒaₒGₛ^{m}CₛGₛtₛaₛaₛaₛgₛaₛgₛAₛGₛG-3′
SEQ ID NO: 16
5-GN2-C6ₒcₒaₒGₛ^{m}CₛGₛtₛaₛaₛaₛgₛaₛgₛaₛGₛGₛT-3′
SEQ ID NO: 17
5′-GN2-C6ₒcₒaₒ^{m}CₛGₛ^{m}CₛgₛtₛaₛaₛaₛgₛaₛgₛaₛGₛGₛT-3′
SEQ ID NO: 18
5′-GN2-C6ₒcₒaₒGₛAₛGₛaₛaₛgₛgₛcₛaₛcₛaₛgₛaₛ^{m}CₛGₛG-3′
SEQ ID NO: 19
5′-GN2-C6ₒcₒaₒGₛAₛGₛaₛaₛgₛgₛcₛaₛcₛaₛgₛaₛ^{m}CₛGₛG-3′
SEQ ID NO: 20
5′-GN2-C6₀c₀a₀AₛGₛ^{m}Cₛgₛaₛaₛgₛtₛgₛcₛaₛcₛaₛ^{m}CₛGₛG-3
SEQ ID NO: 21
5′-GN2-C6₀c₀a₀GₛAₛAₛgₛtₛgₛcₛaₛcₛaₛ^{m}cₛGₛG-3′
SEQ ID NO: 21
5′-GN2-C6₀c₀a₀GsAₛAₛgₛtₛgₛcₛaₛcₛaₛ^{m}CₛGₛG-3′
SEQ ID NO: 22
5′-GN2-C6₀c₀a₀Gₛ^{m}CₛGₛaₛaₛgₛtₛgₛcₛaₛcₛaₛ^{m}CₛGₛG-3′
SEQ ID NO: 23
5′-GN2-C6ₒcₒaₒAₛGₛ^{m}CₛgₛaₛaₛgₛtₛgₛcₛaₛcₛAₛ^{m}CₛG-3′;
SEQ ID NO: 24
5′-GN2-C6₀c₀a₀^{m}CₛGₛAₛaₛgₛtₛgₛcₛaₛcₛaₛ^{m}CₛG-3′
SEQ ID NO: 25
5′-GN2-C6ₒcₒaₒAₛGₛGₛtₛgₛaₛaₛgₛ^{m}cₛgₛaₛaₛgₛTₛGₛ^{m}c-3′
SEQ ID NO: 26
5′-GN2-C6ₒcₒaₒAₛGₛgₛtₛgₛaₛaₛgₛ^{m}cₛgₛaₛAₛGₛTₛG-3′
SEQ ID NO: 26
5′-GN2-C6₀c₀a₀AₛGₛGₛtₛgₛaₛaₛgₛ^{m}cₛgₛaₛaₛGₛTₛG-3′;
and
SEQ ID NO: 27
5′-GN2-C6ₒcₒaₒAₛGₛGₛtₛgₛaₛaₛgₛ^{m}cₛgₛaₛAₛGₛT-3′

wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker, or a pharmaceutically acceptable salt thereof.

70. The pharmaceutical combination of any one of embodiments 47 to 69, wherein the GalNAc conjugated antisense oligonucleotide is 5′-FIG. 1J- ₒG_{S}C_{S}A_{S}g_{S}g_{S}t_{S}g_{S}a_{S}a_{S}g_{S}c_{S}g_{S}a_{S}A_{S}G_{S}T_{S}G_{S}C-3′ (FIG. 2), wherein underlined uppercaSe underlined letters denote MOE units; lowercase letters denote DNA units; subscript “o” denotes a phosphodiester linkage; subscript “s” denotes a phosphorothioate linkage.

71. The pharmaceutical combination of any one of embodiments 1 to 70, wherein the TLR7 agonist is of formula (III):

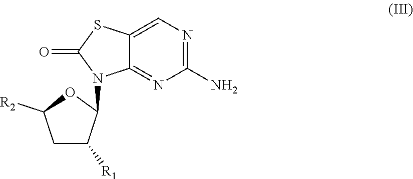

wherein R₁ is —OH or acetoxy and R₂ is 1-acetoxypropyl or 1-hydroxypropyl or 1-hydroxymethyl
   or a pharmaceutically acceptable salt, enantiomer or diastereomer thereof.

72. The pharmaceutical combination of any one of embodiments 1 to 70, wherein the TLR7 agonist is of formula (IV):

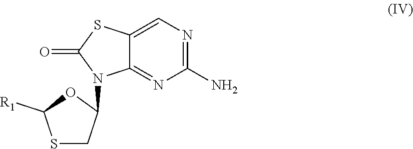

wherein R₁ is acetoxy(cyclopropyl)methyl or acetoxy(propyn-1-yl)methyl.

73. The pharmaceutical combination of any one of embodiments 1 to 70, wherein the TLR7 agonist is of formula (V):

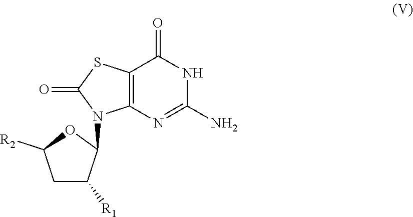

wherein R₁ is —OH and R₂ is 1-hydroxypropyl or hydroxymethyl or a pharmaceutically acceptable salt, enantiomer or diastereomer thereof.

74. The pharmaceutical combination of any one of embodiments 0 to 73, wherein the TLR7 agonist is selected from the group consisting of:
[(1S)-1-[(2S,4R,5R)-5-(5-amino-2-oxo-thiazolo[4,5-d]pyrimidin-3-yl)-4-hydroxy-tetrahydrofuran-2-yl]propyl] acetate (CMP ID NO: VI);
5-amino-3-[(2R,3R,5S)-3-hydroxy-5-[(15)-1-hydroxypropyl]tetrahydrofuran-2-yl]-6H-thiazolo[4,5-d]pyrimidine-2,7-dione (CMP ID NO: VII);
5-amino-3-[(2R,3R,5S)-3-hydroxy-5-[(15)-1-hydroxypropyl]tetrahydrofuran-2-yl]thiazolo[4,5-d]pyrimidin-2-one (CMP ID NO: VIII);
5-amino-3-(3′-deoxy-β-D-ribofuranosyI)-3H-thiazolo[4,5-d]pyrimidin-2-one (CMP ID NO: IX);
5-amino-3-(2′-O-acetyl-3′-deoxy-β-D-ribofuranosyl)-3H-thiazolo[4,5-d]pyrimidin-2-one (CMP ID NO: X);
5-amino-3-(3′-deoxy-p-D-ribofuranosyI)-3H,6H-thiazolo[4,5-d]pyrimidin-2,7-dione (CMP ID NO: XI);
[(S)-[(2S,5R)-5-(5-amino-2-oxo-thiazolo[4,5-d]pyrimidin-3-yl)-1,3-oxathiolan-2-yl]-cyclopropyl-methyl] acetate (CMP ID NO: XII); and
(1S)-1-[(2S,5R)-5-(5-amino-2-oxo-thiazolo[4,5-d]pyrimidin-3-yl)-1,3-oxathiolan-2-yl]but-2-ynyl] acetate (CMP ID NO: XIII);

or a pharmaceutically acceptable salt, enantiomer or diastereomer thereof.

75. The pharmaceutical combination of any one of embodiments 2-46 and 71-74, wherein the combination comprising an RNAi oligonucleotide and a TLR7 agonist is selected from the group consisting of the following combinations:

RNAi ID NO: 1 and CMP ID NO: VI; RNAi ID NO: 2 and CMP ID NO: VI; RNAi ID NO: 3 and CMP ID NO: VI; RNAi ID NO: 4 and CMP ID NO: VI; RNAi ID NO: 5 and CMP ID NO: VI; RNAi ID NO: 6 and CMP ID NO: VI; RNAi ID NO: 7 and CMP ID NO: VI; RNAi ID NO: 8 and CMP ID NO: VI; RNAi ID NO: 9 and CMP ID NO: VI;

RNAi ID NO: 1 and CMP ID NO: VII, RNAi ID NO: 2 and CMP ID NO: VII; RNAi ID NO: 3 and CMP ID NO: VII; RNAi ID NO: 4 and CMP ID NO: VII; RNAi ID NO: 5 and CMP ID NO: VII; RNAi ID NO: 6 and CMP ID NO: VII; RNAi ID NO: 7 and CMP ID NO: VII; RNAi ID NO: 8 and CMP ID NO: VII; RNAi ID NO: 9 and CMP ID NO: VII;

RNAi ID NO: 1 and CMP ID NO: VIII, RNAi ID NO: 2 and CMP ID NO: VIII; RNAi ID NO: 3 and CMP ID NO: VIII; RNAi ID NO: 4 and CMP ID NO: VIII; RNAi ID NO: 5 and CMP ID NO: VIII; RNAi ID NO: 6 and CMP ID NO: VIII; RNAi ID NO: 7 and CMP ID NO: VIII; RNAi ID NO: 8 and CMP ID NO: VIII; RNAi ID NO: 9 and CMP ID NO: VIII;

RNAi ID NO: 1 and CMP ID NO: XIII, RNAi ID NO: 2 and CMP ID NO: XIII; RNAi ID NO: 3 and CMP ID NO: XIII; RNAi ID NO: 4 and CMP ID NO: XIII; RNAi ID NO: 5 and CMP ID NO: XIII; RNAi ID NO: 6 and CMP ID NO: XIII; RNAi ID NO: 7 and CMP ID NO: XIII; RNAi ID NO: 8 and CMP ID NO: XIII, or RNAi ID NO: 9 and CMP ID NO: XIII;

or a pharmaceutically acceptable salt, enantiomer or diastereomer thereof.

76. The pharmaceutical combination of any one of embodiments 2-46 and 71-74, wherein the RNAi oligonucleotide is RNAi ID NO: 7:

An oligonucleotide comprising a sense strand forming a duplex region with an antisense strand, wherein:

the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GaINac moiety, wherein the -GAAA- sequence comprises the structure:

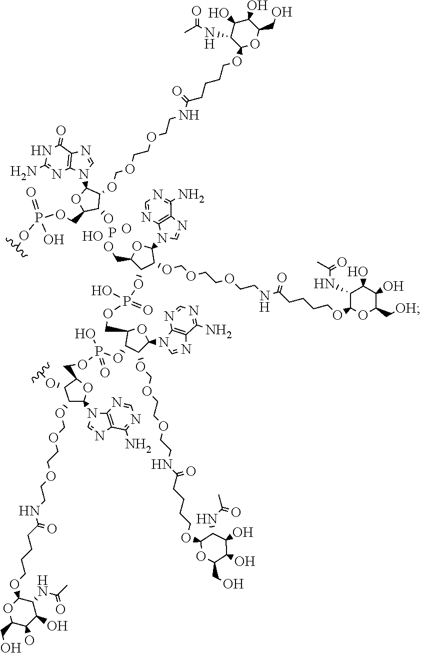 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and

the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

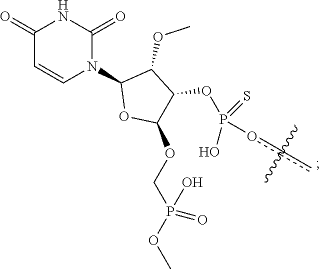

and the TLR7 agonist is CMP ID NO: VI:

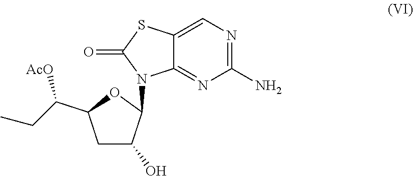 [SMILES: CC[C@H](OC(C)=O)[C@@H]1C[C@@H](O)[C@H](n2c(=O)sc3cnc(N)nc32)O1]

or a pharmaceutically acceptable salt, enantiomer or diastereomer thereof.

77. The pharmaceutical combination of any one of embodiments 47 to 74, wherein the combination comprising a GalNAc conjugated antisense oligonucleotide and a TLR7 agonist is selected from the group consisting of the following combinations: CMP ID NO: 15_ 1 and VI, CMP ID NO: 15_ 2 and VI; CMP ID NO: 16_ 1 and VI; CMP ID NO: 20_ 1 and VI; CMP ID NO: 23_ 1 and VI; CMP ID NO: 26_ 1 and VI; CMP ID NO: 29_ 1 and VI; CMP ID NO: 15_ 1 and VII, CMP ID NO: 15_ 2 and VII; CMP ID NO: 16_ 1 and VII; CMP ID NO: 20_ 1 and VII; CMP ID NO: 23_ 1 and VII; CMP ID NO: 26_ 1 and VII; CMP ID NO: 29_ 1 and VII; CMP ID NO: 15_ 1 and VIII, CMP ID NO: 15_ 2 and VIII; CMP ID NO: 16_ 1 and VIII; CMP ID NO: 20_ 1 and VIII; CMP ID NO: 23_ 1 and VII; CMP ID NO: 26_ 1 and VIII; CMP ID NO: 29_ 1 and VIII; CMP ID NO: 15_ 1 and XIII, CMP ID NO: 15_ 2 and XIII; CMP ID NO: 16_ 1 and XIII; CMP ID NO: 20_ 1 and XIII; CMP ID NO: 23_ 1 and XIII; CMP ID NO: 26_ 1 and XIII; and CMP ID NO: 29_ 1 and XIII, or a pharmaceutically acceptable salt, enantiomer or diastereomer thereof.

78. The pharmaceutical combination of any one of embodiments 47 to 74, wherein the GalNAc conjugated antisense oligonucleotide is CMP ID NO: 15_1 as shown in FIG. 5 and the TLR7 agonist is CMP ID NO: VI:

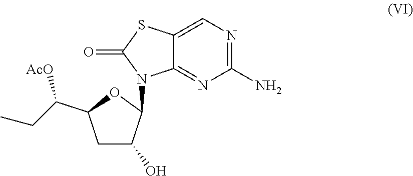 [SMILES: CC[C@H](OC(C)=O)[C@@H]1C[C@@H](O)[C@H](n2c(=O)sc3cnc(N)nc32)O1]

or a pharmaceutically acceptable salt, enantiomer or diastereomer thereof.

79. The pharmaceutical combination of any one of embodiments 1 to 78 wherein the therapeutic oligonucleotide is formulated with a pharmaceutically acceptable salt.

80. The pharmaceutical combination of embodiment 79 wherein the pharmaceutically acceptable salt is a metal cation ion, preferably wherein the pharmaceutically acceptable salt is Na⁺ or K⁺.

81. The pharmaceutical combination of any one of embodiments 1 to 80, wherein the therapeutic oligonucleotide and TLR7 agonist according to any one of embodiments 1 to 79 are formulated with a pharmaceutically acceptable carrier.

82. The pharmaceutical combination of embodiment 81, wherein the pharmaceutically acceptable carrier is water.

83. The pharmaceutical combination of any one of embodiments 1 to 82, wherein the therapeutic oligonucleotide is formulated in phosphate buffered saline.

84. The pharmaceutical combination of any one of embodiments 1 to 83, wherein the therapeutic oligonucleotide is formulated for subcutaneous injection and the TLR7 agonist is formulated for oral administration.

85. The pharmaceutical combination of any one of embodiments 1 to 83, wherein the therapeutic oligonucleotide is formulated for intravenous injection and the TLR7 agonist is formulated for oral administration.

86. The pharmaceutical combination of any one of embodiments 2 to 46, 75, 76 and 79-83, wherein the therapeutic oligonucleotide is siRNA formulated for subcutaneous injection and the TLR7 agonist is formulated for oral administration.

87. The pharmaceutical combination of any one of embodiments 1-86, wherein the pharmaceutical combination comprises an RNAi oligonucleotide and a TLR7 agonist, wherein the pharmaceutical combination further comprises a CpAM (core protein allosteric modulator).

88. The pharmaceutical combination of embodiment 87, wherein the CpAM has a formula according to Compound (CpAM1) shown below:

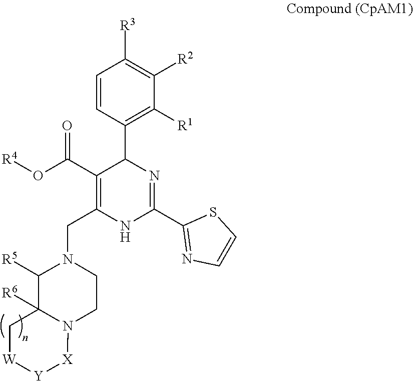

wherein

R¹ is hydrogen, halogen or C₁₋₆alkyl;

R² is hydrogen or halogen;

R³ is hydrogen or halogen;

R⁴ is C₁₋₆ alkyl;

R⁵ is hydrogen, hydroxyC₁₋₆alkyl, aminocarbonyl, C₁₋₆alkoxycarbonyl or carboxy;

R⁶ is hydrogen, C₁₋₆alkoxycarbonyl or carboxy-CₘH₂ₘ—,

X is carbonyl or sulfonyl;

Y is —CH₂—, —O— or —N(R⁷)—,

wherein R⁷ is hydrogen, C₁₋₆alkyl, haloC₁₋₆alkyl, C₃₋₇cycloalkyl-CₘH₂ₘ—, C₁₋₆alkoxycarbonyl-CₘH₂ₘ—, —CₜH₂ₜ—COOH, -haloC₁₋₆ alkyl-COOH, —(C₁₋₆alkoxy)C₁₋₆alkyl-COOH, —C₁₋₆alkyl-O—C₁₋₆alkyl-COOH, —C₃₋₇ cycloalkyl-CₘH₂ₘ—COOH, —CₘH₂ₘ—C₃₋₇ cycloalkyl-COOH, hydroxy-CₜH₂ₜ, carboxyspiro[3.3]heptyl or carboxyphenyl-CₘH₂ₘ—, carboxypyridinyl-CₘH₂ₘ—;

W is —CH₂—, —C(C₁₋₆ alkyl)₂—, —O— or carbonyl;

n is 0 or 1;

m is 0-7;

t is 1-7;

or pharmaceutically acceptable salts, or enantiomers or diastereomers thereof.

89. The pharmaceutical combination of embodiment 87 or 88, wherein the CpAM is Compound (CpAM2)

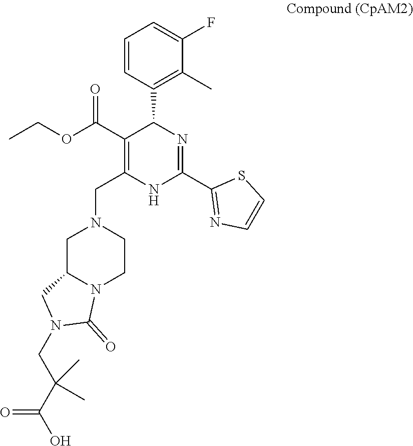 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

or a pharmaceutically acceptable salt, enantiomer or diastereomer thereof.

90. A pharmaceutical combination comprising an RNAi oligonucleotide, a TLR7 agonist and a CpAM, wherein the RNAi oligonucleotide is RNAi ID NO: 7:

An oligonucleotide comprising a sense strand forming a duplex region with an antisense strand, wherein:

the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GalNAc moiety, wherein the -GAAA- sequence comprises the structure:

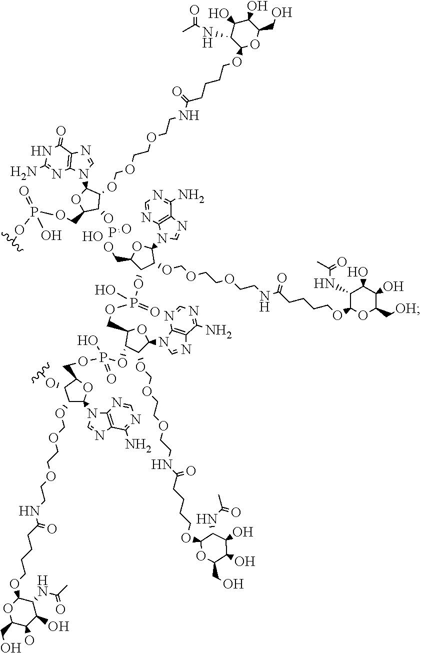 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and

the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

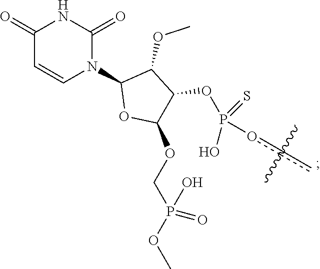

wherein the TLR7 agonist is CMP ID NO: VI:

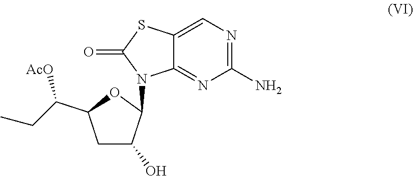 [SMILES: CC[C@H](OC(C)=O)[C@@H]1C[C@@H](O)[C@H](n2c(=O)sc3cnc(N)nc32)O1]

or a pharmaceutically acceptable salt, enantiomer or diastereomer thereof;

and wherein the CpAM is Compound (CpAM2):

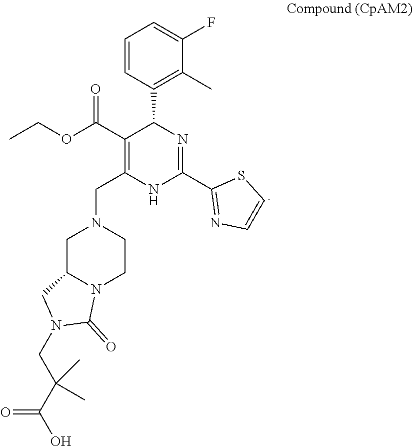 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

or a pharmaceutically acceptable salt, enantiomer or diastereomer thereof.

91. A pharmaceutical composition comprising the pharmaceutical combination of any one of any one of embodiments 1-90.

92. A kit of parts comprising a therapeutic oligonucleotide according to any one of embodiments 1 to 90 and a package insert with instruction for administration with a TLR7 agonist to treat a hepatitis B virus infection.

93. The kit of parts of embodiment 92, wherein the TLR7 agonist mentioned in the package insert is a TLR7 agonist according to any one of embodiments 1 to 90.

94. The kit of parts of embodiment 92 or 93, wherein the kit comprises a therapeutic oligonucleotide according to any one of embodiments 1 to 90 and a TLR7 agonist according to any one of embodiments 1 to 90.

95. The kit of parts of any one of embodiments 92 to 94, wherein the therapeutic oligonucleotide is formulated for subcutaneous injection and the TLR7 agonist is formulated for oral administration.

96. The kit of parts of any one of embodiments 92 to 95, wherein the package insert describes the treatment of a chronic hepatitis B virus infection.

97. The pharmaceutical combination, composition or kit of any one of embodiments 1 to 96, wherein the therapeutic oligonucleotide is in the form of a transgene that is engineered to express the oligonucleotide in a cell.

98. The use of the pharmaceutical combination, composition or kit of any one of embodiments 1 to 97 for treating a hepatitis B virus infection.

99. The use of embodiment 98, wherein the hepatitis B virus infection to be treated is a chronic hepatitis B virus infection.

100. The use of embodiment 98 or 99, wherein the therapeutic oligonucleotide and the TLR7 agonist are administered in pharmaceutically effective amounts.

101. The use of any one of embodiments 98 to 100, wherein the therapeutic oligonucleotide is administered weekly and the TLR7 agonist is administered every other day.

102. The use of any one of embodiments 98 to 101, wherein the therapeutic oligonucleotide is dosed at 1 to 4 mg/kg pr. administration and the TLR7 agonist is dosed at 150 to 170 mg pr. administration.

103. The use of any one of embodiments 98 to 102, wherein the therapeutic oligonucleotide is administered for 48 weeks and 84 doses of TLR7 agonist are administered.

104. The use of any one of embodiments 98 to 103, wherein the administration of the therapeutic oligonucleotide and the TLR7 agonist starts in the same week.

105. The use of any one of embodiments 98 to 104, wherein the therapeutic oligonucleotide is in a dosage form for subcutaneous administration and the TLR7 agonist is in a dosage form for oral administration.

106. The use of any one of embodiments 98 to 105, wherein the dose of the therapeutic oligonucleotide is 100 to 150 mg/ml and the dose of the TLR7 agonist is 150 to 170 mg.

107. The use of any one of embodiments 98 to 106, wherein the therapeutic oligonucleotide is administered in the absence of treatment with an RNAi oligonucleotide targeting a non-surface antigen encoding HBV mRNA transcript.

108. The use of any one of embodiments 98 to 107, wherein the subject is not administered an RNAi oligonucleotide that selectively targets HBxAg mRNA transcript.

109. The use of any one of embodiments 98 to 108, further comprising administering to the subject an effective amount of Entecavir.

110. The use of any one of embodiments 98 to 109, wherein the therapeutic oligonucleotide is delivered in the form of a transgene that is engineered to express the oligonucleotide in a cell.

111. The pharmaceutical combination, composition or kit of any one of embodiments 1 to 97, for use in medicine.

112. The pharmaceutical combination, composition or kit of any one of embodiments 1 to 97, for use in treatment of a hepatitis B virus infection.

113. The pharmaceutical combination, composition or kit for use of embodiment 111 or 112, wherein the hepatitis B virus infection to be treated is a chronic hepatitis B virus infection.

114. The pharmaceutical combination, composition or kit for use of any one of embodiments 111 to 113, wherein the therapeutic oligonucleotide and the TLR7 agonist are administered in pharmaceutically effective amounts.

115. The pharmaceutical combination, composition or kit for use of any one of embodiments 111 to 114, wherein the therapeutic oligonucleotide is administered weekly and the TLR7 agonist is administered every other day.

116. The pharmaceutical combination, composition or kit for use of any one of embodiments 111 to 115, wherein the therapeutic oligonucleotide is dosed at 1 to 4 mg/kg pr. administration and the TLR7 agonist is dosed at 150 to 170 mg pr. administration.

117. The pharmaceutical combination, composition or kit for use of any one of embodiments 111 to 116, wherein the therapeutic oligonucleotide is administered for 48 weeks and 84 doses of TLR7 agonist are administered.

118. The pharmaceutical combination, composition or kit for use of any one of embodiments 111 to 117, wherein the administration of the therapeutic oligonucleotide and the TLR7 agonist starts in the same week.

119. The pharmaceutical combination, composition or kit for use of any one of embodiments 111 to 118, wherein the therapeutic oligonucleotide is in a dosage form for subcutaneous administration and the TLR7 agonist is in a dosage form for oral administration.

120. The pharmaceutical combination, composition or kit for use of any one of embodiments 111 to 119, wherein the dose of the therapeutic oligonucleotide is 100 to 150 mg/ml and the dose of the TLR7 agonist is 150 to 170 mg.

121. The pharmaceutical combination, composition or kit for use of any one of embodiments 111 to 120, wherein the therapeutic oligonucleotide is administered in the absence of treatment with an RNAi oligonucleotide targeting a non-surface antigen encoding HBV mRNA transcript.

122. The pharmaceutical combination, composition or kit for use of any one of embodiments 111 to 121, wherein the subject is not administered an RNAi oligonucleotide that selectively targets HBxAg mRNA transcript.

123. The pharmaceutical combination, composition or kit for use of any one of embodiments 111 to 122, further comprising administering to the subject an effective amount of Entecavir.

124. The pharmaceutical combination, composition or kit for use of any one of embodiments 111 to 123, wherein the therapeutic oligonucleotide is delivered in the form of a transgene that is engineered to express the oligonucleotide in a cell.

125. Use of a therapeutic oligonucleotide in the manufacture of a first medicament for treating a hepatitis B virus infection, wherein the first medicament is a therapeutic oligonucleotide according to any one of embodiments 1 to 97 and wherein the first medicament is to be administered in combination with a second medicament, wherein the second medicament is a TLR7 agonist according to any one of embodiments 1 to 97.

126. Use of the pharmaceutical combination, composition or kit of any one of embodiments 1 to 97 in the manufacture of a medicament.

127. Use of the pharmaceutical combination, composition or kit of any one of embodiments 1 to 97 in the manufacture of a medicament for treating a hepatitis B virus infection.

128. The use of any one of embodiments 125 to 127, wherein the hepatitis B virus infection to be treated is a chronic hepatitis B virus infection.

129. The use of any one of embodiments 125 to 128, wherein the therapeutic oligonucleotide and the TLR7 agonist are administered in pharmaceutically effective amounts.

130. The use of any one of embodiments 125 to 129, wherein the therapeutic oligonucleotide is administered weekly and the TLR7 agonist is administered every other day.

131. The use of any one of embodiments 125 to 130, wherein the therapeutic oligonucleotide is dosed at 1 to 4 mg/kg pr. administration and the TLR7 agonist is dosed at 150 to 170 mg pr. administration.

132. The use of any one of embodiments 125 to 131, wherein the therapeutic oligonucleotide is administered for 48 weeks and 84 doses of TLR7 agonist are administered.

133. The use of any one of embodiments 125 to 132, wherein the administration of the therapeutic oligonucleotide and the TLR7 agonist starts in the same week.

134. The use of any one of embodiments 125 to 133, wherein the therapeutic oligonucleotide is in a dosage form for subcutaneous administration and the TLR7 agonist is in a dosage form for oral administration.

135. The use of any one of embodiments 125 to 134, wherein the dose of the therapeutic oligonucleotide is 100 to 150 mg/ml and the dose of the TLR7 agonist is 150 to 170 mg.

136. The use of any one of embodiments 125 to 135, wherein the therapeutic oligonucleotide is administered in the absence of treatment with an RNAi oligonucleotide targeting a non-surface antigen encoding HBV mRNA transcript.

137. The use of any one of embodiments 125 to 136, wherein the subject is not administered an RNAi oligonucleotide that selectively targets HBxAg mRNA transcript.

138. The use of any one of embodiments 125 to 137, further comprising administering to the subject an effective amount of Entecavir.

139. The use of any one of embodiments 125 to 138, wherein the therapeutic oligonucleotide is delivered in the form of a transgene that is engineered to express the oligonucleotide in a cell.

140. A method for treating a hepatitis B virus infection comprising administering a therapeutically effective amount of a therapeutic oligonucleotide of any one of embodiments Error! Reference source not found. to 97 in combination with a therapeutically effective amount of TLR7 agonist of any one of embodiments Error! Reference source not found. to 91 or 94 to 97 to a subject infected with a hepatitis B virus infection.

141. A method for treating a hepatitis B virus infection comprising administering a therapeutically effective amount of the pharmaceutical combination, composition or kit of any one of embodiments Error! Reference source not found. to 97 to a subject infected with a hepatitis B virus infection.

142. The method of embodiment 140 or 141, wherein the hepatitis B virus infection to be treated is a chronic hepatitis B virus infection.

143. The method of any one of embodiments 140 to 142, wherein the therapeutic oligonucleotide and the TLR7 agonist are administered in pharmaceutically effective amounts.

144. The method of any one of embodiments 140 to 143, wherein the therapeutic oligonucleotide is administered weekly and the TLR7 agonist is administered every other day.

145. The method of any one of embodiments 140 to 144, wherein the therapeutic oligonucleotide is dosed at 1 to 4 mg/kg pr. administration and the TLR7 agonist is dosed at 150 to 170 mg pr. administration.

146. The method of any one of embodiments 140 to 145, wherein the therapeutic oligonucleotide is administered for 48 weeks and 84 doses of TLR7 agonist are administered.

147. The method of any one of embodiments 140 to 146, wherein the administration of the therapeutic oligonucleotide and the TLR7 agonist starts in the same week.

148. The method of any one of embodiments 140 to 147, wherein the therapeutic oligonucleotide is in a dosage form for subcutaneous administration and the TLR7 agonist is in a dosage form for oral administration.

149. The method of any one of embodiments 140 to 148, wherein the dose of the therapeutic oligonucleotide is 100 to 150 mg/ml and the dose of the TLR7 agonist is 150 to 170 mg.

150. The method of any one of embodiments 140 to 149, wherein the therapeutic oligonucleotide is administered in the absence of treatment with an RNAi oligonucleotide targeting a non-surface antigen encoding HBV mRNA transcript.

151. The method of any one of embodiments 140 to 150, wherein the subject is not administered an RNAi oligonucleotide that selectively targets HBxAg mRNA transcript.

152. The method of any one of embodiments 140 to 151, further comprising administering to the subject an effective amount of Entecavir.

153. The method of any one of embodiments 140 to 152, wherein the therapeutic oligonucleotide is delivered in the form of a transgene that is engineered to express the oligonucleotide in a cell.

154. A method of reducing expression of hepatitis B virus surface antigen in a cell, the method comprising delivering to the cell the pharmaceutical combination or composition of any one of embodiments 1 to 91.

155. The method of embodiment 154, wherein the cell is a hepatocyte. 156. The method of embodiment 154 or 155, wherein the cell is in vivo.

157. The method of embodiment 154 or 155, wherein the cell is in vitro.

158. The method of any one of embodiments 154 to 157, wherein the therapeutic oligonucleotide is delivered in the form of a transgene that is engineered to express the oligonucleotide in the cell.

159. The pharmaceutical combination, composition, kit, use or method substantially as described herein and with reference to the accompanying drawings.

### EXAMPLES

Part A: Effects of an RNAi Oligonucleotide

### Example A1. Development of Potent Oligonucleotide Inhibitors of HBsAg Expression

HBV surface antigen was identified as a target for RNAi-based therapy to treat HBV infection. As depicted in the HBV genome organization shown in FIG. 20, HBsAg is encoded by three RNA molecules transcribed from a single ORF. Oligonucleotides were designed for purposes of silencing one or more RNA transcripts that contribute to HBsAg assembly (example RNAi target site indicated by “X” in FIG. 20). An HBsAg-targeting oligonucleotide, HBV-254, was designed and evaluated in vitro and in vivo. HBV-254 was selected and designed based on an ability to directly target mRNA transcripts for four HBV RNA species. The HBV-254 duplex oligonucleotide used in the experiments included a sense strand of a sequence as set forth in (shown 5′ to 3′): GUGGUGGACUUCUCUCAAUAGCAGCCGAAAGGCUGC (SEQ ID NO: 55); and an antisense strand of a sequence as set forth in (shown 5′ to 3′):

(SEQ ID NO: 56)
UAUUGAGAGAAGUCCACCACGG.

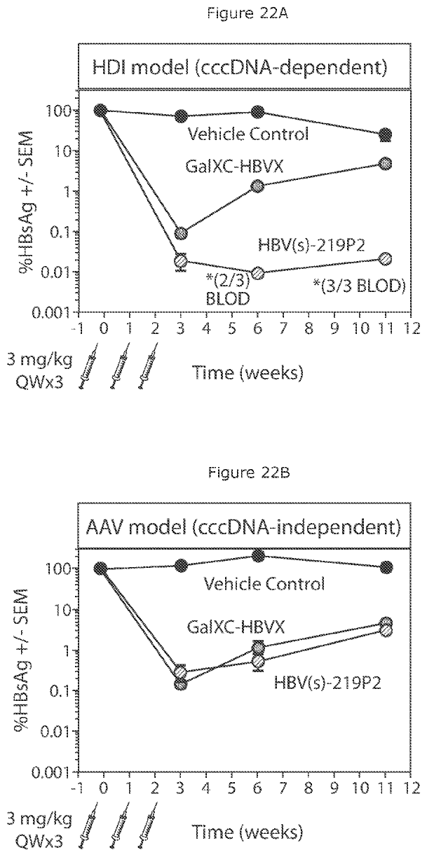

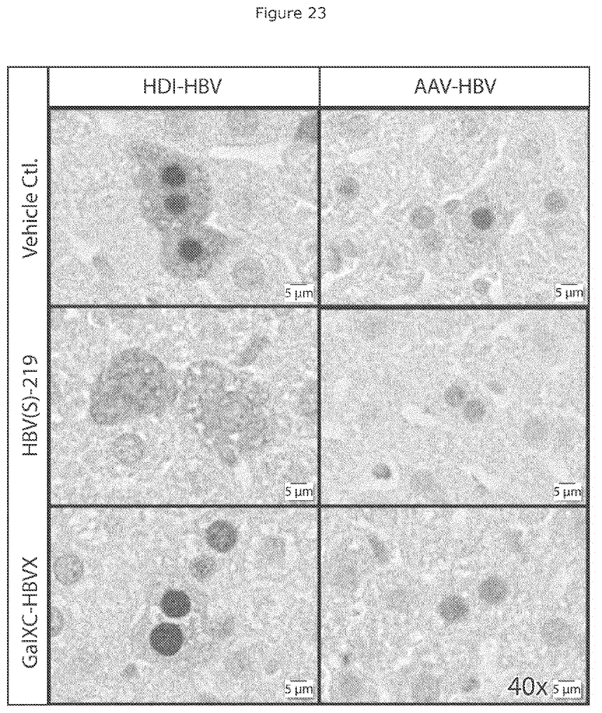

A single dose evaluation of oligonucleotide HBV-254 in HDI-mice was conducted, demonstrating the ability to subcutaneously target HBsAg viral transcript (FIG. 20). As shown, HBV-254 systematically reduced HBsAg levels in mice with increasing dosage. Preclinical potency was further evaluated in mice following a QW×3 dosing regimen in which HBV-254 was subcutaneously administered at 3 mg/kg (FIG. 23). The administration points are indicated by arrows in the figure. HBsAg levels were monitored in both oligonucleotide treated and untreated control mice for a period spanning 147 days. Diminished HBsAg levels persisted in treated mice throughout the entirety of the study, with expression levels (relative to control) appearing to settle at a reduced baseline at approximately two months following the first administration.

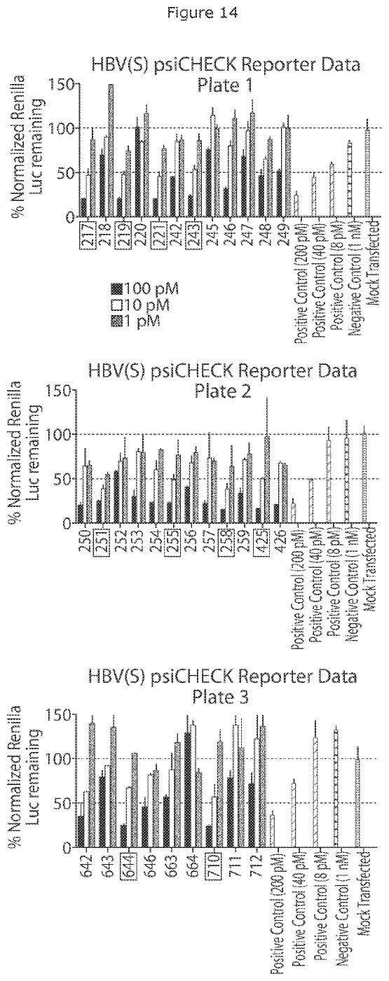

Additional potent HBsAg-targeting oligonucleotides were identified by in vitro screening using a psiCHECK reporter assay with oligonucleotides in unmodified tetraloop form. The results from three different plates are shown in FIG. 14. Each oligonucleotide, including HBV-254, was evaluated at three concentrations (1, 10, and 100 pM) in HeLa cells using the fluorescence-based reporter assay. The results reported for each plate are further shown in comparison with positive control (8, 40, and 200 pM), negative control (1 nM), and mock transfection. Oligonucleotides shown highlighted with boxes were scaled up for in vivo testing, in which HBV-219 and HBV-258 were found to be the most potent oligonucleotides among HBV-254 and those identified from the screening. HBV-219 exhibited a multi-log improvement in potency over HBV-254 and was selected for additional evaluation.

### Example A2. Sequence Conservation Analysis and Engineering Mismatches to Increase Global Therapeutic Utility

Several of the most potent oligonucleotides evaluated in Example A1 were compared against genome sequences for HBV genotypes A-I. The results of an initial conservation analysis are listed in Table 10. As shown, HBV-219 has relatively low percent conservation across these genomes. However, percent conservation increases significantly (from 66% to 96%) if a mismatch (MM) is introduced at position 15 of the guide strand. Genotyped hepatitis B virus (HBV) sequence data from the GenBank public database, incorporated herein by reference, was used for bioinformatics curation and alignment.

TABLE 10
Initial conservation analysis with top HBV sequences

| Oligo- | Guide | % conservation | % conservation if |
|---|---|---|---|
| nucleotide | Strand with MM in bold | across genomes | MM is tolerated |
| HBV-0217 | UUUGUGAGGAUUUUUGUCAAGG | 66 | 97 |
| HBV-0219 | UUAUUGUGAGGAUUUUUGUCGG | 66 | 96 |
| HBV-0254 | UCUGAGAGAAGUCCACCACGGG | 94 | 98 |
| HBV-0255 | UACUGAGAGAAGUCCACCACGG | 95 | 99 |
| HBV-0258 | UAAAACUGAGAGAAGUCCACGG | 94 | 98 |

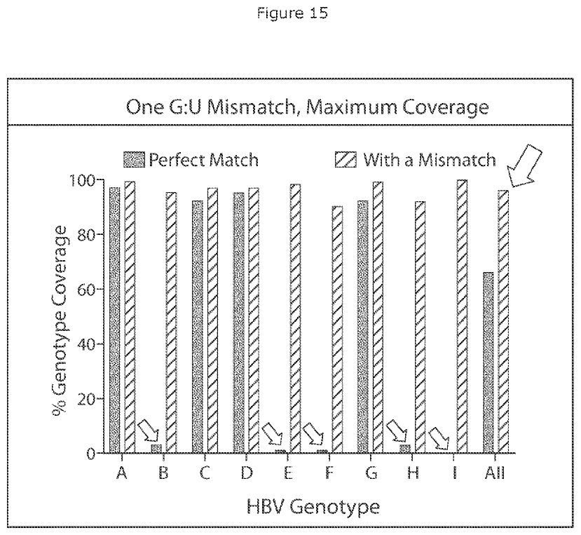

A subsequent conservation analysis was undertaken, which focused on several of the oligonucleotides from Table 10 and involved broader searching parameters. For example, whereas the initial analysis included only full-length genome sequences, the focused analysis included full-length and partial (>80% identity to target site) sequences. Additionally, the number of genomes examined increased from 5,628 in the initial analysis to more than 17,000 genomes in the focused analysis. Results from the focused analysis were in general agreement with the trends observed in the initial analysis (Table 11). As shown—and further illustrated in FIG. 15— HBV-219 was predicted to be inactive against HBV genotypes B, E, F, H, and I unless mismatch at position 15 of the guide strand is tolerated.

TABLE 11
Focused conservation analysis

| | | HBV-219 | HBV-254 | HBV-258 |
|---|---|---|---|---|
| ORF Target | | S | S | S |
| Sense 19-mer | | GACAAGAATCCTCA | CGTGGTGGACTTCTCTC | GTGGACTTCTCTCAATT |
| | | CAATA | AA | TT |
| Sense 19-mer | | GACAANAATCCTCA | CGTGGTGGACTTCTCTC | GTGGACTTCTCTCANTT |
| w/ambiguous | | CAATA | AN | TT |
| Base | | | | |
| Guide Position of | | 15 | 2 | 6 |
| Mismatch | | | | |
| Geno- | Genotype A | 97/99 [3278] | 94/97 [4002] | 94/97 [4005] |
| type | Genotype B | 03/95 [2563] | 81/97 [2700] | 82/99 [2700] |
| conser- | Genotype C | 92/97 [4783] | 95/97 [4938] | 96/98 [4938] |
| vation* | Genotype D | 95/97 [4311] | 96/99 [4395] | 96/98 [4398] |
| | Genotype E | 01/98 [1039] | 93/95 [1234] | 93/95 [1232] |
| | Genotype F | 01/90 [425] | 94/96 [501] | 95/96 [501] |
| | Genotype G | 92/99 [83] | 98/98 [85] | 99/99 [85] |
| | Genotype H | 03/92 [71] | 86/97 [78] | 87/99 [78] |
| | Genotype I | 00/100 [18] | 95/100 [22] | 95/100 [22] |

| | | | |
|---|---|---|---|
| TOTAL | 72/97 [17021] | 93/97 [17995] | 93/98 [17959] |
| (focused analysis) | | | |
| TOTAL | 66/96 [5628] | 94/98 [5628] | 94/98 [5628] |

(initial analysis)

*Percent conservation reported as (perfect/MM), with values <90% s own in bold; [Total N#]

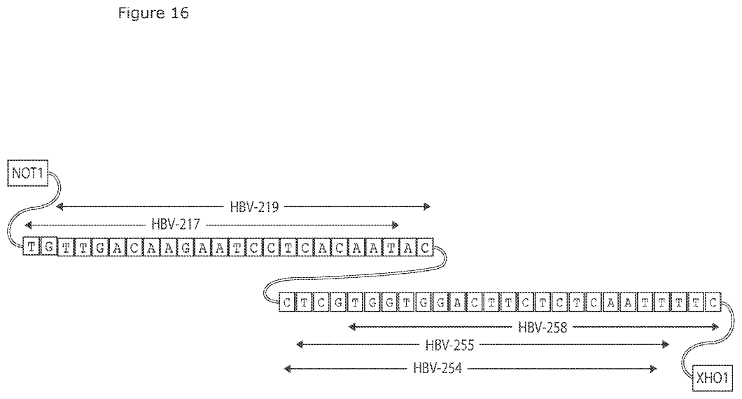

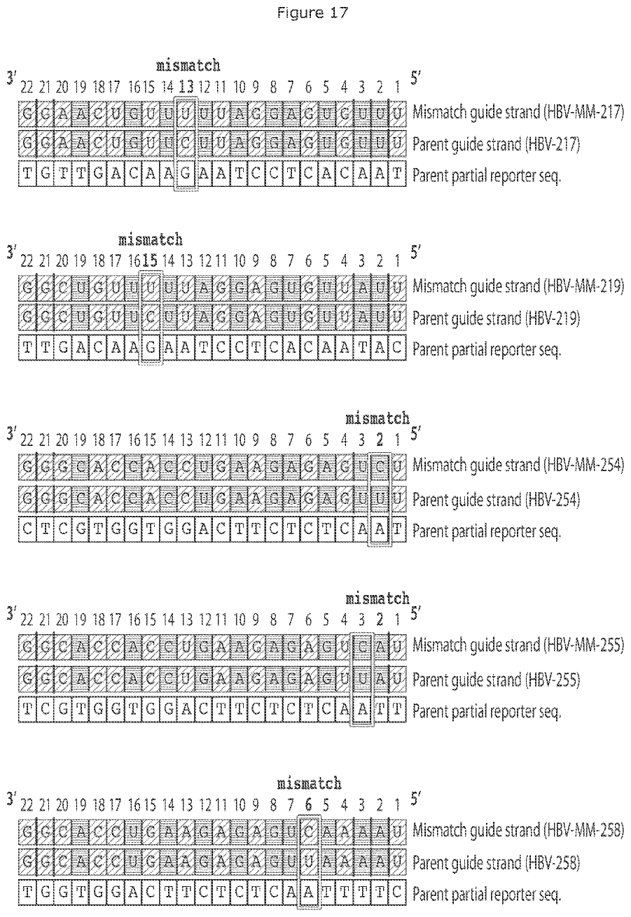

A psiCHECK-2 dual-luciferase reporter system was utilized to evaluate the effects of a mismatch at a selected position in each of HBV-217, HBV-219, HBV-254, HBV-255, and HBV-258. The psiCHECK vector enables monitoring of changes in expression of a target gene fused to a reporter gene, where active RNAi degrades the fusion construct to produce a corresponding decrease in reporter signal. The diagram in FIG. 16 generically depicts the vector utilized in these assays. The parent partial reporter sequence contained 120 base-pair fragments from Genotype A (GenBank: AM282986.1) around target sites of interest in the S ORF. Parent oligonucleotide duplex sequences have 100% homology to the reporter plasmid at corresponding sites shown in FIG. 16, whereas the mismatch oligonucleotide duplex sequences have a single mismatch to the reporter plasmid. Parent and mismatch sequences for the oligonucleotides tested are shown in FIG. 17 aligned to corresponding parent partial reporter sequences.

For the example mismatch assays, the tested oligonucleotides included the same modification patterns. According to the numbering scheme shown for each oligonucleotide in FIG. 17, modifications were as follows: 5′-Methoxy, Phosphonate-4′-oxy-2′-O-methyluridine at position 1; 2′-fluoro modified nucleotides at positions 2, 3, 5, 7, 8, 10, 12, 14, 16, and 19; 2′-O-methyl modified nucleotides at positions 1, 4, 6, 9, 11, 13, 15, 17, 18, and 20-22; and phosphorothioate internucleotide linkages between nucleotides at positions 1 and 2, 2 and 3, 3 and 4, 20 and 21, and 21 and 22. Mismatched positions were different for each parent and mismatch set, and are shown in boxes in FIG. 17.

The psiCHECK2 reporter assays with each oligonucleotide were conducted over a three-day period using a 6-point, 5-fold serial dilution starting at 1 nM transfected in HeLa cells. On day 1, 10,000 HeLa cells/well (96-well) were seeded in a black-walled, clear bottom plate (80-90% confluent). On day 2, vector DNA and RNAi molecule were diluted in the appropriate amount of Opti-MEM® I Medium without serum and gently mixed. After gently mixing Lipofectamine® 2000, 0.2 μL were diluted into 25 μL of Opti-MEM® I Medium without serum for each reaction. The dilution was mixed gently and incubated for 5 minutes at room temperature. After the 5 minute incubation, equal volumes of the diluted DNA and RNAi molecule were combined with the diluted Lipofectamine® 2000. The combined mixture was mixed gently and incubated for 20 minutes at room temperature to allow complex formation to occur. Following this, the DNA-RNAi molecule-Lipofectamine® 2000 complexes were added to each well containing cells and medium and mixed gently by rocking the plate back and forth. The cells were then incubated at 37° C. in a CO₂ incubator until the cells were ready to harvest and assay for the target gene. On day 3, 100 μL of Dual-Glo Reagent was added to each well, mixed and incubated for 10 minutes before reading the luminescence. A further 100 μL of Dual-Glo Stop & Glo was added to each well, mixed and incubated for 10 minutes before reading the luminescence. Dose-response curves were generated for each parent and mismatch oligonucleotide to evaluate the effects of mismatches on activity. The EC₅₀s values determined for each oligonucleotide are shown in Table 12 with additional specifications.

TABLE 12
Mismatch Evaluation of HBsAg-targeting oligonucleotides

| | HBV-217 | HBV-219 | HBV-254 | HBV-255 | HBV-258 |
|---|---|---|---|---|---|
| ORF Target | S | S | S | S | S |
| Sense 19-mer | TGTTGACAAGA | GACAAGAATCC | CGTGGTGGACT | TCGTGGTGGAC | GTGGACTTCTC |
| | ATCCTCACAAT | TCACAATA | TCTCTCAA | TTCTCTCAAT | TCAATTTT |
| Sense 19-mer | TGTTGACAANA | GACAANAATCC | CGTGGTGGACT | TCGTGGTGGAC | GTGGACTTCTC |
| w/ambiguous | ATCCTCACAAT | TCACAATA | TCTCTCAN | TTCTCTCANT | TCANTTTT |
| Base | | | | | |
| Guide | 13 | 15 | 2 | 3 | 6 |
| Position of | | | | | |
| Mismatch | | | | | |
| Parent EC₅₀s | 20 | 5 | 37 | 35 | 10 |
| (pM) | | | | | |
| MM EC₅₀s | 25 | 8 | 96 | 366 | >1000 |
| (pM) | | | | | |

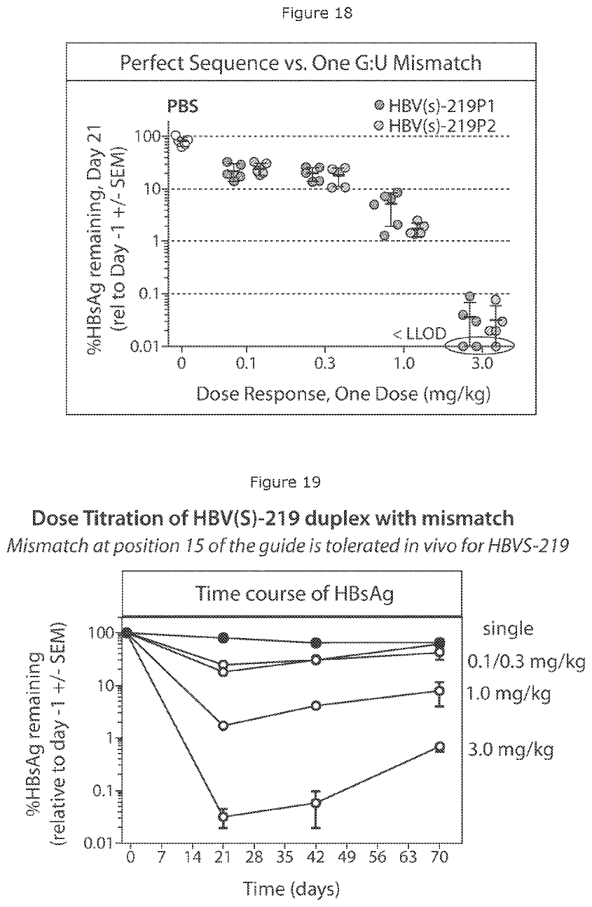

As demonstrated by the relative EC₅₀s values, the in vitro dose-response curves for HBV-219 duplexes showed no loss of activity with a single mismatch at position 15 of the guide strand. Subsequent in vivo analysis comparing HBV-219 parent (herein designated HBV(s)-219P1) and mismatch oligonucleotides (herein designated HBV(s)-219P2) confirmed that the introduction of the mismatch produced no loss of activity (FIG. 18). As shown in the single-dose titration plot depicted in FIG. 19, the HBV-219 mismatch oligonucleotide duplex (HBV(s)-219P2) was tolerated in vivo over a 70-day period following administration.

FIG. 20 illustrates an example of a modified duplex structure for HBV-219 with the incorporated mismatch (herein designated HBV(s)-219). According to the numbering scheme shown for each oligonucleotide in FIG. 17, the sense strand spans nucleotides 1 through 36 and the antisense strand spans oligonucleotides 1 through 22, the latter strand shown numbered in right-to-left orientation. The duplex form is shown with a nick between nucleotides at position 36 in the sense strand and position 1 in the antisense strand. Modifications in the sense strand were as follows: 2′-fluoro modified nucleotides at positions 3, 8-10, 12, 13, and 17; 2′-O-methyl modified nucleotides at positions 1, 2, 4-7, 11, 14-16, 18-26, and 31-36; a phosphorothioate internucleotide linkage between nucleotides at positions 1 and 2; 2′-OH nucleotides at positions 27-30; a 2′-aminodiethoxymethanol-Guanidine-GalNAc at position 27; and a 2′-aminodiethoxymethanol-Adenine-GalNAc at each of positions 28, 29, and 30. Modifications in the antisense strand were as follows: 5′-Methoxy, Phosphonate-4′-oxy-2′-O-methyluridine phosphorothioate at position 1; 2′-fluoro modified nucleotides at positions 2, 3, 5, 7, 8, 10, 12, 14, 16, and 19; 2′-O-methyl modified nucleotides at positions 1, 4, 6, 9, 11, 13, 15, 17, 18, and 20-22; and phosphorothioate internucleotide linkages between nucleotides at positions 1 and 2, 2 and 3, 3 and 4, 20 and 21, and 21 and 22. The antisense strand included an incorporated mismatch at position 15. Also as shown, the antisense strand of the duplex included a “GG” overhang spanning positions 21-22.

The details about HBV(s)-219 and the two precursors referred to above (HBV(s)-219P1 and HBV(s)-219P2) are shown in Table 13.

TABLE 13
HBV(s)-219 and precursors

| RNAi | Length | |
|---|---|---|
| oligo- | (sense/ | |
| nucleotides | antisense) | Sequence/Chemical Modifications |
| HBV(s)-219 | 36/22mer | Contains mismatch at position 15 of antisense |
| | | strand. An acetal based GalNAc linker is |
| | | used. Methoxy, Phosphonate-4′oxy-2′-O- |
| | | methyluridine (MeMOP) is used at position |
| | | 1 of antisense strand. |
| | | See Figures 20 and 29A |
| HBV(s)-219P2 | 36/22mer | Contains mismatch at position 15 of antisense |
| | | strand. A click chemistry based conjugation |
| | | incorporates a triazole based GalNAc linker. |
| | | Fully deprotected 5′-Phosphonate-4′oxy-2′-O- |
| | | methyluridine (MOP) is used at position 1 of |
| | | antisense strand. See Figure 29B |
| HBV(s)-219P1 | 36/22mer | Does not contain the mismatch at position 15 |
| | | of antisense strand. Same chemical |
| | | modifications as HBV(s)-219P2. |

### Example A3: Antiviral Activity of HBV(s)-219 Precursors

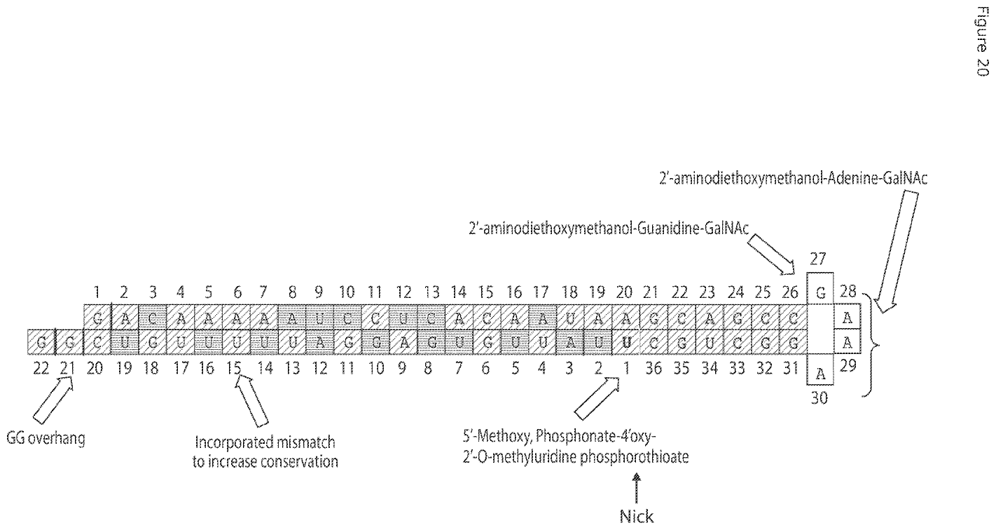

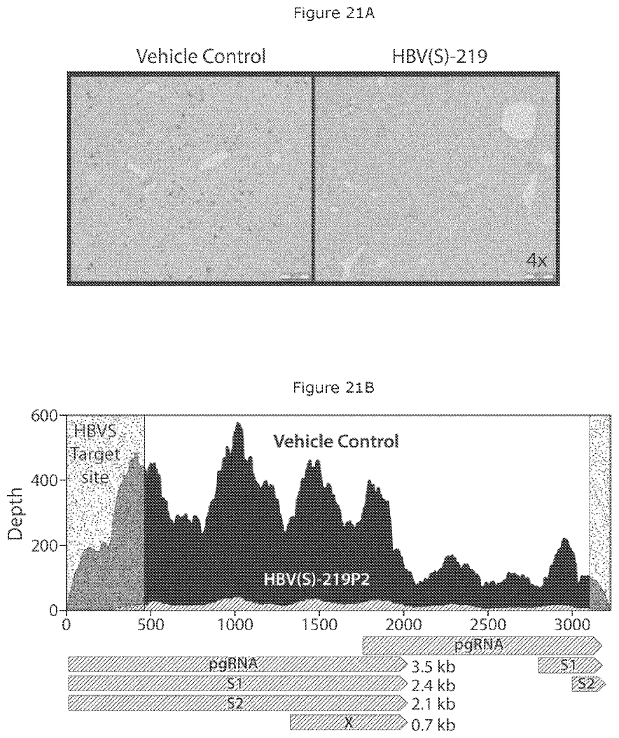

The effects of treatment with the HBV(s)-219 precursors on the subcellular localization of HBV core antigen (HBcAg) were evaluated. NOD_{scid} mice were subjected to a hydrodynamic injection (HDI) of a head-tail dimer of HBV genome. Treatment with the oligonucleotide was initiated 2 weeks post-HDI. Immunohistochemical staining of hepatocytes isolated from the mice following treatment showed a sharp reduction in HBV core antigen (HBcAg) expression. RNA sequencing was performed to examine the effects of HBsAg knockdown on overall expression of HBV viral transcripts. Hepatocytes were isolated from HDI mice four days following three, once-weekly doses at 3 mg/kg each. Total RNA was extracted from the hepatocytes and subjected to Illumina sequencing using the HiSeq Platform. FIG. 21B depicts RNA sequencing results in which detected RNA transcript sequences were mapped against the HBV RNAs. The target site of the HBV(s)-219 and its precursors is also depicted, showing that the oligonucleotide targets pgRNA (3.5 kb), S₁ (2.4 kb), and S₂ (2.1 kb) transcripts. The results show that, compared with vehicle controls, treatment with the HBV(s)-219P1 resulted in greater than 90% silencing of all HBV viral transcripts.

The durational effects of the HBV(s)-219P1 oligonucleotide were examined in two different mouse models of HBV— an HDI model, which is cccDNA-dependent, and an AAV model, which is cccDNA independent. A time course (12 weeks) analysis of HBsAg mRNA expression was performed in the context of a treatment involving three once-weekly doses of 3 mg/kg with the HBV(s)-219P1 oligonucleotide targeting HBsAg mRNA compared with vehicle control and an RNAi oligonucleotide targeting HBxAg mRNA in the HDI model of HBV (FIG. 22A). The HBV(s)-219P1 oligonucleotide produced a log reduction, with a relatively long duration of activity persisting for greater than 7 weeks; whereas by comparison an HBV(x) targeting oligonucleotide produced about a 3.0 log reduction, that persisted for a shorter duration.

A further time course (12 weeks) analysis of HBsAg mRNA expression was performed in the context of a treatment involving three once-weekly doses of 3 mg/kg with the HBV(s)-219P2 oligonucleotide targeting HBsAg mRNA compared with vehicle control and an RNAi oligonucleotide targeting HBxAg mRNA in an AAV-HBV model (FIG. 22B). In this model, the HBV(s)-219P2 oligonucleotide produced a comparable log reduction and duration as an HBV(x) targeting oligonucleotide. The RNAi oligonucleotide targeting HBxAg mRNA used in FIGS. 22A and 22B has a sense strand sequence of UGCACUUCGCGUCACCUCUAGCAGCCGAAAGGCUGC and an antisense strand sequence of UAGAGGUGACGCGAAGUGCAGG. This RNAi oligonucleotide targeting HBxAg is herein designated GalXC—HBVX.

Immunohistochemical staining was performed to examine the subcellular distribution of HBcAg in hepatocytes obtained from AAV-HBV model and HDI model of HBV following treatment with the HBV(s)-219 precursor oligonucleotides as indicated above targeting HBsAg mRNA compared with vehicle control and an RNAi oligonucleotide targeting HBxAg mRNA, as described above. (FIG. 23) Residual Core protein (HBcAg) after treatment exhibited notable differences in subcellular localization between the two RNAi oligonucleotides in the HDI model, but not in AAV model.

### Example A4: Evaluation of HBV(s)-219P1 in the PXB—HBV Chimeric Human Liver Model Genotype C

The antiviral activity of HBV(s)-219P1 was evaluated in the PXB—HBV model, also known in the HBV literature as the chimeric human liver model. This technology is based on grafting human hepatocytes into severely immunocompromised mice, then using a genetic mechanism to poison the host murine hepatocytes (Tateno et al., 2015). This process results in mice containing livers derived from >70% human tissue, which, unlike wild type mice, can be infected with HBV (Li et al., 2014). The PXB—HBV model serves several purposes in the context of HBV(s)-219 pharmacology: (1) to confirm that the oligonucleotide can engage the human RNAi machinery (RISC) in vivo, (2) to confirm that the GalNAc-targeting ligand configuration can internalize into hepatocytes via human ASGR in vivo, and (3) to confirm efficacy in a true model of HBV infection (as opposed to an engineered model of HBV expression). Despite the limitation that the grafted human hepatocytes result in an irregular chimeric liver physiology (Tateno et al., 2015), significant antiviral efficacy can be observed in this model.

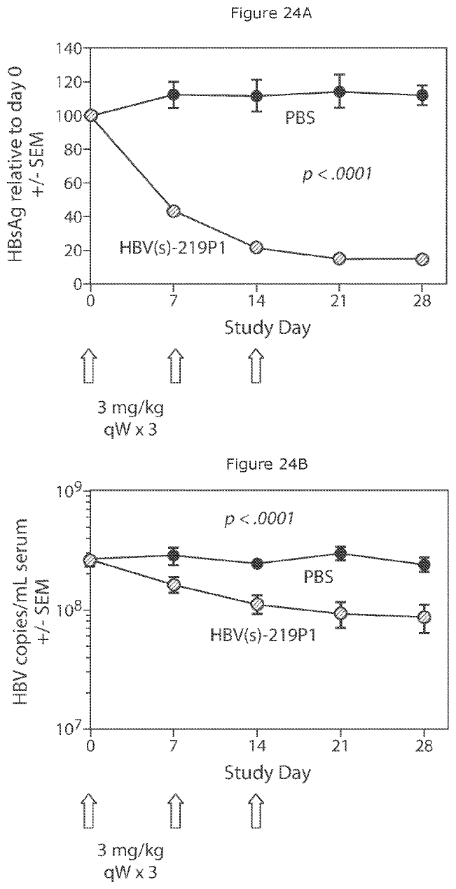

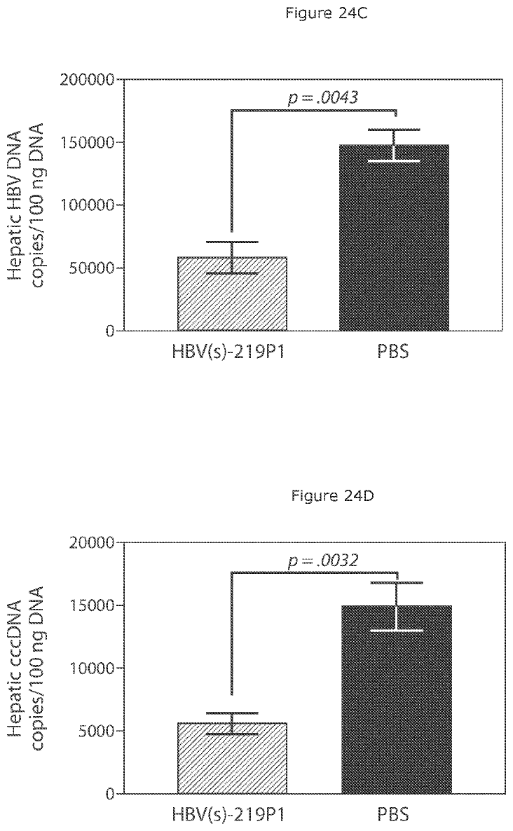

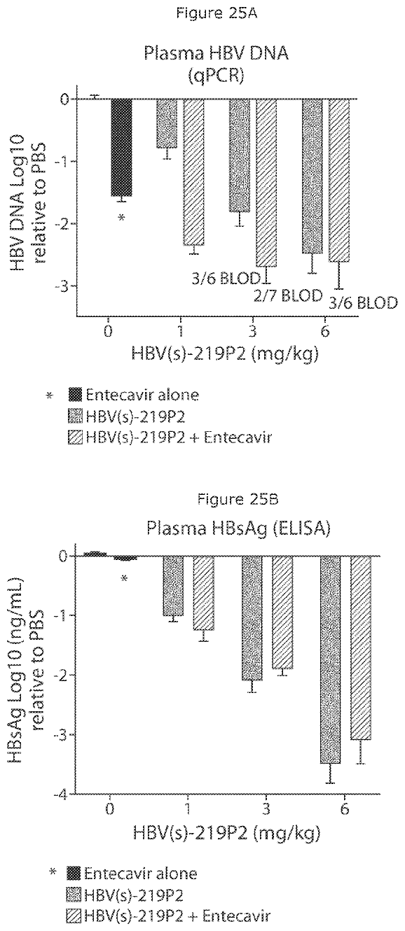

Approximately 8 weeks after the initial infection of the mice with HBV Genotype C, plasma are collected for each mouse to serve as a baseline HBsAg measurement. Then, cohorts of 9 mice each (n=3 for PK, n=6 for PD) received 3 weekly SC injections of 0 (PBS) or 3 mg/kg HBV(s)-219P1. The first day of dosing is considered Day 0. Non-terminal blood collections were performed weekly to determine the serum HBsAg and circulating HBV DNA levels in each mouse (FIGS. 24A-24D). Mice were euthanized for terminal tissue endpoints on Day 28. Day 28 liver samples were analyzed for intrahepatic HBV DNA and cccDNA levels. Significant antiviral activity was observed in all endpoints that were analyzed for mice treated with HBV(s)-219P1, including >80% reduction of HBsAg, as well as significant decreases in circulating HBV DNA, intrahepatic HBV DNA, and cccDNA (FIGS. 24A-24D). These data demonstrate that HBV(s)-219 treatment results in antiviral activity in infected human hepatocytes after systemic administration.

### Example A5: HBV(s)-219P2 Potentiates the Antiviral Activity of Entecavir

The current standard of care, nucleo(s)tide analogs (e.g., Entecavir) are effective at reducing circulating HBV genomic DNA, but do not reduce circulating HBsAg. While this results in controlled viremia while on such treatment, lifelong treatment is required and a functional cure is rarely achieved. The RNAi oligonucleotides targeting the S antigen impact both the viral polymerase and HBsAg protein. In this study, the combinational effects of HBV(s)-219P2 as a monotherapy and combinational treatment with entecavir was explored in an HBV-expressing mouse (HDI model) for antiviral activity.

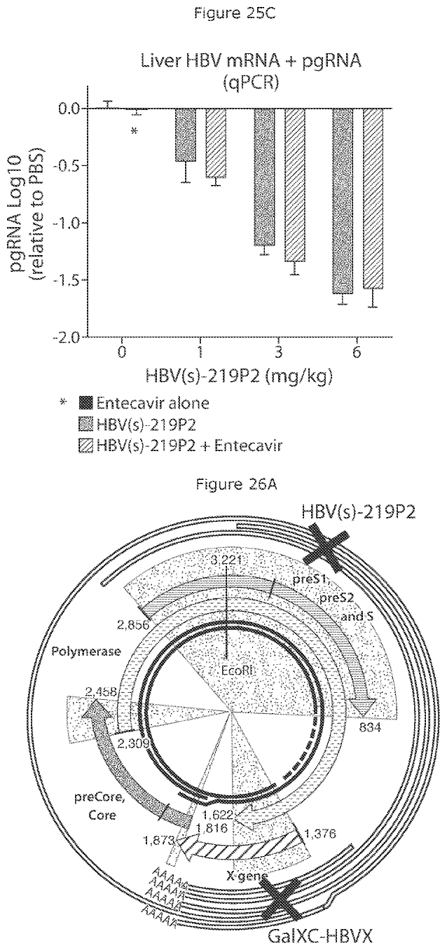

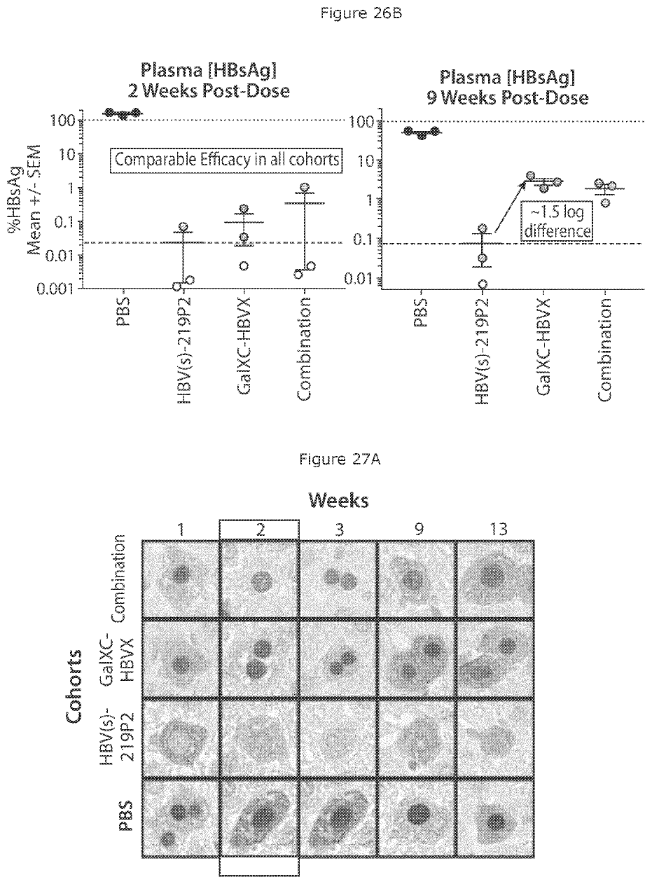

Mice were administered daily oral dosing of 500 ng/kg Entecavir (ETV) for 14 days. A single subcutaneous administration of HBV(s)-219P2 took place. Circulating viral load (HBV DNA) was measured by qPCR (FIG. 25A), plasma HBsAg level was measured by ELISA (FIG. 25B), and liver HBV mRNA and pgRNA levels were measured by qPCR. Clear additive effects were observed with combination therapy with HBV(s)-219P2 and ETV. The results show that ETV therapy alone shows no efficacy against circulating HBsAg or liver viral RNAs. Further, the antiviral activity of HBV(s)-219P2 as measured by HBsAg or HBV RNA is not impacted by codosing of ETV (FIGS. 25B-25C).

As shown in FIGS. 25A-25C, monotherapy of entecavir dosed 500 ng/kg PO daily for 14 days resulted in a mean ˜1.6 log decrease in HBV DNA detected in plasma relative to PBS treated mice (n=6). No significant decrease in either circulating HBsAg, or hepatic viral RNAs was observed. Monotherapy of a single 1 mg/kg, or 3 mg/kg SC dose of HBV(s)-219P2 at day 0 resulted in a mean ˜0.8 log, or ˜1.8 log decrease in HBV DNA detected in plasma relative to PBS respectively (n=7). Monotherapy of a single 6 mg/kg SC dose of HBV(s)-219P2 at day 0 resulted in a mean ˜2.5 log decrease in HBV DNA in plasma as well the levels in two mice falling below limit of detection (n=7). Monotherapy of a single SC dose of HBV(s)-219P2 on day 0 resulted in dose dependent decreases in in both circulating HBsAg, as well as hepatic viral RNAs. Combination therapy of entecavir dosed 500 ng/kg PO daily for 14 days and a single 1 mg/kg SC dose of HBV(s)-219P2 on day 0 resulted in additive reduction in HBV DNA detected in the plasma by a mean of ˜2.3 log. Similar reductions in levels of plasma HBsAg and hepatic viral transcripts as observed with a monotherapy of a single 1 mg/kg SC dose of HBV(s)-219P2 indicating additivity in reducing plasma HBV DNA, but not circulating HBsAg, or hepatic viral transcript.

### Example A6. Comparison of the Antiviral Activity of HBV(s)-219P2 and GalXC—HBVX

In this study, HBV-expressing mice (HDI model) were administered HBV(s)-219P2, GalXC—HBVX (same sequence as the GalXC—HBVX used in FIGS. 22A and 22B), or a combination of the two RNAi oligonucleotides and plasma HBsAg level two weeks or nine weeks post dose were monitored. As shown in FIG. 26B, similar levels of HBsAg suppression were observed 2 weeks after treatment with a single saturating 9 mg/kg SC dose of either HBV(s)-219P2, GalXC—HBVX, or a combination of both. Prolonged suppression of HBsAg was observed in mice treated with the S-targeting HBV(s)-219P2 treatment, whereas mice treated with the GalXC—HBVX, or a combination of both, had significant recovery of HBsAg 9 weeks after treatment (n=3).

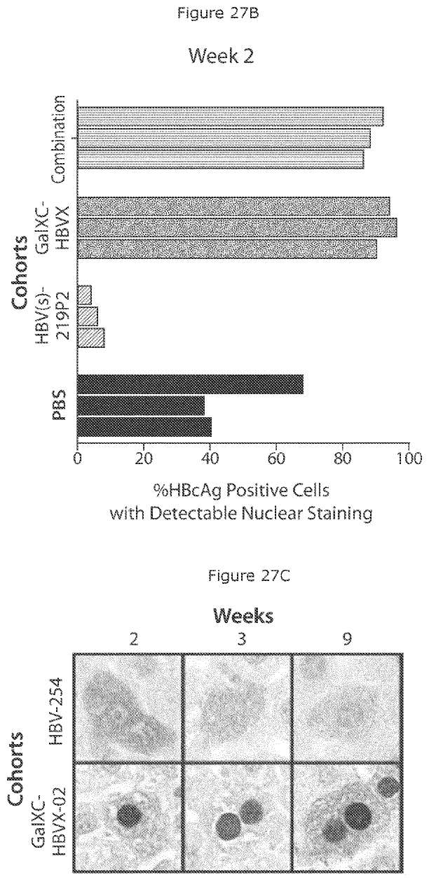

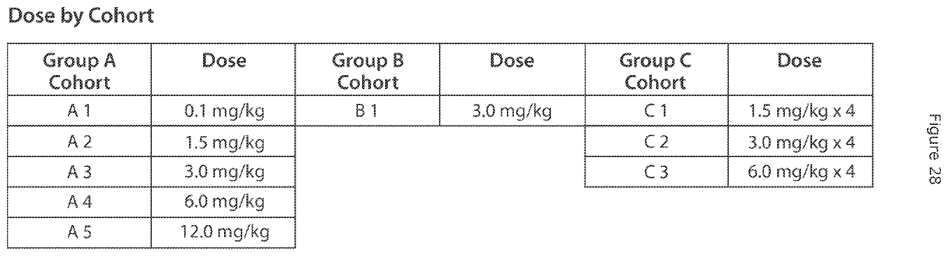

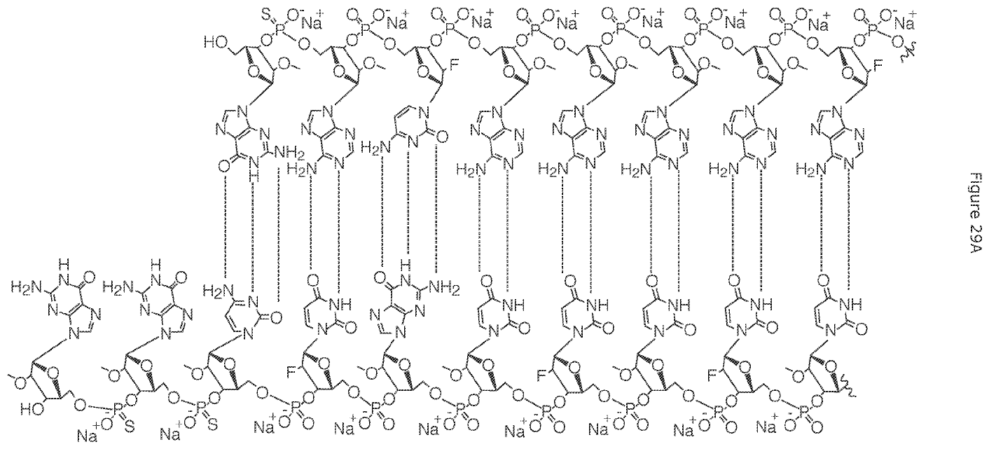

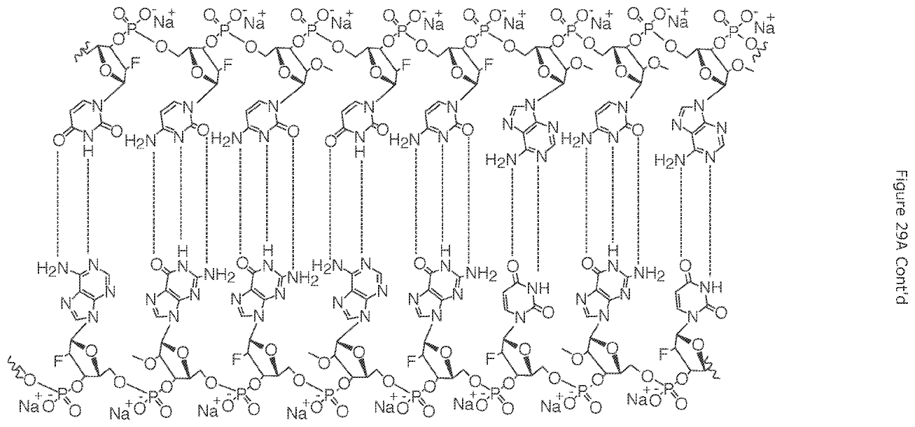

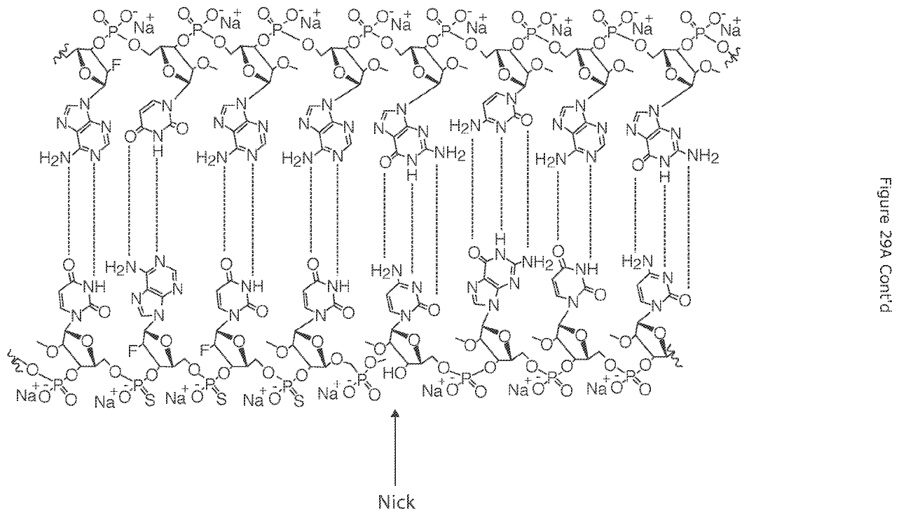

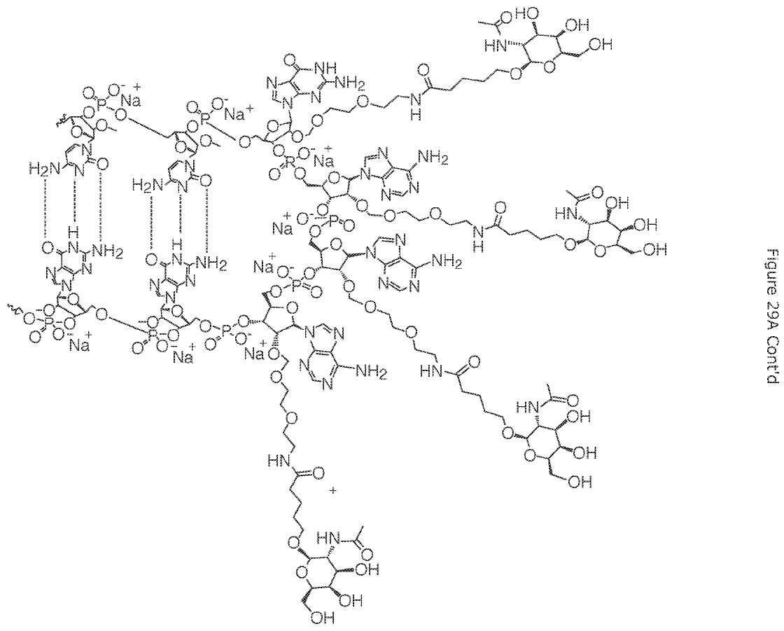

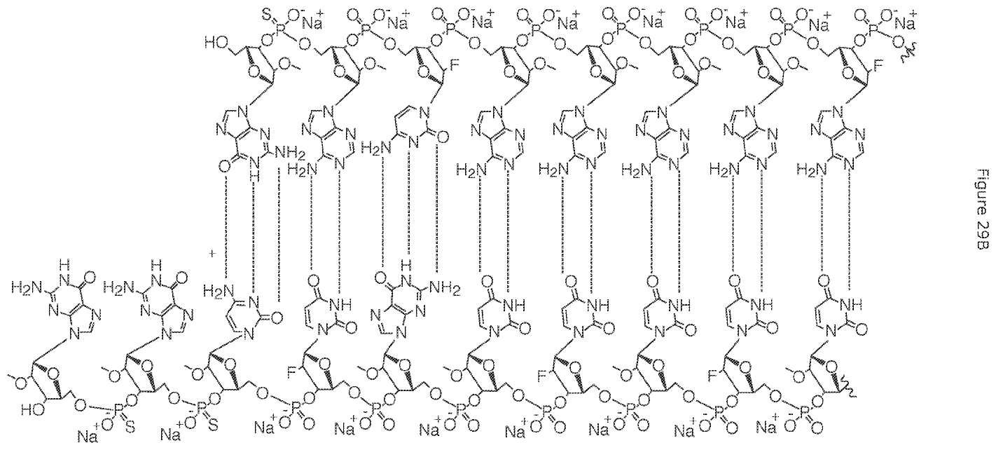

The subcellular localization of HBV Core Antigen (HBcAg) in HBV-expressing mice was also evaluated in mice receiving HBV(s)-219P2, GalXC—HBVX, or a combination of the two RNAi oligonucleotides. HBV-expressing mice (HDI model) were treated with a single saturating dose (9 mg/kg, s.c.) of HBV(s)-219P2, GalXC—HBVX or a 1:1 combination. At the time points indicated in FIG. 27A, liver sections were stained for HBcAg; representative hepatocytes are shown. Cohorts treated with HBV(s)-219P2, either as a monotherapy or in combination with GalXC—HBVX, feature nuclear HBcAg. Cohorts treated with only GalXC—HBVS show only cytosolic localization of HBcAg, reported as a favorable prognostic indicator of treatment response (Huang et al. J. Cell. Mol. Med. 2018). The percentage of HBcAg-positive-cells with nuclear staining in each animal is shown in FIG. 27B (n=3/group, 50 cells counted per animal, 2 weeks after dosing). To confirm that the effect on HBcAg subcellular localization is due to the region of the HBV transcriptome, and not to an unknown property of the RNAi sequence, alternative sequences were designed and tested, targeting within the X and S open reading frames (see FIG. 27C). HBV-254 was used in FIG. 27C. The sequence of HBV-254 is described in Example A1. The alternative oligonucleotide targeting HBxAg used in FIG. 27C has a sense strand sequence of GCACCUCUCUUUACGCGGAAGCAGCCGAAAGGCUGC and an antisense sequence of UUCCGCGUAAAGAGAGGUGCGG. The two alternative RNAi oligonucleotides have different RNAi target sequences in the S or X antigen than the RNAi oligonucleotides used in FIG. 26B. However, they display the same differential effect on plasma level HBcAg, indicating that the effect is specific to targeting the S antigen per se, but not specific the oligonucleotide used.

### Example A7 Evaluation of the Safety, Tolerability in Healthy Human Subjects and Efficacy of HBV(s)-219 in HBV Patients

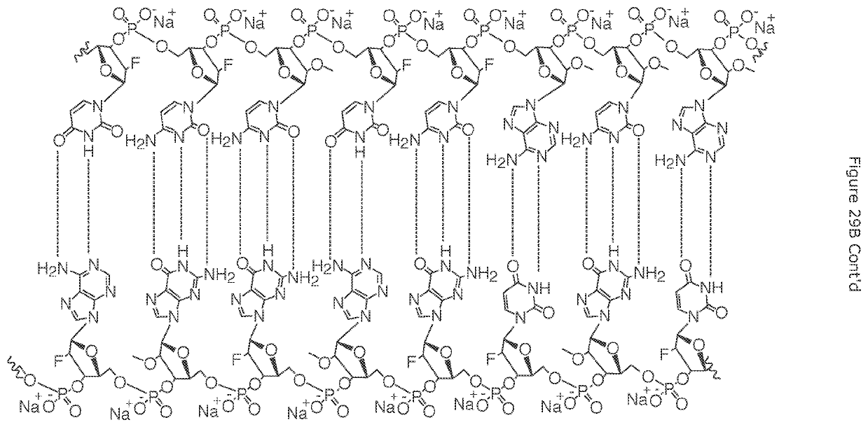

This study is designed to evaluate the safety and tolerability in healthy subjects (Group A) and efficacy of HBV(s)-219 in HBV patients (Group B). The dose by cohort information is shown in FIG. 28. The molecular structure of HBV(s)-219 is shown in FIG. 20, FIG. 29A, and also illustrated below:
Sense Strand: 5′ mG-S-mA-fC-mA-mA-mA-mA-fA-fU-fC-mC-fU-fC-mA-mC-mA-fA-mU-mA-mA-mG-mC-mA-mG-mC-mC-[ademG-GalNAc]-[ademA-GalNAc]-[ademA-GalNAc]-[ademA-GalNAc]-mG-mG-mC-mU-mG-mC 3′
   Hybridized to:
   Antisense Strand: 5′ [MePhosphonate-4O-mU]—S-fU-S-fA-S-mU-fU-mG-fU-fG-mA-fG-mG-fA-mU-fU-mU-fU-mU-mG-fU-mC-S-mG-S-mG 3′

Legend:
mX: 2′-O-methyl ribonucleotide
fX: 2′-fluoro-deoxyribonucleotide
[ademA-GalNAc]: 2′-modified -GalNAc adenosine
[ademG-GalNAc]: 2′-modified -GalNAc guanosine
[MePhosphonate-4O-mU]: 4′-O-monomethylphosphonate-2′-O-methy uridine
Linkages: “—” denotes phosphodiester
   “—S—” denotes phosphorothioate

The Patient selection criterial are shown below.

Group A—Healthy Subjects

Inclusion Criteria:

1. Age 18 (or age of legal consent, whichever is older) to 65 years inclusive, at the time of signing the informed consent.

2. Overtly healthy at the time of screening as determined by medical evaluation including medical history, physical examination, and laboratory tests

a. No symptoms of ongoing illness

b. No clinically significant abnormalities in body temperature, pulse rate, respiratory rate, blood pressure

c. No clinically significant cardiovascular or pulmonary disease, and no cardiovascular or pulmonary disease requiring pharmacologic medication.

3. 12-lead electrocardiogram (ECG) within normal limits or with no clinically significant abnormalities at screening and Day-1 in the opinion of the Investigator

4. Negative screen for alcohol or drugs of abuse at Screening Visit 1 and admission (Day-1)

5. Non-smokers for at least 5 years preceding Screening Visit 1, with a negative urinary cotinine concentration at Screening Visit 1

6. Body mass index (BMI) within the range 18.0-32.0 kg/m² (inclusive).

7. Male or Female:

a. Male participants:

A male participant must agree to use contraception, during the treatment period and for at least two weeks after the dose of study intervention and refrain from donating sperm during this period.

b. Female participants:

A female participant is eligible to participate if she is not pregnant, not breastfeeding, and at least one of the following conditions applies: Not a woman of childbearing potential (WOCBP), OR, depending on region; a WOCBP who agrees to follow the contraceptive guidance, beginning at post-screen study enrollment continuing throughout the treatment period and for at least 12 weeks after the dose of study intervention.

8. Capable of giving signed informed consent 1, which includes compliance with the requirements and restrictions.

Exclusion Criteria, Group A

1. History of any medical condition that may interfere with the absorption, distribution or elimination of study drug, or with the clinical and laboratory assessments in this study, including (but not limited to); chronic or recurrent renal disease, functional bowel disorders (e.g., frequent diarrhea or constipation), GI tract disease, pancreatitis, seizure disorder, mucocutaneous or musculoskeletal disorder, history of suicidal attempt(s) or suicidal ideation, or clinically significant depression or other neuropsychiatric disorder requiring pharmacologic intervention

2. Poorly controlled or unstable hypertension; or sustained systolic BP >150 mmHg or diastolic BP >95 mmHg at Screen

3. History of diabetes mellitus treated with insulin or hypoglycemic agents

4. History of asthma requiring hospital admission within the preceding 12 months

5. Evidence of G-6-PD deficiency as determined by the Screen result at the central study laboratory

6. Currently poorly controlled endocrine conditions, with the exception of thyroid conditions (hyper/hypothyroidism, etc.) where any pharmacologically treated thyroid conditions are excluded

7. A history of malignancy is allowed if the participant's malignancy has been in complete remission off chemotherapy and without additional medical or surgical interventions during the preceding three years

8. History of multiple drug allergies or history of allergic reaction to an oligonucleotide or GalNAc

9. History of intolerance to SC injection(s) or significant abdominal scarring that could potentially hinder study intervention administration or evaluation of local tolerability

10. Clinically relevant surgical history

11. History of persistent ethanol abuse (>40 gm ethanol/day) or illicit drug use within the preceding 3 years.

12. Clinically significant illness within the 7 days prior to the administration of study intervention

13. Donation of more than 500 mL of blood within the 2 months prior to administration of study intervention or plasma donation within 7 days prior to Screen

14. Significant infection or known inflammatory process ongoing at Screening (in the opinion of the Investigator)

15. History of chronic or recurrent urinary tract infection (UTI), or UTI within one month prior to Screen

16. Scheduled for an elective surgical procedure during the conduct of this study

17. Use of prescription medications within 4 weeks prior to the administration of study intervention

18. Use of over-the-counter (OTC) medication or herbal supplements, excluding routine vitamins, within 7 days of first dosing, unless agreed as not clinically relevant by the Investigator and Sponsor.

19. Has received an investigational agent within the 3 months prior to dosing or is in followup of another clinical study prior to study enrollment.

20. Seropositive for HBV, HIV, HCV, or HDV antibody at Screening (historical testing may be used if performed within the 3 months prior to screening)

21. Alanine aminotransferase (ALT), aspartate aminotransferase (AST), gamma-glutamyl transferase (GGT), total bilirubin, alkaline phosphatase (ALP), or albumin outside of the reference range at the Screening Visit or on admission (Day-1)

22. Complete blood count test abnormalities that are considered clinically relevant and unacceptable by the Investigator; hemoglobin <12.0 g/dL (equivalent to 120 g/L); platelets outside of the normal range.

23. Hemoglobin A1C (HbA1C)>7%

24. Any other safety laboratory test result considered clinically significant and unacceptable by the Investigator

25. Has undertaken, or plans to undertake, a significant change in exercise levels from 48 hours prior to entrance into the clinical research center until the end of study.

26. Any condition that, in the opinion of the Investigator, would make the participant unsuitable for enrollment or could interfere with participation in or completion of the study.

Group B Adults with Hepatitis B

Inclusion Criteria, Group B

1. Age 18 (or age of legal consent, whichever is older) to 65 years inclusive, at the time of signing the informed consent.

2. Chronic hepatitis B infection, documented by:

a. clinical history compatible with CHB, based on compatible clinical information, and previous seropositivity for HBsAg and potentially other HBV serologic markers (HBeAg, HBV DNA)

b. Serum HBsAg>1000 IU/mL at Screening for HBeAg-positive patients, or >500 IU/mL for HBeAg-negative patients

c. Serum HBV DNA>20,000 IU/mL at Screening for treatment-naïve patients, as determined by the TaqMan™ HBV DNA v2.0 assay at the central study laboratory

d. Serum IgM anti-HBc negative

3. Clinical history compatible with compensated liver disease, with no evidence of cirrhosis:

a. No history of bleeding from esophageal or gastrointestinal varices

b. No history of ascites

c. No history of jaundice attributed to chronic liver disease

d. No history of hepatic encephalopathy

e. No physical stigmata of portal hypertension—spider angiomata, etc.

f. No previous liver biopsy, hepatic imaging study, or elastography result indicating cirrhosis

4. Treatment-naïve for hepatitis B: no previous antiviral therapy for hepatitis B (no previous HBV nucleos[t]ide or interferon-containing treatment) OR continuously on nucleos(t)ide therapy (entecavir or tenofovir) for at least 12 weeks prior to the Screening visit, with satisfactory tolerance and compliance

5. Serum ALT>60 U/L (males) or >38 U/L (females) (2×ULN by American Association for the Study of Liver Diseases (AASLD) HBV guidance criteria, Terrault et al., 2016)

6.12-lead ECG with no clinically significant abnormalities at Screening and Day-1 (in the opinion of the Investigator)

7. No other known cause of liver disease

8. No other medical condition that requires persistent medical management or chronic or recurrent pharmacologic intervention, other than well-controlled hypertension and statin management of hypercholesterolemia

9. BMI within the range 18.0-32.0 kg/m² (inclusive)

10. Male or female

a. Male participants:

A male participant must agree to use contraception during the treatment period and for at 12 weeks after the last dose of study intervention and refrain from donating sperm during this period.

b. Female participants:

A female participant is eligible to participate if she is not pregnant, not breastfeeding, and at least one of the following conditions applies: Not a WOCBP OR, depending on region A WOCBP who agrees to follow the contraceptive guidance during the treatment period and for at least 12 weeks after the dose of study intervention.

11. Capable of giving signed informed consent, which includes compliance with the requirements and restrictions.

Exclusion Criterial, Group B

1. History of any medical condition that may interfere with the absorption, distribution or elimination of study drug, or with the clinical and laboratory assessments in this study, including (but not limited to); chronic or recurrent renal disease, functional bowel disorders (e.g., frequent diarrhea or constipation), GI tract disease, pancreatitis, seizure disorder, mucocutaneous or musculoskeletal disorder, history of suicidal attempt(s) or suicidal ideation, or clinically significant depression or other neuropsychiatric disorder requiring pharmacologic intervention

2. Poorly controlled or unstable hypertension

3. History of diabetes mellitus treated with insulin or hypoglycemic agents

4. History of asthma requiring hospital admission within the preceding 12 months

5. Evidence of G-6-PD deficiency as determined by the Screen result at the central study laboratory

6. Currently poorly controlled endocrine conditions, with the exception of thyroid conditions (e.g. hyper/hypothyroidism, etc.) where any pharmacologically treated thyroid conditions are excluded

7. History of chronic or recurrent UTI, or UTI within one month prior to Screen

8. History of HCC

9. History of malignancy other than HCC is allowable if the patient's malignancy has been in complete remission off chemotherapy and without additional medical or surgical interventions during the preceding three years

10. History of persistent ethanol abuse (>40 gm ethanol/day) or illicit drug use within the preceding 3 years.

11. History of intolerance to SC injection(s) or significant abdominal scarring that could potentially hinder study intervention administration or evaluation of local tolerability.

12. Receipt of a transfusion in the last 6 weeks prior to therapy or anticipated transfusions through the post-trial follow-up.

13. Donated or lost>500 mL of blood within 2 months prior to Screening, or plasma donation within 7 days prior to Screening

14. Antiviral therapy (other than entecavir or tenofovir) within 3 months of Screening or treatment with interferon in the last 3 years

15. Use within the last 6 months of (or an anticipated requirement for) anticoagulants, systemically administered corticosteroids, systemically administered immunomodulators, or systemically administered immunosuppressants

16. Use of prescription medication within 14 days prior to administration of study intervention that, in the opinion of the PI or the Sponsor, would interfere with study conduct. Topical products without systemic absorption, statins (except rosuvastatin), hypertensive medications, OTC and prescription pain medication or hormonal contraceptives (females) are acceptable.

17. Depot injection or implant of any drug within 3 months prior to administration of study intervention, with the exception of injectable/implantable birth control.

18. Persistent use of herbal supplements or systemic over-the-counter medications; participants must be willing to stop for the duration of the study period

19. Has received an investigational agent within the 3 months prior to dosing or is in follow-up of another clinical study prior to study enrollment.

20. Liver Elastography (i.e. FibroScan®) kPa>10.5 at Screening

21. Systolic blood pressure>150 mmHg and a diastolic blood pressure of >95 mmHg after 10 minutes supine rest, at Screening.

22. Hepatic transaminases (ALT or AST) confirmed >7×ULN at Screening

23. History of persistent or recurrent hyperbilirubinemia, unless known Gilbert's Disease or Dubin-Johnson Syndrome

24. Seropositive for antibodies to human immunodeficiency virus (HIV) or hepatitis C virus (HCV) or hepatitis delta virus (HDV)

25. Hgb<12 g/dL (males) or <11 g/dL (females)

26. Serum albumin<3.5 g/dL at screening.

27. Total WBC count<4,000 cells/μL or absolute neutrophil count (ANC)<1800 cells/μL at screening.

28. Platelet count 100,000 per μL at screening.

29. International normalized ratio (INR) or prothrombin time (PT) above the upper limit of the normal reference range (as per the local laboratory reference range) at screening.

30. Serum BUN or creatinine>ULN

31. Serum amylase or lipase>1.25×ULN

32. Serum HbA1c>7.0%

33. Serum alpha fetoprotein (AFP) value>100 ng/mL. If AFP at screening is >ULN but <100 ng/mL, patient is eligible if a hepatic imaging study reveals no lesions suspicious of possible HCC

34. Any other safety laboratory test result considered clinically significant and unacceptable by the Investigator

35. Has undertaken, or plans to undertake, a significant change in exercise levels from 48 hours prior to entrance into the clinical research center until the end of study.

36. Any condition that, in the opinion of the Investigator, would make the participant unsuitable for enrollment or could interfere with participation in or completion of the study.

Part B: Effects of a GalNAc Conjugated Antisense Oligonucleotide

Materials and Methods
AAV/HBV Mouse Models

The AAV-HBV mouse model is generated by injecting C57BL/6 mice with recombinant adeno-associated virus harboring a replicable HBV genome (AAV-HBV). The rAAV8-1.3 HBV ayw virus stock was purchased from Beijing FivePlus Molecular Medicine Institute (Beijing, China).

The animals (male, aged 4-5 weeks upon arrival) were purchased from SLAC Laboratory Animal Co. Ltd (Shanghai, China), acclimatized in the animal facility for 5-7 days, and then injected with 1×10¹¹ vector genome of AAV-HBV (diluted in 200 μL of PBS) through the tail vein. Persistence expression of HBV genome can be established after three weeks, with high levels of HBV viral markers including HBV DNA, HBsAg, and HBeAg in mouse serum. With stable HBV viremia and competent immune system of the C57BL/6 mice, the AAV-HBV mouse model was used to evaluate the in vivo anti-HBV efficacy of the compounds. The in-life part of the AAV-HBV study was conducted through contracted service at Covance Pharmaceutical Research and Development (Shanghai) Co. Ltd. (Covance Shanghai) and the post-life analysis using the serum was performed internally at Roche Innovation Center Shanghai (RICS).

Seven days before compound treatment, blood samples were collected for serum preparation (˜15 μL), and based on HBV DNA, HBsAg levels in serum and body weight, the AAV-HBV infected animals were stratified into the different treatment groups.

Saline (Group 01) and anti-HBV ASO of CMP ID NO: 15_1 at 1.5 or 7.5 mg/kg were dosed sub subcutaneously once a week during Day 0-49 on Days 0, 7, 14, 21, 28, 35, 42 and 49. TLR7 agonist of CMP ID NO: VI 100 mg/kg was administered by oral gavage once every other day during Day 0-55 (QOD) or once weekly during Day 0-49 on Days 0, 7, 14, 21, 28, 35, 42 and 49 (QW). The animals were weekly bled via retro-orbital sinus for sample collection throughout the study.
Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorothioate linkages is carried out by using xanthane hydride (0.01 M in acetonitrile/pyridine 9:1). Phosphodiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phosphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter C18 10p 150×10 mm column. 0.1 M ammonium acetate pH 8 and acetonitrile is used as buffers at a flow rate of 5 mL/min. The collected fractions are lyophilized to give the purified compound typically as a white solid.

Abbreviations:

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography
Tₘ Assay

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Na phosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.
Tissue Specific In Vitro Linker Cleavage Assay

FAM-labeled oligonucleotides with the biocleavable linker to be tested (e.g. a DNA phosphodiester linker (PO linker)) are subjected to in vitro cleavage using homogenates of the relevant tissues (e.g. liver or kidney) and Serum.

The tissue and serum samples are collected from a suitable animal (e.g. mice, monkey, pig or rat) and homogenized in a homogenisation buffer (0.5% Igepal CA-630, 25 mM Tris pH 8.0, 100 mM NaCl, pH 8.0 (adjusted with 1 N NaOH)). The tissue homogenates and Serum are spiked with oligonucleotide to concentrations of 200 μg/g tissue. The samples are incubated for 24 hours at 37° C. and thereafter the samples are extracted with phenol-chloroform. The solutions are subjected to AlE HPLC analyses on a Dionex Ultimate 3000 using a Dionex DNApac p-100 column and a gradient ranging from 10 mM-1 M sodium perchlorate at pH 7.5. The content of cleaved and non-cleaved oligonucleotides are determined against a standard using both a fluorescence detector at 615 nm and a UV detector at 260 nm.
S1 Nuclease Cleavage Assay

FAM-labelled oligonucleotides with 51 nuclease susceptible linkers (e.g. a DNA phosphodiester linker (PO linker)) are subjected to in vitro cleavage in 51 nuclease extract or Serum.

100 μM of the oligonucleotides are subjected to in vitro cleavage by 51 nuclease in nuclease buffer (60 U pr. 100 μL) for 20 and 120 minutes. The enzymatic activity is stopped by adding EDTA to the buffer solution. The solutions are subjected to AlE HPLC analyses on a Dionex Ultimate 3000 using a Dionex DNApac p-100 column and a gradient ranging from 10 mM-1 M sodium perchlorate at pH 7.5. The content of cleaved and non-cleaved oligonucleotide is determined against a standard using both a fluorescence detector at 615 nm and a UV detector at 260 nm.
HBsAg Antigen Measurements

Serum HBsAg levels were determined in the serum of infected AAV-HBV mouse using the HBsAg chemoluminescence immunoassay (CLIA) (Autobio diagnostics Co. Ltd., Zhengzhou, China, Cat. no.CL0310-2), according to the manufacturer's protocol. Briefly, 50μl of serum was transferred to the antibody coated microtiter plate and 50 μl of enzyme conjugate reagent was added. The plate was incubated for 60 min on a shaker at room temperature before all wells were washed six times with washing buffer using an automatic washer. 25 μl of substrate A and then 25 μl of substrate B was added to each well. The plate was incubated for 10 min at RT before luminescence was measured using an Envision luminescence reader (Perkin Elmer). HBsAg is given in the unit IU/ml; where 1 ng HBsAg=1.14 IU.

HBeAg levels, can likewise be measured using CLIA ELISA Kits (Autobio Diagnostic #CL0310-2), according to the manufacturer's protocol and the brief description given for HBsAg above.
Real-Time PCR for Intracellular HBV mRNA from HBV Infected Cells

HBV mRNA can be quantified in technical duplicate by qPCR using a QuantStudio 12K Flex (Applied Biosystems), the TaqMan RNA-to-CT 1-Step Kit (Applied Biosystems, #4392938), Human ACTB endogenous control (Applied Biosystems, #4310881E). Taqman reagents are used together with the following commercial ThermoFisher Scientific primers (HBV Pa03453406_s1, ACTB 4310881E). The mRNA expression is analyzed using the comparative cycle threshold 2-ΔΔCt method normalized to the reference gene ACTB and to PBS treated cells.
HBV DNA Extraction and qPCR

Initially mice serum was diluted by a factor of 10 (1:10) with Phosphate buffered saline (PBS). DNA was extracted using the MagNA Pure 96 (Roche) robot. 50μl of the diluted serum was mixed in a processing cartridge with 200 ul MagNA Pure 96 external lysis buffer (Roche, Cat. no. 06374913001) and incubated for 10 minutes. DNA was then extracted using the “MagNA Pure 96 DNA and Viral Nucleic Acid Small Volume Kit” (Roche, Cat. no. 06543588001) and the “Viral NA Plasma SV external lysis 2.0” protocol. DNA elution volume was 50μl.

Quantification of extracted HBV DNA was performed using a Taqman qPCR machine (ViiA7, life technologies). Each DNA sample was tested in duplicate in the PCR. 5 μl of DNA sample was added to 15 μl of PCR mastermix containing 10 μl TaqMan Gene Expression Master Mix (Applied Biosystems, Cat. no. 4369016), 0.5 μl PrimeTime XL qPCR Primer/Probe (IDT) and 4.5 μl distilled water in a 384 well plate and the PCR was performed using the following settings: UDG Incubation (2 min, 50° C.), Enzyme Activation (10 min, 95° C.) and PCR (40 cycles with 15 sec, 95° for Denaturing and 1 min, 60° C. for annealing and extension). DNA copy numbers were calculated from Cₜ values based on a HBV plasmid DNA standard curve by the ViiA7 software.

Sequences for TaqMan primers are shown in Table 14.

TABLE 14
HBV core specific TaqMan probes

| | | | | SEQ ID |
|---|---|---|---|---|
| | Name | Dye | Sequence | NO |
| HBV | Forward | | CTG TGC CTT GGG TGG CTT T | 30 |
| | (F3_HBVcore) | | | |
| core | Reverse | | AAG GAA AGA AGT CAG AAG GCA AAA | 31 |
| | (R3_HBVcore) | | | |
| Primer | TaqMan Probe | 56- | 56-FAM/AGC TCC AAA/ZEN/TTC TTT ATA | 32 |
| | (P3_HBVcore) | FAM | AGG GTC GAT GTC CAT G/3IABkFQ | |

ZEN is an internal quencher

### Example B1

This study aims to provide evidence that the combination of a GalNAc conjugated antisense oligonucleotide targeting HBV (anti-HBV ASO) and a TLR7-agonist would have a beneficial anti-viral affect using a HBV in vivo efficacy mouse model.

The combination of a direct-acting antiviral, such as a GalNAc conjugated antisense oligonucleotide targeting HBV (anti-HBV ASO) and an immune-modulator, such as an agonist of the toll-like receptor 7 (TLR7 agonist), in chronic HBV treatment may affect the combined effect in a manner not predictable from the activity of each individual compound alone monotherapy).

To evaluate the combination of the anti-HBV ASO and the TLR7 agonist in an in vivo system, a mouse model of chronic HBV infection was used. In the AAV/HBV mouse model described in the Materials and Method a persistent HBV infection is established resulting in expression of viral markers (HBsAg, HBeAg, HBV DNA) detectable in plasma. The effect on these viral markers upon treatment with the anti-HBV ASO of CMP ID NO: 15_1 (table 2, and FIG. 4) dosed at 1.5 mg/kg or 7.5 mg/kg and the TLR7 agonist of CMP ID NO: VI (table 3) dosed with 100 mg every other day (QOD) or once weekly (QW) in monotherapy or in combination has been evaluated.

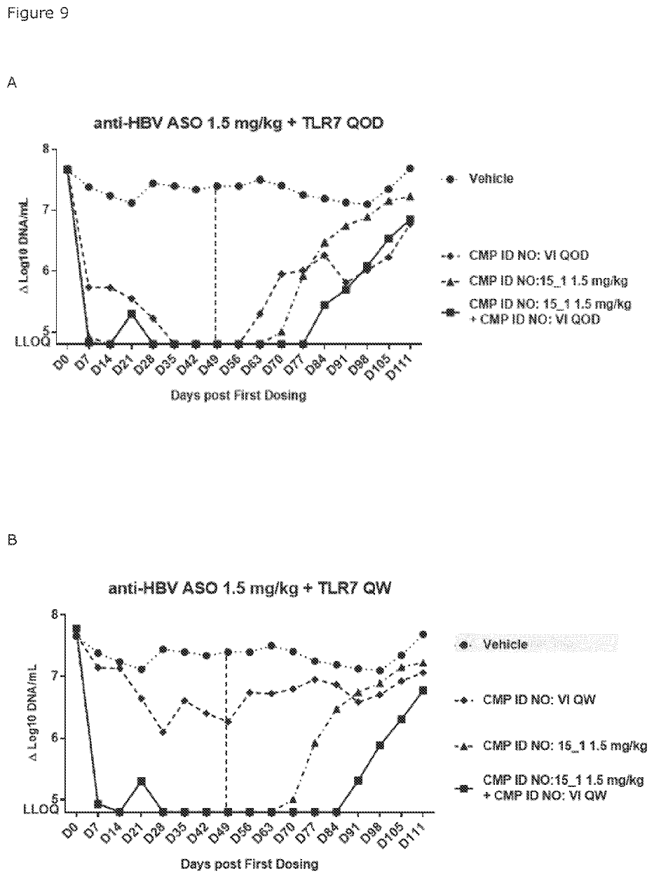

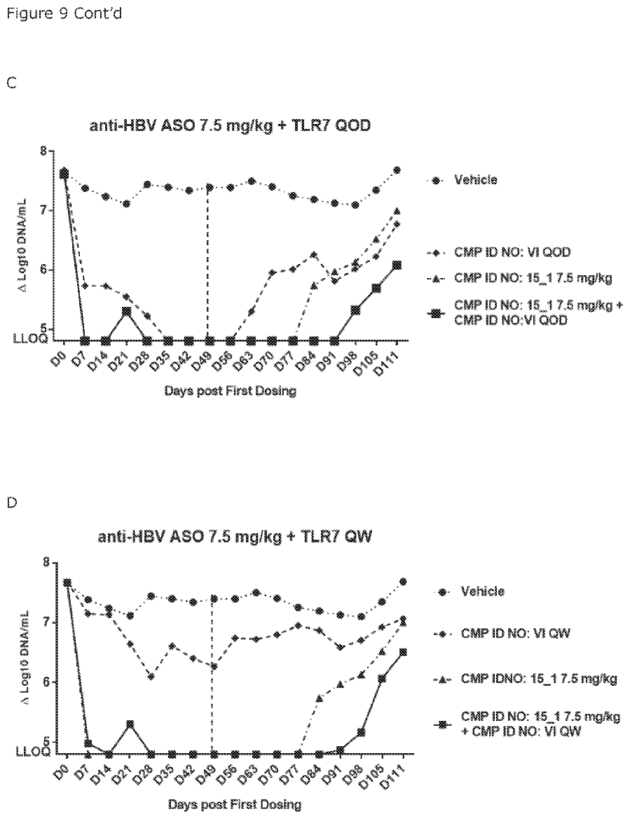

Tables 15 to 18 show HBV-DNA levels in serum AAV/HBV mice following treatment with different dosages. The data are also represented in FIGS. 9A to 9D.

TABLE 15
HBV-DNA levels in serum AAV/HBV mice following treatment with either Saline (Vehicle);
CMP ID NO: 15_1 (anti-HBV ASO) dosed at 1.5 mg/kg weekly; CMP ID NO: VI (TLR7)
administered at 100 mg/kg every other day (QOD); or the combination of both; p-value
calculated for the combination in comparison to anti-HBV ASO 1.5 mg/kg and TLR7 QOD;

| | | anti-HBV ASO | | anti-HBV ASO 1.5 mg/kg + TLR7 QOD | | |
|---|---|---|---|---|---|---|
| | Vehicle | 1.5 mg/kg | TLR7 QOD | | p-value of combination | |
| | Average ± SD | Average ± SD | Average ± SD | Average ± SD | vs. anti-HBV ASO | vs. TLR7 |
| D 0 | 7.66 ± 0.39 | 7.69 ± 0.15 | 7.69 ± 0.19 | 7.67 ± 0.34 | n/a | n/a |
| D 7 | 7.38 ± 0.6 | 4.92 ± 0.19 | 5.74 ± 0.62 | 4.82 ± 0.09 | ns | *** |
| D 14 | 7.24 ± 0.68 | 4.30† ± 0 | 5.73 ± 1.2 | 4.30† ± 0 | ns | *** |
| D 21 | 7.12 ± 0.43 | 5.3† ± 0 | 5.55 ± 0.32 | 5.3† ± 0 | ns | *** |
| D 28 | 7.44 ± 0.48 | 4.30† ± 0 | 5.23 ± 1.02 | 4.30† ± 0 | ns | *** |
| D 35 | 7.4 ± 0.44 | 4.30† ± 0 | 4.69 ± 0.71 | 4.30† ± 0 | ns | * |
| D 42 | 7.34 ± 0.46 | 4.30† ± 0 | 4.72 ± 0.76 | 4.30† ± 0 | ns | * |
| D 49 | 7.4 ± 0.44 | 4.30† ± 0 | 4.5 ± 0.55 | 4.30† ± 0 | ns | ns |
| D 56 | 7.4 ± 0.44 | 4.30† ± 0 | 4.59 ± 0.65 | 4.30† ± 0 | ns | ns |
| D 63 | 7.5 ± 0.41 | 4.61 ± 0.7 | 5.3 ± 1.21 | 4.30† ± 0 | ns | *** |
| D 70 | 7.41 ± 0.45 | 5 ± 1.02 | 5.95 ± 1.17 | 4.30† ± 0 | ** | *** |
| D 77 | 7.25 ± 0.59 | 5.92 ± 0.98 | 6.01 ± 1.17 | 4.52 ± 0.61 | *** | *** |
| D 84 | 7.2 ± 0.55 | 6.47 ± 0.69 | 6.26 ± 1.08 | 5.44 ± 1 | *** | *** |
| D 91 | 7.13 ± 0.4 | 6.75 ± 0.51 | 5.81 ± 1.09 | 5.7 ± 1.02 | *** | ns |
| D 98 | 7.1 ± 0.21 | 6.89 ± 0.59 | 6.02 ± 1.17 | 6.08 ± 1.04 | ** | ns |
| D 105 | 7.35 ± 0.33 | 7.16 ± 0.5 | 6.23 ± 1.29 | 6.54 ± 0.66 | * | ns |
| D 111 | 7.69 ± 0.27 | 7.23 ± 0.55 | 6.77 ± 0.64 | 6.85 ± 0.6 | ns | ns |

* p-value ≤ 0.05;
** p-value ≤ 0.01;
*** p-value ≤ 0.001;
ns non-significant;
†lower limit of quantification.

TABLE 16
HBV-DNA levels in serum AAV/HBV mice following treatment with either Saline (Vehicle);
CMP ID NO: 15_1 (anti-HBV ASO) dosed at 1.5 mg/kg weekly; CMP ID NO: VI (TLR7)
administered at 100 mg/kg weekly (QW); or the combination of both; p-value calculated
for the combination in comparison to anti-HBV ASO 1.5 mg/kg and TLR7 QW.

| | | anti-HBV ASO | | anti-HBV ASO 1.5 mg/kg + TLR7 QW | | |
|---|---|---|---|---|---|---|
| | Vehicle | 1.5 mg/kg | TLR7 QW | | p-value of combination | |
| | Average ± SD | Average ± SD | Average ± SD | Average ± SD | vs. anti-HBV ASO | vs. TLR7 |
| D 0 | 7.66 ± 0.39 | 7.69 ± 0.15 | 7.68 ± 0.2 | 7.78 ± 0.23 | n/a | n/a |
| D 7 | 7.38 ± 0.6 | 4.92 ± 0.19 | 7.15 ± 0.67 | 4.94 ± 0.23 | ns | *** |
| D 14 | 7.24 ± 0.68 | 4.30† ± 0 | 7.14 ± 0.47 | 4.30† ± 0 | ns | *** |
| D 21 | 7.12 ± 0.43 | 5.3† ± 0 | 6.65 ± 0.75 | 5.3† ± 0 | ns | *** |
| D 28 | 7.44 ± 0.48 | 4.30† ± 0 | 6.1 ± 1.25 | 4.30† ± 0 | ns | *** |
| D 35 | 7.4 ± 0.44 | 4.30† ± 0 | 6.61 ± 0.98 | 4.30† ± 0 | ns | *** |
| D 42 | 7.34 ± 0.46 | 4.30† ± 0 | 6.4 ± 0.84 | 4.5 ± 0.55 | ns | *** |
| D 49 | 7.4 ± 0.44 | 4.30† ± 0 | 6.27 ± 0.89 | 4.30† ± 0 | ns | *** |
| D 56 | 7.4 ± 0.44 | 4.30† ± 0 | 6.74 ± 0.64 | 4.30† ± 0 | ns | *** |
| D 63 | 7.5 ± 0.41 | 4.61 ± 0.7 | 6.72 ± 0.7 | 4.30† ± 0 | ns | *** |
| D 70 | 7.41 ± 0.45 | 5 ± 1.02 | 6.8 ± 0.64 | 4.30† ± 0 | ** | *** |
| D 77 | 7.25 ± 0.59 | 5.92 ± 0.98 | 6.95 ± 0.53 | 4.44 ± 0.41 | *** | *** |
| D 84 | 7.2 ± 0.55 | 6.47 ± 0.69 | 6.87 ± 0.49 | 4.68 ± 0.7 | *** | *** |
| D 91 | 7.13 ± 0.4 | 6.75 ± 0.51 | 6.59 ± 0.52 | 5.31 ± 0.75 | *** | *** |
| D 98 | 7.1 ± 0.21 | 6.89 ± 0.59 | 6.7 ± 0.4 | 5.88 ± 0.77 | ** | *** |
| D 105 | 7.35 ± 0.33 | 7.16 ± 0.5 | 6.93 ± 0.43 | 6.31 ± 0.97 | ** | ** |
| D 111 | 7.69 ± 0.27 | 7.23 ± 0.55 | 7.07 ± 0.45 | 6.77 ± 0.55 | * | * |

* p-value ≤ 0.05;
** p-value ≤ 0.01;
*** p-value ≤ 0.001;
ns non-significant;
†lower limit of quantification.

TABLE 17
HBV-DNA levels in serum AAV/HBV mice following treatment with either Saline (Vehicle);
CMP ID NO: 15_1 (anti-HBV ASO) dosed at 7.5 mg/kg weekly; CMP ID NO: VI (TLR7)
administered at 100 mg/kg every other day (QOD); or the combination of both; p-value
calculated for the combination in comparison to anti-HBV ASO 1.5 mg/kg and TLR7 QOD;

| | | anti-HBV ASO | | anti-HBV ASO 7.5 mg/kg + TLR7 QOD | | |
|---|---|---|---|---|---|---|
| | Vehicle | 7.5 mg/kg | TLR7 QOD | | p-value of combination | |
| | Average ± SD | Average ± SD | Average ± SD | Average ± SD | vs. anti-HBV ASO | vs. TLR7 |
| D 0 | 7.66 ± 0.39 | 7.67 ± 0.29 | 7.69 ± 0.19 | 7.62 ± 0.29 | n/a | n/a |
| D 7 | 7.38 ± 0.60 | 4.80 ± 0.00 | 5.74 ± 0.62 | 4.80 ± 0.00 | ns | *** |
| D 14 | 7.24 ± 0.68 | 4.30† ± 0.00 | 5.73 ± 1.20 | 4.30† ± 0.00 | ns | *** |
| D 21 | 7.12 ± 0.43 | 5.30† ± 0.00 | 5.55 ± 0.32 | 5.30† ± 0.00 | ns | *** |
| D 28 | 7.44 ± 0.48 | 4.30† ± 0.00 | 5.23 ± 1.02 | 4.30† ± 0.00 | ns | *** |
| D 35 | 7.40 ± 0.44 | 4.30† ± 0.00 | 4.69 ± 0.71 | 4.30† ± 0.00 | ns | * |
| D 42 | 7.34 ± 0.46 | 4.30† ± 0.00 | 4.72 ± 0.76 | 4.30† ± 0.00 | ns | * |
| D 49 | 7.40 ± 0.44 | 4.30† ± 0.00 | 4.50 ± 0.55 | 4.30† ± 0.00 | ns | ns |
| D 56 | 7.40 ± 0.44 | 4.30† ± 0.00 | 4.59 ± 0.65 | 4.30† ± 0.00 | ns | ns |
| D 63 | 7.50 ± 0.41 | 4.30† ± 0.00 | 5.30 ± 1.21 | 4.30† ± 0.00 | ns | *** |
| D 70 | 7.41 ± 0.45 | 4.30† ± 0.00 | 5.95 ± 1.17 | 4.30† ± 0.00 | ns | *** |
| D 77 | 7.25 ± 0.59 | 4.72 ± 0.60 | 6.01 ± 1.17 | 4.30† ± 0.00 | ns | *** |
| D 84 | 7.20 ± 0.55 | 5.74 ± 0.17 | 6.26 ± 1.08 | 4.30† ± 0.00 | *** | *** |
| D 91 | 7.13 ± 0.40 | 5.97 ± 0.33 | 5.81 ± 1.09 | 4.70 ± 0.67 | *** | *** |
| D 98 | 7.10 ± 0.21 | 6.13 ± 0.26 | 6.02 ± 1.17 | 5.32 ± 0.93 | ** | *** |
| D 105 | 7.35 ± 0.33 | 6.52 ± 0.44 | 6.23 ± 1.29 | 5.89 ± 0.88 | ** | ** |
| D 111 | 7.69 ± 0.27 | 7.00 ± 0.26 | 6.77 ± 0.64 | 6.33 ± 0.65 | *** | *** |

* p-value ≤ 0.05;
** p-value ≤ 0.01;
*** p-value ≤ 0.001;
ns non-significant;
†lower limit of quantification.

TABLE 18
HBV-DNA levels in serum AAV/HBV mice following treatment with either Saline (Vehicle);
CMP ID NO: 15_1 (anti-HBV ASO) dosed at 7.5 mg/kg weekly; CMP ID NO: VI (TLR7)
administered at 100 mg/kg weekly (QW); or the combination of both; p-value calculated
for the combination in comparison to anti-HBV ASO 1.5 mg/kg and TLR7 QW;

| | | anti-HBV ASO | | anti-HBV ASO 7.5 mg/kg + TLR7 QW | | |
|---|---|---|---|---|---|---|
| | Vehicle | 7.5 mg/kg | TLR7 QW | | p-value of combination | |
| | Average ± SD | Average ± SD | Average ± SD | Average ± SD | vs. anti-HBV ASO | vs. TLR7 |
| D 0 | 7.66 ± 0.39 | 7.67 ± 0.29 | 7.68 ± 0.20 | 7.67 ± 0.38 | n/a | n/a |
| D 7 | 7.38 ± 0.60 | 4.80 ± 0.00 | 7.15 ± 0.67 | 4.98 ± 0.28 | ns | *** |
| D 14 | 7.24 ± 0.68 | 4.30† ± 0.00 | 7.14 ± 0.47 | 4.30† ± 0.00 | ns | *** |
| D 21 | 7.12 ± 0.43 | 5.30† ± 0.00 | 6.65 ± 0.75 | 5.30† ± 0.00 | ns | *** |
| D 28 | 7.44 ± 0.48 | 4.30† ± 0.00 | 6.10 ± 1.25 | 4.30† ± 0.00 | ns | *** |
| D 35 | 7.40 ± 0.44 | 4.30† ± 0.00 | 6.61 ± 0.98 | 4.30† ± 0.00 | ns | *** |
| D 42 | 7.34 ± 0.46 | 4.30† ± 0.00 | 6.40 ± 0.84 | 4.30† ± 0.00 | ns | *** |
| D 49 | 7.40 ± 0.44 | 4.30† ± 0.00 | 6.27 ± 0.89 | 4.30† ± 0.00 | ns | *** |
| D 56 | 7.40 ± 0.44 | 4.30† ± 0.00 | 6.74 ± 0.64 | 4.30† ± 0.00 | ns | *** |
| D 63 | 7.50 ± 0.41 | 4.30† ± 0.00 | 6.72 ± 0.70 | 4.30† ± 0.00 | ns | *** |
| D 70 | 7.41 ± 0.45 | 4.30† ± 0.00 | 6.80 ± 0.64 | 4.30† ± 0.00 | ns | *** |
| D 77 | 7.25 ± 0.59 | 4.72 ± 0.60 | 6.95 ± 0.53 | 4.46 ± 0.44 | ns | *** |
| D 84 | 7.20 ± 0.55 | 5.74 ± 0.17 | 6.87 ± 0.49 | 4.55 ± 0.69 | *** | *** |
| D 91 | 7.13 ± 0.40 | 5.97 ± 0.33 | 6.59 ± 0.52 | 4.87 ± 0.85 | *** | *** |
| D 98 | 7.10 ± 0.21 | 6.13 ± 0.26 | 6.70 ± 0.40 | 5.17 ± 1.06 | *** | *** |
| D 105 | 7.35 ± 0.33 | 6.52 ± 0.44 | 6.93 ± 0.43 | 6.07 ± 0.99 | * | *** |
| D 111 | 7.69 ± 0.27 | 7.00 ± 0.26 | 7.07 ± 0.45 | 6.51 ± 0.69 | * | ** |

* p-value ≤ 0.05;
** p-value ≤ 0.01;
*** p-value ≤ 0.001;
ns non-significant;
†lower limit of quantification.

Tables 15 to 18 and FIGS. 9A-D show the change in the viral marker HBV-DNA over the duration of the study, for the indicated combinations of administration of CMP ID NO: 15_1 and CMP ID NO: VI. A rapid reduction in HBV-DNA to below the lower level of quantification of the assay (LLOQ) was seen for CMP ID NO: 15_1 (anti-HBV ASO) monotherapy at both 1.5 mg/kg and at 7.5 mg/kg (FIGS. 9A and C), as well as for any combination containing the anti-HBV ASO at any concentration (FIGS. 9A-D, solid line). In contrast, when treating with the TLR7 agonist (CM ID NO: VI) alone, the reduction of HBV-DNA only reached the LLOQ when dosed every other day (QOD) (FIGS. 9A and C). At QW dosing (FIGS. 9B and D), a maximal reduction of around 1.5-log was achieved with the TLR7 agonist monotherapy.

After end of dosing, the HBV-DNA levels partially rebounded in all treatment groups, with the greatest absolute rebound seen for anti-HBV ASO at the 1.5 mg/kg dose monotherapy (FIGS. 9A and B). The HBV DNA plasma levels in this group returned to within % log of the control group. Similarly, the rebound of the TLR7 agonist treated animals, whether dosed QOD or QW monotherapy, returned to within 1 log of the control group during the follow-up period. This rebound, while not of the same magnitude as for the anti-HBV ASO, occurred sooner after the end of treatment than it did in the anti-HBV ASO treated groups.

The rebound, as measured by HBV DNA, in the groups treated with combinations between the anti-HBV ASO and the TLR7 agonist was invariably delayed compared to the treatment with each single compound. Notably, the delay in onset and the kinetics of the rebound for the high-dose anti-HBV-ASO was similar between the combination with the frequent and the less-frequent dosing of the TLR7 agonist, with the rebound starting on day 91 and 84, respectively. Interestingly at the lowest combination dose (FIG. 8B) the rebound seem to start at day 84 which is later than for the low anti-HBV ASO with high TLR7 agonist dose (FIG. 8A) where the rebound is observed at day 77. So it seems that when combining anti-HBV ASO and TLR7 agonist the therapeutic window for TLR7 is increased since a 3 times reduction of the dose does not negatively affect the time at which the rebound is observed, when compared to what is observed with the TLR7 agonist mono treatments.

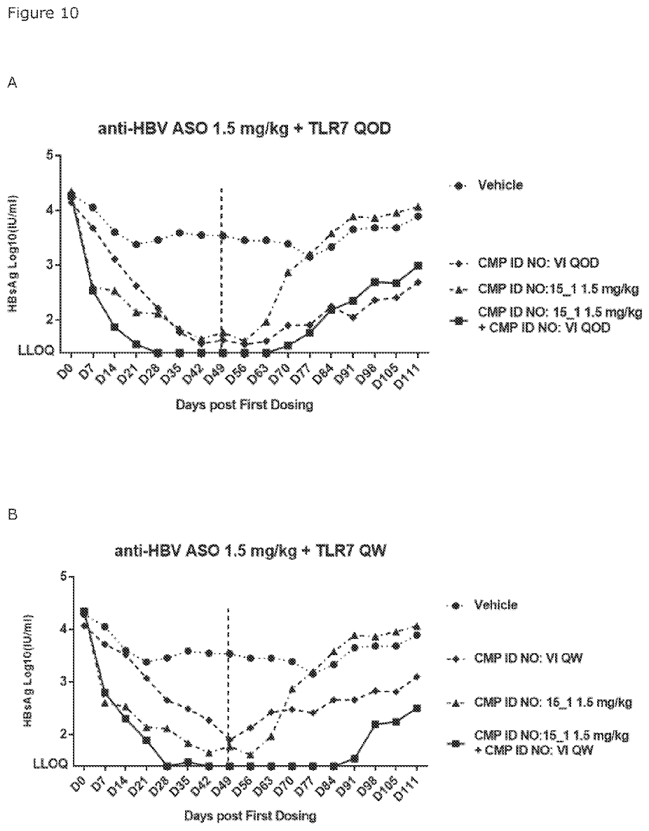

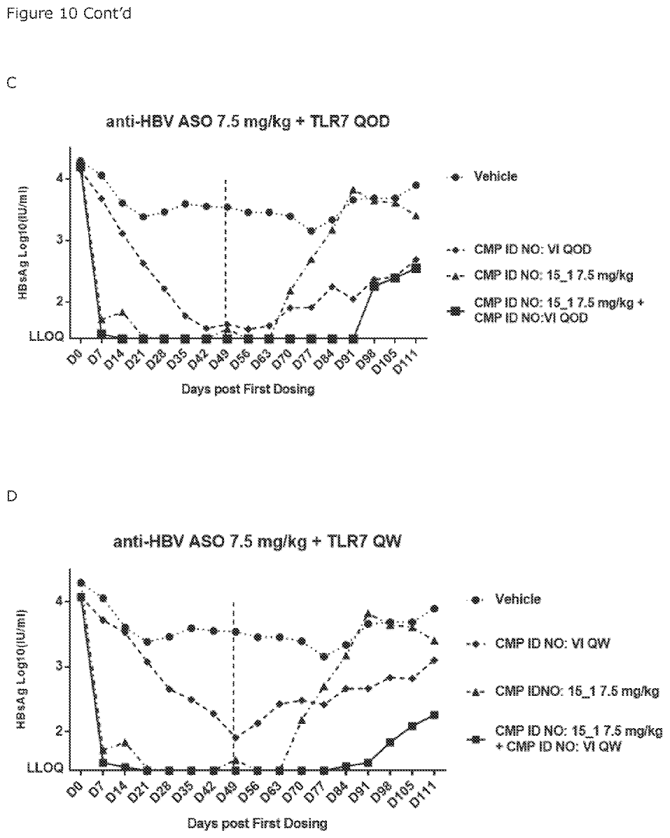

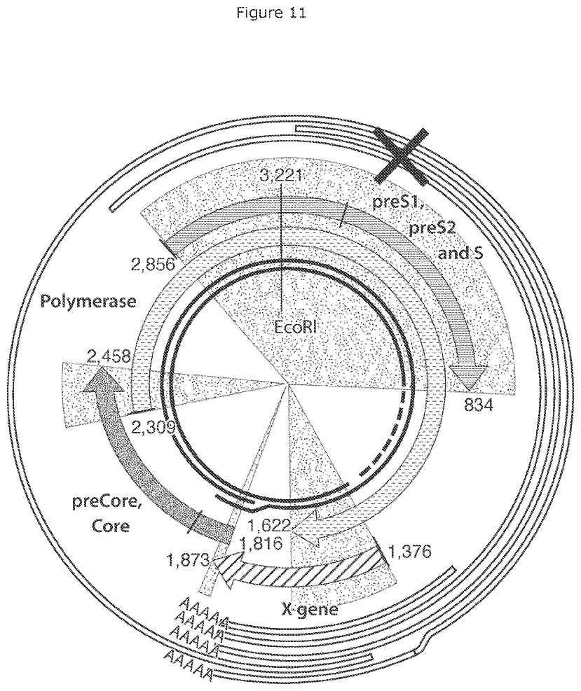

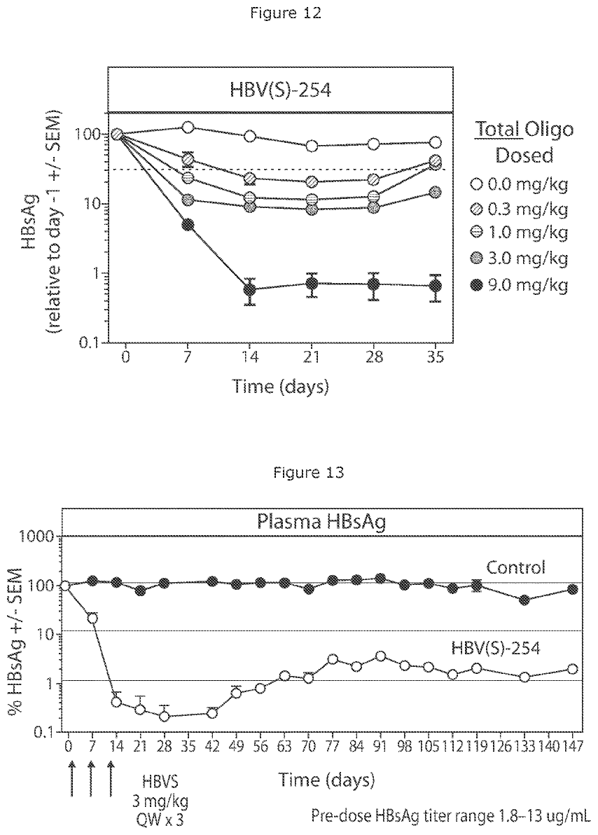

Tables 19 to 22 show HBsAg levels in serum AAV/HBV mice following treatment with different dosages. The data are also represented in FIG. 10A to 10D.

TABLE 19
HBsAg levels in serum AAV/HBV mice following treatment with either Saline (Vehicle);
CMP ID NO: 15_1 (anti-HBV ASO) dosed at 1.5 mg/kg weekly; CMP ID NO: VI (TLR7)
administered at 100 mg/kg every other day (QOD); or the combination of both; p value
calculated for combination in comparison to a) anti-HBV ASO 1.5 mg/kg and b) TLR7 QOD.

| | | anti-HBV ASO | | anti-HBV ASO 1.5 mg/kg + TLR7 QOD | | |
|---|---|---|---|---|---|---|
| | Vehicle | 1.5 mg/kg | TLR7 QOD | | p-value of combination | |
| | Average ± SD | Average ± SD | Average ± SD | Average ± SD | vs. anti-HBV ASO | vs. TLR7 |
| D 0 | 4.29 ± 0.36 | 4.34 ± 0.26 | 4.14 ± 0.37 | 4.27 ± 0.4 | n/a | n/a |
| D 7 | 4.05 ± 0.61 | 2.6 ± 0.45 | 3.68 ± 1.03 | 2.53 ± 0.9 | ns | *** |
| D 14 | 3.6 ± 0.91 | 2.53 ± 0.34 | 3.11 ± 1.35 | 1.87 ± 0.63 | * | *** |
| D 21 | 3.38 ± 0.99 | 2.14 ± 0.35 | 2.62 ± 1.41 | 1.56 ± 0.52 | * | *** |
| D 28 | 3.46 ± 1.18 | 2.12 ± 0.82 | 2.21 ± 1.28 | 1.33 ± 0.51 | ** | ** |
| D 35 | 3.59 ± 1.05 | 1.84 ± 0.46 | 1.78 ± 1.18 | 1.33 ± 0.49 | ns | ns |
| D 42 | 3.55 ± 1.18 | 1.66 ± 0.51 | 1.57 ± 0.97 | 1.18 ± 0.42 | ns | ns |
| D 49 | 3.54 ± 1.08 | 1.78 ± 0.43 | 1.63 ± 0.86 | 0.97 ± 0.58 | ** | * |
| D 56 | 3.45 ± 0.99 | 1.62 ± 0.43 | 1.56 ± 0.77 | 1.27 ± 0.41 | ns | ns |
| D 63 | 3.45 ± 0.96 | 1.97 ± 0.88 | 1.61 ± 0.96 | 1.14 ± 0.66 | ** | ns |
| D 70 | 3.39 ± 1.12 | 2.87 ± 1.14 | 1.9 ± 1.23 | 1.53 ± 0.97 | ** | ns |
| D 77 | 3.15 ± 1.17 | 3.2 ± 1.03 | 1.91 ± 1.18 | 1.77 ± 1.09 | ** | ns |
| D 84 | 3.33 ± 1 | 3.58 ± 1.03 | 2.25 ± 1.16 | 2.18 ± 1.22 | ** | ns |
| D 91 | 3.65 ± 1 | 3.89 ± 1.12 | 2.05 ± 1.35 | 2.35 ± 1.46 | *** | ns |
| D 98 | 3.69 ± 0.84 | 3.87 ± 1.07 | 2.36 ± 1.26 | 2.7 ± 1.32 | ** | ns |
| D 105 | 3.69 ± 0.85 | 3.96 ± 1.11 | 2.41 ± 1.18 | 2.68 ± 1.19 | ** | ns |
| D 111 | 3.89 ± 0.84 | 4.07 ± 1.1 | 2.69 ± 1.38 | 2.99 ± 1.33 | * | ns |

* p-value ≤ 0.05;
** p-value ≤ 0.01;
*** p-value ≤ 0.001;
ns non-significant.

TABLE 20
HBsAg levels in serum AAV/HBV mice following treatment with either Saline (Vehicle);
CMP ID NO: 15_1 (anti-HBV ASO) dosed at 1.5 mg/kg weekly; CMP ID NO: VI (TLR7)
administered at 100 mg/kg weekly (QW); or the combination of both; p value calculated
for combination in comparison to a) anti-HBV ASO 1.5 mg/kg and b) TLR7 QW.

| | | anti-HBV ASO | | anti-HBV ASO 1.5 mg/kg + TLR7 QW | | |
|---|---|---|---|---|---|---|
| | Vehicle | 1.5 mg/kg | TLR7 QW | | p-value of combination | |
| | Average ± SD | Average ± SD | Average ± SD | Average ± SD | vs. anti-HBV ASO | vs. TLR7 |
| D 0 | 4.29 ± 0.36 | 4.34 ± 0.26 | 4.07 ± 0.44 | 4.35 ± 0.14 | n/a | n/a |
| D 7 | 4.05 ± 0.61 | 2.6 ± 0.45 | 3.72 ± 0.85 | 2.8 ± 0.39 | ns | *** |
| D 14 | 3.6 ± 0.91 | 2.53 ± 0.34 | 3.52 ± 0.96 | 2.3 ± 0.49 | ns | *** |
| D 21 | 3.38 ± 0.99 | 2.14 ± 0.35 | 3.07 ± 1.22 | 1.89 ± 0.53 | ns | *** |
| D 28 | 3.46 ± 1.18 | 2.12 ± 0.82 | 2.65 ± 1.37 | 1.38 ± 0.52 | * | *** |
| D 35 | 3.59 ± 1.05 | 1.84 ± 0.46 | 2.49 ± 1.29 | 1.48 ± 0.36 | ns | *** |
| D 42 | 3.55 ± 1.18 | 1.66 ± 0.51 | 2.28 ± 1.28 | 1.28 ± 0.41 | ns | *** |
| D 49 | 3.54 ± 1.08 | 1.78 ± 0.43 | 1.9 ± 1.07 | 1.12 ± 0.45 | * | ** |
| D 56 | 3.45 ± 0.99 | 1.62 ± 0.43 | 2.13 ± 1 | 1.26 ± 0.35 | ns | ** |
| D 63 | 3.45 ± 0.96 | 1.97 ± 0.88 | 2.43 ± 1.14 | 1.26 ± 0.64 | * | *** |
| D 70 | 3.39 ± 1.12 | 2.87 ± 1.14 | 2.48 ± 1.23 | 1.15 ± 0.36 | *** | *** |
| D 77 | 3.15 ± 1.17 | 3.2 ± 1.03 | 2.41 ± 1.16 | 1.28 ± 0.36 | *** | *** |
| D 84 | 3.33 ± 1 | 3.58 ± 1.03 | 2.66 ± 1.15 | 1.34 ± 0.47 | *** | *** |
| D 91 | 3.65 ± 1 | 3.89 ± 1.12 | 2.66 ± 1.44 | 1.55 ± 0.96 | *** | *** |
| D 98 | 3.69 ± 0.84 | 3.87 ± 1.07 | 2.83 ± 1.16 | 2.2 ± 0.91 | *** | * |
| D 105 | 3.69 ± 0.85 | 3.96 ± 1.11 | 2.81 ± 1.1 | 2.25 ± 0.9 | *** | * |
| D 111 | 3.89 ± 0.84 | 4.07 ± 1.1 | 3.1 ± 1.12 | 2.51 ± 0.99 | *** | * |

* p-value ≤ 0.05;
** p-value ≤ 0.01;
*** p-value ≤ 0.001;
ns non-significant.

TABLE 21
HBsAg levels in serum AAV/HBV mice following treatment with either Saline (Vehicle);
CMP ID NO: 15_1 (anti-HBV ASO) dosed at 7.5 mg/kg weekly; CMP ID NO: VI (TLR7)
administered at 100 mg/kg every other day (QOD); or the combination of both; p value
calculated for combination in comparison to a) anti-HBV ASO 7.5 mg/kg and b) TLR7 QOD.

| | | anti-HBV ASO | | anti-HBV ASO 7.5 mg/kg + TLR7 QOD | | |
|---|---|---|---|---|---|---|
| | Vehicle | 7.5 mg/kg | TLR7 QOD | | p-value of combination | |
| | Average ± SD | Average ± SD | Average ± SD | Average ± SD | vs. anti-HBV ASO | vs. TLR7 |
| D 0 | 4.29 ± 0.36 | 4.30 ± 0.30 | 4.14 ± 0.37 | 4.20 ± 0.31 | n/a | n/a |
| D 7 | 4.05 ± 0.61 | 1.71 ± 0.24 | 3.68 ± 1.03 | 1.48 ± 0.45 | ns | *** |
| D 14 | 3.60 ± 0.91 | 1.83 ± 0.21 | 3.11 ± 1.35 | 1.31 ± 0.34 | ns | *** |
| D 21 | 3.38 ± 0.99 | 1.43 ± 0.17 | 2.62 ± 1.41 | 1.19 ± 0.31 | ns | *** |
| D 28 | 3.46 ± 1.18 | 1.30 ± 0.19 | 2.21 ± 1.28 | 1.07 ± 0.36 | ns | *** |
| D 35 | 3.59 ± 1.05 | 1.36 ± 0.26 | 1.78 ± 1.18 | 0.79 ± 0.35 | * | *** |
| D 42 | 3.55 ± 1.18 | 1.30 ± 0.16 | 1.57 ± 0.97 | 0.90 ± 0.32 | ns | * |
| D 49 | 3.54 ± 1.08 | 1.56 ± 0.16 | 1.63 ± 0.86 | 1.05 ± 0.27 | ns | * |
| D 56 | 3.45 ± 0.99 | 1.32 ± 0.24 | 1.56 ± 0.77 | 1.08 ± 0.27 | ns | ns |
| D 63 | 3.45 ± 0.96 | 1.33 ± 0.47 | 1.61 ± 0.96 | 0.92 ± 0.28 | ns | ** |
| D 70 | 3.39 ± 1.12 | 2.18 ± 0.91 | 1.90 ± 1.23 | 1.07 ± 0.24 | ** | ** |
| D 77 | 3.15 ± 1.17 | 2.70 ± 0.85 | 1.91 ± 1.18 | 1.08 ± 0.20 | *** | ** |
| D 84 | 3.33 ± 1.00 | 3.17 ± 0.60 | 2.25 ± 1.16 | 1.18 ± 0.24 | *** | *** |
| D 91 | 3.65 ± 1.00 | 3.82 ± 0.38 | 2.05 ± 1.35 | 1.38 ± 0.85 | *** | * |
| D 98 | 3.69 ± 0.84 | 3.64 ± 0.81 | 2.36 ± 1.26 | 2.26 ± 0.92 | *** | ns |
| D 105 | 3.69 ± 0.85 | 3.61 ± 0.97 | 2.41 ± 1.18 | 2.39 ± 0.93 | ** | ns |
| D 111 | 3.89 ± 0.84 | 3.40 ± 1.49 | 2.69 ± 1.38 | 2.55 ± 1.06 | * | ns |

* p-value ≤ 0.05;
** p-value ≤ 0.01;
*** p-value ≤ 0.001;
ns non-significant.

TABLE 22
HBsAg levels in serum AAV/HBV mice following treatment with either Saline (Vehicle);
CMP ID NO: 15_1 (anti-HBV ASO) dosed at 7.5 mg/kg weekly; CMP ID NO: VI (TLR7)
administered at 100 mg/kg weekly (QW); or the combination of both; p value calculated
for combination in comparison to a) anti-HBV ASO 7.5 mg/kg and b) TLR7 agonist QW.

| | | anti-HBV ASO | | anti-HBV ASO 7.5 mg/kg + TLR7 QW | | |
|---|---|---|---|---|---|---|
| | Vehicle | 7.5 mg/kg | TLR7 QW | | p-value of combination | |
| | Average ± SD | Average ± SD | Average ± SD | Average ± SD | vs. anti-HBV ASO | vs. TLR7 |
| D 0 | 4.29 ± 0.36 | 4.30 ± 0.30 | 4.07 ± 0.44 | 4.07 ± 0.41 | n/a | n/a |
| D 7 | 4.05 ± 0.61 | 1.71 ± 0.24 | 3.72 ± 0.85 | 1.52 ± 0.37 | ns | *** |
| D 14 | 3.60 ± 0.91 | 1.83 ± 0.21 | 3.52 ± 0.96 | 1.45 ± 0.30 | ns | *** |
| D 21 | 3.38 ± 0.99 | 1.43 ± 0.17 | 3.07 ± 1.22 | 1.27 ± 0.31 | ns | *** |
| D 28 | 3.46 ± 1.18 | 1.30 ± 0.19 | 2.65 ± 1.37 | 1.09 ± 0.36 | ns | *** |
| D 35 | 3.59 ± 1.05 | 1.36 ± 0.26 | 2.49 ± 1.29 | 1.30 ± 0.42 | ns | *** |
| D 42 | 3.55 ± 1.18 | 1.30 ± 0.16 | 2.28 ± 1.28 | 1.12 ± 0.39 | ns | *** |
| D 49 | 3.54 ± 1.08 | 1.56 ± 0.16 | 1.90 ± 1.07 | 0.82 ± 0.47 | * | *** |
| D 56 | 3.45 ± 0.99 | 1.32 ± 0.24 | 2.13 ± 1.00 | 1.10 ± 0.29 | ns | *** |
| D 63 | 3.45 ± 0.96 | 1.33 ± 0.47 | 2.43 ± 1.14 | 1.09 ± 0.50 | ns | *** |
| D 70 | 3.39 ± 1.12 | 2.18 ± 0.91 | 2.48 ± 1.23 | 1.12 ± 0.59 | * | *** |
| D 77 | 3.15 ± 1.17 | 2.70 ± 0.85 | 2.41 ± 1.16 | 1.14 ± 0.81 | *** | *** |
| D 84 | 3.33 ± 1.00 | 3.17 ± 0.60 | 2.66 ± 1.15 | 1.47 ± 1.05 | *** | *** |
| D 91 | 3.65 ± 1.00 | 3.82 ± 0.38 | 2.66 ± 1.44 | 1.52 ± 1.47 | *** | *** |
| D 98 | 3.69 ± 0.84 | 3.64 ± 0.81 | 2.83 ± 1.16 | 1.83 ± 1.39 | *** | ** |
| D 105 | 3.69 ± 0.85 | 3.61 ± 0.97 | 2.81 ± 1.10 | 2.08 ± 1.30 | *** | * |
| D 111 | 3.89 ± 0.84 | 3.40 ± 1.49 | 3.10 ± 1.12 | 2.25 ± 1.24 | ** | ** |

* p-value ≤ 0.05;
** p-value ≤ 0.01;
*** p-value ≤ 0.001;
ns non-significant.

Tables 19 to 22 and FIGS. 10A to 10D show that the effect on HBsAg was in general similar to the effect on HBV-DNA. Unlike for HBV DNA, the treatment with 1.5 mg/kg anti-HBV ASO (CMP ID NO: 15) was not capable of suppressing HBsAg to a level below the detection limit (FIGS. 10A and 10B), neither was the TLR7 agonist (CMP ID NO: VI) at any of the doses (FIGS. 10A-10D). The combination of the anti-HBV ASO and TLR7 agonist on the other hand was capable of reducing HBsAg to below the detection limit at all doses and delayed the rebound compared to the mono treatments. Like for the HBV DNA an increased therapeutic window for at least the TLR7 agonist is also observed in relation to HBsAg reduction, and for the HBsAg it is even more remarkable since the combination at the lowest dose (FIG. 10B) is essentially as effective as the combination at the highest dose (FIG. 100), both in terms of HBsAg reduction and delay in rebound, an indication that there also may be an increase in the therapeutic window for the anti-HBV ASO.

### CONCLUSION ON STUDY

The data in the study show the benefit of the combination of an anti-HBV ASO and a TLR7 agonist in an in vivo model of chronic HBV infection. These benefits can most clearly be seen as a delay in the rebound after end of treatment, as measured both by HBV DNA and HBsAg. There is no indication that the combination changes the risk profile of these compounds, and the lower doses of each active component in the clinical setting can achieve the same antiviral effect as the combination at the higher doses. This positive increase in the therapeutic window for the combination is a clear benefit for the patient.

Part C: Comparing Effects of RNAi and Antisense Oligonucleotides

### Example C1

The purpose of this study was to evaluate the in vivo pharmacology and efficacy of certain compounds in AAV-HBV mouse model.

Compounds tested: Negative control siRNA (DCR-AUD1, a siRNA which does not target the HBV genome); HBV(s)-219 (anti-HBV siRNA); and CMP ID NO: 15_1 (anti-HBV ASO).

Recombinant adeno-associated virus (AAV) carrying the Hepatitis B Virus (HBV) genome rAAV8-1.3HBV ayw (Lot No: 2019032703) was purchased from Beijing FivePlus Molecular Medicine Institute, and stored at −70° C. before use.

One hundred and fifteen (115) male C57BL/6 mice were obtained. On Pre-dose Day 0, all the animals were subjected to injection through tail vein with 1×10¹¹ vector genome of AAV-HBV for model induction. Based on baseline serum viral markers and body weight on Pre-dose Day 24, 80 qualified HBV-infected mice were selected.

The 80 selected mice were randomized into 4 groups for compound treatment. Sterile water, DCR-AUD1, DCR-5219 (9 mg/kg), and CMP ID NO: 15_1 (6.6 mg/kg) were subcutaneously injected once at 5 mL/kg on Day 0. The dosing volume was 2 mL.

Body weight was measured once weekly during Day 0˜21. No significant difference in body weight growth was observed among the study groups during the study phase.

Whole blood was collected to prepare serum (15 μL per mouse) twice weekly during Day 0˜21. On Day 21, the mice were euthanized. In addition to serum samples for viral marker assays, extra serum samples (120 μL per mouse) were prepared and stored at −70° C. The whole liver was collected, cut into halves, snap frozen and stored at −70° C. The remaining dosing formulations as well as the terminal serum and tissue samples were disposed on 16 and 20 Nov. 2019, respectively.

Baseline serum levels of HBsAg were determined by ARCHITECT i2000 (Abbott Laboratories, Lake Bluff, IL, USA) and supporting reagents. Baseline serum HBV DNA level was measured by using ABI7500 (Applied Biosystems, Foster City, Calif., USA) and detection kit (Sansure Biotech Inc., Changsha, Hunan, China).

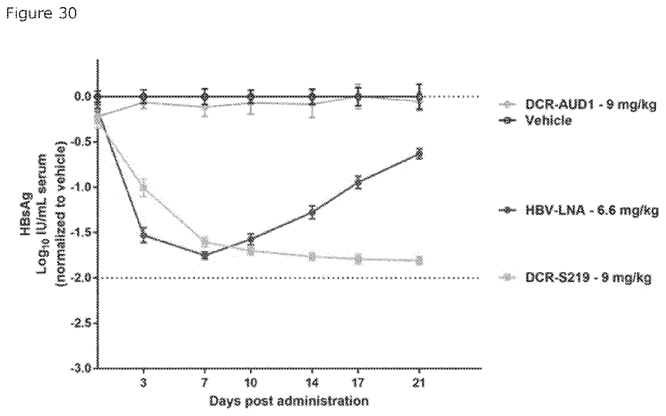

The results are shown in FIG. 30: The anti-HBV ASO (HBV-LNA) gave a rapid decrease in HBsAg level which was maintained until about 10 days, after which the HBsAg levels rebounded. The siRNA compound targeting HBV (DCR-5219) gave a slightly slower but still very rapid rate of initial reduction in HBsAg levels. Moreover, with the siRNA compound an impressive level of reduction was maintained over the 21 days of the experiment, with no sign of rebound. An even further benefit can be seen in FIG. 30 for the siRNA compound in that excellent results are achieved with a much lower molar dose than the LNA compound. FIG. 30 shows results from mice which were dosed with 9 mg/kg of siRNA and 6.6 mg/kg of LNA, However, due to the difference in molecular weight between these compounds, the molar dose of siRNA is only around 0.3× that of LNA (the M_{w} of DCR-5219 is 22262 Da, whilst the M_{w} of CMP ID NO: 15_1 is 6638 Da). Thus, excellent results can be achieved with a molar dose of the siRNA of the present invention which is far lower than that of an antisense oligonucleotide.

The data when combined with the data on the anti-HBV ASO and TLR7 agonist, for example as shown in Example B and FIG. 9, indicate a benefit of combining a TLR7 agonist with an RNAi oligonucleotide such as a siRNA targeting HBV.

As illustrated in FIG. 10A, a TLR7 agonist alone provided HBsAg reduction, but with a slow initial rate of reduction in HBsAg (lowest HBsAg seen at Day 42). There is therefore a synergy for using an RNAi oligonucleotide such as a siRNA targeting HBV with a TLR7 agonist, as the siRNA targeting HBV in Example C/FIG. 30 achieved rapid effective HBsAg knockdown, i.e. by 10 days. Furthermore, as shown by FIG. 30, the siRNA targeting HBV provided very effective long term knock-down, superior to that of an anti-HBV ASO.

From the data disclosed herein, it can be determined that the effect of a combination comprising 1) an RNAi oligonucleotide such as a siRNA targeting HBV, and 2) a TLR7 agonist will therefore be a rapidly induced, long duration of effective HBsAg knock down, indicative of effective anti-viral control over a prolonged period of time. Thus, a combination comprising an RNAi oligonucleotide and a TLR7 agonist is the most preferable combination of the present invention.

Such beneficial effects could not have been expected prior to the findings of parts A, B and C of the Examples disclosed herein.

1. A pharmaceutical combination which comprises a therapeutic RNAi oligonucleotide, and a TLR7 agonist of formula (I) or (II): 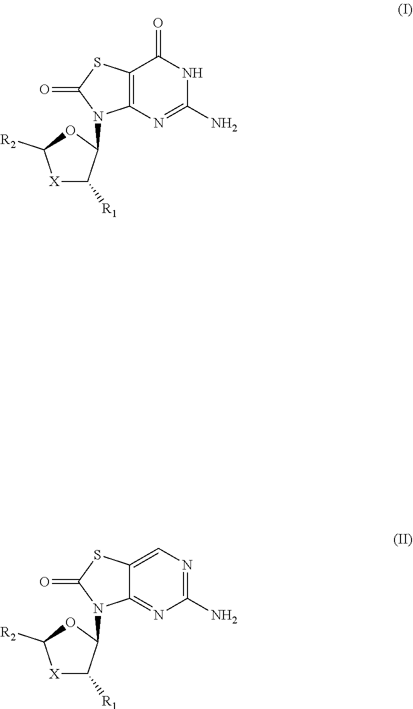
wherein X is CH₂ or S;
for formula (I) R₁ is —OH or —H and R₂ is 1-hydroxypropyl or hydroxymethyl,
for formula (II) R₁ is —OH or —H or acetoxy and R₂ is 1-acetoxypropyl or 1-hydroxypropyl or 1-hydroxymethyl or acetoxy(cyclopropyl)methyl or acetoxy(propyn-1-yl)methyl,
or a pharmaceutically acceptable salt, enantiomer or diastereomer thereof.

2. (canceled)

3. The pharmaceutical combination of claim 1, wherein the RNAi oligonucleotide is an oligonucleotide targeting HBV (RNAi ID NO: 1) or HBsAg mRNA (RNAi ID NO: 2).

4. (canceled)

5. The pharmaceutical combination of claim 1, wherein the RNAi oligonucleotide is an oligonucleotide which reduces expression of HBsAg mRNA (RNAi ID NO: 3).

6. The pharmaceutical combination of claim 1, wherein the RNAi oligonucleotide is an oligonucleotide comprising an antisense strand of 19 to 30 nucleotides in length, wherein the antisense strand comprises a region of complementarity to a sequence of HBsAg mRNA as set forth in ACAANAAUCCUCACAAUA (SEQ ID NO: 33) (RNAi ID NO: 4 or RNAi ID NO: 5).

7. (canceled)

8. The pharmaceutical combination of claim 6, wherein the RNAi oligonucleotide further comprises a sense strand of 19 to 50 nucleotides in length, wherein the sense strand forms a duplex region with the antisense strand.

9. The pharmaceutical combination of claim 8, wherein the sense strand comprises a region of complementarity to a sequence as set forth in UUNUUGUGAGGAUUN (SEQ ID NO: 34), UUAUUGUGAGGAUUNUUGUC (SEQ ID NO: 35), ACAANAAUCCUCACAAUAA (SEQ ID NO: 39), GACAANAAUCCUCACAAUAAGCAGCCGAAAGGCUGC (SEQ ID NO: 40), GACAAAAAUCCUCACAAUAAGCAGCCGAAAGGCUGC (SEQ ID NO: 41), or GACAAGAAUCCUCACAAUAAGCAGCCGAAAGGCUGC (SEQ ID NO: 42).

10. (canceled)

11. The pharmaceutical combination of claim 9, wherein the antisense strand comprises a sequence as set forth in UUAUUGUGAGGAUUNUUGUCGG (SEQ ID NO: 36), UUAUUGUGAGGAUUCUUGUCGG (SEQ ID NO: 37), or UUAUUGUGAGGAUUUUUGUCGG (SEQ ID NO: 38.

12-17. (canceled)

18. The pharmaceutical combination of claim 1, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein the sense strand comprises a sequence as set forth in GACAAAAAUCCUCACAAUAAGCAGCCGAAAGGCUGC (SEQ ID NO: 41), wherein the antisense strand comprises a sequence as set forth in UUAUUGUGAGGAUUUUUGUCGG(SEQ ID NO: 38),
wherein each of the antisense strand and the sense strand comprises one or more 2′-fluoro and 2′-O-methyl modified nucleotides and at least one phosphorothioate linkage, wherein the 4′-carbon of the sugar of the 5′-nucleotide of the antisense strand comprises a phosphate analog, and wherein the sense strand is conjugated to one or more N-acetylgalactosamine (GalNAc) moiety.

19. The pharmaceutical combination of claim 1, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13 and 17; 2′-O-methyl modified nucleotides at positions 1, 2, 4-7, 11, 14-16, 18-26 and 31-36, and at least one phosphorothioate internucleotide linkage, wherein the sense strand is conjugated to one or more N-acetylgalactosamine (GalNAc) moiety; and
the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16 and 19; 2′-O-methyl modified nucleotides at positions 1, 4, 6, 9, 11, 13, 15, 17, 18 and 20-22; and at least three phosphorothioate internucleotide linkages, wherein the 4′-carbon of the sugar of the 5′-nucleotide of the antisense strand comprises a phosphate analog.

20-22. (canceled)

23. The pharmaceutical combination of claim 19, wherein one or more of the nucleotides of the -GAAA- sequence on the sense strand is conjugated to a monovalent GalNAc moiety.

24-28. (canceled)

29. The pharmaceutical combination of claim 8, wherein the sense strand comprises at its 3′-end a stem-loop set forth as: S₁-L-S₂, wherein S₁ is complementary to S₂, and wherein L forms a loop between S₁ and S₂ of up to 6 nucleotides in length.

30-33. (canceled)

34. The pharmaceutical combination of claim 6, wherein the RNAi oligonucleotide comprises at least one modified nucleotide.

35-37. (canceled)

38. The pharmaceutical combination of claim 6, wherein the RNAi oligonucleotide comprises at least one modified internucleotide linkage.

39-42. (canceled)

43. The pharmaceutical combination of claim 1, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:
the sense strand consists of a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein each of the nucleotides of the -GAAA- sequence on the sense strand is conjugated to a monovalent GaINac moiety; and
the antisense strand consists of a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
wherein the 4′-carbon of the sugar of the 5′-nucleotide of the antisense strand comprises a methoxy phosphonate (MOP)(RNAi ID NO: 6).

44. The pharmaceutical combination of claim 1, wherein the RNAi oligonucleotide is an oligonucleotide for reducing expression of hepatitis B virus surface antigen (HBsAg) mRNA, the oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13 and 17; 2′-O-methyl modified nucleotides at positions 1, 2, 4-7, 11, 14-16, 18-26 and 31-36, and one phosphorothioate internucleotide linkage between the nucleotides at positions 1 and 2, wherein each of the nucleotides of the -GAAA- sequence on the sense strand is conjugated to a monovalent GalNAc moiety, wherein the -GAAA- sequence comprises the structure: 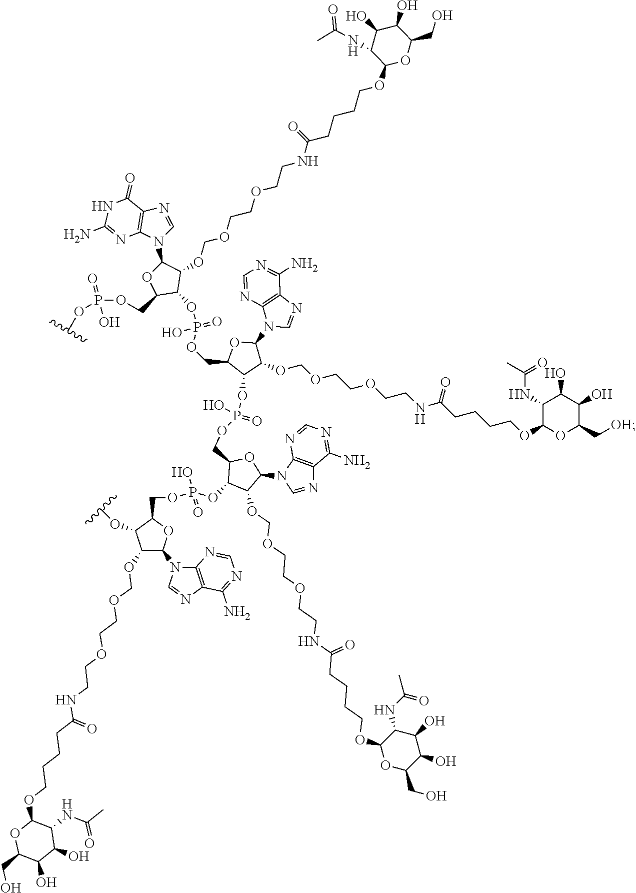 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
and
the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16 and 19; 2′-O-methyl modified nucleotides at positions 1, 4, 6, 9, 11, 13, 15, 17, 18 and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure:

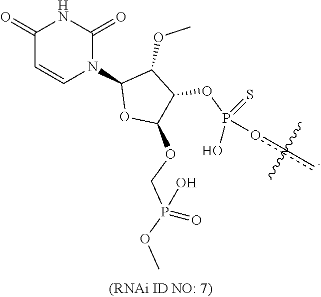

45. (canceled)

46. The pharmaceutical combination of claim 1, wherein the RNAi oligonucleotide is the oligonucleotide HBV(s)-219 (RNAi ID NO: 9).

47. The pharmaceutical combination of claim 1, wherein the therapeutic oligonucleotide is a GalNAc conjugated antisense oligonucleotide of 13 to 22 nucleotides in length with a contiguous nucleotide sequence of at least 12 nucleotides which is 100% complementary to a contiguous sequence from position 1530 to 1602 of SEQ ID NO: 1.

48. The pharmaceutical combination of claim 47, wherein the contiguous nucleotide sequence is 100% complementary to a target sequence selected from the group consisting of position 1530 to 1598; 1530-1543; 1530-1544; 1531-1543; 1551-1565; 1551-1566; 1577-1589; 1577-1591; 1577-1592; 1578-1590; 1578-1592; 1583-1598; 1584-1598; 1585-1598 and 1583-1602 of SEQ ID NO: 1.

49. (canceled)

50. The pharmaceutical combination of claim 47, wherein the contiguous nucleotide sequence of the GalNAc conjugated antisense oligonucleotide is selected from the group consisting of
(SEQ ID NO: 2)
gcgtaaagagagg;
(SEQ ID NO: 3)
gcgtaaagagaggt;
(SEQ ID NO 4)
cgcgtaaagagaggt;
(SEQ ID NO 5)
agaaggcacagacgg;
(SEQ ID NO 6)
gagaaggcacagacgg;
(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc,
or a pharmaceutically acceptable salt thereof.

51-60. (canceled)

61. The pharmaceutical combination of claim 47, wherein the contiguous nucleotide sequence of the GalNAc conjugated antisense oligonucleotide is selected from the group consisting of:
(SEQ ID NO: 2)
gcgtaaagagagg;
(SEQ ID NO: 3)
gcgtaaagagaggt;
(SEQ ID NO 4)
cgcgtaaagagaggt;
(SEQ ID NO 5)
agaaggcacagacgg;
(SEQ ID NO 6)
gagaaggcacagacgg;
(SEQ ID NO 7)
agcgaagtgcacacgg;
(SEQ ID NO 8)
gaagtgcacacgg;
(SEQ ID NO 9)
gcgaagtgcacacgg;
(SEQ ID NO: 10)
agcgaagtgcacacg;
(SEQ ID NO 11)
cgaagtgcacacg;
(SEQ ID NO: 12)
aggtgaagcgaagtgc
(SEQ ID NO: 13)
aggtgaagcgaagtg;
(SEQ ID NO 14)
aggtgaagcgaagt;
and
(SEQ ID NO: 29)
gcagaggtgaagcgaagtgc,
wherein uppercase letters denote LNA or MOE nucleosides and lower case letters denote DNA nucleosides.

62-68. (canceled)

69. The pharmaceutical combination of claim 47, wherein the GalNAc conjugated antisense oligonucleotide is selected from the group consisting of:
SEQ ID NO: 15
5′-GN2-C6ₒcₒaₒGₛ^{m}CₛGₛtₛaₛaₛaₛgₛaₛgₛaₛGₛG-3′
SEQ ID NO: 15
5′-GN2-C6ₒcₒaₒGₛ^{m}CₛGₛtₛaₛaₛaₛgₛaₛgₛAₛGₛG-3′
SEQ ID NO: 16
5-GN2-C6ₒcₒaₒGₛ^{m}CₛGₛtₛaₛaₛaₛgₛaₛgₛaₛGₛGₛT-3′
SEQ ID NO: 17
5′-GN2-C6ₒcₒaₒ^{m}CₛGₛ^{m}CₛgₛtₛaₛaₛaₛgₛaₛgₛaₛGₛGₛT-3′
SEQ ID NO: 18
5′-GN2-C6ₒcₒaₒGₛAₛGₛaₛaₛgₛgₛcₛaₛcₛaₛgₛaₛ^{m}CₛGₛG-3′
SEQ ID NO: 19
5′-GN2-C6ₒcₒaₒGₛAₛGₛaₛaₛgₛgₛcₛaₛcₛaₛgₛaₛ^{m}CₛGₛG-3′
SEQ ID NO: 20
5′-GN2-C6₀c₀a₀AₛGₛ^{m}Cₛgₛaₛaₛgₛtₛgₛcₛaₛcₛaₛ^{m}CₛGₛG-3
SEQ ID NO: 21
5′-GN2-C6₀c₀a₀GₛAₛAₛgₛtₛgₛcₛaₛcₛaₛ^{m}cₛGₛG-3′
SEQ ID NO: 21
5′-GN2-C6₀c₀a₀GsAₛAₛgₛtₛgₛcₛaₛcₛaₛ^{m}CₛGₛG-3′
SEQ ID NO: 22
5′-GN2-C6₀c₀a₀Gₛ^{m}CₛGₛaₛaₛgₛtₛgₛcₛaₛcₛaₛ^{m}CₛGₛG-3′
SEQ ID NO: 23
5′-GN2-C6ₒcₒaₒAₛGₛ^{m}CₛgₛaₛaₛgₛtₛgₛcₛaₛcₛAₛ^{m}CₛG-3′;
SEQ ID NO: 24
5′-GN2-C6₀c₀a₀^{m}CₛGₛAₛaₛgₛtₛgₛcₛaₛcₛaₛ^{m}CₛG-3′
SEQ ID NO: 25
5′-GN2-C6ₒcₒaₒAₛGₛGₛtₛgₛaₛaₛgₛ^{m}cₛgₛaₛaₛgₛTₛGₛ^{m}c-3′
SEQ ID NO: 26
5′-GN2-C6ₒcₒaₒAₛGₛgₛtₛgₛaₛaₛgₛ^{m}cₛgₛaₛAₛGₛTₛG-3′
SEQ ID NO: 26
5′-GN2-C6₀c₀a₀AₛGₛGₛtₛgₛaₛaₛgₛ^{m}cₛgₛaₛaₛGₛTₛG-3′;
and
SEQ ID NO: 27
5′-GN2-C6ₒcₒaₒAₛGₛGₛtₛgₛaₛaₛgₛ^{m}cₛgₛaₛAₛGₛT-3′
wherein uppercase bold letters denote beta-D-oxy-LNA units; lowercase letters denote DNA units; subscript “o” denotes a phosphodiester linkage; subscript “s” denotes a phosphorothioate linkage; superscript m denotes a DNA or beta-D-oxy-LNA unit containing a 5-methylcytosine base; GN2-C6 denotes a GalNAc2 conjugate with a C6 linker, or a pharmaceutically acceptable salt thereof.

70. The pharmaceutical combination of claim 47, wherein the GalNAc conjugated antisense oligonucleotide is 5′-FIG. 1J-ₒG_{S}C_{S}A_{S}g_{S}g_{S}t_{S}g_{S}a_{S}a_{S}g_{S}c_{S}g_{S}a_{S}A_{S}G_{S}T_{S}G_{S}C-3′ (FIG. 2), wherein underlined uppercase underlined letters denote MOE units; lowercase letters denote DNA units; subscript “o” denotes a phosphodiester linkage; subscript “s” denotes a phosphorothioate linkage.

71. The pharmaceutical combination of claim 1, wherein (a) the TLR7 agonist is of formula (III): 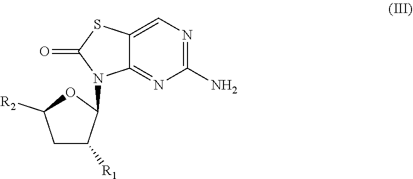
wherein R₁ is —OH or acetoxy and R₂ is 1-acetoxypropyl or 1-hydroxypropyl or 1-hydroxymethyl or a pharmaceutically acceptable salt, enantiomer or diastereomer thereof;
(b) the TLR7 agonist is of formula (IV): 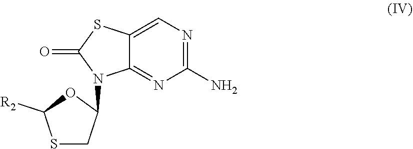
wherein R₁ is acetoxy(cyclopropyl)methyl or acetoxy(propyn-1-yl)methyl; or
(c) the TLR7 agonist is of formula (V): 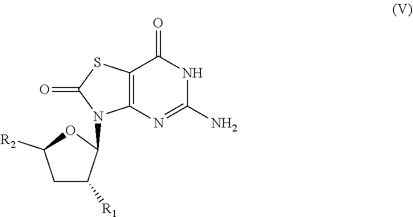
wherein R₁ is —OH and R₂ is 1-hydroxypropyl or hydroxymethyl or a pharmaceutically acceptable salt, enantiomer or diastereomer thereof.

72-74. (canceled)

75. The pharmaceutical combination of claim 1, wherein the combination comprising an RNAi oligonucleotide and a TLR7 agonist is selected from the group consisting of the following combinations:
RNAi ID NO: 1 and CMP ID NO: VI; RNAi ID NO: 2 and CMP ID NO: VI; RNAi ID NO: 3 and CMP ID NO: VI; RNAi ID NO: 4 and CMP ID NO: VI; RNAi ID NO: 5 and CMP ID NO: VI; RNAi ID NO: 6 and CMP ID NO: VI; RNAi ID NO: 7 and CMP ID NO: VI; RNAi ID NO: 8 and CMP ID NO: VI; RNAi ID NO: 9 and CMP ID NO: VI;
RNAi ID NO: 1 and CMP ID NO: VII, RNAi ID NO: 2 and CMP ID NO: VII; RNAi ID NO: 3 and CMP ID NO: VII; RNAi ID NO: 4 and CMP ID NO: VII; RNAi ID NO: 5 and CMP ID NO: VII; RNAi ID NO: 6 and CMP ID NO: VII; RNAi ID NO: 7 and CMP ID NO: VII; RNAi ID NO: 8 and CMP ID NO: VII; RNAi ID NO: 9 and CMP ID NO: VII;
RNAi ID NO: 1 and CMP ID NO: VIII, RNAi ID NO: 2 and CMP ID NO: VIII; RNAi ID NO: 3 and CMP ID NO: VIII; RNAi ID NO: 4 and CMP ID NO: VIII; RNAi ID NO: 5 and CMP ID NO: VIII; RNAi ID NO: 6 and CMP ID NO: VIII; RNAi ID NO: 7 and CMP ID NO: VIII; RNAi ID NO: 8 and CMP ID NO: VIII; RNAi ID NO: 9 and CMP ID NO: VIII;
RNAi ID NO: 1 and CMP ID NO: XIII, RNAi ID NO: 2 and CMP ID NO: XIII; RNAi ID NO: 3 and CMP ID NO: XIII; RNAi ID NO: 4 and CMP ID NO: XIII; RNAi ID NO: 5 and CMP ID NO: XIII; RNAi ID NO: 6 and CMP ID NO: XIII; RNAi ID NO: 7 and CMP ID NO: XIII; RNAi ID NO: 8 and CMP ID NO: XIII, or RNAi ID NO: 9 and CMP ID NO: XIII;
or a pharmaceutically acceptable salt, enantiomer or diastereomer thereof.

76. The pharmaceutical combination of claim 1, wherein the RNAi oligonucleotide is an
oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GaINac moiety, wherein the -GAAA- sequence comprises the structure: 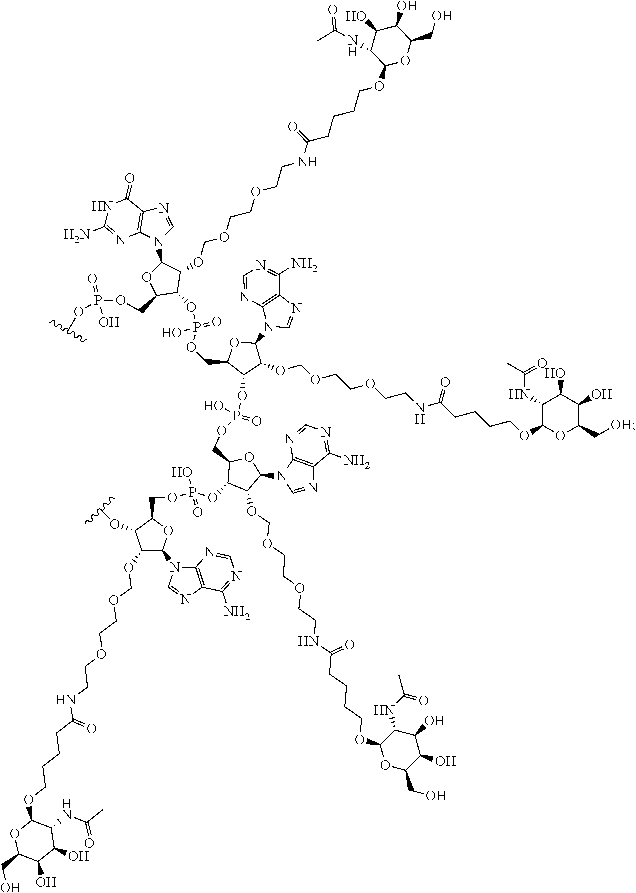 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
and
the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure: 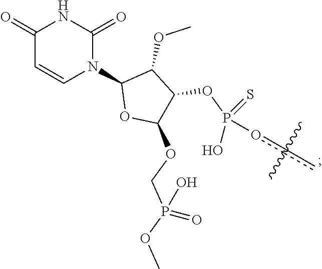
and the TLR7 agonist is CMP ID NO: VI: 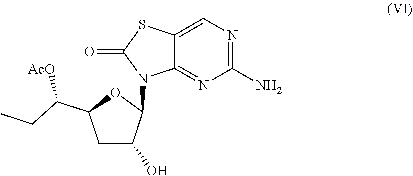 [SMILES: CC[C@H](OC(C)=O)[C@@H]1C[C@@H](O)[C@H](n2c(=O)sc3cnc(N)nc32)O1]
or a pharmaceutically acceptable salt, enantiomer or diastereomer thereof.

77. The pharmaceutical combination of claim 47, wherein the combination comprising a GalNAc conjugated antisense oligonucleotide and a TLR7 agonist is selected from the group consisting of the following combinations: CMP ID NO: 15_ 1 and VI, CMP ID NO: 15_ 2 and VI; CMP ID NO: 16_ 1 and VI; CMP ID NO: 20_ 1 and VI; CMP ID NO: 23_ 1 and VI; CMP ID NO: 26_ 1 and VI; CMP ID NO: 29_ 1 and VI; CMP ID NO: 15_ 1 and VII, CMP ID NO: 15_ 2 and VII; CMP ID NO: 16_ 1 and VII; CMP ID NO: 20_ 1 and VII; CMP ID NO: 23_ 1 and VII; CMP ID NO: 26_ 1 and VII; CMP ID NO: 29_ 1 and VII; CMP ID NO: 15_ 1 and VIII, CMP ID NO: 15_ 2 and VIII; CMP ID NO: 16_ 1 and VIII; CMP ID NO: 20_ 1 and VIII; CMP ID NO: 23_ 1 and VII; CMP ID NO: 26_ 1 and VIII; CMP ID NO: 29_ 1 and VIII; CMP ID NO: 15_ 1 and XIII, CMP ID NO: 15_ 2 and XIII; CMP ID NO: 16_ 1 and XIII; CMP ID NO: 20_ 1 and XIII; CMP ID NO: 23_ 1 and XIII; CMP ID NO: 26_ 1 and XIII; and CMP ID NO: 29_ 1 and XIII, or a pharmaceutically acceptable salt, enantiomer or diastereomer thereof.

78. The pharmaceutical combination of claim 47, wherein the GalNAc conjugated antisense oligonucleotide is CMP ID NO: 15_1 as shown in FIG. 5 and the TLR7 agonist is CMP ID NO: VI: 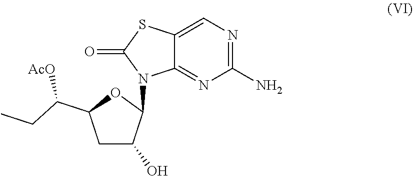 [SMILES: CC[C@H](OC(C)=O)[C@@H]1C[C@@H](O)[C@H](n2c(=O)sc3cnc(N)nc32)O1]
or a pharmaceutically acceptable salt, enantiomer or diastereomer thereof.

79-86. (canceled)

87. The pharmaceutical combination of claim 1, wherein the pharmaceutical combination comprises an RNAi oligonucleotide and a TLR7 agonist, wherein the pharmaceutical combination further comprises a CpAM (core protein allosteric modulator).

88-89. (canceled)

90. A pharmaceutical combination comprising an RNAi oligonucleotide, a TLR7 agonist and a CpAM, wherein the RNAi oligonucleotide is an
oligonucleotide comprising a sense strand forming a duplex region with an antisense strand, wherein:
the sense strand comprises a sequence as set forth in GACAAAAAUCCUCACAAUAAGCAGCCGAAAGGCUGC (SEQ ID NO: 41) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GaINac moiety, wherein the -GAAA- sequence comprises the structure: 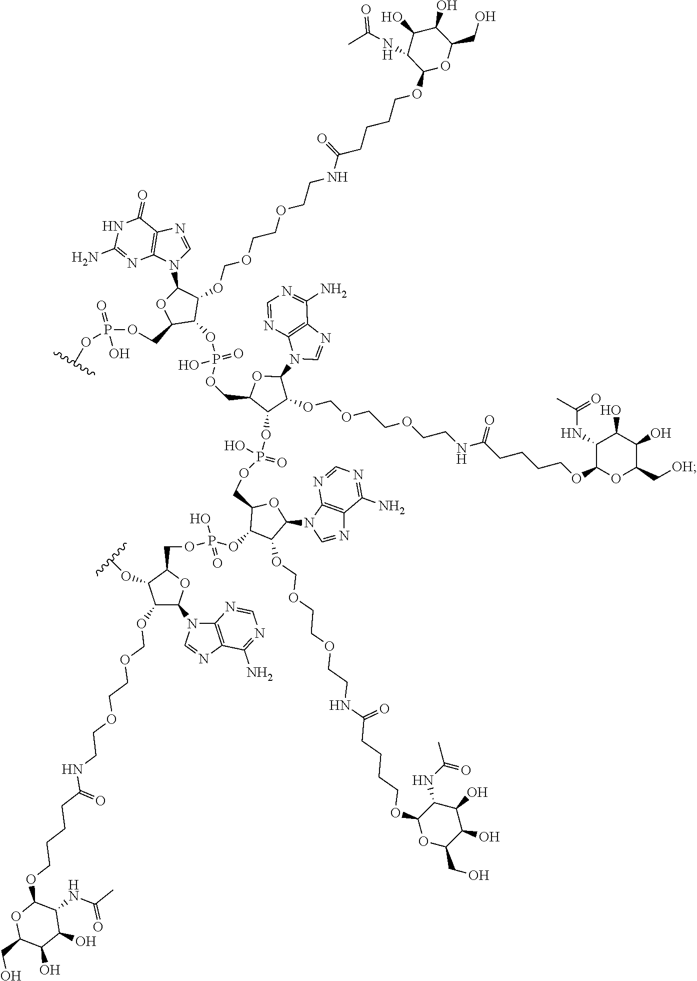 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
and
the antisense strand comprises a sequence as set forth in UUAUUGUGAGGAUUUUUGUCGG (SEQ ID NO: 38) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 4′-carbon of the sugar of the 5′-nucleotide of the antisense strand has the following structure: 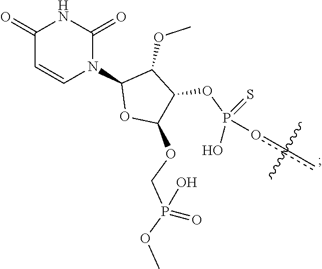
wherein the TLR7 agonist is CMP ID NO: VI: 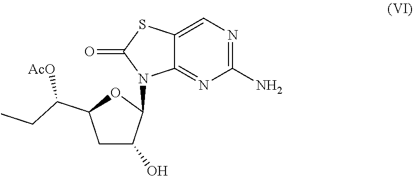 [SMILES: CC[C@H](OC(C)=O)[C@@H]1C[C@@H](O)[C@H](n2c(=O)sc3cnc(N)nc32)O1]
or a pharmaceutically acceptable salt, enantiomer or diastereomer thereof;
and wherein the CpAM is Compound (CpAM2): 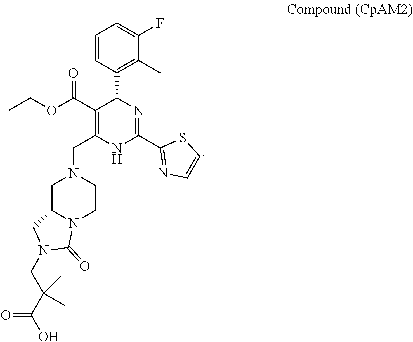 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]
or a pharmaceutically acceptable salt, enantiomer or diastereomer thereof.

91. A pharmaceutical composition comprising the pharmaceutical combination of claim 1.

92. A kit comprising a therapeutic RNAi oligonucleotide and a package insert with instruction for administration with a TLR7 agonist to treat a hepatitis B virus infection.

93-139. (canceled)

140. A method for treating a subject having a hepatitis B virus infection comprising administering to the subject a therapeutically effective amount of the pharmaceutical combination of claim 1.

141. A method for treating a subject having a hepatitis B virus infection comprising administering to the subject a therapeutically effective amount of the pharmaceutical composition of claim 91.

142-153. (canceled)

154. A method of reducing expression of hepatitis B virus surface antigen in a cell, the method comprising delivering to the cell the pharmaceutical combination of claim 1.

155-159. (canceled)
